(19) United States
(12) Patent Application Publication
(10) Pub. No.: US 2026/0200892 A1
(43) Pub. Date: Jul. 16, 2026

(54) CANNABINOID RECEPTOR 2 MODULATORS

(71) Applicant: Hoffmann-La Roche Inc., Little Falls, NJ (US)

(72) Inventors: Miroslav Kosar, Urdorf (CH); Erick Moran Carreira, Zuerich (CH); Uwe Michael Grether, Efringen-Kirchen (DE); Roman Clà Sarott, Heidelberg (DE); Wolfgang Guba, Muellheim (DE); Bilal Kicin, Visp (CH)

(73) Assignee: Hoffmann-La Roche Inc., Little Falls, NJ (US)

(21) Appl. No.: 19/417,298

(22) Filed: Dec. 11, 2025

Related U.S. Application Data
(63) Continuation of application No. PCT/EP2024/066299, filed on Jun. 13, 2024.

(30) Foreign Application Priority Data
Jun. 15, 2023 (EP) 23179506.3

(51) Int. Cl. C07D 401/12; A61K 31/04; A61K 31/164; A61K 31/34; A61K 31/404; A61K 31/415; A61K 31/4245; A61K 31/4433; A61K 31/4439; A61K 31/69; C07C 33/50; C07C 205/06; C07D 231/12; C07D 271/12; C07D 403/06; C07D 405/06; C07D 493/10; C07F 5/02

(52) U.S. Cl. CPC C07D 401/12; A61K 31/04; A61K 31/164; A61K 31/34; A61K 31/404; A61K 31/415; A61K 31/4245; A61K 31/4433; A61K 31/4439; A61K 31/69; C07C 33/50; C07C 205/06; C07D 231/12; C07D 271/12; C07D 403/06; C07D 405/06; C07D 493/10; C07F 5/027

(57) ABSTRACT

The present invention provides compounds that are useful as modulators of the cannabinoid receptor 2 (CB₂R) having the general formula (I)

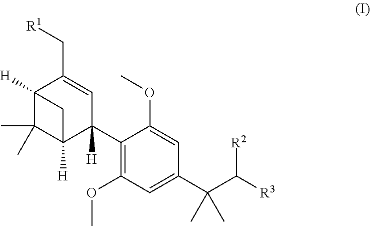

wherein R¹ to R³ are as described herein, compositions including the compounds, processes of manufacturing the compounds and methods of using the compounds.

### FIELD OF THE INVENTION

The present invention relates to organic compounds that are useful as modulators of the cannabinoid receptor 2 (CB₂R). The present invention further relates to organic compounds that are useful as selective fluorescent probes for the cannabinoid receptor 2.

### BACKGROUND OF THE INVENTION

Addressing dysregulation of signaling pathways of the endocannabinoid system has been suggested as a promising target to treat a plethora of diseases. Modulation of CB₂R to leverage its therapeutic potential has primarily focused on receptor activation with agonists. In contrast, application of CB₂R antagonists and inverse agonists to ameliorate disease states remains vastly unexplored despite promising results in models of arthritis, neuroinflammation and renal fibrosis (C. A. Lunn, et. al. Br. J. Pharmacol. 2008, 153, 226-39; S. S. Alghamdi, et al. Bioorg. Med. Chem. 2021, 33, 116035; L. Zhou, et. al. Kidney Int. 2018, 94, 756-772.). Investigation of CB₂R pharmacology with fluorescent probes had significantly contributed to the understanding of receptor localization and expression in endogenous living cells and in vivo (R. C. Sarott, et. al., J. Am. Chem. Soc., 2020, 142, 16953-16964; T. Gazzi, et. al., Chem. Sci. 2022, 13, 5539-5545). However, these validated probes feature a potent agonist targeting ligand which upon binding activates CB₂R, triggers downstream signaling pathways and prompts receptor internalization leading to desensitization. Previous efforts to develop CB₂R inverse agonists demonstrated how challenging these efforts are. For example, fluorophore conjugation to known selective inverse agonists of CB₂R completely abolished affinity for the receptor or triggered a dramatic 260-fold drop in affinity (A. G. Cooper, et. al. Eur. J. Med. Chem. 2018, 145, 770-789; Sexton, M., et. al. Chem Biol 2011, 18, 563-568). A report of an inverse agonist that has high affinity and selectivity for CB₂R stems from a series of agonists and the reported functionality is linked to a specific linker and fluorophore as all other entities reported with the identical ligand are agonists (S. Singh, et. al., ACS Med. Chem. Lett. 2019, 10, 209-214). It would be highly advantageous to discover a universal high-affinity, CB₂R-selective ligand that would be amenable to functionalization with a wide range of fluorophores and functional groups while maintaining its favorable pharmacological and, specifically, functional profile. Moreover, it would be yet more significant if the functional response (% inverse agonism, antagonism, partial agonism, agonism) could be specifically tailored to fit the CB₂R dysregulation identified in a disease in order to treat it.

### SUMMARY OF THE INVENTION

The limitations described above are addressed with this invention that reports rationally designed, high-affinity, CB₂R-selective antagonist and inverse agonist ligands that can be functionalized with a wide range of functionalities while maintaining their excellent affinity, selectivity and functionality. The novel compounds are proposed to interact with and modulate the movement of Trp258 that has been designated the single residue ‘toggle switch’ for receptor activation (T. Hua, et. al. Cell. 2020, 180, 655-665). The fluorescent probes reported herein can find application in conventional and imaging flow cytometry experiments as well as fluorescence-activated cell sorting (FACS). Additionally, these probes can be applied in fluorescent confocal microscopy. The inverse agonistic properties of the probes reported herein allow to visualize CB₂R and monitor localization and receptor trafficking without receptor activation and associated internalization. Additionally, the wide range of fluorophores that span various molecular sizes, physicochemical properties, absorption and emission wavelengths, and Stoke's shifts allows to specifically tailor the selection of probe to a desired application. For example, a non-permeable probe (with AF488) would target CB₂R at the plasma membrane and a cell permeable probe (with NBD) would visualize cytoplasmic receptor populations. Additionally, investigation of structural modifications to the targeting ligand allowed to achieve distinct pharmacological response at CB₂R and span a range of inverse agonist, antagonist, partial and full agonist. These ligands might find application in targeting disease states in which a precise level of receptor activation or inactivation is required. The probes can also support the translation of preclinical pharmacological animal data to clinic.

In a first aspect, the present invention provides a compound of Formula (I)

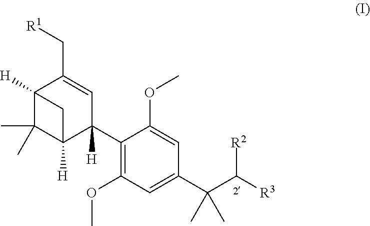

or a pharmaceutically acceptable salt thereof, wherein R¹ to R³ are as defined herein.

### BRIEF DESCRIPTION OF THE FIGURES

FIG. 1 shows a crystal structure of (S)-5-(2-methyl-8-((4-nitrobenzoyl)oxy)-3-phenyloctan-2-yl)-1,3-phenylene bis(4-nitrobenzoate) (Example 32).

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 6 carbon atoms, e.g., 1, 2, 3, 4, 5, or 6 carbon atoms (“C₁—C-alkyl”). In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. A particularly preferred, yet non-limiting example of haloalkyl is trifluoromethyl (CF₃).

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. A particularly preferred, yet non-limiting example of haloalkoxy is trifluoromethoxy (—OCF₃).

The term “aryl” refers to a monocyclic, bicyclic, or tricyclic carbocyclic ring system having a total of 6 to 10 ring members (“C₆-C₁₀-aryl”), and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic, bicyclic or tricyclic, preferably monocyclic ring system having a total of 5 to 14 ring members (“5- to 14-membered heteroaryl”), preferably, 5 to 12 ring members, more preferably 5 to 10 ring members, yet more preferably 5 to 9 ring members, wherein at least one ring in the system is aromatic, and at least one ring in the system contains one or more heteroatoms. Preferably, “heteroaryl” refers to a 5-9 membered heteroaryl comprising 1, 2, 3 or 4 heteroatoms independently selected from O, S and N. Most preferably, “heteroaryl” refers to a 5-8 membered or a 5-6 membered heteroaryl comprising 1 to 3, preferably 1 to 2 heteroatoms independently selected from O, S and N. Some non-limiting examples of heteroaryl include spiro[cyclopropane-1,3′-indoline](e.g., spiro[cyclopropane-1,3′-indoline]-1′-yl), 2-pyridyl, 3-pyridyl, 4-pyridyl, pyrazin-2-yl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, 1,2,4-oxadiazolyl, 1,3,4-oxadiazolyl, thiazol-2-yl, thiazol-4-yl, thiazol-5-yl, pyridazin-3-yl, pyridazin-4-yl, 1,2,4-triazol-4-yl, 1,2,4-triazol-1-yl, 4H-1,2,4-triazol-3-yl, 4,5,6,7-tetrahydroindazol-2-yl, 6,7-dihydro-4H-pyrano[4,3-c]pyrazol-2-yl, thiazolyl, benzofurazan-4-yl, tetrazolyl, isoxazolyl, and morpholinyl. Particularly preferred, yet non-limiting examples of heteroaryl are pyridyl, pyrazinyl, pyrimidinyl, pyrazolyl, imidazolyl, oxazolyl, oxadiazolyl and triazolyl.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “hydroxy” refers to a —OH group.

The term “amino” refers to a —NH₂ group.

The term “cyano” refers to a —CN (nitrile) group.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid.

Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride and trifluoroacetatesalts.

The compounds of formula (I) can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “CB2” refers to the cannabinoid receptor 2.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

### Compounds of the Invention

In a first aspect, the present invention provide a compound of formula (I)

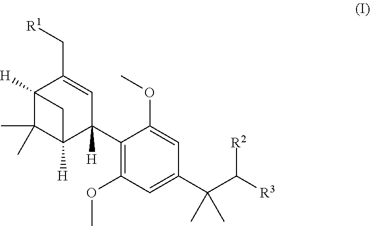

or a pharmaceutically acceptable salt thereof, wherein:
R¹ is selected from hydroxy, amino, and a group

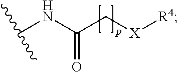

X is selected from a covalent bond, —O—,-NR^{x}—, —NRC(O)—(CH₂)_{q}—, —C(O)NR^{x}—, —C(O)—(CH₂)_{q}—, —C(O)—(CH₂)_{q}—NRC(O)—, —C(O)—CH₂-(CH₂)_{q}—C(O)O—, —C(O)—CH₂—O—, and —C(O)—CH₂—S—;
R^{x} is selected from hydrogen and methyl;
p is an integer selected from 1, 2, 3, 4, 5, and 6;
q is an integer selected from 0, 1, 2, 3, 4, and 5;
R² and R³ are each independently selected from C₁-C₆-alkyl, halo-C₁-C₆-alkyl, C₁-C₆-alkoxy, halo-C₁-C₆-alkoxy, C₆-C₁₀-aryl, and 5- to 14-membered heteroaryl; wherein said C₆-C₁₀-aryl and 5- to 14-membered heteroaryl are optionally substituted with 1-3 substituents independently selected from halogen, cyano, hydroxy, amino, C₁-C₆-alkyl, halo-C₁-C₆-alkyl, C₁-C₆-alkoxy, and halo-C₁-C₆-alkoxy;
R⁴ is selected from:

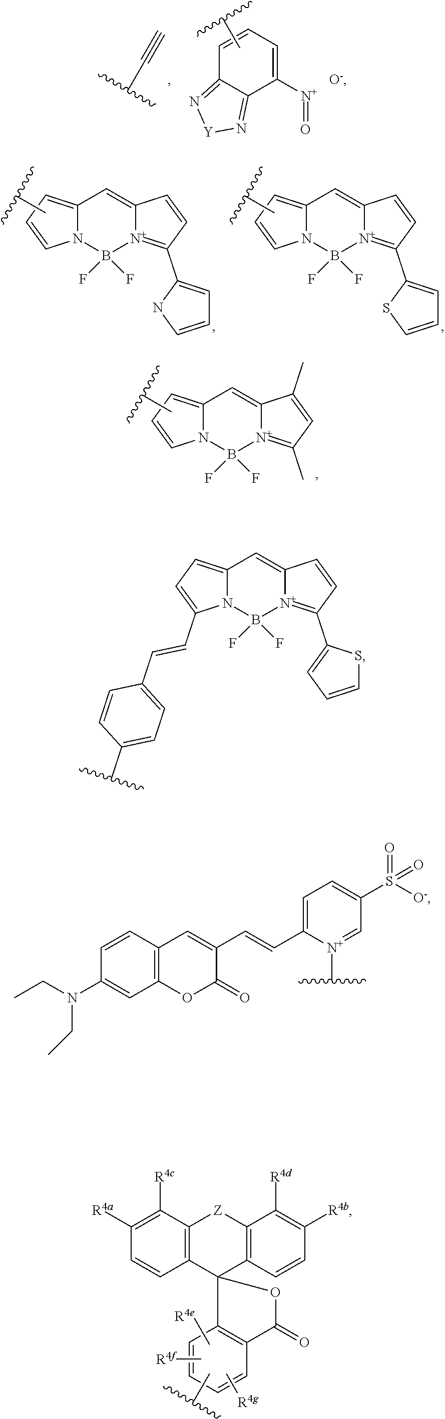

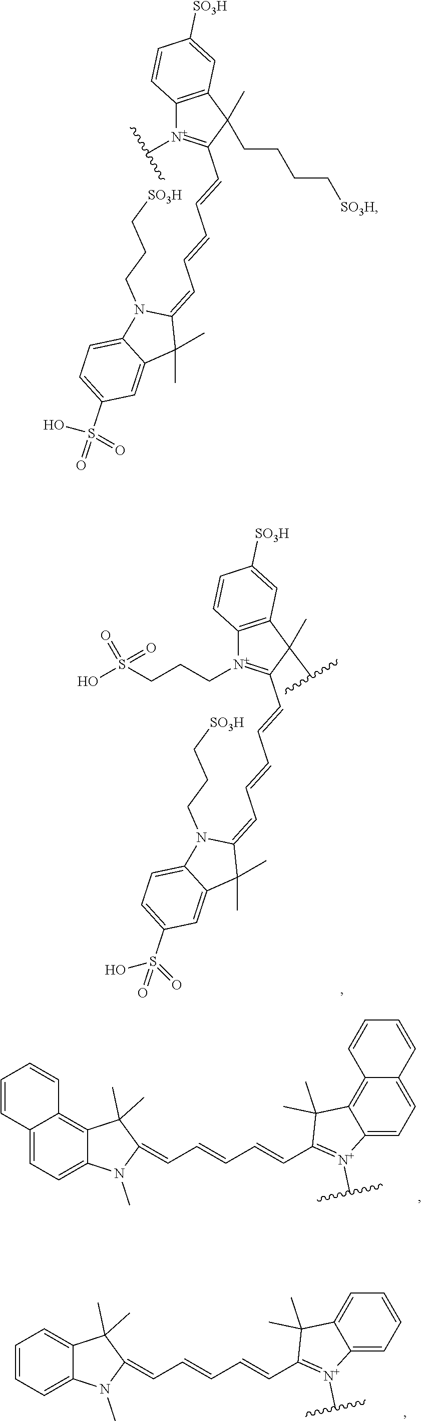 [SMILES: *[N+]1=C(/C=C/C=C/C=C2\N(CCCS(=O)(=O)O)c3ccc(S(=O)(=O)O)cc3C2(C)C)C(C)(CCCCS(=O)(=O)O)c2cc(S(=O)(=O)O)ccc21 ; *C1(C)C(/C=C/C=C/C=C2\N(CCCS(=O)(=O)O)c3ccc(S(=O)(=O)O)cc3C2(C)C)=[N+](CCCS(=O)(=O)O)c2ccc(S(=O)(=O)O)cc21 ; *[N+]1=C(/C=C/C=C/C=C2/N(C)c3ccc4ccccc4c3C2(C)C)C(C)(C)c2c1ccc1ccccc21 ; *[N+]1=C(/C=C/C=C/C=C2/N(C)c3ccccc3C2(C)C)C(C)(C)c2ccccc21]

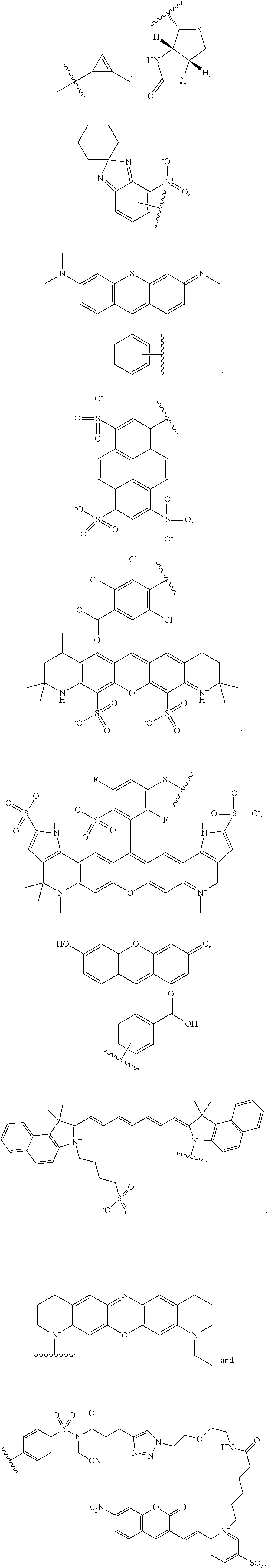

R^{4a} and R^{4b} are each independently selected from hydroxy, amino, dimethylamino, and azetidinyl;
R^{4c} and R^{4d} are each independently selected from hydrogen and —SO₃H;
R^{4c}, R^{4f}, and R^{4g} are each independently selected from hydrogen and halogen;
Y is selected from Se, S, O, C(CH₃)₂, C(CD₃)₂, NCH₂C≡CH; and
Z is selected from 0, S and Si(CH₃)₂.

In a preferred embodiment, the present invention provides a compound of formula (I) as described herein, or a pharmaceutically acceptable salt thereof, wherein R¹ is selected from hydroxy and amino.

In one embodiment, the present invention provides a compound of formula (I) as described herein, or a pharmaceutically acceptable salt thereof, wherein:
R¹ is a group

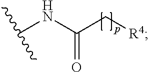

R⁴ is selected from:

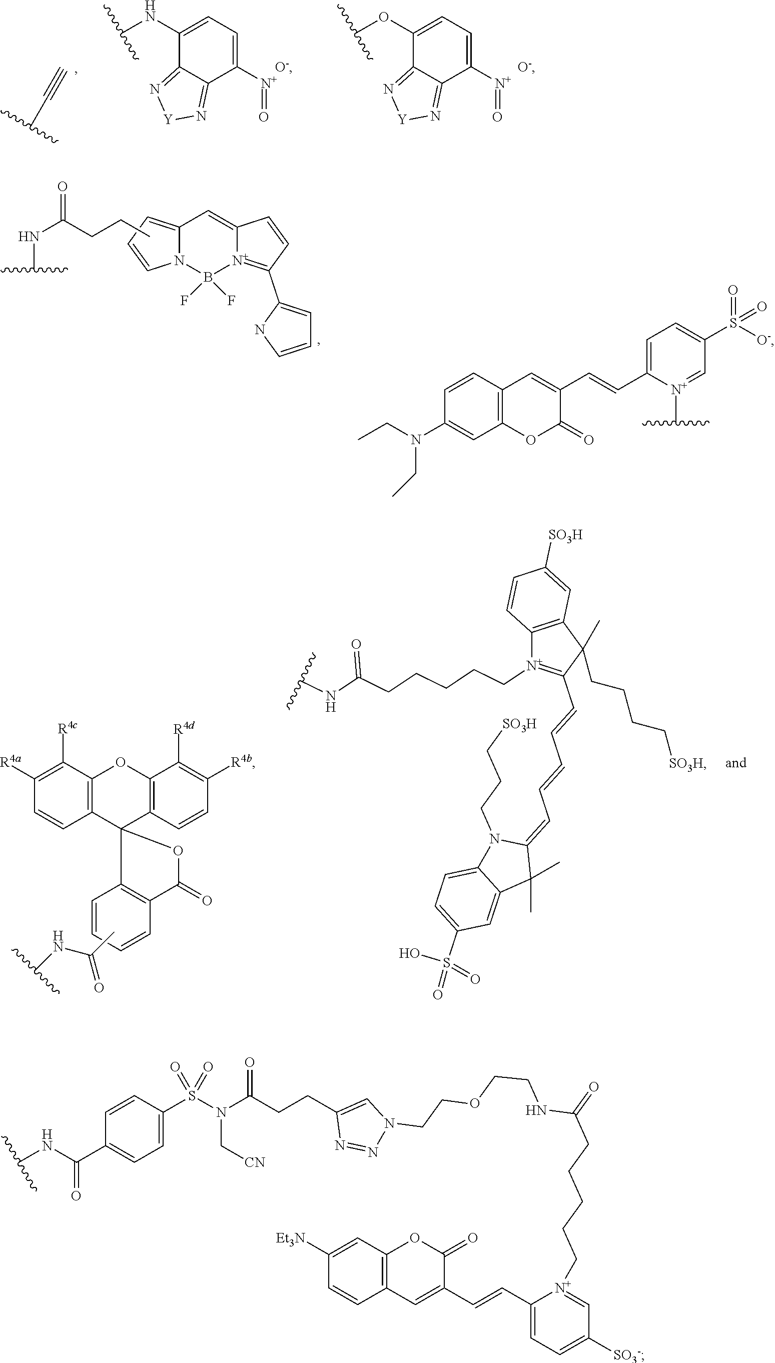

p is an integer selected from 2, 3, 4, 5, and 6;
R^{4a} and R^{4b} are each independently selected from hydroxy, amino and dimethylamino;
R^{4c} and R^{4d} are each independently selected from hydrogen and —SO₃H; and
Y is O.

In a preferred embodiment, the present invention provides a compound of formula (I) as described herein, or a pharmaceutically acceptable salt thereof, wherein:
R¹ is a group

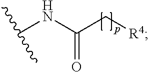

R⁴ is selected from:

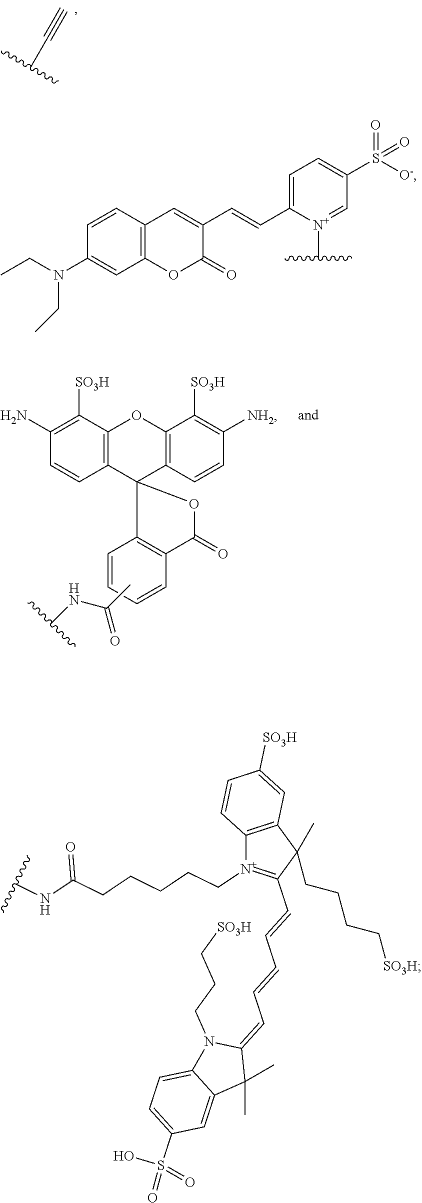

and
p is an integer selected from 2 and 5.

In a particularly preferred embodiment, the present invention provides a compound of formula (I) as described herein, or a pharmaceutically acceptable salt thereof, wherein R¹ is selected from:

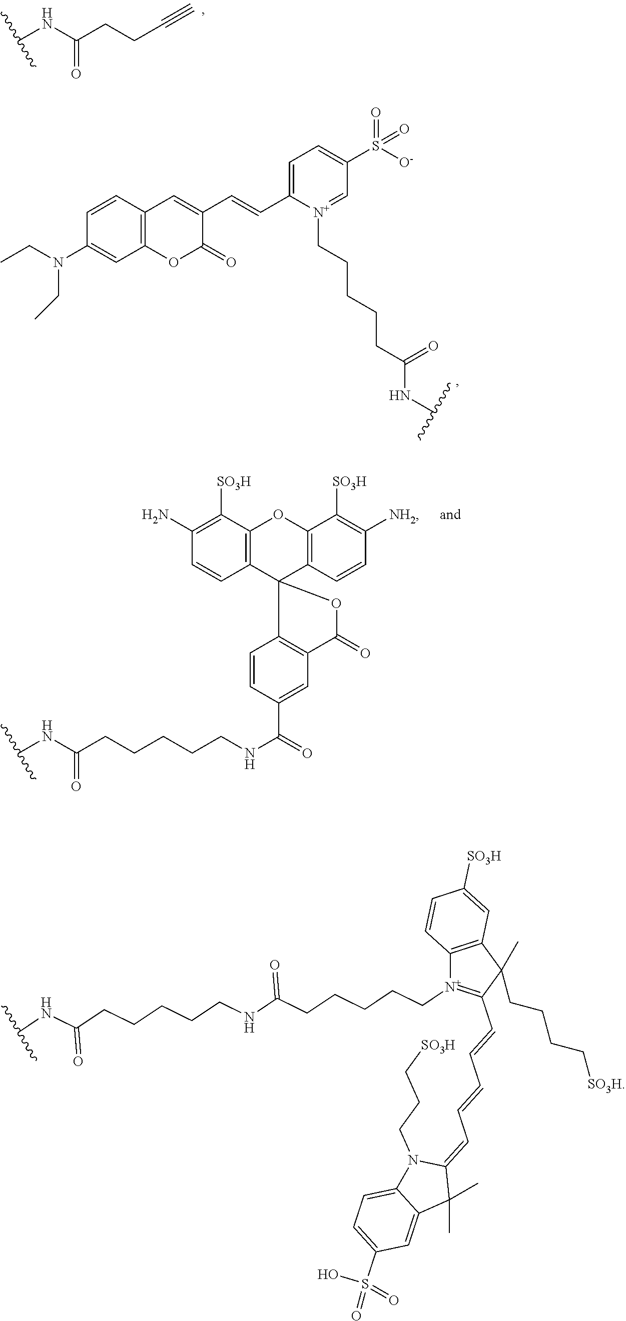

In one embodiment, the present invention provides a compound of formula (I) as described herein, or a pharmaceutically acceptable salt thereof, wherein R² is selected from C₁-C₆-alkyl, halo-C₁-C₆-alkyl, C₆-C₁₀-aryl, and 5- to 14-membered heteroaryl; wherein said C₆-C₁₀-aryl and 5- to 14-membered heteroaryl are optionally substituted with 1 substituent selected from halogen and C₁-C₆-alkyl.

In one embodiment, the present invention provides a compound of formula (I) as described herein, or a pharmaceutically acceptable salt thereof, wherein R² is selected from C₁-C₆-alkyl, halo-C₁-C₆-alkyl, phenyl, and 5- to 6-membered heteroaryl comprising 1-3 heteroatoms independently selected from N, O and S, the remaining atoms being carbon; wherein said phenyl and 5- to 6-membered heteroaryl are optionally substituted with 1 substituent selected from halogen and C₁-C₆-alkyl.

In one embodiment, the present invention provides a compound of formula (I) as described herein, or a pharmaceutically acceptable salt thereof, wherein R² is selected from ethyl, 1-propyl, 2-propyl, CF₃, phenyl, 4-fluorophenyl, 3-fluorophenyl, and 1-methylpyrazol-3-yl.

In a preferred embodiment, the present invention provides a compound of formula (I) as described herein, or a pharmaceutically acceptable salt thereof, wherein R² is C₆-C₁₀-aryl.

In a particularly preferred embodiment, the present invention provides a compound of formula (I) as described herein, or a pharmaceutically acceptable salt thereof, wherein R² is phenyl.

In a preferred embodiment, the present invention provides a compound of formula (I) as described herein, or a pharmaceutically acceptable salt thereof, wherein R³ is selected from C₁-C₆-alkyl and azido-C₁-C₆-alkyl.

In a particularly preferred embodiment, the present invention provides a compound of formula (I) as described herein, or a pharmaceutically acceptable salt thereof, wherein R³ is selected from pentyl and 5-azidopentyl.

In one embodiment, the present invention provides a compound of formula (I) as described herein, or a pharmaceutically acceptable salt thereof, wherein:
R¹ is selected from hydroxy, amino, and a group

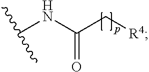

R² is selected from C₁-C₆-alkyl, halo-C₁-C₆-alkyl, C₆-C₁₀-aryl, and 5- to 14-membered heteroaryl; wherein said C₆-C₁₀-aryl and 5- to 14-membered heteroaryl are optionally substituted with 1 substituent selected from halogen and C₁-C₆-alkyl;
R³ is selected from C₁-C₆-alkyl and azido-C₁-C₆-alkyl;
R⁴ is selected from:

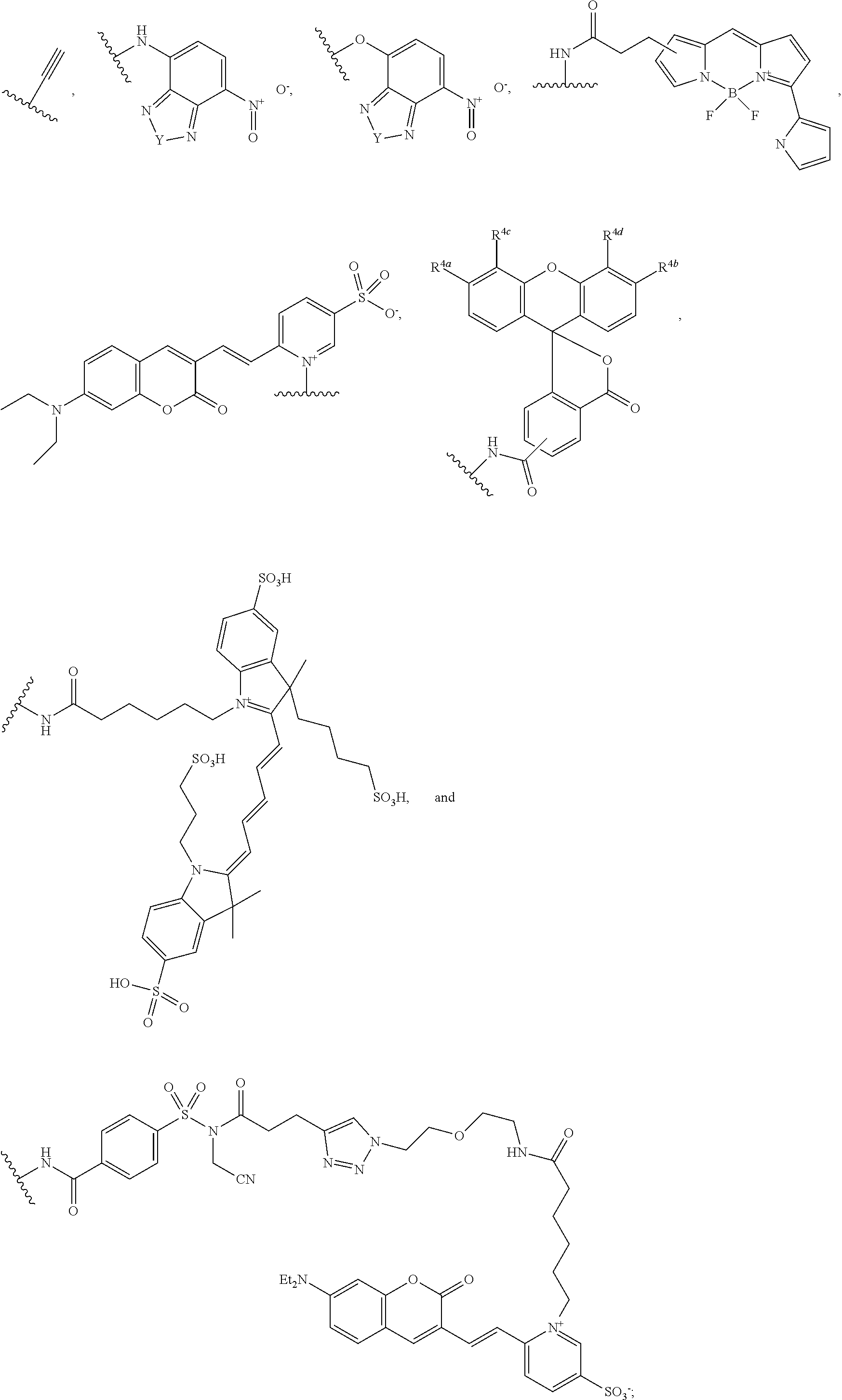

p is an integer selected from 2, 3, 4, 5, and 6;
R^{4a} and R^{4b} are each independently selected from hydroxy, amino and dimethylamino;
R^{4c} and R^{4d} are each independently selected from hydrogen and —SO₃H; and
Y is O.

In one embodiment, the present invention provides a compound of formula (I) as described herein, or a pharmaceutically acceptable salt thereof, wherein:
R¹ is selected from hydroxy, amino, and a group

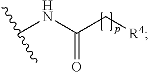

R² is selected from C₁-C₆-alkyl, halo-C₁-C₆-alkyl, phenyl, and 5- to 6-membered heteroaryl comprising 1-3 heteroatoms independently selected from N, O and S, the remaining atoms being carbon; wherein said phenyl and 5- to 6-membered heteroaryl are optionally substituted with 1 substituent selected from halogen and C₁-C₆-alkyl;
R³ is selected from C₁-C₆-alkyl and azido-C₁-C₆-alkyl;
R⁴ is selected from:

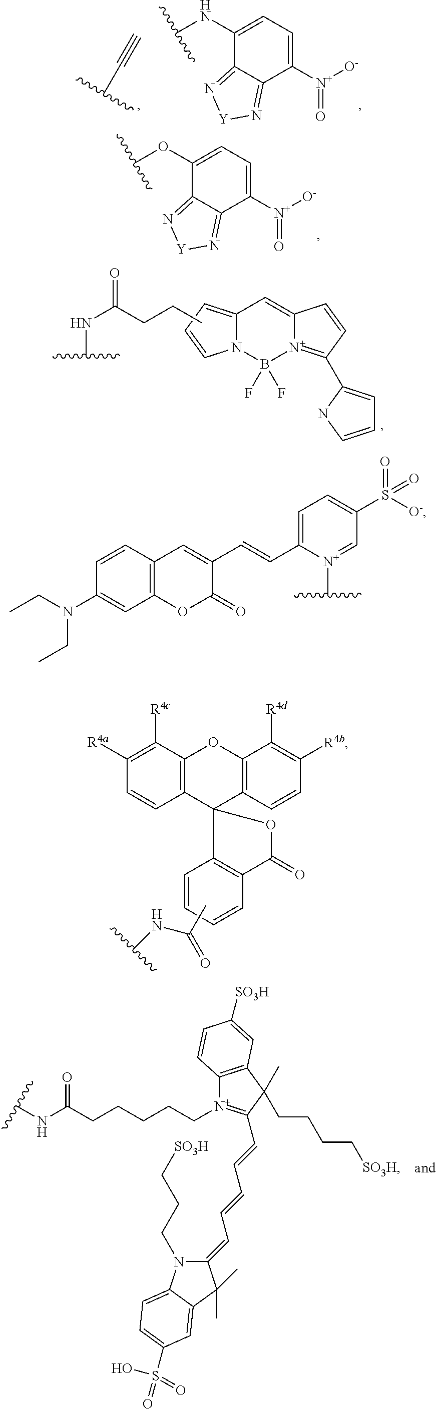

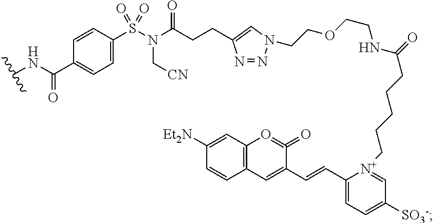

p is an integer selected from 2, 3, 4, 5, and 6;
R^{4a} and R^{4b} are each independently selected from hydroxy, amino and dimethylamino;
R^{4c} and R^{4d} are each independently selected from hydrogen and —SO₃H; and
Y is O.

In one embodiment, the present invention provides a compound of formula (I) as described herein, or a pharmaceutically acceptable salt thereof, wherein:
R¹ is selected from hydroxy, amino, and a group

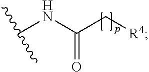

R² is selected from ethyl, 1-propyl, 2-propyl, CF₃, phenyl, 4-fluorophenyl, 3-fluorophenyl, and 1-methylpyrazol-3-yl;
R³ is selected from pentyl and 5-azidopentyl;
R⁴ is selected from:

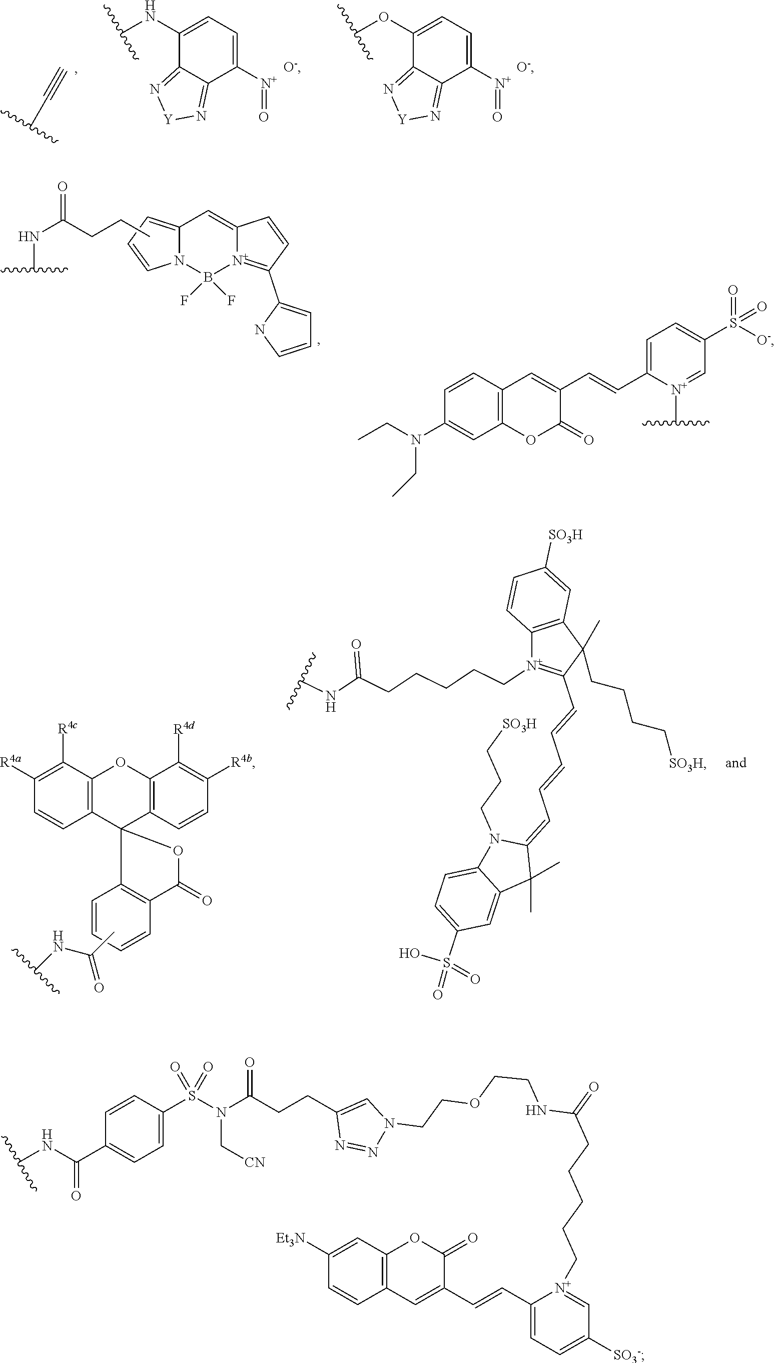

p is an integer selected from 2, 3, 4, 5, and 6;
R^{4a} and R^{4b} are each independently selected from hydroxy, amino and dimethylamino;
R^{4c} and R^{4d} are each independently selected from hydrogen and —SO₃H; and
Y is O.

In a preferred embodiment, the present invention provides a compound of formula (I) as described herein, or a pharmaceutically acceptable salt thereof, wherein:
R¹ is a group

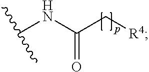

R² is C₆-C₁₀-aryl;
R³ is selected from C₁-C₆-alkyl and azido-C₁-C₆-alkyl;
R⁴ is selected from:

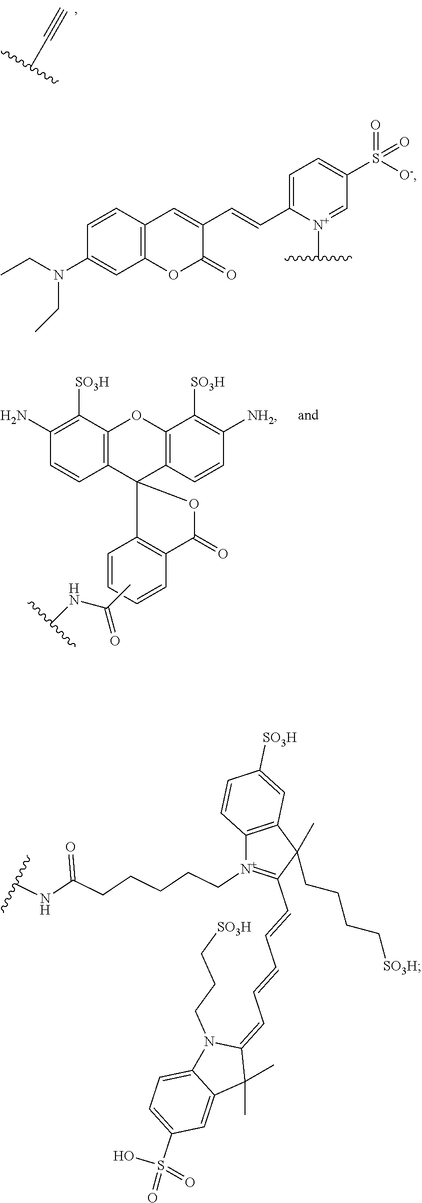

and
p is an integer selected from 2 and 5.

In a particularly preferred embodiment, the present invention provides a compound of formula (I) as described herein, or a pharmaceutically acceptable salt thereof, wherein:
R¹ is selected from:

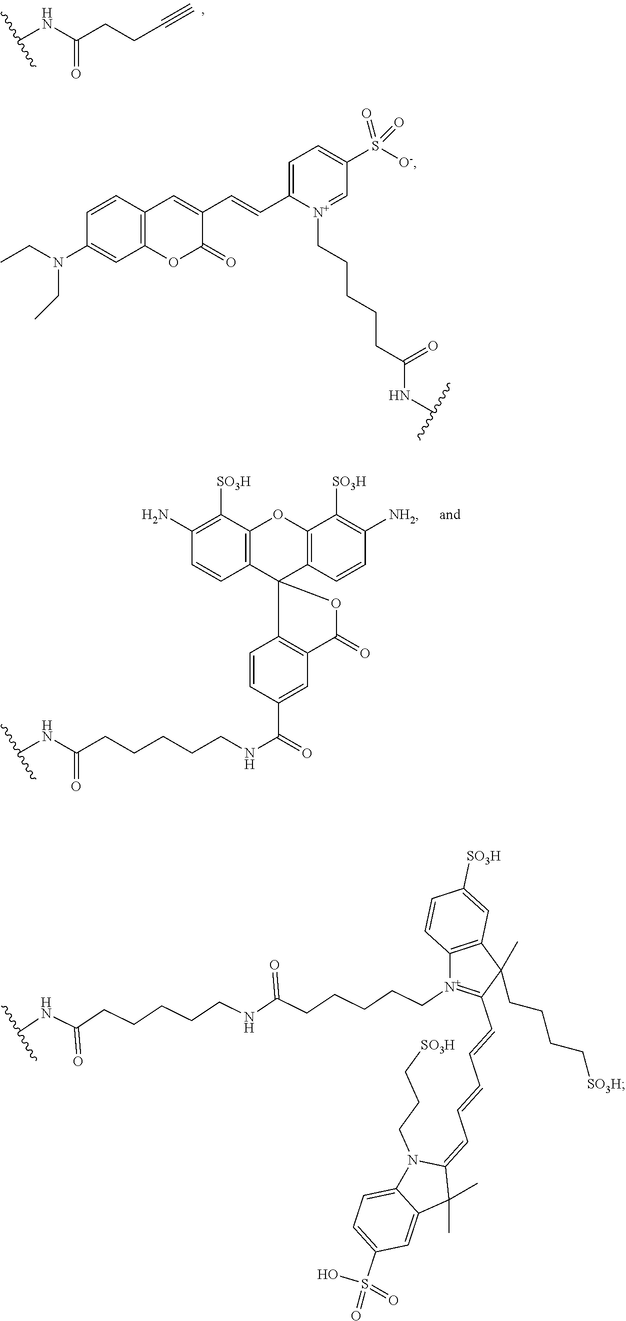

R² is phenyl; and
R³ is selected from pentyl and 5-azidopentyl.

In one embodiment, the present invention provides a compound of formula (I) as described herein, or a pharmaceutically acceptable salt thereof, wherein the compound of formula (I) is selected from:
1-(6-((((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate;
6-((E)-2-(7-(diethylamino)-2-oxo-2H-chromen-3-yl)vinyl)-1-(6-((((1S,4S,5S)-4-(2,6-dimethoxy-4-(2-methyl-3-phenyloctan-2-yl)phenyl)-6,6-dimethylbicyclo[3.1.1]hept-2-en-2-yl)methyl)amino)-6-oxohexyl)pyridin-1-ium-3-sulfonate;
N-(((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)pent-4-ynamide;
((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine; ((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methanamine;
N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)amino)heptanamide;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)amino)heptanamide;
N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)pent-4-ynamide;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)pent-4-ynamide;
1-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate;
1-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate;
N-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-bis(dimethylamino)-3-oxo-3H-spiro[isobenzofuran-1,9′-xanthene]-5-carboxamide;
N-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-dihydroxy-3-oxo-3H-spiro[isobenzofuran-1,9′-xanthene]-6-carboxamide;
1-(6-((6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)amino)-6-oxohexyl)-2-((1E,3E)-5-((E)-3,3-dimethyl-5-sulfo-1-(3-sulfopropyl)indolin-2-ylidene)penta-1,3-dien-1-yl)-3-methyl-3-(4-sulfobutyl)-3H-indol-1-ium-5-sulfonate;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-6-(3-(5,5-difluoro-7-(1H-pyrrol-2-yl)-5H-514,614-dipyrrolo[1,2-c:2′,1′-f][1,3,2]diazaborinin-3-yl)propanamido)hexanamide;
3′,6′-diamino-5-((6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamoyl)-3-oxo-3H-spiro[isobenzofuran-1,9′-xanthene]-4′,5′-disulfonic acid;
((1S,4S,5S)-4-(4-((R)-3-(4-fluorophenyl)-2-methyloctan-2-yl)-2,6-dimethoxyphenyl)-6,6-dimethylbicyclo[3.1.1]hept-2-en-2-yl)methanol;
   ((1S,4S,5S)-4-(4-((S)-3-(4-fluorophenyl)-2-methyloctan-2-yl)-2,6-dimethoxyphenyl)-6,6-dimethylbicyclo[3.1.1]hept-2-en-2-yl)methanol;
((1S,4S,5S)-4-(4-((R)-3-(3-fluorophenyl)-2-methyloctan-2-yl)-2,6-dimethoxyphenyl)-6,6-dimethylbicyclo[3.1.1]hept-2-en-2-yl)methanol;
((1S,4S,5S)-4-(4-((S)-3-(3-fluorophenyl)-2-methyloctan-2-yl)-2,6-dimethoxyphenyl)-6,6-dimethylbicyclo[3.1.1]hept-2-en-2-yl)methanol;
((1S,4S,5S)-4-(4-((R)-3-ethyl-2-methyloctan-2-yl)-2,6-dimethoxyphenyl)-6,6-dimethylbicyclo[3.1.1]hept-2-en-2-yl)methanol;
((1S,4S,5S)-4-(4-(3-ethyl-2-methyloctan-2-yl)-2,6-dimethoxyphenyl)-6,6-dimethylbicyclo[3.1.1]hept-2-en-2-yl)methanol;
((1S,4S,5S)-4-(2,6-dimethoxy-4-(2-methyl-3-propyloctan-2-yl)phenyl)-6,6-dimethylbicyclo[3.1.1]hept-2-en-2-yl)methanol;
((1S,4S,5S)-4-(4-(3-isopropyl-2-methyloctan-2-yl)-2,6-dimethoxyphenyl)-6,6-dimethylbicyclo[3.1.1]hept-2-en-2-yl)methanol;
((1S,4S,5S)-4-(2,6-dimethoxy-4-((R)-2-methyl-3-(trifluoromethyl)octan-2-yl)phenyl)-6,6-dimethylbicyclo[3.1.1]hept-2-en-2-yl)methanol;
((1S,4S,5S)-4-(2,6-dimethoxy-4-((S)-2-methyl-3-(trifluoromethyl)octan-2-yl)phenyl)-6,6-dimethylbicyclo[3.1.1]hept-2-en-2-yl)methanol;
((1S,4S,5S)-4-(2,6-dimethoxy-4-((R)-2-methyl-3-phenyloctan-2-yl)phenyl)-6,6-dimethylbicyclo[3.1.1]hept-2-en-2-yl)methanol;
((1S,4S,5S)-4-(2,6-dimethoxy-4-((S)-2-methyl-3-phenyloctan-2-yl)phenyl)-6,6-dimethylbicyclo[3.1.1]hept-2-en-2-yl)methanol;
((1S,4S,5S)-4-(2,6-dimethoxy-4-(2-methyl-3-(1-methyl-1H-pyrazol-3-yl)octan-2-yl)phenyl)-6,6-dimethylbicyclo[3.1.1]hept-2-en-2-yl)methanol;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)oxy)heptanamide; and
1-(6-((2-(2-(4-(3-((4-((7-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate.

In a particular embodiment, the present invention provides pharmaceutically acceptable salts of the compounds according to formula (I) as described herein. In a further particular embodiment, the present invention provides compounds according to formula (I) as described herein as free bases.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, 13C ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from, for example, greater metabolic stability, increased in vivo half-life or reduced dosage requirements. Thus, in one embodiment, the present invention provides compounds of formula (I) as described herein, or pharmaceutically acceptable salts thereof, wherein one or more hydrogen atoms are replaced by deuterium. In a preferred embodiment, the present invention provides compounds of formula (I) as described herein, or pharmaceutically acceptable salts thereof, wherein 1-10 hydrogen atoms are replaced by deuterium. In a particularly preferred embodiment, the present invention provides compounds of formula (I) as described herein, or pharmaceutically acceptable salts thereof, wherein 1-6 hydrogen atoms, e.g. 1, 2, 3, 4, 5 or 6 hydrogen atoms, are replaced by deuterium.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protective groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (I) will typically lead to the respective diastereomerically/enantiomerically enriched compounds of formula (I).

A person skilled in the art will acknowledge that in the synthesis of compounds of formula (I)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, NY. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:

AcOH=acetic acid, ACN=acetonitrile, AIBN=Azobisisobutyronitrile, Boc=tert-butyloxycarbonyl, BuLi=butyllithium CAS RN=chemical abstracts registration number, CH₂Cl₂=dichloromethane, COMU=(1-Cyano-2-ethoxy-2-oxoethylidenaminooxy)dimethylamino-morpholino-carbenium hexafluorophosphate, DCC=N,N′-Dicyclohexylcarbodiimide, DIBAL=Diisobutylaluminium hydride, DIC=N,N′-Diisopropylcarbodiimide, N,N′-Dicyclohexylcarbodiimide, DMAP=4-dimethylaminopyridine, DMA=N,N-dimethylacetamide, DMF=N,N-dimethylformamide, DMP=3-Oxo-1λ⁵,2-benziodoxole-1,1,1(3H)-triyl triacetate, DMSO=dimethylsulfoxide, i-Pr₂NEt=N,N-diisopropylethylamine, EDCI=N-(3-dimethylaminopropyl)-N′-ethylcarbodiimide, ESI=electrospray ionization, Et₃N=triethylamine, Et₂O=diethyl ether, EtOAc=ethyl acetate, EtOH=ethanol, h=hour(s), H₂O=water, HATU=1-[bis(dimethylamino)methylene]-1H-1,2,3-triazolo[4,5-b]pyridinium-3-oxide hexafluorophosphate, HCl=hydrochloric acid, HPLC=high performance liquid chromatography, K₂CO₃=potassium carbonate, KHMDS=Potassium bis(trimethylsilyl)amide, MeOH=methanol, MgSO₄=magnesium sulfate, min=minute(s), mL=milliliter, MS=mass spectrum, NaH=sodium hydride, NaHCO₃=sodium hydrogen carbonate, NaOH=sodium hydroxide, Na₂SO₄=sodium sulfate, NEt₃=triethylamine (TEA), NH₄Cl=ammonium chloride, PG=protective group, R=any group, Rochelle's salt=Sodium potassium L(+)-tartrate tetrahydrate, rt=room temperature, T3P=Propylphosphonic anhydride, TBAF=Tetra-n-butylammonium fluoride, TBTU=0-(Benzotriazol-1-yl)-N,N,N′,N′-tetramethyluronium tetrafluoroborate, TFA=trifluroacetic acid, THF=tetrahydrofuran, TLC=thin layer chromatography, SFC=Supercritical fluid chromatography.

Compound of formula I wherein R¹, R², R⁴, R⁵ and p are as described herein can be synthesized in analogy to literature and/or as depicted for example in Scheme 1.

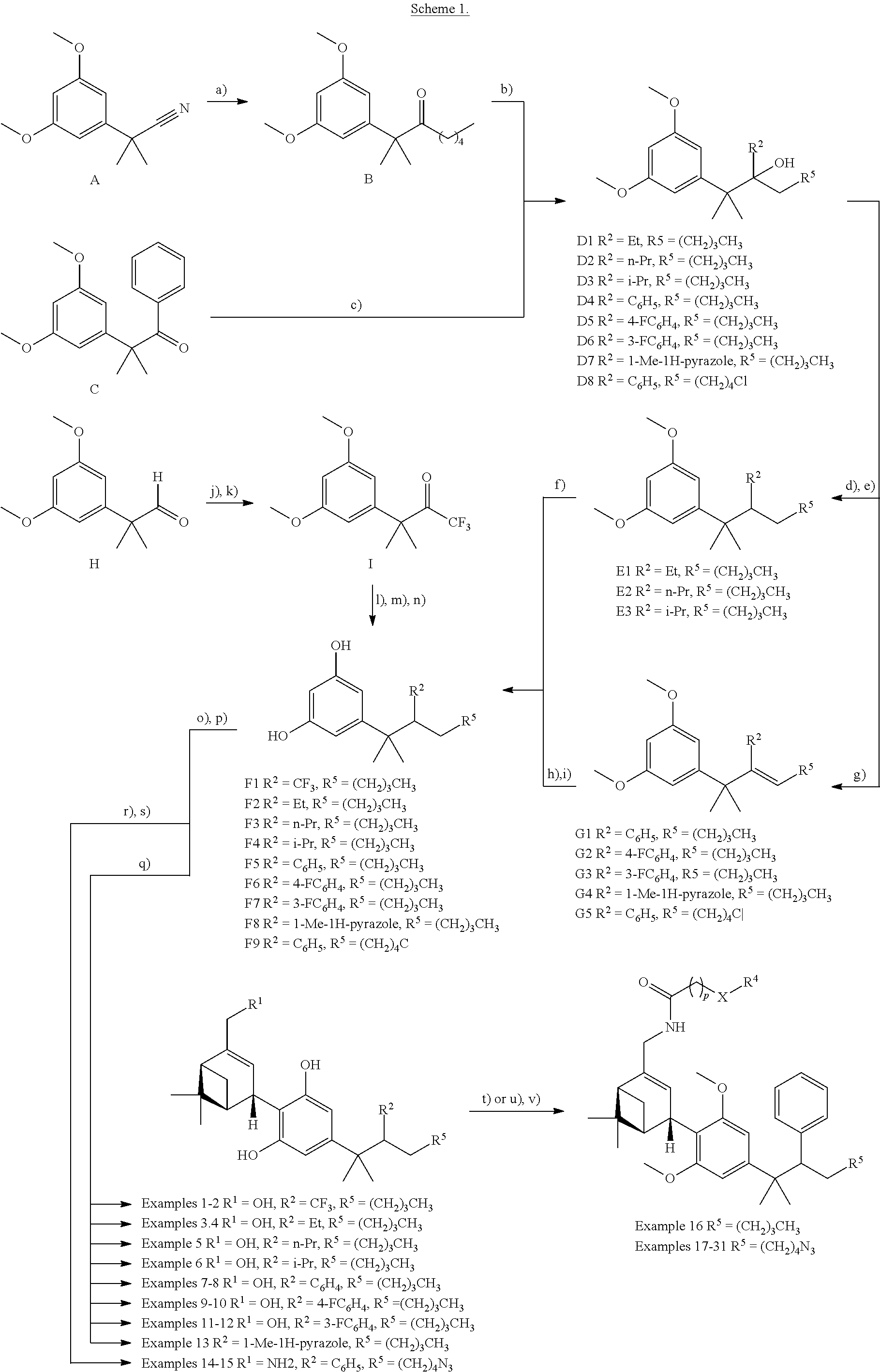

Accordingly, nitriles type A are reacted with organometallic reagents, such as alkyl lithiums to yield B (step a). Subsequently, B is reacted with organometallic reagents such as organolithiums, optinally catalyzed with a Lewis acid such as CeCl₃ or LaCl₃ to yield alcohols D1-D7 (step b). In an analogous fashion starting from ketone C the alcohol D8 can be achieved by treatment with an organometallic reagent (step c). Alcohols D1-D3 were subject to elimination followed by hydrogenation to yield the saturated intermediates E1-E3 (steps d and e). Finally, double demethylation of E1-E3 afforded resorcinols F2-F4 (step f). Alcohols D4-D8 were eliminated to yield the alkenes G1-G5 (step g) followed by demethylation and hydrogenation to furnish the resorcinols F5-F9 (steps h and i). Subjecting aldehyde H to a nucleophilic trifluoromethylation reaction followed by oxidation yielded ketone I (steps j and k). Ketone I can be subjected to a Wittig reaction with a phosphonium ylide followed by hydrogenation and double demethylation to yield resorcinol F1. The racemic resorcinols F1-F9 could be optionally separated by a chiral resolution method, such as semi-preparative chiral SFC to yield optically enriched enantiomers. A person skilled in the art will acknowledge that other methods of racemic resolution exist and include for example chiral derivatization, crystallization or resolution. Finally, an acid catalyzed allylation of F1-F8 followed by methylation and deprotection yielded Examples 1-13 (steps o, p and q). Examples 14 and 15 can be achieved from resorcinol F9 by a similar sequence of allylation (step o), double methylation (step p), substitution with a desired terminal functional group (step r), such as an azide, and finally deprotection (step s). Examples 16-31 can be achieved by functionalization of the primary amine, such as found in Examples 14-15, into an amide either by direct reaction with the desired carboxylic acid (step t), its equivalent or optionally by a prior installation of an appropriate linker element (steps u and v). Amide couplings of this type can be achieved by using one of the well-known coupling reagents such as, DCC, HATU, EDCI, TBTU, T3P, etc. and a base like i-Pr₂NEt, Et₃N or DMAP in a suitable solvent like DMF, DMA, CH₂Cl₂ or dioxane, preferably between 0° C. and room temperature.

The absolute configuration at the C(2′) homobenzylic position α to the gem-dimethyl group of Examples 14, 15, 17-31, which contain resorcinol F9 was determined as depicted in Scheme 2.

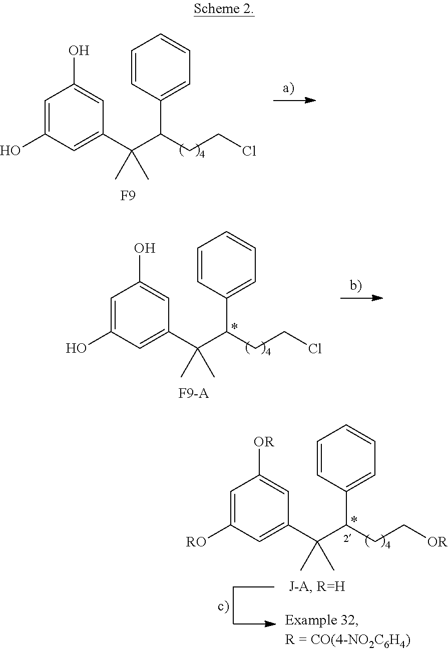

Accordingly, racemate F9 was resolved into individual enantiomers (step a) by the means of supercritical fluid chromatography (SFC) using a chiral stationary phase. The first eluting enantiomer A, F9-A, was hydrolyzed (step b) to J-A and derivatized with p-nitrobenzoyl chloride to afford Example 32 (step c). Nitrobenzoate Example 32 had its structure elucidated by X-ray crystallography allowing to assessing the C(2′) configuration as S.

TR-FRET hCB₂R Binding Assay

Cell culture: Cells were maintained in a humidified environment at 37° C. and 5% CO₂ in Dulbecco's modified Eagle's medium (DMEM) with 10% fetal bovine serum (FBS) containing blasticidin (5 μg/mL; Invitrogen) and (Zeocin; 20 μg/mL; Invitrogen). For inducible expression, SNAP-tagged human CB₂ receptor cDNAs, in pcDNA4/TO were introduced through transfection, using PEI into HEK293TR cells (Invitrogen, which express Tet repressor protein to allow inducible expression). A mixed population stable line was selected by resistance to blasticidin (TR vector, 5 μg/mL) and Zeocin; (receptor plasmid, 20 μg/mL). For receptor-inducible expression, cells were seeded into t175 cm² flasks, grown to 70% confluence and DMEM containing 1 μg/ml tetracycline added. 24 h later cells were labelled with SNAP-Lumi4-Tb (CisBio) and membranes prepared as described in detail below.

Terbium labeling of SNAP-tagged CB2R HEK293-TR cells: Cell culture medium was removed from the t175 cm2 flasks containing confluent adherent CB₂ HEK293-TR cells. Cells were washed 1× in PBS (GIBCO Carlsbad, CA) followed by 1× Tag-lite labeling medium (LABMED, CisBio) to remove the excess cell culture media, then ten millilitre of LABMED containing 100 nM of SNAP-Lumi4-Tb was added to the flask and incubated for 1 h at 37° C. under 5% CO₂. Cells were washed 1× in PBS (GIBCO Carlsbad, CA) to remove the excess of SNAP-Lumi4-Tb then detached using 5 mL of GIBCO enzyme-free Hank's-based cell dissociation buffer (GIBCO, Carlsbad, CA) and collected in a vial containing 5 ml of DMEM (Sigma-Aldrich) supplemented with 10% fetal calf serum. Cells were pelleted by centrifugation (5 min at 1500 rpm) and the pellets were frozen to −80° C. To prepare membranes, homogenization steps were conducted at 4° C. (to avoid receptor degradation) as described in C. K. Herenbrink, et. al., Nat. Commun., 2016, 7, 10842.

Fluorescent ligand-binding assays: All fluorescent ligand binding experiments were conducted in white 384-well Optiplate plates, in assay binding buffer, either Hanks Balanced Salt Solution (HBSS), 5 mM HEPES, 0.5% BSA, 0.02% pluronic F-127 pH 7.4, and 100 μM GppNHp. GppNHp was included to remove the G protein-coupled population of receptors that can result in two distinct populations of binding sites in membrane preparations, since the Motulsky-Mahan model is only appropriate for ligands competing at a single site. In all cases, nonspecific binding was determined by the presence of 1 μM SR144528. Data are presented in mean±SEM from a representative of 3-8 experiments.

Determination of fluorescent ligand binding kinetics and equilibrium affinity: To accurately determine association rate (kon) and dissociation rate (koff) values, the observed rate of association (kob) was calculated using at least five different concentrations 8-SiR or fluorescent tracer D77 (for 8-SiR and assay description see T. Gazzi et al. Chem. Sci., 2022, 13, 5539-5545).

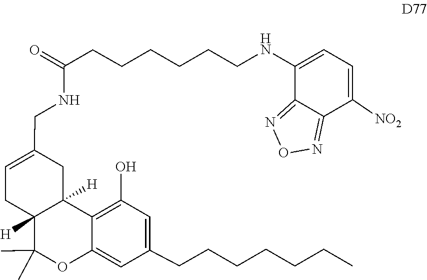 [SMILES: CCCCCCCc1cc(O)c2c(c1)OC(C)(C)[C@@H]1CC=C(CNC(=O)CCCCCCNc3ccc([N+](=O)[O-])c4nonc34)C[C@@H]21]

The appropriate concentration of fluorescent ligand binding was incubated with human CB2R HEK293-TR cell membranes (4 μg per well) in assay binding buffer (final assay volume, 40 μL). The degree of fluorescent ligand bound to the receptor was assessed at multiple time points by HTRF detection to allow construction of association kinetic curves. The resulting data were globally fitted to the association kinetic model (Eq. 1, see signal detection and data analysis section below) to derive a single best-fit estimate for kon and koff as described under data analysis. Saturation analysis was performed at equilibrium, by simultaneously fitting total and Nonspecific (NSB) binding data (Eq. 2, see signal detection and data analysis section below) allowed the determination of fluorescent ligand binding affinity.

Competition binding: To determine the affinity of CB₂R-specific ligands, we used a simple competition kinetic binding assay. This approach involves the simultaneous addition of both fluorescent ligand and competitor to the CB₂R preparation. 62.5 nM 8-SiR or 900 nM D77, concentration which avoid ligand depletion in this assay volume, were added simultaneously with increasing concentrations of the unlabeled compound to CB₂R cell membranes (4 μg per well) in 40 μL of assay buffer in a 384-well plate incubated at room temperature with orbital mixing. The degree of fluorescent ligand bound to the receptor was assessed at equilibrium by HTRF detection. Nonspecific binding was determined as the amount of HTRF signal detected in the presence of SR144528 (1 μM) and was subtracted from total binding, to calculate specific binding for construction of IC₅₀ curves. The kinetic parameters of D77 plus those of unlabeled compounds were determined using a start time of ~60 sec and an interval time of 60 sec. Nonspecific binding was determined as the amount of HTRF signal detected in the presence of (SR144,528, 10 μM) and was subtracted from total binding, to calculate specific binding, meaning that t=0 was always equal to zero.

Signal detection and data analysis: Signal detection was performed on a Pherastar FSX (BMG Labtech, Offenburg, Germany). The terbium donor was always excited with eight laser flashes at a wavelength of 337 nm. TR-FRET signals were collected at 665 (acceptor) and 620 nm (donor) when using the red acceptor fluorescent ligand 8-SiR. HTRF ratios were obtained by dividing the acceptor signal by the donor signal and multiplying this value by 10′000. All experiments were analyzed by non-regression using Prism 8.0 (GraphPad Software, San Diego, USA).

Fluorescent ligand association data were fitted as follows to a global fitting model using GraphPad Prism 8.0 to simultaneously calculate kₒₙ and k_{off} using the following equation,

$$Y = {Y}_{max} * ( 1 - exp ( - 1 * {k}_{ob} * X ) ) \qquad ( Eq . 1 )$$

wherein, k_{ob}=[L]*kₒₙ+k_{off}

Where, k_{ob} equals the observed rate of ligand association and kₒₙ and k_{off} are the association and dissociation-rate constants respectively of the fluorescent ligand. In this globally fitted model of tracer binding, tracer concentrations [L] are fixed, kₒₙ and k_{off} are share parameters whilst k_{obs} is allowed to vary. Here, Y is the level of receptor-bound tracer, Yₘₐₓ is the level of tracer binding at equilibrium, X is in units of time (eg. min) and k_{obs} is the rate in which equilibrium is approached (eg·min⁻¹).

$$Total binding = Specific + NSB = \frac{B max * [ L ]}{Rd + [ L ]} + slope * [ L ] + background \qquad ( Eq . 2 )$$

$$NSB = slope * [ L ] + Background$$

Saturation binding data were analyzed by non-linear regression according to a one-site equation by globally fitting total and NSB. Individual estimates for the fluorescent ligand dissociation constant (K_{d}) were calculated using the following equations where L is the is fluorescent ligand concentration:

Fitting the total and NSB data sets globally (simultaneously), sharing the value of slope, provides one best-fit value for both the K_{d} and the Bₘₐₓ.

Competition displacement binding data were fitted to sigmoidal (variable slope) curves using a ‘four-parameter logistic equation’:

$$Y = Bottom + ( Top - Bottom ) / ( 1 + {10}^{( logIC50 - X ) \times Hill coefficient} ) \qquad ( Eq . 3 )$$

IC₅₀ values obtained from the inhibition curves were converted to Kᵢ values using the method of Cheng and Prusoff (C. Yung-Chi, W. H. Prusoff, Biochem. Pharmacol. 1973, 22, 3099-3108).

Association and dissociation rates for unlabeled antagonists were calculated using the following equations first described in Motulsky, H. J.; Mahan, L. C. Mol. Pharmacol. 1984, 25, 1-9 and modified in Schiele, F. et. al. Anal. Biochem. 2015, 468, 42-9 to account for photobleaching.

$${K}_{A} = {k}_{1} [ L ] + {k}_{2} \qquad ( Eq . 4 )$$

$${K}_{B} = {k}_{3} [ I ] + {k}_{4}$$

$$S = \sqrt{( {( {K}_{A} - {K}_{A} )}^{2} + 4 \cdot {k}_{1} \cdot {k}_{3} \cdot L \cdot I \cdot {10}^{- 18} )}$$

$${K}_{F} = 0.5 * ( {K}_{A} + {K}_{B} + S )$$

$${K}_{S} = 0.5 * ( {K}_{A} + {K}_{B} - S )$$

$$Q = \frac{{B}_{max} * {K}_{1} * L * 1 {0}^{- 9}}{{K}_{F} - {K}_{S}}$$

$$Mot = \cdot ( \frac{{k}_{4} \cdot ( {K}_{F} - {K}_{S} )}{{K}_{F} \cdot {K}_{S}} + \frac{{k}_{4} - {K}_{F}}{{K}_{F}} {exp}^{( - {K}_{F} \cdot X )} - \frac{{k}_{4} {K}_{S}}{{K}_{S}} {exp}^{( - {K}_{S} \cdot X )} )$$

$$Bleaching = {B}_{max} * {exp}^{( - {K}_{drift} * X )}$$

$$Y = Mot * Bleaching$$

Where: X=Time (min), Y=Specific binding (e.g. CPM or HTRF units e.g. HTRF ratio 520 nm/620 nm×10′000), k₁=kₒₙ tracer (M⁻¹ min⁻¹), k₂=k_{off} tracer (min⁻¹), L=Concentration of tracer used (nM), I=Concentration unlabeled ligand (nM). Fixing the above parameters allows the following to be calculated: k₃=Association-rate constant of unlabeled ligand (M⁻¹ min⁻¹), k₄=Dissociation-rate constant of unlabeled ligand (min⁻¹), Bₘₐₓ=Maximal specific binding of the system at equilibrium binding (e.g. CPM or HTRF units, e.g. HTRF ratio 520 nm/620 nm×10′000), K_{drift}=Signal drift.

TABLE 1

| | K_{d} hCB₂R | K_{d} hCB₁R |
|---|---|---|
| Example | [nM] | [nM] |
| 1 | 606 | 11820 |
| 2 | 9.9 | 6526 |
| 3 | 0.56 | 12960 |
| 4 | 0.97 | 2186 |
| 5 | 74 | 6736 |
| 6 | 6.1 | 7036 |
| 7 | 197 | 1037 |
| 8 | 4.5 | 716 |
| 9 | 1149 | 1477 |
| 10 | 301 | 1786 |
| 11 | 355 | 2687 |
| 12 | 30 | 1946 |
| 14 | 476 | — |
| 15 | 39.1 | — |
| 16 | 214 | — |
| 18 | 2.1 | — |
| 19 | 0.42 | — |
| 20 | 67.9 | — |
| 21 | 162 | — |
| 22 | 10.2 | 1740 |
| 23 | 158 | — |
| 24 | 12.3 | 840 |
| 25 | 2.78 | 396 |
| 26 | 30.3 | 1280 |
| 27 | 25.9 | 7050 |
| 28 | 44.7 | — |
| 29 | 24.9 | 3300 |

cAMP hCB₂R Functional Assay

### The Homogeneous Time Resolved Fluorescence (HTRF) Assays

The homogeneous time-resolved fluorescence (HTRF) cAMP assay was conducted according to the manufacturer's protocol for the cAMP-Gs Dynamic kit (Cisbio). Briefly, the CHO cell line stably overexpressing CB2 receptor were maintained in Ham's F12 supplemented with 10% FBS, 100 U/ml penicillin and 100 μg/ml streptomycin (Gibco-ThermoFisher) and 400 μg/ml G418 (ThermoFisher). For CB2 compounds test, harvest the cells with Cell Dissociation Buffer and re-suspend the cells in F-12 K at 0.4*10⁶ cells/mL, and dispense cells into 384-well low volume plates at 2000 cells/5 μL per well. The cells were stimulated with compounds diluted in Stimulation Buffer (2.5 μL/well) for 15 min at room temperature. And then followed by the addition of 2.5 μL 25 μM forsklin. After 15 mins incubation, reactions were stopped by 5 μL/well cAMP-d2 conjugate working solution followed by 5 μL/well of anti-cAMP cryptate working solution. After incubation for 1 h at room temperature, the plates were read in a PerkinElmer Envision reader for time-resolved fluorescence resonance energy transfer detection at 620 nm and 665 nm.

### Quantification and Statistical Analysis

All HTRF ratio data sets of test compounds were normalized to the Emax of CP55940 (100%) and obtained the means±standard error of the mean (SEM) of 2-3 independent experiments performed in technical replicates.

TABLE 2

| Example | pEC₅₀ | Functionality |
|---|---|---|
| 1 | — | antagonist |
| 2 | 7.93 ± 0.11 | agonist |
| 3 | 7.00 ± 0.11 | partial agonist |
| 4 | — | antagonist |
| 5 | 6.95 ± 0.31 | inverse agonist |
| 6 | — | antagonist |
| 7 | 6.38 ± 0.35 | inverse agonist |
| 8 | 6.52 ± 0.17 | inverse agonist |
| 9 | — | antagonist |
| 10 | — | antagonist |
| 11 | 5.63 ± 0.63 | inverse agonist |
| 12 | 6.68 ± 0.18 | inverse agonist |
| 14 | 5.57 ± 0.45 | inverse agonist |
| 15 | 6.95 ± 0.13 | inverse agonist |
| 18 | 7.39 ± 0.14 | inverse agonist |
| 19 | 7.48 ± 0.12 | inverse agonist |
| 21 | 6.47 ± 0.24 | inverse agonist |
| 22 | 7.15 ± 0.12 | inverse agonist |
| 23 | — | Antagonist |
| 24 | 5.91 ± 0.22 | inverse agonist |

### Using the Compounds of the Invention

The compounds of formula (I) are modulators of CB₂R pharmacology and fluorescent probes with high affinity for CB₂R. The fluorescent probes retain excellent affinity and specificity for CB₂R regardless of the specific fluorophore used and hence are exquisite high-resolution tools to investigate localization, expression levels and protein distribution in health and disease, structure, dynamics and function of CB₂R in vitro, in living cells and in vivo. The inverse agonist functionality of the fluorescent probes promises to investigate receptor populations without triggering receptor activation and associated agonist-mediated internalization events at CB₂R. Additionally, a person skilled in the art can select a specific is fluorophore from the reported examples (cell permeable/impermeable, environment sensitive/insensitive, tailored wavelength of absorption and emission, etc.) to investigate a specific receptor-related event and/or target an membrane bound, intracellular, compartmentalized receptor population. The probes may also be applied in flow cytometry fluorescence-activated cell sorting (FACS) experiments or cellular trafficking studies using fluorescent confocal live cell imaging. Certain probes (such as Examples 30 and 31) are especially well suited to conduct these experiments without disturbance to the cellular homeostasis due to their functionalization with a cleavable motif that allows dissociation of the ligand following covalent labeling. Due to their covalent nature of labelling, the probes are especially well suited for imaging of low abundance target proteins. The probes can be used to construct FRET sensors of membrane-bound and intracellular protein targets and subsequently used to investigate ligand-protein interaction in real time by the means of TR-FRET. The novel CB₂R modulators demonstrate a potent response and possess a functional profile spanning full agonism, partial agonism, antagonism, and inverse agonism. These compounds promise to deliver a fine-tuned pharmacological intervention at CB₂R that ameliorates diseases by addressing the underlying disregulation.

In one aspect, the present invention provides a compound of formula (I) described herein, is wherein R¹ is selected from hydroxy and amino, or a pharmaceutically acceptable salt thereof, for use as a medicament.

In one aspect, the present invention provides method of treating or preventing a disease or disorder that is associated with CB₂R in a subject in need thereof, said method comprising administering a therapeutically effective amount of a compound of formula (I) described herein, wherein R¹ is selected from hydroxy and amino, or a pharmaceutically acceptable salt thereof, to said subject in need.

In one aspect, the present invention provides a compound of formula (I) described herein, wherein R¹ is selected from hydroxy and amino, or a pharmaceutically acceptable salt thereof, for use in the treatment or prophylaxis of a disease or disorder that is associated with CB₂R.

In one aspect, the present invention provides the use of a compound of formula (I) described herein, wherein R¹ is selected from hydroxy and amino, or a pharmaceutically acceptable salt thereof, in the treatment or prophylaxis of a disease or disorder that is associated with CB₂R.

In one aspect, the present invention provides the use of a compound of formula (I) described herein, wherein R¹ is selected from hydroxy and amino, or a pharmaceutically acceptable salt thereof, in the manufacture of a medicament for the treatment or prophylaxis of a disease or disorder that is associated with CB₂R.

In a preferred embodiment, said disease or disorder that is associated with CB₂R is selected from hypertension, inflammation, peripheral pain, neuropathic pain, gastrointenstinal disorders, autoimmune diseases, pain, atherosclerosis, age-related macular degeneration, diabetic retinopathy, glaucoma, diabetes mellitus, inflammatory bowel disease, ischemia-reperfusion injury, acute liver failure, liver fibrosis, lung fibrosis, kidney fibrosis, systemic fibrosis, acute allograft rejection, chronic allograft nephropathy, diabetic nephropathy, glomerulonephropathy, cardiomyopathy, heart failure, myocardial ischemia, myocardial infarction, systemic sclerosis, thermal injury, burning, hypertrophic scars, keloids, gingivitis pyrexia, liver cirrhosis, tumors, regulation of bone mass, neurodegeneration, stroke, transient ischemic attack, uveitis, renal fibrosis, arthritis, neuroinflammation, asthma, is osteoporosis, psychiatric diseases, psychosis, cancer, encephalitis, malaria, immunological disorders, rheumatoid arthritis, and allergies.

In one aspect, the present invention provides a compound of formula (I) described herein,

wherein R¹ is a group

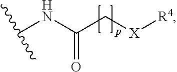

wherein X, p and R⁴ are as described herein, or a pharmaceutically acceptable salt thereof, for use as a fluorescent probe for the cannabinoid receptor 2 (CB₂R).

In a further aspect, the present invention provides the use of a compound of formula (I) described herein, wherein R¹ is a group

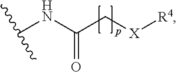

wherein X, p and R⁴ are as described herein, or a pharmaceutically acceptable salt thereof, as a fuorescent or a bioorthogonal probe for the cannabinoid receptor 2 (CB₂R).

In a further aspect, the present invention provides a method of imaging cannabinoid receptor 2 (CB₂R), comprising contacting said cannabinoid receptor 2 (CB₂R) with a compound of formula (I) described herein, wherein R¹ is a group

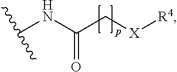

wherein X, p and R⁴ are as described herein, or a pharmaceutically acceptable salt thereof.

### Pharmaceutical Compositions and Administration

In one aspect, the present invention provides a pharmaceutical composition comprising a compound of formula (I) as described herein, or a pharmaceutically acceptable salt thereof, and a therapeutically inert carrier.

In one embodiment, there is provided a pharmaceutical composition according to Example 33 or 34.

The compounds of formula (I) and their pharmaceutically acceptable salts and esters can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts and esters can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

### GENERAL SYNTHETIC METHODS

### Procedure

Unless otherwise noted, all reactions were carried out under nitrogen atmosphere.

### Chemicals

All chemicals and solvents were purchased from commercial suppliers and were used without further purification. DMF, DMSO, THF, Et₂O, MeCN, toluene, benzene and CH₂Cl₂ were dried using 4 Å molecular sieves or using an LC Technology Solutions solvent purification system (SP-1) under an atmosphere of dry nitrogen. Pyridine and i-Pr₂NEt were distilled from KOH under an atmosphere of dry nitrogen. Et₃N was distilled from CaH₂ under an atmosphere of dry nitrogen.

### Chromatography

Analytical thin-layer chromatography (TLC) was performed on Merck silica gel 60 F₂₅₄ TLC glass plates. Purification of reaction products was carried out by flash column chromatography (FCC) using Sigma Aldrich silica 230-400 mesh particle size, 60 Å under 0.3-0.5 bar overpressure.

### Nuclear Magnetic Resonance Spectroscopy

NMR spectra were acquired on Bruker AVIII HD 600 MHz, 500 MHz and 400 MHz spectrometers operating at the denoted spectrometer frequency given in MHz for the specified nucleus. ¹H NMR spectra are reported with the solvent resonance as the reference (CDCl₃ at 7.26 ppm, CD₃OD at 3.31 ppm, CD₂Cl₂ at 5.32 ppm, C₆D₆ at 7.16 ppm, DMSO-d₆ at 2.50 ppm). Peaks are reported as (s=singlet, bs=broad singlet, d=doublet, bt=broad triplet, t=triplet, q=quartet, m=multiplet or unresolved, coupling constant(s) in Hz, integral). ¹³C NMR spectra were recorded with ¹H-decoupling and are reported in ppm with the solvent resonance as the reference (CDCl₃ at 77.16 ppm, CD₃OD at 49.00 ppm, CD₂Cl₂ at 54.00 ppm, C₆D₆ at 128.06 ppm, DMSO-d₆ at 39.52 ppm). Service measurements were performed by the NMR service team of the Laboratorium für Organische Chemie at ETH Zürich.

### High-Resolution Mass Spectrometric

High-resolution mass spectrometric data were obtained at ETH Zürich mass spectrometry service on Bruker Daltonics maXis ESI-QTOF or a Bruker Daltonics maXiS II ESI-QTOF spectrometers and are reported as (m z).

### Infrared Spectroscopy

Infrared (IR) spectra were measured neat on a Perkin-Elmer UATR Two FT-IR Spectrometer and the band maxima are reported in wavenumbers (cm⁻¹).

### Optical Rotation

Optical rotations ([u]_{D}T) were determined using a Jasco P-2000 Polarimeter (10 cm, 1.5 mL cell).

### X-ray Diffraction

The X-ray diffraction was measured on a XtaLAB Synergy R, HyPix-Arc 150 diffractometer.

Semi preparative chiral SFC: The separations were performed using a Prep-SFC-100 semi preparative system with columns (20×250 mm, 5 μm) supplied by Daicel, Chiral Technology and 90 mL/min flow of solvent A (CO₂) and co-solvent B, specified by one of the methods below:
Method 1. Column: Chiralpak IC, B=25% i-PrOH
Method 2. Column: Chiralpak IK, B=18% i-PrOH
Method 3. Column: Chiralcel IK, B=15% MeOH
Method 4. Column: Chiralcel IK, B=16% MeOH
Method 5. Column: Chiralcel OD-H, B=25% (EtOH:n-heptane, 1:1)
Method 6. Column: Chiralcel OD-H, B=20% (EtOH:n-heptane, 1:1)

The absolute stereochemistry at the C(2′), homobenzylic position, α to the gem-dimethyl group was arbitrarily assigned as R for the enantiomer eluting first and S for the enantiomer eluting second for Examples 1-12, when separated by the methods of chiral SFC described above.

The absolute stereochemistry at the C(2′), homobenzylic position, α to the gem-dimethyl group was S for the enantiomer eluting first (A) of the racemate F9-A, when separated by the method of chiral SFC described above, as determined by X-ray crystallographic analysis of its derivative Example 32. Accordingly, Examples 14,15, 17-31, which incorporate F9-A or F9-B, have their C(2′) configuration assigned as S or R, respectively.

The alphanumeric sequencing of the synthetic steps in the following experimental descriptions is based on the alphanumeric sequencing used in Scheme 1 above.

### Example 1

### ((1S,4S,5S)-4-(2,6-dimethoxy-4-((R)-2-methyl-3-(trifluoromethyl)octan-2-yl)phenyl)-6,6-dimethylbicyclo[3.1.1]hept-2-en-2-yl)methanol

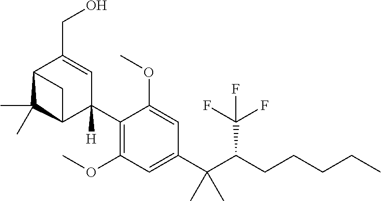 [SMILES: CCCCC[C@@H](C(F)(F)F)C(C)(C)c1cc(OC)c([C@H]2C=C(CO)[C@H]3C[C@@H]2C3(C)C)c(OC)c1]

### Step j) 3-(3,5-dimethoxyphenyl)-1,1,1-trifluoro-3-methylbutan-2-ol

To a solution of 2-(3,5-dimethoxyphenyl)-2-methylpropanal (2.00 g, 9.60 mmol, 1.0 equiv, CAS RN: 120078-30-0) in anhydrous THF (16 mL) at −78° C. was added Me₃SiCF₃ (1.85 mL, 12.5 mmol, 1.3 equiv, CAS RN: 81290-20-2) followed by a solution of TBAF (1.0 M in THF, 960 μL, 960 μmol, 0.1 equiv). The mixture was allowed to warm up to rt and stirred overnight. Additional Me₃SiCF₃ (710 μL, 4.80 mmol, 0.5 equiv) and a solution of TBAF (1.0 M in THF, 960 μL, 960 μmol, 0.1 equiv) were added dropwise and stirred for 1 h to bring the reaction to completion. The mixture was quenched with water (25 mL), extracted with Et₂O (3×25 mL) and the organic extracts concentrated in vacuo. The crude mixture was dissolved in THF (10 mL) and aq. 1 M HCl (10 mL) and refluxed for 1 h. The mixture was cooled down to rt extracted with Et₂O (3×25 mL), combined organic extracts dried with MgSO₄, filtered and concentrated in vacuo. The crude product was purified by flash column chromatography (SiO₂; using 0-30% Et₂O in hexanes) to yield the final product as a colorless liquid (2.50 g, 94%).

¹H NMR (400 MHz, CDCl₃) δ 6.55 (d, J=2.2 Hz, 2H), 6.37 (t, J=2.2 Hz, 1H), 4.06 (td, J=7.5, 5.6 Hz, 1H), 3.80 (s, 6H), 2.28 (d, J=5.6 Hz, 1H), 1.49-1.42 (m, 6H). ¹³C NMR (101 MHz, CDCl₃) δ 160.9, 148.0, 125.4 (q, J=284.4 Hz), 105.4, 98.0, 77.1 (q, J=28.1 Hz), 55.4, 41.2, 24.8 (q, J=2.4 Hz), 23.7 (q, J=2.0 Hz). ¹⁹F NMR (377 MHz, CDCl₃) δ−71.2. IR (neat, vₘₐₓ/cm⁻¹): 3472, 2942, 2841, 1597, 1458, 1425, 1270, 1156. HRMS (ESI): m/z=279.1204 [M+H]⁺ (calc. for C₁₃H₁₈F₃O₃ m/z=279.1203)

### Step k) 3-(3,5-dimethoxyphenyl)-1,1,1-trifluoro-3-methylbutan-2-one

To a solution of 3-(3,5-dimethoxyphenyl)-1,1,1-trifluoro-3-methylbutan-2-ol (2.70 g, 9.70 mmol, 1.0 equiv) in anhydrous CH₂Cl₂(29 mL) at 0° C. was added DMP (5.35 g, 12.6 mmol, 1.3 equiv) in one portion. The mixture was allowed to warm up to rt and stirred for 2 h. The mixture was concentrated in vacuo and the residue triturated with pentane (50 mL). The white solid was filtered off and washed with pentane (2×50 mL). The organic washings were concentrated in vacuo and the crude product was purified by flash column chromatography (SiO₂; using 0-15% Et₂O in hexanes) to yield the final product as a colorless liquid (2.10 g, 78%).

¹H NMR (400 MHz, CDCl₃) δ 6.39 (t, J=2.2 Hz, 1H), 6.34 (d, J=2.2 Hz, 2H), 3.78 (s, 6H), 1.58 (q, J=0.8 Hz, 6H). ¹³C NMR (101 MHz, CDCl₃) δ 193.8 (q, J=31.2 Hz), 161.4, 142.7, 116.2 (q, J=295.1 Hz), 104.3, 99.1, 55.4, 50.2, 24.7 (q, J=1.4 Hz). ¹⁹F NMR (377 MHz, CDCl₃) δ−71.0. IR (neat, vₘₐₓ/cm⁻¹): 2941, 2841, 1745, 1596, 1458, 1426, 1371, 1341, 1299, 1261, 1203. HRMS (ESI): m/z=277.1047 [M+H]⁺ (calc. for C₁₃H₁₆F₃O₃ m/z=277.1046).

### Step 1) 1, 3-dimethoxy-5-(2-methyl-3-(trifluoromethyl)oct-3-en-2-yl)benzene

To a suspension of pentyltriphenylphosphonium bromide (1.92 g, 4.63 mmol, 3.2 equiv) at −78° C. in dry THF (40 mL) n-BuLi (1.6 M in hexane, 2.71 mL, 4.34 mmol, 3.0 equiv) was added dropwise. The solution was stirred for 10 min at −78° C., allowed to warm up to rt. and stirred for additional 30 min. Subsequently, 3-(3,5-dimethoxyphenyl)-1,1,1-trifluoro-3-methylbutan-2-one (400 mg, 1.45 mmol, 1.0 equiv) was dissolved in dry THF (1 mL) and added dropwise to the mixture at 0° C. The mixture was stirred overnight at rt, quenched with aq. sat. NH₄Cl solution (25 mL), diluted with Et₂O (25 mL) and the phases were separated. The aqueous phase was extracted with Et₂O (2×25 mL). Combined organic extracts were dried over MgSO₄, filtered and concentrated in vacuo. The crude product was purined by flash column chromatography (SiO₂; using 0-10% Et₂O in hexanes) to yield separable E-1,3-dimethoxy-5-(2-methyl-3-(trifluoromethyl)oct-3-en-2-yl)benzene (210 mg, 44%) and Z-1,3-dimethoxy-5-(2-methyl-3-(trifluoromethyl)oct-3-en-2-yl)benzene (145 mg, 30%) isomers, respectively, as light yellow liquids.

### E-Isomer

¹H NMR (500 MHz, CDCl₃) δ 6.48 (d, J=2.2 Hz, 2H), 6.30 (t, J=2.2 Hz, 1H), 6.14 (tq,J=7.6, 2.0 Hz, 1H), 3.78 (s, 6H), 1.64-1.57 (m, 2H), 1.51 (q, J=1.2 Hz, 6H), 1.13-0.98 (m, 4H), 0.70 (t, J=7.1 Hz, 3H). ¹³C NMR (126 MHz, CDCl₃) δ 160.8, 153.5, 138.18 (q, J=7.7 Hz), 135.30 (q, J=24.4 Hz), 125.17 (q, J=274.4 Hz), 104.5, 97.2, 55.4, 41.8, 30.5, 29.91 (q, J=1.9 Hz), 28.3, 22.4, 13.9. ¹⁹F NMR (471 MHz, CDCl₃) δ−60.9. IR (neat, vₘₐₓ/cm⁻¹): 2958, 2931, 2874, 2838, 1649, 1596, 1457, 1423, 1290. HRMS (ESI): m/z=331.1882 [M+H]⁺ (calc. for C₁₈H₂₆F₃O₂ m/z=331.1879).

### Z-isomer

¹H NMR (500 MHz, CDCl₃) δ 6.43 (d, J=2.2 Hz, 2H), 6.32 (t, J=2.2 Hz, 1H), 6.04 (t, J=7.7 Hz, 1H), 3.78 (s, 6H), 2.41-2.33 (m, 2H), 1.53-1.48 (m, 2H), 1.47 (s, 6H), 1.45-1.36 (m, 2H), 0.96 (t, J=7.2 Hz, 3H). ¹³C NMR (126 MHz, CDCl₃) δ 160.8, 150.7, 137.4 (q, J=25.2 Hz), 137.0 (q, J=3.5 Hz), 124.9 (q, J=279.4 Hz), 104.1, 97.4, 55.3, 43.1, 31.8, 29.6, 28.5, 22.5, 14.0. ¹⁹F NMR (471 MHz, CDCl₃) δ−53.9. IR (neat, vₘₐₓ/cm⁻¹): 2959, 2933, 2874, 2838, 1652, 1596, 1457, 1424, 1204. HRMS (ESI): m/z=331.1881 [M+H]⁺ (calc. for C₁₈H₂₆F₃O₂ m/z=331.1879).

### Step m) 1,3-dimethoxy-5-(2-methyl-3-(trifluoromethyl)octan-2-yl)benzene

Attention: Pd/C in MeOH can spontaneously combust in contact with air.

Pd (10 wt % on C, 644 mg, 605 μmol, 1.0 equiv) was placed in a flask and the atmosphere was exchanged with argon three times. Subsequently the catalyst was carefully wet with MeOH (8 mL) and a solution of 1,3-dimethoxy-5-(2-methyl-3-(trifluoromethyl)oct-3-en-2-yl)benzene (200 mg, 605 μmol, 1.0 equiv) in MeOH (2.0 mL) was added. The mixture was stirred for 7 days in an autoclave under 7 bar H₂ pressure. The atmosphere was exchanged with argon and the suspension was carefully filtered over a pad of Celite, washed with EtOAc and concentrated in vacuo. Purification by flash column chromatography (SiO₂; 0-5% Et₂O in hexanes) afforded the product as a faint yellow oil (180 mg, 89% yield).

¹H NMR (500 MHz, CDCl₃) δ 6.49 (d, J=2.2 Hz, 2H), 6.34 (t, J=2.2 Hz, 1H), 3.80 (s, 6H), 2.37 (qdd, J=10.3, 7.7, 2.7 Hz, 1H), 1.54-1.43 (m, 1H), 1.41 (d, J=2.0 Hz, 3H), 1.35 (s, 3H), 1.29-1.20 (m, 1H), 1.18-0.97 (m, 6H), 0.78 (t, J=7.2 Hz, 3H). ¹³C NMR (126 MHz, CDCl₃) δ 160.7, 151.5, 129.07 (q, J=283.3 Hz), 105.0, 97.4, 55.4, 52.50 (q, J=22.1 Hz), 40.4, 31.7, 29.4, 28.33 (q, J=2.5 Hz), 26.26 (q, J=2.4 Hz), 23.80 (q, J=1.8 Hz), 22.3, 14.0. ¹⁹F NMR (471 MHz, CDCl₃) δ−62.1. IR (neat, vₘₐₓ/cm¹): 2957, 2873, 1597, 1458, 1206, 1156. HRMS (ESI): m/z=333.2033 [M+H]⁺ (calc. for C₁₈H₂₈F₃O₂ m/z=333.2036).

### Step n) 5-(2-methyl-3-(trifluoromethyl)octan-2-yl)benzene-1,3-diol

To a solution of 1,3-dimethoxy-5-(2-methyl-3-(trifluoromethyl)octan-2-yl)benzene (100 mg, 301 μmol, 1.0 equiv) in anhydrous CH₂Cl₂ (3.0 mL) at 0° C. was added BBr₃ (86 μL, 903 μmol, 3.0 equiv) dropwise and the solution was stirred at 0° C. for 2 h. The mixture was carefully quenched with aq. sat. NaHCO₃ (5 mL) and diluted with Et₂O (10 mL). The layers were separated and the aqueous phase was extracted with Et₂O (2×10 mL). Combined organic extracts were dried over MgSO₄ and concentrated in vacuo. Purification by flash column chromatography (SiO₂; using 0-30% EtOAc in hexanes) afforded the product as a light red oil (91 mg, 99%). The racemate was resolved by chiral SFC Method 2. to afford enantiomers (R)-5-(2-methyl-3-(trifluoromethyl)octan-2-yl)benzene-1,3-diol A (38 mg, 42%, % ee>99%) and (S)-5-(2-methyl-3-(trifluoromethyl)octan-2-yl)benzene-1,3-diol B (35 mg, 38%, % ee=98%).

¹H NMR (400 MHz, CDCl₃) δ 6.42 (d, J=2.2 Hz, 2H), 6.23 (t, J=2.2 Hz, 1H), 5.51 (s, 2H), 2.30 (qdd, J=10.2, 7.5, 2.6 Hz, 1H), 1.51-0.96 (m, 8H), 1.34 (q, J=1.9 Hz, 3H), 1.28 (s, 3H), 0.79 (t, J=7.1 Hz, 3H). ¹³C NMR (101 MHz, CDCl₃) δ 156.4, 152.6, 128.93 (q, J=283.4 Hz), 106.3, 100.8, 52.52 (q, J=22.1 Hz), 40.1, 31.7, 29.4, 27.6, 26.20 (q, J=2.4 Hz), 24.3, 22.3, 14.0. ¹⁹F NMR (376 MHz, CDCl₃) δ−62.1. IR (neat, vₘₐₓ/cm⁻¹): 3349, 2957, 2928, 2872, 1598, 1459, 1256, 1148. HRMS (ESI): m/z=305.1721 [M+H]⁺ (calc. for C₁₆H₂₄F₃O₂ m/z=305.1723). (R)-5-(2-methyl-3-(trifluoromethyl)octan-2-yl)benzene-1,3-diol A [α]²′D=+17.570±0.110 (c=1.0, CHCl₃). (S)-5-(2-methyl-3-(trifluoromethyl)octan-2-yl)benzene-1,3-diol B [α]²⁵=−16.414 k 0.153 (c=1.0, CHCl₃).

### Step o) ((1S,4S,5S)-4-(2,6-dihydroxy-4-((R)-2-methyl-3-(trifluoromethyl)octan-2-yl)phenyl)-6,6-dimethylbicyclo[3.1.1]hept-2-en-2-yl)methyl pivalate

To a solution of (R)-5-(2-methyl-3-(trifluoromethyl)octan-2-yl)benzene-1,3-diol A (17.9 mg, 58.8 μmol, 1.0 equiv) and ((1S,4R,5R)-4-hydroxy-6,6-dimethylbicyclo[3.1.1]hept-2-en-2-yl)methyl pivalate (16.3 mg, 64.7 μmol, 1.1 equiv, pn=CA3171726A1) in CH₂Cl₂ (2.3 mL) was added pTsOH·H₂O (3.1 mg. 16.4 μmol, 0.28 equiv) and the solution was stirred for 30 min. The reaction was stopped by addition on sat. aq. NaHCO₃ (4 mL) and diluted with Et₂O (5 mL). The layers were separated and the aqueous phase was extracted with Et₂O (3×5 mL). Combined organic extracts were dried over MgSO₄, filtered and concentrated in vacuo. Purification by flash column chromatography (SiO₂; using 0-15% EtOAc in hexanes) afforded the product as a colourless foam (23.3 mg, 74%).

¹H NMR (500 MHz, CDCl₃) δ 6.34 (s, 2H), 5.99 (dt, J=3.0, 1.5 Hz, 1H), 5.68 (bs, 2H), 4.64 (ddd, J=13.5, 2.1, 1.4 Hz, 1H), 4.51 (ddd, J=13.6, 2.4, 1.6 Hz, 1H), 4.04-3.97 (m, 1H), 2.36 (dt, J=9.7, 5.6 Hz, 1H), 2.33-2.22 (m, 3H), 1.47 (d, J=9.7 Hz, 1H), 1.45-1.38 (m, 1H), 1.35 (s, 3H), 1.34 (s, 3H), 1.32-1.20 (m, 2H), 1.28 (s, 3H), 1.23 (s, 9H), 1.14-1.00 (m, 5H), 0.98 (s, 3H), 0.76 (t, J=7.1 Hz, 3H). ¹³C NMR (126 MHz, CDCl₃) δ 178.7, 155.2, 149.8, 149.3, 129.1 (q, J=283.3 Hz), 120.1, 112.5, 106.7, 66.6, 52.4 (q, J=22.1 Hz), 47.5, 44.2, 41.1, 39.8, 39.1, 37.9, 31.6, 29.4, 28.1, 28.1, 27.4, 26.1, 26.0, 23.6, 22.2, 20.9, 14.0. ¹⁹F NMR (471 MHz, CDCl₃) δ−62.2. IR (neat, vₘₐₓ/cm⁻¹): 3445, 2942, 2872, 1707, 1626, 1578, 1481, 1254, 1149. HRMS (ESI): m/z=561.3147 [M+Na]⁺ (calc. for C₃₁H₄₅F₃NaO₄ m/z=561.3162). [α]²⁵D=+62.961±0.119 (c=1.0, CHCl₃).

### Step p) ((1S,4S,5S)-4-(2,6-dimethoxy-4-((R)-2-methyl-3-(trifluoromethyl)octan-2-yl)phenyl)-6,6-dimethylbicyclo[3.1.1]hept-2-en-2-yl)methyl pivalate

To a solution of ((1S,4S,5S)-4-(2,6-dihydroxy-4-((R)-2-methyl-3-(trifluoromethyl)octan-2-yl)phenyl)-6,6-dimethylbicyclo[3.1.1]hept-2-en-2-yl)methyl pivalate (23.3 mg, 43.2 mol, 1.0 equiv) in acetone (0.5 mL) was added (MeO)₂SO₂ (20.5 μL. 0.216 mmol, 5.0 equiv) and K₂CO₃ (35.9 mg. 0.260 mmol, 6.0 equiv) and the light purple suspension was stirred overnight at rt. The reaction mixture was filtered and concentrated in vacuo. Purification by flash column chromatography (SiO₂; using 0-10% Et₂O in hexanes) afforded the product as a light red wax (23.1 mg, 94%).

¹H NMR (500 MHz, CDCl₃) δ 6.47 (s, 2H), 5.76 (dt, J=2.9, 1.5 Hz, 1H), 4.61-4.54 (m, 1H), 4.51 (ddd, J=12.2, 1.7, 1.2 Hz, 1H), 4.04-3.97 (m, 1H), 3.74 (s, 6H), 2.31 (dddd, J=20.6, 10.3, 7.4, 2.5 Hz, 1H), 2.20-2.10 (m, 2H), 2.02 (tt, J=5.8, 1.9 Hz, 1H), 1.73-1.66 (m, 1H), 1.50-1.41 (m, 1H), 1.43 (s, 3H), 1.36 (s, 3H), 1.34-1.18 (m, 2H), 1.29 (s, 3H), 1.21 (s, 9H), 1.12-0.88 (m, 5H), 0.97 (s, 3H), 0.74 (t, J=7.1 Hz, 3H). ¹³C NMR (126 MHz, CDCl₃) δ 178.6, 158.6, 148.1, 137.5, 129.1 (q, J=283.3 Hz), 126.1, 118.7, 103.1, 67.6, 56.0, 52.7 (q, J=22.1 Hz), 47.6, 44.0, 41.1, 40.5, 39.0, 37.7, 31.5, 30.5, 29.4, 28.8, 27.6, 27.4, 26.4, 26.2, 22.2, 21.2, 14.0. ¹⁹F NMR (471 MHz, CDCl₃) δ−62.1. IR (neat, vₘₐₓ/cm⁻¹): 2957, 2932, 2870, 1727, 1605, 1575, 1461, 1412, 1150. HRMS (ESI): m/z=589.3470 [M+Na]⁺ (calc. for C₃₃H₄₉F₃NaO₄ m/z=589.3475). [α]²sD=+73.052±0.280 (c=1.0, CHCl₃).

### Step q) ((1S,4S,5S)-4-(2,6-dimethoxy-4-((R)-2-methyl-3-(trifluoromethyl)octan-2-yl)phenyl)-6,6-dimethylbicyclo[3.1.1]hept-2-en-2-yl)methanol

Solution of ((1S,4S,5S)-4-(2,6-dimethoxy-4-((R)-2-methyl-3-(trifluoromethyl)octan-2-yl)phenyl)-6,6-dimethylbicyclo[3.1.1]hept-2-en-2-yl)methyl pivalate (26.1 mg, 46.0 mol, 1.0 equiv) in CH₂Cl₂ (0.4 mL) was cooled to 0° C. and DIBAL (1.0 M in hexanes, 97 μL. 96.7 μmol, 2.1 equiv) was added dropwise. The mixture was stirred at 0° C. for 15 min. and subsequently quenched with sat. aq. Rochelle's salt (4 mL) and diluted with Et₂O (5 mL). The mixture was vigorously stirred at rt until the phases cleanly separated. The phases were separated and the aqueous phase extracted with Et₂O (2×5 mL). Combined organic fractions were dried over MgSO₄, filtered and concentrated in vacuo. Purification by flash column chromatography (SiO₂; using 5-20% EtOAc in hexanes) afforded the product as a clear oil (18.3 mg, 82%).

¹H NMR (500 MHz, CDCl₃) δ 6.47 (s, 2H), 5.69 (dt, J=2.8, 1.4 Hz, 1H), 4.07 (ddd, J=1.9, 1.3, 0.6 Hz, 2H), 4.03-3.98 (m, 1H), 3.75 (s, 6H), 2.31 (qdd, J=10.3, 7.4, 2.6 Hz, 1H), 2.25-2.16 (m, 2H), 2.04 (tt, J=5.8, 1.9 Hz, 1H), 1.69 (d, J=8.3 Hz, 1H), 1.50-1.41 (m, 1H), 1.43 (s, 3H), 1.36 (s, 3H), 1.34-1.16 (m, 2H), 1.31 (s, 3H), 1.12-0.89 (m, 5H), 0.97 (s, 3H), 0.74 (t, J=7.1 Hz, 3H). ¹³C NMR (126 MHz, CDCl₃) δ 158.6, 148.1, 142.2, 129.1 (q, J=283.4 Hz), 123.6, 118.8, 103.2, 66.8, 56.1, 52.7 (q, J=22.2 Hz), 47.6, 44.0, 41.0, 40.5, 37.6, 31.6, 29.4, 28.8, 28.0, 26.4, 26.3, 23.3, 22.2, 21.2, 14.0. ¹⁹F NMR (471 MHz, CDCl₃) δ−62.1. IR (neat, vₘₐₓ/cm⁻¹): 3373, 2929, 2866, 1605, 1574, 1460, 1412, 1239, 1119.

HRMS (ESI): m/z=505.2903 [M+Na]⁺ (calc. for C₂₈H₄₁F₃NaO₃ m/z=505.2900). [α]²s_=+97.242±0.182 (c=1.0, CHCl₃).

### Example 2

### ((1S,4S,5S)-4-(2,6-dimethoxy-4-((S)-2-methyl-3-(trifluoromethyl)octan-2-yl)phenyl)-6,6-dimethylbicyclo[3.1.1]hept-2-en-2-yl)methanol

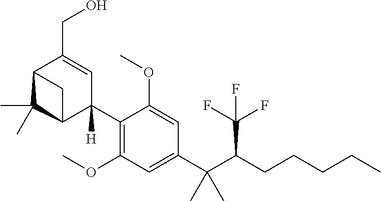 [SMILES: CCCCC[C@H](C(F)(F)F)C(C)(C)c1cc(OC)c([C@H]2C=C(CO)[C@H]3C[C@@H]2C3(C)C)c(OC)c1]

### Step o) ((1S,4S,5S)-4-(2,6-dihydroxy-4-((S)-2-methyl-3-(trifluoromethyl)octan-2-yl)phenyl)-6,6-dimethylbicyclo[3.1.1]hept-2-en-2-yl)methyl pivalate

To a solution of (S)-5-(2-methyl-3-(trifluoromethyl)octan-2-yl)benzene-1,3-diol B (16.6 mg, 54.5 μmol, 1.0 equiv) and ((1S,4R,5R)-4-hydroxy-6,6-dimethylbicyclo[3.1.1]hept-2-en-2-yl)methyl pivalate (15.1 mg, 60.0 μmol, 1.1 equiv) in CH₂Cl₂(2.3 mL) was added pTsOH·H₂O (2.9 mg. 15.2 μmol, 0.28 equiv) and the solution was stirred for 30 min. The reaction was stopped by addition on sat. aq. NaHCO₃ (4 mL) and diluted with Et₂O (5 mL). The layers were separated and the aqueous phase was extracted with Et₂O (3×5 mL). Combined organic extracts were dried over MgSO₄, filtered and concentrated in vacuo. Purification by flash column chromatography (SiO₂; using 0-15% EtOAc in hexanes) afforded the product as a faint yellow foam (23.0 mg, 78%).

¹H NMR (500 MHz, CDCl₃) δ 6.34 (s, 2H), 6.00 (dt, J=3.1, 1.6 Hz, 1H), 5.76 (bs, 2H), 4.64 (ddd, J=13.5, 2.0, 1.4 Hz, 1H), 4.51 (ddd, J=13.5, 2.3, 1.5 Hz, 1H), 4.02-3.98 (m, 1H), 2.36 (dt, J=9.7, 5.6 Hz, 1H), 2.33-2.22 (m, 3H), 1.46 (d, J=9.7 Hz, 1H), 1.45-1.38 (m, 1H), 1.35 (s, 3H), 1.34 (s, 3H), 1.33-1.19 (m, 2H), 1.28 (s, 3H), 1.23 (s, 9H), 1.16-0.96 (m, 5H), 0.98 (s, 3H), 0.76 (t, J=7.1 Hz, 3H). ¹³C NMR (126 MHz, CDCl₃) δ 178.7, 155.2, 149.8, 149.3, 129.1 (q, J=283.5 Hz), 120.1, 112.4, 106.7, 66.6, 52.5 (q, J=22.2 Hz), 47.5, 44.2, 41.1, 39.8, 39.1, 37.9, 31.6, 29.4, 28.1, 28.1, 27.4, 26.1, 26.0, 23.6, 22.2, 20.9, 14.0. ¹⁹F NMR (471 MHz, CDCl₃) δ−62.2. IR (neat, vₘₐₓ/cm⁻¹): 3446, 2942, 2872, 1707, 1626, 1578, 1481, 1254, 1149. HRMS (ESI): m/z=561.3154 [M+Na]⁺ (calc. for C₃₁H₄₅F₃NaO₄ m/z=561.3162). [α]²⁵D=+38.128±0.330 (c=1.0, CHCl₃).

### Step p) ((1S,4S,5S)-4-(2,6-dimethoxy-4-((S)-2-methyl-3-(trifluoromethyl)octan-2-yl)phenyl)-6,6-dimethylbicyclo[3.1.1]hept-2-en-2-yl)methyl pivalate

To a solution of ((1S,4S,5S)-4-(2,6-dihydroxy-4-((S)-2-methyl-3-(trifluoromethyl)octan-2-yl)phenyl)-6,6-dimethylbicyclo[3.1.1]hept-2-en-2-yl)methyl pivalate (23.0 mg, 42.7 mol, 1.0 equiv) in acetone (0.5 mL) was added (MeO)₂SO₂ (20.2 μL. 0.214 mmol, 5.0 equiv) and K₂CO₃ (35.4 mg. 0.256 mmol, 6.0 equiv) and the light purple suspension was stirred overnight at rt. The reaction mixture was filtered and concentrated in vacuo. Purification by flash column chromatography (SiO₂; using 0-10% Et₂O in hexanes) afforded the product as a light red wax (23.5 mg, 97%).

¹H NMR (500 MHz, CDCl₃) δ 6.47 (s, 2H), 5.88-5.64 (m, 1H), 4.61-4.55 (m, 1H), 4.51 (ddd, J=12.2, 1.7, 1.3 Hz, 1H), 4.02-3.95 (m, 1H), 3.74 (s, 6H), 2.31 (dddd, J=20.4, 10.1, 7.2, 2.5 Hz, 1H), 2.19-2.11 (m, 2H), 2.06-1.96 (m, 1H), 1.73-1.64 (m, 1H), 1.49-1.41 (m, 1H), 1.42 (s, 3H), 1.36 (s, 3H), 1.35-1.19 (m, 2H), 1.29 (s, 3H), 1.21 (s, 9H), 1.12-0.86 (m, 5H), 0.97 (s, 3H), 0.74 (t, J=7.1 Hz, 3H). ¹³C NMR (126 MHz, CDCl₃) δ 178.6, 158.6, 148.1, 137.5, 129.1 (q, J=283.3 Hz), 126.2, 118.7, 103.1, 67.6, 56.0, 52.7 (q, J=22.1 Hz), 47.5, 43.9, 41.0, 40.5, 39.0, 37.7, 30.5, 29.4, 28.8, 27.6, 27.4, 26.4, 26.2, 23.3, 22.1, 21.2, 14.0. ¹⁹F NMR (471 MHz, CDCl₃) δ−62.1. IR (neat, vₘₐₓ/cm⁻¹): 2956, 2932, 2870, 1727, 1605, 1575, 1461, 1412, 1149. HRMS (ESI): m/z=589.3460 [M+Na]⁺ (calc. for C₃₃H₄₉F₃NaO₄ m/z=589.3475). [α]²⁵D=+58.259±0.119 (c=1.0, CHCl₃).

### Step q) ((1S,4S,5S)-4-(2,6-dimethoxy-4-((S)-2-methyl-3-(trifluoromethyl)octan-2-yl)phenyl)-6,6-dimethylbicyclo[3.1.1]hept-2-en-2-yl)methanol

Solution of ((1S,4S,5S)-4-(2,6-dimethoxy-4-((S)-2-methyl-3-(trifluoromethyl)octan-2-yl)phenyl)-6,6-dimethylbicyclo[3.1.1]hept-2-en-2-yl)methyl pivalate (24.0 mg, 42.3 mol, 1.0 equiv) in CH₂Cl₂ (0.4 mL) was cooled to 0° C. and DIBAL (1.0 M in hexanes, 89 μL. 88.9 μmol, 2.1 equiv) was added dropwise. The mixture was stirred at 0° C. for 15 min. and subsequently quenched with sat. aq. Rochelle's salt (4 mL) and diluted with Et₂O (5 mL). The mixture was vigorously stirred at rt. until the phases cleanly separated. The phases were separated and the aqueous phase extracted with Et₂O (2×5 mL). Combined organic fractions were dried over MgSO₄, filtered and concentrated in vacuo. Purification by flash column chromatography (SiO₂; using 5-20% EtOAc in hexanes) afforded the product as a clear oil (16.6 mg, 81%).

¹H NMR (400 MHz, CDCl₃) δ 6.47 (s, 2H), 5.69 (dt, J=2.8, 1.4 Hz, 1H), 4.11-4.04 (m, 2H), 4.04-3.96 (m, 1H), 3.75 (s, 6H), 2.32 (qdd, J=10.2, 7.4, 2.5 Hz, 1H), 2.25-2.16 (m, 2H), 2.04 (tt, J=5.9, 1.9 Hz, 1H), 1.68 (d, J=8.2 Hz, 1H), 1.51-1.45 (m, 1H), 1.43 (s, 3H), 1.36 (s, 3H), 1.34-1.16 (m, 2H), 1.31 (s, 3H), 1.12-0.87 (m, 5H), 0.97 (s, 3H), 0.74 (t, J=7.0 Hz, 3H). ¹³C NMR (101 MHz, CDCl₃) δ 158.7, 148.1, 142.2, 129.1 (d, J=283.3 Hz), 123.6, 118.8, 103.2, 66.8, 56.0, 52.7 (q, J=22.0 Hz), 47.6, 44.0, 41.0, 40.5, 37.6, 31.6, 29.4, 28.8, 28.0, 26.4, 26.2, 23.3, 22.2, 21.2, 14.0. ¹⁹F NMR (376 MHz, CDCl₃) δ−62.1. IR (neat, vₘₐₓ/cm⁻¹): 3371, 2929, 2866, 1605, 1574, 1459, 1412, 1239, 1119. HRMS (ESI): m/z=505.2902 [M+Na]⁺ (calc. for C₂₈H₄₁F₃NaO₃ m/z=505.2900). [α]²⁵D=+78.198±0.132 (c=1.0, CHCl₃).

### Example 3

((1S,4S,5S)-4-(4-(3-ethyl-2-methyloctan-2-yl)-2,6-dimethoxyphenyl)-6,6-dimethylbicyclo[3.1.1]hept-2-en-2-yl)methanol

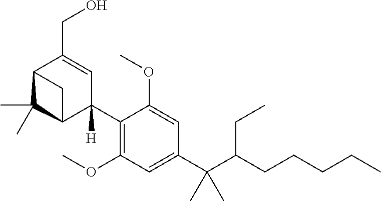 [SMILES: CCCCCC(CC)C(C)(C)c1cc(OC)c([C@H]2C=C(CO)[C@H]3C[C@@H]2C3(C)C)c(OC)c1]

### Step a) 2-(3,5-dimethoxyphenyl)-2-methyloctan-3-one

Solution of 1-iodopentane (3.82 mL, 29.2 mmol, 1.20 equiv) in 3:2 pentane-Et₂O (300 mL) was cooled to −78° C. under argon atmosphere and t-BuLi (1.7 M in pentane, 38.0 mL, 64.6 mmol, 2.65 equiv) was added dropwise. The solution was stirred at −78° C. for 5 min following addition, and then the mixture was allowed to reach rt. and was left standing for 1 h. The reaction mixture was cooled back to −78° C., and a solution of 2-(3,5-dimethoxyphenyl)-2-methylpropanenitrile (5.00 g, 24.4 mmol, 1.00 equiv, CAS RN: 22972-63-0) in pentane (10 mL) was added in one portion. The mixture was stirred for 5 min. at −78° C., allowed to warm up to rt. and stirred for 1.5 h. The reaction was quenched by addition of aq. HCl (0.5 M, 50 mL). The organic solvents were removed in vacuo, THF (150 mL) and aq. HCl (0.5 M, 100 mL) were added and the mixture stirred vigorously overnight at rt. The mixture was diluted with EtOAc (150 mL), layers were separated and the aqueous phase was extracted with EtOAc (2×150 mL). Combined organic extracts were washed with 5% aq. Na₂S₂O₃ solution (150 mL), followed by brine, dried over MgSO₄ and concentrated in vacuo. Purification by flash column chromatography (SiO₂; using 0-15% Et₂O in hexanes) afforded the product as a light-yellow liquid (6.10 g, 90%).

¹H NMR (400 MHz, CDCl₃) δ 6.36 (d, J=2.2 Hz, 2H), 6.33 (t, J=2.2 Hz, 1H), 3.75 (s, 6H), 2.20 (t, J=7.4 Hz, 2H), 1.50-1.43 (m, 2H), 1.42 (s, 6H), 1.23-1.02 (m, 4H), 0.79 (t, J=7.2 Hz, 3H). ¹³C NMR (101 MHz, CDCl₃) δ 213.0, 161.1, 146.7, 104.6, 98.3, 55.3, 52.4, 37.2, 31.4, 25.1, 24.1, 22.5, 13.9. IR (neat, vₘₐₓ/cm⁻¹): 2956, 2932, 2872, 1708, 1594, 1456, 1423, 1205, 1155. HRMS (ESI): m/z=279.1955 [M+H]⁺ (calc. for C₁₇H₂₇O₃ m/z=279.1955).

### Step b) 2-(3,5-dimethoxyphenyl)-3-ethyl-2-methyloctan-3-ol

To a solution of LaCl₃ 2LiCl (0.6 M in THF, 5.57 mL, 3.34 mmol, 1.0 equiv) was added 2-(3,5-dimethoxyphenyl)-2-methyloctan-3-one (930 mg, 3.34 mmol, 1.0 equiv) in dry THF (3.0 mL) and the resulting mixture was stirred at rt for 1 h. The reaction mixture was cooled to 0° C. and a solution of EtLi (0.3 M in benzene:cyclohexane, 12.2 mL, 3.68 mmol, 1.1 equiv) was added dropwise and the mixture was allowed to stir at the same temperature for 1.5 h. The mixture was quenched with sat. NH₄Cl solution (10 mL) and H₂O (10 mL) and diluted with Et₂O (10 mL). The layers were separated and the aqueous phase was extracted with Et₂O (3×10 mL). The combined organic extracts were dried with MgSO₄, filtered and concentrated in vacuo. The crude material was purified by flash column chromatography (SiO₂; 0-15% EtOAc in hexanes) to afford the product as a colourless oil (932 mg, 99%).

¹H NMR (400 MHz, CDCl₃) δ 6.62 (d, J=2.3 Hz, 2H), 6.34 (t, J=2.2 Hz, 1H), 3.76 (s, 6H), 1.60-1.41 (m, 4H), 1.38 (s, 6H), 1.30 (s, 1H), 1.29-1.15 (m, 6H), 0.85 (t, J=6.8 Hz, 3H), 0.81 (t, J=7.4 Hz, 3H). ¹³C NMR (101 MHz, CDCl₃) δ 160.1, 149.7, 107.0, 97.3, 77.0, 55.1, 46.7, 34.9, 33.0, 27.8, 24.8, 24.8, 24.5, 22.6, 14.1, 9.2. IR (neat, vₘax/cm¹): 3568, 2954, 2872, 1594, 1456, 1421, 1204, 1154, 1021. HRMS (ESI): m/z=331.2244 [M+Na]⁺ (calc. for C₁₉H₃₂NaO₃ m/z=331.2244).

### Steps d and e) 1-(3-ethyl-2-methyloctan-2-yl)-3,5-dimethoxybenzene

To a solution of 2-(3,5-dimethoxyphenyl)-3-ethyl-2-methyloctan-3-ol (932 mg, 3.02 mmol, 1.0 equiv) in dry pyridine (12.0 mL) was added DMAP (73.8 mg, 604 μmol, 0.2 equiv). The mixture was cooled to 0° C., SOCl₂ (661 μL, 9.07 mmol, 3.0 equiv) was added dropwise and the solution was allowed to warm up to rt. and stirred for 30 min. The reaction was quenched by addition of aq. 1 M HCl (30 mL) and diluted with EtOAc (50 mL). The phases were separated and the aqueous phase was extracted with EtOAc (50 mL). Combined organic phases were washed with 0.5 M HCl (2×20 mL), dried over MgSO₄, filtered and concentrated in vacuo. The crude material was dissolved in dry EtOAc (6.0 mL), Pd (10 wt % on C, 161 mg, 151 μmol, 0.05 equiv) was added and the resulting mixture was stirred in an autoclave at rt for 16 h under 55 bar H₂ pressure. Then the reaction mixture was filtered through Celite and the crude material was purified by flash column chromatography (SiO₂; 0-10% EtOAc in hexanes) to afford the product as a colourless oil (830 mg, 94%).

¹H NMR (400 MHz, CDCl₃) δ 6.54 (d, J=2.2 Hz, 2H), 6.32 (t, J=2.2 Hz, 1H), 3.81 (s, 6H), 1.47-1.00 (m, 11H), 1.23 (s, 6H), 0.86 (t, J=7.2, 3H), 0.86 (t, J=6.2 Hz, 3H). ¹³C NMR (101 MHz, CDCl₃) δ 160.4, 153.7, 105.2, 96.6, 55.3, 50.9, 42.1, 32.5, 31.5, 30.0, 25.9, 25.3, 24.6, 22.7, 14.6, 14.2. IR (neat, vₘₐₓ/cm⁻¹): 2957, 2932, 2872, 1595, 1457, 1421, 1205, 1155, 1055. HRMS (ESI): m/z=315.2294 [M+Na]⁺ (calc. for C₁₉H₃₂NaO₂m/z=315.2295).

### Step f) 5-(3-ethyl-2-methyloctan-2-yl)benzene-1,3-diol

To a solution of 1-(3-ethyl-2-methyloctan-2-yl)-3,5-dimethoxybenzene (890 mg, 3.04 mmol, 1.0 equiv) in anhydrous CH₂Cl₂ (30 mL) at 0° C. was added BBr₃ (866 μL, 9.13 mmol, 3.0 equiv) dropwise and the green solution was stirred at 0° C. for 3 h. The mixture was diluted with CH₂Cl₂ (30 mL) and carefully quenched with aq. sat. NaHCO₃ (30 mL). The layers were separated and the aqueous phase was extracted with CH₂Cl₂ (2×50 mL). Combined organic extracts were dried over MgSO₄ and concentrated in vacuo. Purification by flash column chromatography (SiO₂; using 0-40% EtOAc in hexanes) afforded the product as a golden oil (777 mg, 97%).

¹H NMR (400 MHz, CDCl₃) δ 6.43 (d, J=2.2 Hz, 2H), 6.19 (t, J=2.2 Hz, 1H), 5.38 (bs, 2H), 1.37-1.10 (m, 9H), 1.15 (s, 3H), 1.14 (s, 3H), 1.07-0.95 (m, 2H), 0.84 (t, J=7.1 Hz, 3H), 0.80 (t, J=7.3 Hz, 3H). ¹³C NMR (101 MHz, CDCl₃) δ 156.1, 154.8, 106.5, 100.0, 50.8, 41.9, 32.6, 31.4, 30.0, 25.9, 25.1, 24.6, 22.7, 14.5, 14.2. IR (neat, vₘₐₓ/cm⁻¹): 3322, 2959, 2931, 2873, 1596, 1465, 1325, 1150, 991. HRMS (ESI): m/z=265.2164 [M+H]+(calc. for C₁₇H₂₉O₂m/z=264.2162).

### Step o) ((1S,4S,5S)-4-(4-(3-ethyl-2-methyloctan-2-yl)-2,6-dihydroxyphenyl)-6,6-dimethylbicyclo[3.1.1]hept-2-en-2-yl)methyl pivalate

To a solution of 5-(3-ethyl-2-methyloctan-2-yl)benzene-1,3-diol (52.0 mg, 197 mol, 1.0 equiv) and ((1S,4R,5R)-4-hydroxy-6,6-dimethylbicyclo[3.1.1]hept-2-en-2-yl)methyl pivalate (54.6 mg, 216 μmol, 1.1 equiv) in CH₂Cl₂ (6.7 mL) was added pTsOH·H₂O (10.5 mg. 55.0 μmol, 0.28 equiv) and the solution was stirred for 30 min. The reaction was stopped by addition on sat. aq. NaHCO₃ (5 mL) and diluted with Et₂O (10 mL). The layers were separated and the aqueous phase was extracted with Et₂O (3×10 mL). Combined organic extracts were dried over MgSO₄, filtered and concentrated in vacuo. Purification by flash column chromatography (SiO₂; using 0-20% EtOAc in hexanes) afforded the product as a colourless foam (72.0 mg, 73%).

¹H NMR (400 MHz, CDCl₃) δ 6.35 (s, 2H), 6.01 (dt, J=3.1, 1.7 Hz, 1H), 5.63 (bs, 2H), 4.63 (ddd, J=13.5, 2.0, 1.2 Hz, 1H), 4.51 (ddd, J=13.5, 2.4, 1.6 Hz, 1H), 4.07-3.94 (m, 1H), 2.40-2.33 (m, 1H), 2.32-2.27 (m, 2H), 1.49 (d, J=9.6 Hz, 1H), 1.42-0.79 (m, 11H), 1.34 (s, 3H), 1.23 (s, 9H), 1.15 (s, 3H), 1.14 (s, 3H), 0.98 (s, 3H), 0.83 (t, J=6.8 Hz, 3H), 0.82 (t, J=7.3 Hz, 3H). ¹³C NMR (101 MHz, CDCl₃) δ 178.6, 154.9, 151.6, 149.5, 120.5, 111.5, 107.0, 66.6, 50.8, 47.5, 44.3, 41.5, 41.1, 39.1, 37.9, 32.5, 31.3, 30.0, 28.2, 27.4, 26.0, 25.7, 25.2, 24.6, 22.7, 20.9, 14.5, 14.2. IR (neat, vₘₐₓ/cm⁻¹): 3453, 2958, 2931, 2872, 1730, 1708, 1625, 1575, 1480, 1157. HRMS (ESI): m/z=521.3596 [M+Na]⁺ (calc. for C₃₂H₅₀NaO₄ m/z=521.3601). [α]²⁵D=+53.699±0.155 (c=1.0, CHCl₃).

### Step p) ((1S,4S,5S)-4-(4-(3-ethyl-2-methyloctan-2-yl)-2,6-dimethoxyphenyl)-6,6-dimethylbicyclo[3.1.1]hept-2-en-2-yl)methyl pivalate

To a solution of ((1S,4S,5S)-4-(4-(3-ethyl-2-methyloctan-2-yl)-2,6-dihydroxyphenyl)-6,6-dimethylbicyclo[3.1.1]hept-2-en-2-yl)methyl pivalate (43.0 mg, 86.2 mol, 1.0 equiv) in acetone (0.9 mL) was added (MeO)₂SO₂ (40.9 μL. 431 μmol, 5.0 equiv) and K₂CO₃(71.5 mg. 517 μmol, 6.0 equiv) and the light purple suspension was stirred overnight at rt. The reaction mixture was filtered and concentrated in vacuo. Purification by flash column chromatography (SiO₂; using 0-5% Et₂O in hexanes) afforded the product as a light red wax (40.0 mg, 88%).

¹H NMR (500 MHz, CDCl₃) δ 6.49 (s, 2H), 5.78 (dt, J=2.8, 1.4 Hz, 1H), 4.60-4.56 (m, 1H), 4.52-4.49 (m, 1H), 4.02-3.98 (m, 1H), 3.73 (s, 6H), 2.22-2.11 (m, 2H), 2.11-1.99 (m, 1H), 1.77-1.66 (m, 1H), 1.44-1.00 (m, 11H), 1.29 (s, 3H), 1.22 (s, 3H), 1.21 (s, 9H), 1.21 (s, 3H), 0.97 (s, 3H), 0.84 (t, J=7.3 Hz, 3H), 0.83 (t, J=7.1 Hz, 3H). ¹³C NMR (126 MHz, CDCl₃) δ 178.6, 158.4, 150.4, 137.3, 126.6, 117.7, 103.5, 67.7, 56.0, 51.1, 47.5, 44.0, 42.1, 41.0, 39.0, 37.7, 32.5, 31.5, 30.0, 27.7, 27.5, 26.5, 25.7, 25.7, 24.7, 22.7, 21.2, 14.6, 14.2. IR (neat, vₘₐₓ/cm⁻¹): 2957, 2930, 2871, 1728, 1605, 1573, 1461, 1410, 1151. HRMS (ESI): m/z=549.3917 [M+Na]⁺ (calc. for C₃₄H₅₄NaO₄m/z=549.3914). [α]²⁵D=+67.051 0.163 (c=1.0, CHCl₃).

### Step q) ((—1S,4S,5S)-4-(4-(3-ethyl-2-methyloctan-2-yl)-2,6-dimethoxyphenyl)-6,6-dimethylbicyclo[3.1.1]hept-2-en-2-yl)methanol

Solution of ((1S,4S,5S)-4-(4-(3-ethyl-2-methyloctan-2-yl)-2,6-dimethoxyphenyl)-6,6-dimethylbicyclo[3.1.1]hept-2-en-2-yl)methyl pivalate (20.0 mg, 37.9 mol, 1.0 equiv) in CH₂Cl₂ (0.3 mL) was cooled to 0° C. and DIBAL (1.0 M in hexanes, 80 μL. 79.7 μmol, 2.1 equiv) was added dropwise. The mixture was stirred at 0° C. for 15 min. and subsequently quenched with sat. aq. Rochelle's salt (4 mL) and diluted with Et₂O (5 mL). The mixture was vigorously stirred at rt. until the phases cleanly separated. The phases were separated and the aqueous phase extracted with Et₂O (3×5 mL). Combined organic fractions were dried over MgSO₄, filtered and concentrated in vacuo. Purification by flash column chromatography (SiO₂; using 5-20% EtOAc in hexanes) afforded the product as a clear wax (15.0 mg, 89%).

¹H NMR (500 MHz, CDCl₃) δ 6.50 (s, 2H), 5.71 (dt, J=2.8, 1.4 Hz, 1H), 4.08-4.06 (m, 2H), 4.01-3.99 (m, 1H), 3.74 (s, 6H), 2.25-2.17 (m, 2H), 2.06 (tt, J=5.8, 1.8 Hz, 1H), 1.71 (d, J=8.1 Hz, 1H), 1.45-0.98 (m, 11H), 1.31 (s, 3H), 1.22 (s, 3H), 1.21 (s, 3H), 0.97 (s, 3H), 0.84 (t, J=7.4 Hz, 3H), 0.83 (t, J=7.1 Hz, 3H). ¹³C NMR (126 MHz, CDCl₃) δ 158.4, 150.4, 142.0, 124.0, 117.7, 103.5, 66.8, 56.0, 51.1, 47.6, 44.0, 42.1, 41.0, 37.6, 32.5, 31.5, 30.0, 28.0, 26.4, 25.7, 25.6, 24.7, 22.7, 21.2, 14.6, 14.2. IR (neat, vₘax/cm¹): 3375, 2956, 2929, 2868, 1605, 1572, 1463, 1410, 1239, 1122. HRMS (ESI): m/z=460.3778 [M+NH₄]⁺ (calc. for C₂₉H₅₀NO₃m/z=460.3785). [α]²⁵D=+88.454±0.171 (c=1.0, CHCl₃).

### Example 4

### ((1S,4S,5S)-4-(4-((R)-3-ethyl-2-methyloctan-2-yl)-2,6-dimethoxyphenyl)-6,6-dimethylbicyclo[3.1.1]hept-2-en-2-yl)methanol

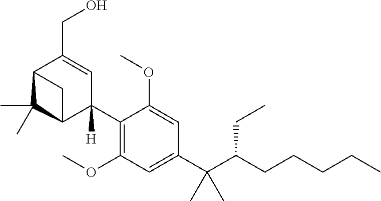 [SMILES: CCCCC[C@@H](CC)C(C)(C)c1cc(OC)c([C@H]2C=C(CO)[C@H]3C[C@@H]2C3(C)C)c(OC)c1]

The diastereomeric mixture of ((1S,4S,5S)-4-(4-((R)-3-ethyl-2-methyloctan-2-yl)-2,6-dimethoxyphenyl)-6,6-dimethylbicyclo[3.1.1]hept-2-en-2-yl)methanol was purified by chiral SFC Method 6. to yield the diastereomer A (4.5 mg, 27%, % ee>99%). [α]²⁵_{D}=+54.405±0.207 (c=1.0, CHCl₃).

### Example 5

### ((1S,4S,5S)-4-(2,6-dimethoxy-4-(2-methyl-3-propyloctan-2-yl)phenyl)-6,6-dimethylbicyclo[3.1.1]hept-2-en-2-yl)methanol

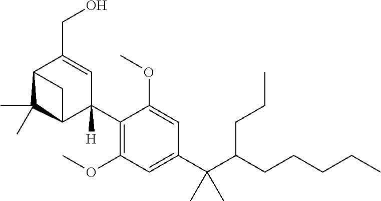 [SMILES: CCCCCC(CCC)C(C)(C)c1cc(OC)c([C@H]2C=C(CO)[C@H]3C[C@@H]2C3(C)C)c(OC)c1]

### Step b) 4-(2-(3,5-dimethoxyphenyl)propan-2-yl)nonan-4-ol

### Preparation of n-propyllithium

To a solution of 1-iodopropane (414 μL, 4.25 mmol, 1.20 equiv) in dry 3:2 pentane-Et₂O (42 mL) at −78° C. was added t-BuLi (1.7 M in pentane, 5.52 mL, 9.38 mmol, 2.65 equiv) dropwise. The solution was stirred at −78° C. for 5 min following addition, and then the mixture was allowed to reach rt and was left standing for 1 h.

To a solution of LaCl₃ 2LiCl (0.6 M in THF, 5.90 mL, 3.54 mmol, 1.00 equiv) was added 2-(3,5-dimethoxyphenyl)-2-methyloctan-3-one (985 mg, 3.54 mmol, 1.00 equiv) in dry THF (3 mL) and the resulting mixture was stirred at rt for 1 h. The reaction mixture was cooled to 0° C. and the previously prepared solution of n-PrLi was added dropwise. The mixture was stirred for 30 min. at 0° C. and then was quenched with sat. NH₄Cl solution (15 mL) and H₂O (15 mL). The mixture was diluted with Et₂O (15 mL), the phases were separated and the aqueous phase was extracted with Et₂O (3×15 mL). Combined organic extracts were dried with MgSO₄, filtered and concentrated in vacuo. The crude material was purified by flash column chromatography (SiO₂; 0-15% EtOAc in hexanes) to afford the product as a colourless oil (1.13 g, 99%).

¹H NMR (400 MHz, CDCl₃) δ 6.62 (d, J=2.2 Hz, 2H), 6.35 (t, J=2.2 Hz, 1H), 3.79 (s, 6H), 1.56-1.40 (m, 4H), 1.37 (s, 6H), 1.31-1.24 (m, 8H), 1.24 (s, 1H), 0.85 (t, J=7.1 Hz, is 3H), 0.84 (t, J=7.2 Hz, 3H). ¹³C NMR (101 MHz, CDCl₃) δ 160.2, 149.8, 107.1, 97.5, 77.1, 55.4, 46.8, 38.1, 35.6, 33.1, 24.9, 24.6, 22.8, 18.2, 15.2, 14.2. IR (neat, vₘax/cm⁻¹): 3572, 2957, 2871, 1595, 1457, 1421, 1205, 1156, 1054. HRMS (ESI): m/z=245.2405 [M+Na]⁺ (calc. for C₂₀H₃₄NaO₃ m/z=345.2400).

### Steps d and e) 1,3-dimethoxy-5-(2-methyl-3-propyloctan-2-yl)benzene

To a solution of 4-(2-(3,5-dimethoxyphenyl)propan-2-yl)nonan-4-ol (840 mg, 2.61 mmol, 1.0 equiv) in dry pyridine (6.5 mL) was added DMAP (63.6 mg, 521 μmol, 0.2 equiv). The mixture was cooled to 0° C., SOCl₂ (570 μL, 7.81 mmol, 3.0 equiv) was added dropwise and the solution was allowed to warm up to rt. and stirred for 30 min. The reaction was quenched by addition of aq. 1 M HCl (30 mL) and diluted with EtOAc (50 mL). The phases were separated and the aqueous phase was extracted with EtOAc (50 mL). Combined organic phases were washed with 0.5 M HCl (2×20 mL), dried over MgSO₄, filtered and concentrated in vacuo. The crude material was dissolved in dry EtOAc (7.0 mL), Pd (10 wt % on C, 278 mg, 261 μmol, 0.1 equiv) was added and the resulting mixture was stirred in an autoclave at rt for 24 h under 75 bar hydrogen pressure. Then the reaction mixture was filtered through Celite and the crude material was purified by flash column chromatography (SiO₂; 0-10% EtOAc in hexanes) to afford the product as a colourless oil (790 mg, 99%).

¹H NMR (400 MHz, CDCl₃) δ 6.56 (d, J=2.3 Hz, 2H), 6.34 (t, J=2.2 Hz, 1H), 3.81 (s, 6H), 1.51 (tt, J=7.4, 2.6 Hz, 1H), 1.47-1.18 (m, 10H), 1.25 (s, 6H), 1.14-1.03 (m, 2H), 0.96-0.78 (m, 6H). ¹³C NMR (101 MHz, CDCl₃) δ 160.4, 153.5, 105.2, 96.6, 55.1, 48.7, 41.9, 34.5, 32.5, 32.0, 29.8, 25.6, 25.4, 23.1, 22.6, 14.7, 14.1. IR (neat, vₘₐₓ/cm⁻¹): 2956, 2931, 2871, 1595, 1456, 1421, 1205, 1155, 1055. GC-MS (E1): m/z=306.2552 [M]⁺ (calc. for C₂₀H₃₄02 m/z=306.2553).

### Steps f) 5-(2-methyl-3-propyloctan-2-yl)benzene-1,3-diol

To a solution of 1,3-dimethoxy-5-(2-methyl-3-propyloctan-2-yl)benzene (890 mg, 2.90 mmol, 1.0 equiv) in anhydrous CH₂Cl₂ (30 mL) at 0° C. was added BBr₃ (827 μL, 8.71 mmol, 3.0 equiv) dropwise and the green solution was stirred at 0° C. for 4 h. The mixture was diluted with CH₂Cl₂ (30 mL) and carefully quenched with aq. sat. NaHCO₃ (30 mL). The layers were separated and the aqueous phase was extracted with CH₂Cl₂ (2×50 mL). Combined organic extracts were dried over MgSO₄ and concentrated in vacuo. Purification by flash column chromatography (SiO₂; using 0-40% EtOAc in hexanes) afforded the product as a golden oil (800 mg, 99%).

¹H NMR (400 MHz, CDCl₃) δ 6.59 (bs, 2H), 6.49 (d, J=2.2 Hz, 2H), 6.23 (t, J=2.1 Hz, 1H), 1.43-1.35 (m, 1H), 1.34-1.16 (m, 10H), 1.12 (d, J=2.0 Hz, 6H), 1.04-0.92 (m, 2H), 0.84 (t, J=7.1 Hz, 3H), 0.78 (t, J=6.9 Hz, 3H). ¹³C NMR (101 MHz, CDCl₃) δ 155.8, 155.0, 106.8, 100.2, 48.6, 41.8, 34.4, 32.5, 31.9, 29.7, 25.6, 25.2, 23.1, 22.7, 14.7, 14.2. IR (neat, vₘₐₓ/cm⁻¹): 3324, 2957, 2930, 2871, 1596, 1466, 1325, 1151, 992. HRMS (ESI): m/z=279.2318 [M+H]⁺ (calc. for C₁₈H₃₁₀₂m/z=279.2319).

### Step o) ((1S,4S,5S)-4-(2,6-dihydroxy-4-(2-methyl-3-propyloctan-2-yl)phenyl)-6,6-dimethylbicyclo[3.1.1]hept-2-en-2-yl)methyl pivalate

((1S,4R,5R)-4-hydroxy-6,6-dimethylbicyclo[3.1.1]hept-2-en-2-yl)methyl pivalate (60.0 mg, 236 mol, 1.3 equiv) was added to the solution of 5-(2-methyl-3-propyloctan-2-yl)benzene-1,3-diol (50.6 mg, 182 mol, 1.0 equiv) and pTsOH·H₂O (9.7 mg, 50.8 mol, 0.28 equiv) in CH₂Cl₂ (7.0 mL), and the mixture was stirred for 30 min at rt. The reaction was quenched by addition of sat. aq. NaHCO₃ (3 mL). The phases were separated, and the aqueous layer was extracted with Et₂O (3×5 mL). Combined organic extracts were dried over MgSO₄, filtered and concentrated in vacuo. Purification by flash column chromatography (SiO₂; 0-10% EtOAc in hexanes) afforded the product as a white foam (82.2 mg, 88%).

¹H NMR (400 MHz, CDCl₃) δ 6.35 (s, 2H), 6.04-5.99 (m, 1H), 5.90-5.51 (bs, 2H), 4.67-4.59 (m, 1H), 4.55-4.47 (m, 1H), 4.04-3.99 (m, 1H), 2.40-2.33 (m, 1H), 2.32-2.27 (m, 2H), 1.49 (d, J=9.7 Hz, 1H), 1.35-0.91 (m, 12H), 1.34 (s, 3H), 1.23 (s, 9H), 1.14 (s, 3H), 1.13 (s, 3H), 0.98 (s, 3H), 0.84-0.80 (m, 1H), 0.82 (t, J=7.0 Hz, 3H), 0.80 (t, J=7.0 Hz, 3H). ¹³C NMR (101 MHz, CDCl₃) δ 178.7, 154.9, 151.5, 149.4, 120.6, 111.5, 107.0, 66.6, 48.7, 47.5, 44.3, 41.4, 41.1, 39.1, 37.9, 34.4, 32.5, 31.9, 29.8, 28.1, 27.4, 26.0, 25.7, 25.1, 23.1, 22.7, 20.9, 14.8, 14.2. IR (neat, vₘₐₓ/cm⁻¹): 3458, 2956, 2929, 2870, 1730, 1708, 1625, 1574, 1479, 1462, 1429, 1366, 1282, 1232, 1159, 1026, 837, 757. HRMS (ESI): m/z=535.375 [M+Na]⁺ (calc. for C₃₃H₅₂NaO₄ m/z=535.3758). [α]²⁵_{D}=+53.675±0.246 (c=1.0, CHCl₃).

### Step p) ((1S,4S,5S)-4-(2,6-dimethoxy-4-(2-methyl-3-propyloctan-2-yl)phenyl)-6,6-dimethylbicyclo[3.1.1]hept-2-en-2-yl)methyl pivalate

To the suspension of ((1S, 4S, 5S)-4-(2,6-dihydroxy-4-(2-methyl-3-propyloctan-2-yl)phenyl)-6,6-dimethylbicyclo[3.1.1]hept-2-en-2-yl)methyl pivalate (82.2 mg, 160 mol, 1.0 equiv) and K₂CO₃ (133 mg, 962 mol, 6.0 equiv) in acetone (1.6 mL) was added (MeO)₂SO₂ (76 L, 80.2 mol, 5.0 equiv) and the mixture was stirred for 19 h at rt. The suspension was filtered and concentrated in vacuo. Purification by flash column chromatography (SiO₂; 0-15% Et₂O in hexanes) afforded the product as a pale pink oil (86.0 mg, 99%).

¹H NMR (400 MHz, CDCl₃) δ 6.49 (s, 2H), 5.81-5.76 (m, 1H), 4.62-4.55 (m, 1H), 4.54-4.48 (m, 1H), 4.03-3.98 (m, 1H), 3.74 (s, 6H), 2.20-2.13 (m, 2H), 2.08-2.01 (m, 1H), 1.75-1.68 (m, 1H), 1.46-0.97 (m, 12H), 1.30 (s, 3H), 1.22 (s, 12H), 1.21 (s, 3H), 0.98 (s, 3H), 0.85-0.80 (m, 1H), 0.82 (t, J=7.1 Hz, 3H), 0.82 (t, J=7.0 Hz, 3H). ¹³C NMR (101 MHz, CDCl₃) δ 178.7, 158.4, 150.4, 137.3, 126.6, 117.7, 103.5, 67.7, 56.0, 48.9, 47.6, 44.0, 42.0, 41.0, 39.0, 37.7, 34.6, 32.5, 32.1, 29.9, 27.7, 27.5, 26.5, 26.0, 25.4, 23.1, 22.7, 21.2, 14.8, 14.2. IR (neat, vₘₐₓ/cm⁻¹): 2955, 2928, 2868, 1728, 1605, 1572, 1461, 1410, 1280, 1239, 1151, 1123. HRMS (ESI): m/z=563.4056 [M+Na]⁺ (calc. for C₃₅H₅₆NaO₄ m/z=563.4071). [α]²⁵_{D}=+65.594±0.383 (c=1.0, CDCl₃).

### Step q) ((1S,4S,5S)-4-(2,6-dimethoxy-4-(2-methyl-3-propyloctan-2-yl)phenyl)-6,6-dimethylbicyclo[3.1.1]hept-2-en-2-yl)methanol

Solution of ((1S,4S,5S)-4-(2,6-dimethoxy-4-(2-methyl-3-propyloctan-2-yl)phenyl)-6,6-dimethylbicyclo[3.1.1]hept-2-en-2-yl)methyl pivalate (86.0 mg, 159 mol, 1.0 equiv) in CH₂Cl₂(1.6 mL) was cooled to 0° C. and DIBAL (1.0 M in hexanes, 334 L, 334 mol, 2.1 equiv) was added dropwise. The mixture was stirred at 0° C. for 20 min, subsequently diluted with CH₂Cl₂ (2 mL) and quenched with sat. aq. Rochelle's salt (5 mL). The mixture was stirred vigorously at rt until the phases cleanly separated. The layers were separated, and the aqueous phase was extracted with Et₂O (5×3 mL). Combined organic extracts were dried over MgSO₄, filtered and concentrated in vacuo. Purification by flash column chromatography (SiO₂; 10-20% EtOAc in hexanes) afforded the product (61.6 mg, 85%) as a pale pink oil.

¹H NMR (400 MHz, CDCl₃) δ 6.50 (s, 2H), 5.73-5.69 (m, 1H), 4.08 (s, 2H), 4.03-3.98 (m, 1H), 3.74 (s, 6H), 2.26-2.17 (m, 2H), 2.09-2.03 (m, 1H), 1.71 (d, J=7.9 Hz, 1H), 1.47-1.39 (m, 1H), 1.38-0.99 (m, 11H), 1.31 (s, 3H), 1.22 (s, 3H), 1.21 (s, 3H), 0.97 (s, 3H), 0.87-0.83 (m, 1H), 0.83 (t, J=7.0 Hz, 3H), 0.82 (t, J=7.1 Hz, 3H). C NMR (101 MHz, CDCl₃) δ 158.4, 150.3, 142.0, 123.9, 117.8, 103.5, 66.8, 56.0, 48.8, 47.6, 43.9, 42.0, 41.0, 37.6, 34.6, 32.5, 32.0, 29.9, 28.0, 26.4, 25.9, 25.4, 23.1, 22.6, 21.2, 14.8, 14.2. IR (neat, vₘₐₓ/cm⁻¹): 3368, 2954, 2930, 2868, 1605, 1571, 1463, 1409, 1238, 1121, 831. HRMS (ESI): m/z=495.3234 [M+K]⁺ (calc. for C₃₀H₄₈KO₃ m/z=495.3235). [α]²⁵_{D}=+92.576±0.103 (c=1.0, CDCl₃).

### Example 6

### ((1S,4S,5S)-4-(4-(3-isopropyl-2-methyloctan-2-yl)-2,6-dimethoxyphenyl)-6,6-dimethylbicyclo[3.1.1]hept-2-en-2-yl)methanol

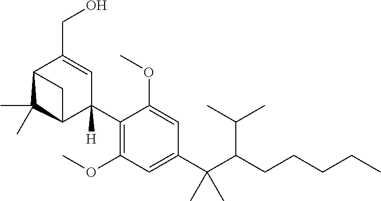 [SMILES: CCCCCC(C(C)C)C(C)(C)c1cc(OC)c([C@H]2C=C(CO)[C@H]3C[C@@H]2C3(C)C)c(OC)c1]

### Step b) 2-(3,5-dimethoxyphenyl)-3-isopropyl-2-methyloctan-3-ol

To a solution of LaCl₃ 2LiCl (0.6 M in THF, 6.00 mL, 3.59 mmol, 1.0 equiv) was added 2-(3,5-dimethoxyphenyl)-2-methyloctan-3-one (1.00 g, 3.59 mmol, 1.0 equiv) in dry THF (3.3 mL) and the resulting mixture was stirred at rt. for 1 h. The reaction mixture was cooled to 0° C. and a solution of i-PrMgCl (1.4 M in THF, 2.82 mL, 3.95 mmol, 1.1 equiv) was added dropwise and the mixture was allowed to stir at the same temperature for 2.5 h. The mixture was quenched with sat. NH₄Cl solution (10 mL) and H₂O (10 mL) and diluted with Et₂O (10 mL). The layers were separated and the aqueous phase was extracted with Et₂O (3×10 mL). Combined organic extracts were dried with MgSO₄, filtered and concentrated in vacuo. The crude material was purified by flash column chromatography (SiO₂; 0-15% EtOAc in hexanes) to afford the product as a colourless oil (820 mg, 71%).

¹H NMR (400 MHz, CDCl₃) δ 6.65 (d, J=2.3 Hz, 2H), 6.34 (t, J=2.2 Hz, 1H), 3.78 (s, 6H), 2.07 (hept, J=7.0 Hz, 1H), 1.62 (ddd, J=13.9, 11.2, 4.9 Hz, 1H), 1.50-1.38 (m, 1H), 1.43 (s, 1H), 1.41 (s, 3H), 1.40 (s, 3H), 1.35-1.12 (m, 6H), 0.90-0.79 (m, 9H). ¹³C NMR (101 MHz, CDCl₃) δ 160.2, 150.5, 107.2, 97.4, 78.4, 55.3, 47.9, 33.7, 33.4, 33.2, 26.1, 25.3, 24.7, 22.8, 20.5, 20.2, 14.2. IR (neat, vₘₐₓ/cm⁻¹): 3573, 2955, 1594, 1457, 1421, 1204, 1155, 1052. HRMS (ESI): m/z=345.2398 [M+Na]⁺ (calc. for C₂₀H₃₄NaO₃ m/z=345.2400).

### Steps d and e) 1-(3-isopropyl-2-methyloctan-2-yl)-3,5-dimethoxybenzene

To a solution of 2-(3,5-dimethoxyphenyl)-3-isopropyl-2-methyloctan-3-ol (550 mg, 1.71 mmol, 1.0 equiv) in dry pyridine (6.0 mL) was added DMAP (41.7 mg, 341 μmol, 0.2 equiv). The mixture was cooled to 0° C., SOCl₂ (373 μL, 5.12 mmol, 3.0 equiv) was added dropwise and the solution was allowed to warm up to rt. and stirred for 30 min. The reaction was quenched by addition of aq. 1 M HCl (30 mL) and diluted with EtOAc (50 mL). The phases were separated and the aqueous phase was extracted with EtOAc (50 mL). Combined organic phases were washed with 0.5 M HCl (2×20 mL), dried over MgSO₄, filtered and concentrated in vacuo. The crude material was dissolved in dry MeOH (3.0 mL), Pd (10 wt % on C, 182 mg, 171 μmol, 0.1 equiv) was added and the resulting mixture was stirred in an autoclave at rt for 48 h under 75 bar H₂ pressure. Then the reaction mixture was filtered through Celite and the crude material was purified by flash column chromatography (SiO₂; 0-10% EtOAc in hexanes) to afford the product as a colourless oil (425 mg, 81%).

¹H NMR (400 MHz, CDCl₃) δ 6.52 (d, J=2.2 Hz, 2H), 6.31 (t, J=2.2 Hz, 1H), 3.80 (s, 6H), 1.76-1.63 (m, 1H), 1.56 (ddd, J=7.2, 3.9, 1.5 Hz, 1H), 1.34-1.21 (m, 8H), 1.25 (s, 3H), 1.21 (s, 3H), 0.90-0.80 (m, 9H). ¹³C NMR (101 MHz, CDCl₃) δ 160.4, 154.0, 105.1, 96.6, 55.3, 53.5, 43.1, 32.7, 31.4, 27.8, 27.8, 26.3, 26.3, 24.9, 22.7, 18.6, 14.2. IR (neat, vₘₐₓ/cm⁻¹): 2955, 2872, 1595, 1456, 1421, 1205, 1155, 1055. GC-MS (E1): m/z=306.2557 [M]⁺ (calc. for C₂₀H₃₄O₂m/z=306.2553).

### Step f) 5-(3-isopropyl-2-methyloctan-2-yl)benzene-1,3-diol

To a solution of 1-(3-isopropyl-2-methyloctan-2-yl)-3,5-dimethoxybenzene (230 mg, 751 μmol, 1.0 equiv) in anhydrous CH₂Cl₂ (7.5 mL) at 0° C. was added BBr₃ (1.0 M in CH₂Cl₂, 2.25 mL, 2.25 mmol, 3.0 equiv) dropwise and the green solution was stirred at 0° C. for 5.5 h. The mixture was diluted with CH₂Cl₂ (15 mL) and carefully quenched with aq. sat. NaHCO₃ (15 mL). The layers were separated and the aqueous phase was extracted with CH₂Cl₂ (2×20 mL). Combined organic extracts were dried over MgSO₄ and concentrated in vacuo. Purification by flash column chromatography (SiO₂; using 0-40% EtOAc in hexanes) afforded the product as a golden oil (220 mg, 96%).

¹H NMR (400 MHz, CDCl₃) δ 6.45 (d, J=2.2 Hz, 2H), 6.20 (t, J=2.2 Hz, 1H), 5.78 (bs, 2H), 1.65 (heptd, J=7.0, 1.3 Hz, 1H), 1.48 (ddd, J=7.2, 4.0, 1.5 Hz, 1H), 1.33-1.09 (m, 8H), 1.16 (s, 3H), 1.13 (s, 3H), 0.86 (t, J=7.0 Hz, 3H), 0.83-0.75 (m, 6H). ¹³C NMR (101 MHz, CDCl₃) δ 156.0, 155.2, 106.5, 100.1, 53.4, 40.8, 32.7, 31.4, 27.8, 27.8, 26.3, 26.2, 24.7, 22.7, 18.6, 14.2. IR (neat, vₘₐₓ/cm⁻¹): 3330, 2958, 2872, 1597, 1469, 1325, 1152, 991.

HRMS (ESI): m/z=279.2319 [M+H]⁺ (calc. for C₁₈H₃₁02m/z=279.2319).

### Step o) ((1S,4S,5S)-4-(2,6-dihydroxy-4-(3-isopropyl-2-methyloctan-2-yl)phenyl)-6,6-dimethylbicyclo[3.1.1]hept-2-en-2-yl)methyl pivalate

((1S,4R,5R)-4-hydroxy-6,6-dimethylbicyclo[3.1.1]hept-2-en-2-yl)methyl pivalate (49.9 mg, 198 mol, 1.1 equiv) was added to the solution of 5-(3-isopropyl-2-methyloctan-2-yl)benzene-1,3-diol (50.1 mg, 180 mol, 1.0 equiv) and pTsOH·H₂O (9.6 mg, 50.4 mol, 0.28 equiv) in CH₂Cl₂ (7.0 mL), and the mixture was stirred for 1 h at rt. The reaction was quenched by addition of sat. aq. NaHCO₃ (3 mL) and diluted with Et₂O (3 mL). The phases were separated, and the aqueous layer was extracted with Et₂O (3×3 mL). Combined organic extracts were dried over MgSO₄, filtered and concentrated in vacuo. Purification by flash column chromatography (SiO₂; 2-8% EtOAc in hexanes) afforded the product as a colourless foam (58.6 mg, 64%).

¹H NMR (400 MHz, CDCl₃) δ 6.35 (s, 2H), 6.02 (m, 1H), 5.90-5.47 (bs, 2H), 4.68-4.59 (m, 1H), 4.55-4.47 (m, 1H), 4.05-3.99 (m, 1H), 2.42-2.33 (m, 1H), 2.32-2.27 (m, 2H), 1.71 (dhept, J=7.0, 1.7 Hz, 1H), 1.49 (d, J=1.6 Hz, 1H), 1.48-1.43 (m, 1H), 1.34 (s, 3H), 1.30-1.05 (m, 8H), 1.23 (s, 9H), 1.18 (s, 3H), 1.14 (s, 3H), 0.98 (s, 3H), 0.85 (t, J=5.2 Hz, 3H), 0.84 (d, J=7.2 Hz, 3H), 0.78 (dd, J=6.9, 1.2 Hz, 3H). ¹³C NMR (101 MHz, CDCl₃) δ 178.7, 154.9, 151.9, 149.4, 120.6, 111.5, 106.9, 66.6, 53.4, 47.5, 44.3, 42.4, 41.0, 39.1, 37.9, 32.6, 31.4, 28.2, 28.1, 27.7, 27.4, 27.4, 26.2, 26.0, 25.0, 22.7, 20.9, 18.6, 14.2. IR (neat, vₘₐₓ/cm⁻¹): 3457, 2956, 2871, 1707, 1625, 1575, 1480, 1429, 1366, 1283, 1233, 1162, 1027, 837. HRMS (ESI): m/z=535.3749 [M+Na]⁺ (calc. for C₃₃H₅₂NaO₄ m/z=535.3758). [α]²⁵D=+63.340±0.159 (c=1.0, CDCl₃).

### Step p) ((1S,4S,5S)-4-(4-(3-isopropyl-2-methyloctan-2-yl)-2,6-dimethoxyphenyl)-6,6-dimethylbicyclo[3.1.1]hept-2-en-2-yl)methyl pivalate

To the suspension of ((1S,4S,5S)-4-(2,6-dihydroxy-4-(3-isopropyl-2-methyloctan-2-yl)phenyl)-6,6-dimethylbicyclo[3.1.1]hept-2-en-2-yl)methyl pivalate (48.1 mg, 93.8 mol, 1.0 equiv) and K₂CO₃ (77.8 mg, 563 μmol, 6.0 equiv) in acetone (1.0 mL) was added (MeO₂)SO₂ (45 μL, 469 μmol, 5.0 equiv). The resulting purple suspension was stirred for 20 h at rt. The reaction was quenched by addition of sat. aq. NaHCO₃ (3 mL). The phases were separated, and the aqueous layer was extracted with Et₂O (3×3 mL). Combined organic extracts were dried over MgSO₄, filtered and concentrated in vacuo. Purification by flash column chromatography (SiO₂; 0-15% Et₂O in hexanes) afforded the product as a pale pink oil (38.2 mg, 75%).

¹H NMR (400 MHz, CDCl₃) δ 6.49 (m, 2H), 5.82-5.75 (m, 1H), 4.61-4.55 (m, 1H), 4.54-4.47 (m, 1H), 4.02-3.98 (m, 1H), 3.74 (s, 6H), 2.21-2.12 (m, 2H), 2.08-2.01 (m, 1H), 1.77-1.65 (m, 2H), 1.55-1.48 (m, 1H), 1.36-1.05 (m, 8H), 1.30 (s, 3H), 1.25 (s, 3H), 1.21 (s, 9H), 1.20 (s, 3H), 0.98 (s, 3H), 0.86 (dd, J=7.1, 1.1 Hz, 3H), 0.85 (t, J=7.2 Hz, 3H), 0.80 (d, J=6.9 Hz, 3H). ¹³C NMR (101 MHz, CDCl₃) δ 178.6, 158.4, 150.7, 137.3, 126.6, 117.7, 103.3, 67.7, 55.9, 53.7, 47.5, 44.0, 43.1, 41.0, 39.0, 37.7, 32.7, 31.4, 27.8, 27.7, 27.6, 27.5, 26.5, 26.3, 25.2, 25.1, 22.7, 21.2, 18.6, 14.2. IR (neat, vₘₐₓ/cm⁻¹): 2946, 2870, 1728, 1605, 1572, 1461, 1410, 1365, 1280, 1239, 1151, 1122, 832. HRMS (ESI): m/z=563.4057 [M+Na]⁺ (calc. for C₃₅H₅₆NaO₄ m/z=563.4071). [α]²⁵_{D}=+89.637±0.233 (c=1.0, CDCl₃).

### Step q) ((1S,4S,5S)-4-(4-(3-isopropyl-2-methyloctan-2-yl)-2,6-dimethoxyphenyl)-6,6-dimethylbicyclo[3.1.1]hept-2-en-2-yl)methanol

Solution of ((1S,4S,5S)-4-(4-(3-isopropyl-2-methyloctan-2-yl)-2,6-dimethoxyphenyl)-6,6-dimethylbicyclo[3.1.1]hept-2-en-2-yl)methyl pivalate (28.2 mg, 52.1 mol, 1.0 equiv) in CH₂Cl₂ (0.5 mL) was cooled to 0° C. and DIBAL (1.0 M in hexanes, 110 L, 110 mol, 2.1 equiv) was added dropwise. The mixture was stirred at 0° C. for 20 min, subsequently diluted with Et₂O (2 mL) and quenched with sat. aq. Rochelle's salt (1.5 mL). The mixture was stirred vigorously at rt. until the phases cleanly separated. The layers were separated, and the aqueous phase was extracted with Et₂O (4×3 mL). Combined organic extracts were washed with brine (5 mL), dried over MgSO₄, filtered and concentrated in vacuo. Purification by flash column chromatography (SiO₂; 0-20% EtOAc in hexanes) afforded the product as a pale pink oil (18.8 mg, 79%).

¹H NMR (400 MHz, CDCl₃) δ 6.50 (s, 2H), 5.74-5.69 (m, 1H), 4.09-4.05 (m, 2H), 4.03-3.98 (m, 1H), 3.74 (s, 6H), 2.25-2.16 (m, 2H), 2.09-2.04 (m, 1H), 1.75-1.66 (m, 2H), 1.55-1.49 (m, 1H), 1.35-1.05 (m, 8H), 1.31 (s, 3H), 1.25 (s, 3H), 1.21 (s, 3H), 0.97 (s, 3H), 0.86 (d, J=7.1 Hz, 3H), 0.84 (t, J=7.1 Hz, 3H), 0.80 (dd, J=6.9, 1.1 Hz, 3H). ¹³C NMR (101 MHz, CDCl₃) δ 158.4, 150.7, 142.0, 123.9, 117.8, 103.4, 66.8, 56.0, 53.7, 47.6, 43.9, 43.1, 41.0, 37.6, 32.7, 31.4, 28.0, 27.8, 27.7, 26.4, 26.3, 26.3, 25.2, 22.7, 21.2, 18.6, 14.2. IR (neat, vₘₐₓ/cm⁻¹): 3363, 2953, 2930, 2869, 1604, 1572, 1464, 1409, 1238, 1121, 832. HRMS (ESI): m/z=479.3495 [M+Na]⁺ (calc. For C₃₀H₄₈NaO₃ m/z=479.3496). [α]²⁵_{D}=+88.948±0.459 (c=1.0, CDCl₃).

### Example 7

### ((1S,4S,5S)-4-(2,6-dimethoxy-4-((R)-2-methyl-3-phenyloctan-2-yl)phenyl)-6,6-dimethylbicyclo[3.1.1]hept-2-en-2-yl)methanol

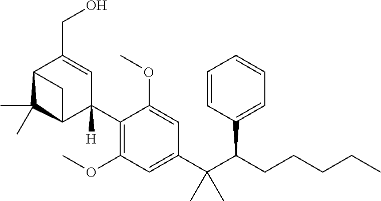 [SMILES: CCCCC[C@H](c1ccccc1)C(C)(C)c1cc(OC)c([C@H]2C=C(CO)[C@H]3C[C@@H]2C3(C)C)c(OC)c1]

### Step b) 2-(3,5-dimethoxyphenyl)-2-methyl-3-phenyloctan-3-ol

To a solution of LaCl₃ 2LiCl (0.6 M in THF, 6.29 mL, 3.77 mmol, 1.0 equiv) was added 2-(3,5-dimethoxyphenyl)-2-methyloctan-3-one (1.05 g, 3.77 mmol, 1.0 equiv) in dry THF (3.0 mL) and the resulting mixture was stirred at rt. for 1 h. The reaction mixture was cooled to 0° C. and PhLi (1.8 M in dibutyl ether, 3.14 mL, 5.66 mmol, 1.5 equiv) was added dropwise. The mixture was stirred for 30 min. at 0° C. and then was quenched with aq. sat. NH₄Cl solution (15 mL) and H₂O (15 mL). The aqueous phase was diluted with Et₂O (20 mL), the phases were separated and the aqueous phase extracted with Et₂O (3×30 mL). Combined organic extracts were dried with MgSO₄, filtered and concentrated in vacuo. The crude material was purified by flash column chromatography (SiO₂; 0-15% EtOAc in hexanes) to afford the product as a light yellow oil (1.33 g, 99%).

¹H NMR (400 MHz, CDCl₃) δ 7.30-7.20 (m, 5H), 6.46 (d, J=2.3 Hz, 2H), 6.37 (t, J=2.2 Hz, 1H), 3.75 (s, 6H), 2.21 (ddd, J=14.0, 11.6, 4.2 Hz, 1H), 1.81 (s, 1H), 1.60 (ddd, J=13.0, 11.6, 3.6 Hz, 1H), 1.39 (s, 3H), 1.27 (s, 3H), 1.24-1.09 (m, 4H), 0.94-0.84 (m, 2H), 0.83-0.77 (m, 3H). ¹³C NMR (101 MHz, CDCl₃) δ 160.0, 148.4, 142.5, 128.0, 127.0, 126.4, 107.5, 98.0, 80.5, 55.3, 46.7, 35.7, 32.6, 25.1, 24.8, 23.8, 22.8, 14.2. IR (neat, vₘₐₓ/cm⁻¹): 3552, 2954, 2870, 1595, 1457, 1205, 1155. HRMS (ESI): m/z=379.2241 [M+Na]⁺ (calc. for C₂₃H₃₂NaO₃ m/z=379.2244).

### Step g) (E)-1,3-dimethoxy-5-(2-methyl-3-phenyloct-3-en-2-yl)benzene

To a solution of 2-(3,5-dimethoxyphenyl)-2-methyl-3-phenyloctan-3-ol (2.65 g, 7.43 mmol, 1.0 equiv) in anhydrous THF (48 mL) at −78° C. was added KHMDS (1.0 M in THF, 18.6 mL, 18.6 mmol, 2.5 equiv) and CS₂ (6.71 mL, 112 mmol, 15 equiv). The yellow solution was stirred at −78° C. for 10 min, the cooling bath was removed and the mixture stirred for another 30 min. Mel (7.40 mL, 119 mmol, 16 equiv) was added and the mixture was first stirred at ambient temperature for 1 h, and then at 40° C. overnight. The mixture was diluted with Et₂O (50 mL) and aq. sat. NaHCO₃ (50 mL) was added. The layers were separated and the aqueous phase was extracted with Et₂O (2×50 mL). Combined organic extracts were washed with brine, dried over MgSO₄ and concentrated in vacuo. Purification by flash column chromatography (SiO₂; using 0.5% Et₂O in pentanes) afforded the product as an amber orange liquid (2.35 g, 93%).

¹H NMR (400 MHz, CDCl₃) δ 7.19-7.11 (m, 3H), 6.75-6.64 (m, 2H), 6.55 (d, J=2.3 Hz, 2H), 6.35 (t, J=2.3 Hz, 1H), 5.71 (t, J=7.2 Hz, 1H), 3.79 (s, 6H), 1.78 (q, J=7.1 Hz, 2H), 1.40 (s, 6H), 1.36-1.18 (m, 4H), 0.83 (t, J=7.2 Hz, 3H). ¹³C NMR (101 MHz, CDCl₃) δ 160.4, 151.2, 148.5, 140.5, 129.7, 127.4, 126.5, 126.1, 105.7, 97.4, 55.3, 44.2, 32.3, 29. 1, 29.0, 22.4, 14.1. IR (neat, vₘₐₓ/cm⁻¹): 2956, 2872, 1595, 1456, 1423, 1204, 1153. HRMS (ESI): m/z=339.2315 [M+H]⁺ (calc. for C₂₃H₃₁02m/z=339.2319).

### Step h) (E)-5-(2-methyl-3-phenyloct-3-en-2-yl)benzene-1,3-diol

To a solution of (E)-1,3-dimethoxy-5-(2-methyl-3-phenyloct-3-en-2-yl)benzene (1.70 g, 5.02 mmol, 1.0 equiv) in anhydrous CH₂Cl₂ (48 mL) at 0° C. was added BBr₃ (1.43 mL, 15.1 mmol, 3.0 equiv) dropwise and the green solution was stirred at 0° C. for 3 h. The mixture was diluted with CH₂Cl₂ (100 mL) and carefully quenched with aq. sat. NaHCO₃ (50 mL). The layers were separated and the aqueous phase was extracted with CH₂Cl₂ (2×100 mL). Combined organic extracts were dried over MgSO₄ and concentrated in vacuo. Purification by flash column chromatography (SiO₂; using 0-30% EtOAc in hexanes) afforded the product as a golden oil (1.54 g, 99%).

¹H NMR (400 MHz, CDCl₃) δ 7.18-7.09 (m, 3H), 6.73-6.63 (m, 2H), 6.40 (d, J=2.3 Hz, 2H), 6.19 (t, J=2.2 Hz, 1H), 5.66 (t, J=7.3 Hz, 1H), 4.87 (s, 2H), 1.72 (q, J=7.2 Hz, 2H), 1.33 (s, 6H), 1.32-1.16 (m, 4H), 0.81 (t, J=7.1 Hz, 3H). ¹³C NMR (101 MHz, CDCl₃) δ 156.2, 152.1, 148.2, 140.5, 129.8, 127.4, 126.6, 126.2, 107.2, 100.4, 43.9, 32.3, 29.0, 28.9, 22.5, 14.1. IR (neat, vₘₐₓ/cm⁻¹): 3337, 3054, 2960, 2928, 2872, 1598, 1492, 1465, 1439, 1322, 1149. HRMS (ESI): m/z=311.2013 [M+H]⁺ (calc. for C₂₁H₂₇02m/z=311.2006).

### Step i) 5-(2-methyl-3-phenyloctan-2-yl)benzene-1,3-diol

To a solution of (E)-5-(2-methyl-3-phenyloct-3-en-2-yl)benzene-1,3-diol (1.00 g, 3.22 mmol, 1.0 equiv) in EtOAc (27 mL) was added Pd (10 wt % on C, 3.43 g, 3.22 mmol, 1.0 equiv) and the mixture was stirred overnight in an autoclave under 7 bar H₂ pressure. The suspension was filtered over a pad of Celite and concentrated in vacuo to afford the product as a pink foam (960 mg, 95% yield). The racemate was resolved by chiral SFC Method 1. to afford (R)-5-(2-methyl-3-phenyloctan-2-yl)benzene-1,3-diol A (430 mg, 43%, % ee>99%) and (S)-5-(2-methyl-3-phenyloctan-2-yl)benzene-1,3-diol B (420 mg, 42%, % ee>99%).

¹H NMR (400 MHz, CDCl₃) δ 7.28-7.14 (m, 3H), 7.11-7.03 (m, 2H), 6.39 (d, J=2.2 Hz, 2H), 6.20 (t, J=2.2 Hz, 1H), 4.69 (bs, 2H), 2.73 (dd, J=12.1, 2.9 Hz, 1H), 1.61 (dddd, J=13.3, 12.1, 9.7, 4.7 Hz, 1H), 1.38 (dddd, J=13.1, 9.6, 6.5, 2.9 Hz, 1H), 1.22 (s, 3H), 1.18-1.01 (m, 4H), 1.07 (s, 3H), 1.00-0.79 (m, 2H), 0.74 (t, J=6.6 Hz, 3H). ¹³C NMR (101 MHz, CDCl₃) δ 156.2, 153.3, 141.8, 130.2, 127.5, 126.2, 106.8, 100.2, 57.1, 41.6, 31.9, 29.4, 29.1, 28.1, 23.5, 22.6, 14.2. IR (neat, vₘₐₓ/cm¹): 3340, 2956, 2931, 2870, 1599, 1496, 1467, 1328, 1151, 992, 704. GC-MS (E1): m/z=312.2084 [M]⁺ (calc. for C₂₁H₂₈O₂ m/z=312.2084). (R)-5-(2-methyl-3-phenyloctan-2-yl)benzene-1,3-diol A [α]²⁵_{D}=−38.557 0.179 (c=1.0, CHCl₃). (S)-5-(2-methyl-3-phenyloctan-2-yl)benzene-1,3-diol B [α]²⁵_{D}=+38.058±0.691 (c=1.0, CHCl₃).

### Step o) ((1S,4S,5S)-4-(2,6-dihydroxy-4-((R)-2-methyl-3-phenyloctan-2-yl)phenyl)-6,6-dimethylbicyclo[3.1.1]hept-2-en-2-yl)methyl pivalate

To a solution of resorcinol (R)-5-(2-methyl-3-phenyloctan-2-yl)benzene-1,3-diol A (17.6 mg, 56.3 μmol, 1.0 equiv) in anhydrous CH₂Cl₂ (1.4 mL) was added pTsOH·H₂O (3.0 mg, 15.7 μmol, 0.28 equiv) and a solution of ((1S,4R,5R)-4-hydroxy-6,6-dimethylbicyclo[3.1.1]hept-2-en-2-yl)methyl pivalate (15.6 mg, 61.9 mol, 1.1 equiv) in anhydrous CH₂Cl₂ (0.7 mL). The reaction was stirred at ambient temperature for 30 min. The reaction was quenched by addition of sat. aq. NaHCO₃ (2 mL). The phases were separated, and the aqueous layer was extracted with CH₂Cl₂ (3×15 mL) and EtOAc (3×15 mL). The combined organic extracts were dried over MgSO₄ and concentrated in vacuo. Purification by flash column chromatography (SiO₂, 5-10% EtOAc in hexanes) afforded the product (23.5 mg, 76%) as a light-yellow foam.

¹H NMR (400 MHz, CDCl₃) δ 7.25-7.14 (m, 3H), 7.12-7.05 (m, 2H), 6.36 (s, 2H), 6.04 (dt, J=3.1, 1.5 Hz, 1H), 5.69 (s, 2H), 4.65 (dt, J=13.3, 1.6 Hz, 1H), 4.53 (dt, J=13.5, 1.9 Hz, 1H), 4.03 (s, 1H), 2.72 (dd, J=12.1, 2.9 Hz, 1H), 2.43-2.35 (m, 1H), 2.35-2.28 (m, 2H), 1.68-1.54 (m, 1H), 1.51 (d, J=9.6 Hz, 1H), 1.41-1.33 (m, 1H), 1.36 (s, 3H), 1.24 (s, 9H), 1.19 (s, 3H), 1.13-1.00 (m, 4H), 1.02 (s, 3H), 1.00 (s, 3H), 0.98-0.82 (m, 2H), 0.74 (t, J=6.5 Hz, 3H). ¹³C NMR (101 MHz, CDCl₃) δ 178.7, 154.9, 150.4, 149.5, 142.0, 130.2, 127.5, 126.1, 120.5, 111.8, 107.3, 66.6, 56.9, 47.5, 44.3, 41.2, 41.1, 39.1, 37.9, 31.8, 29.4, 29.3, 28.2, 28.0, 27.4, 26.0, 22.8, 22.5, 20.9, 14.2. IR (neat, vₘₐₓ/cm⁻¹): 3455, 2957, 2932, 2871, 1728, 1707, 1625, 1575, 1479, 1465, 1452,1429,1367,1283,1230,1163,1040, 1029, 748, 704. HRMS (ESI): m/z=569.3603 [M+Na]⁺ (calc. for C₃₆H₅₀NaO₄ m/z=569.3601). [α]²⁵_{D}=+19.921±0.144 (c=1.0, CHCl₃).

### Step p) ((1S,4S,5S)-4-(2,6-dimethoxy-4-((R)-2-methyl-3-phenyloctan-2-yl)phenyl)-6,6-dimethylbicyclo[3.1.1]hept-2-en-2-yl)methyl pivalate

To a solution of ((1S,4S,5S)-4-(2,6-dihydroxy-4-((R)-2-methyl-3-phenyloctan-2-yl)phenyl)-6,6-dimethylbicyclo[3.1.1]hept-2-en-2-yl)methyl pivalate (23.5 mg, 42.9 mol, 1.0 equiv) in acetone (0.5 mL) was added (MeO)₂SO₂ (12.2 μL. 129 μmol, 3.0 equiv) and K₂CO₃(20.8 mg. 0.150 mmol, 3.5 equiv) and the light red suspension was stirred overnight at rt.

Subsequently, (MeO)₂SO₂ (12.2 μL. 0.129 mmol, 3.0 equiv) was added and the solution stirred for 2 h at rt. The reaction mixture was diluted with Et₂O (5 mL) and water (5 mL). The layers were separated and the aqueous layer extracted with Et₂O (2×5 mL). Combined organic fractions were dried over MgSO₄, filtered and concentrated in vacuo. Purification by flash column chromatography (SiO₂; using 0 to 5% Et₂O in hexanes) afforded the product as a light red wax (21.8 g, 88%).

¹H NMR (500 MHz, CDCl₃) δ 7.25-7.15 (m, 3H), 7.07-6.98 (m, 2H), 6.42 (s, 2H), 5.79 (dt, J=2.7, 1.4 Hz, 1H), 4.65-4.55 (m, 1H), 4.55-4.46 (m, 1H), 4.05-3.97 (m, 1H), 3.69 (s, 6H), 2.72 (dd, J=12.1, 2.9 Hz, 1H), 2.21-2.13 (m, 2H), 2.09-2.02 (m, 1H), 1.73 (d, J=7.8 Hz, 1H), 1.61 (dddd, J=13.5, 11.9, 9.5, 4.6 Hz, 1H), 1.50-1.40 (m, 1H), 1.31 (s, 3H), 1.27 (s, 3H), 1.22 (s, 9H), 1.14 (s, 3H), 1.13-1.02 (m, 4H), 0.98 (s, 3H), 0.97-0.84 (m, 2H), 0.75 (t, J=7.0 Hz, 3H). ¹³C NMR (126 MHz, CDCl₃) δ 178.6, 158.3, 148.7, 142.0, 137.4, 130.2, 127.4, 126.5, 126.1, 118.0, 104.0, 67.6, 57.3, 56.0, 47.6, 44.0, 41.8, 41.0, 39.0, 37.7, 31.9, 29.3, 28.8, 28.1, 27.7, 27.5, 26.5, 24.1, 22.6, 21.2, 14.2. IR (neat, vₘₐₓ/cm¹): 2932, 2870, 1727, 1604, 1573, 1453, 1411, 1151, 1122. HRMS (ESI): m/z=597.3915 [M+Na]⁺ (calc. for C₃₈H₅₄NaO₄ m/z=597.3914). [α]²⁵_{D}=+38.804±0.225 (c=1.0, CHCl₃).

### Step q) ((1S,4S,5S)-4-(2,6-dimethoxy-4-((R)-2-methyl-3-phenyloctan-2-yl)phenyl)-6,6-dimethylbicyclo[3.1.1]hept-2-en-2-yl)methanol

Solution of ((1S,4S,5S)-4-(2,6-dimethoxy-4-((R)-2-methyl-3-phenyloctan-2-yl)phenyl)-6,6-dimethylbicyclo[3.1.1]hept-2-en-2-yl)methyl pivalate (22.0 mg, 38.2 mol, 1.0 equiv) in CH₂Cl₂ (0.4 mL) was cooled to 0° C. and DIBAL (1.0 M in hexanes, 81 μL. 80.7 μmol, 2.1 equiv) was added dropwise. The mixture was stirred at 0° C. for 15 min. and subsequently quenched with sat. aq. Rochelle's salt (4 mL) and diluted with Et₂O (5 mL). The mixture was vigorously stirred at rt. until the phases cleanly separated. The phases were separated and the aqueous phase extracted with Et₂O (2×5 mL). Combined organic fractions were dried over MgSO₄, filtered and concentrated in vacuo. Purification by flash column chromatography (SiO₂; using 10-15% EtOAc in hexanes) afforded the product as a white foam (16.0 mg, 85%).

¹H NMR (400 MHz, CDCl₃) δ 7.25-7.14 (m, 3H), 7.08-6.96 (m, 2H), 6.43 (s, 2H), 5.76-5.68 (m, 1H), 4.08 (s, 2H), 4.05-3.98 (m, 1H), 3.70 (s, 6H), 2.73 (dd, J=12.0, 2.9 Hz, 1H), 2.28-2.18 (m, 2H), 2.11-2.00 (m, 1H), 1.78-1.69 (m, 1H), 1.68-1.54 (m, 1H), 1.52-1.39 (m, 1H), 1.32 (s, 3H), 1.27 (s, 3H), 1.15 (s, 3H), 1.13-1.02 (m, 4H), 0.98 (s, 3H), 0.96-0.81 (m, 2H), 0.75 (t, J=6.8 Hz, 3H). ¹³C NMR (101 MHz, CDCl₃) δ 158.3, 148.7, 142.1, 142.0, 130.2, 127.4, 126.1, 123.9, 118.1, 104.0, 66.8, 57.3, 56.0, 47.6, 44.0, 41.8, 41.0, 37.6, 31.9, 29.4, 28.9, 28.1, 28.0, 26.4, 24.1, 22.6, 21.2, 14.2. IR (neat, vₘₐₓ/cm-¹): 3398, 2931, 2866, 1604, 1573, 1410, 1122. HRMS (ESI): m/z=513.3335 [M+Na]⁺ (calc. for C₃₃H₄₆NaO₃m/z=513.3339). [α]²⁵_{D}=+57.735±0.424 (c=1.0, CHCl₃).

### Example 8

### ((1S,4S,5S)-4-(2,6-dimethoxy-4-((S)-2-methyl-3-phenyloctan-2-yl)phenyl)-6,6-dimethylbicyclo[3.1.1]hept-2-en-2-yl)methanol

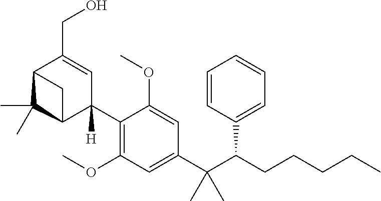 [SMILES: CCCCC[C@@H](c1ccccc1)C(C)(C)c1cc(OC)c([C@H]2C=C(CO)[C@H]3C[C@@H]2C3(C)C)c(OC)c1]

### Step o) ((1S,4S,5S)-4-(2,6-dihydroxy-4-((S)-2-methyl-3-phenyloctan-2-yl)phenyl)-6,6-dimethylbicyclo[3.1.1]hept-2-en-2-yl)methyl pivalate

To a solution of resorcinol (S)-5-(2-methyl-3-phenyloctan-2-yl)benzene-1,3-diol B (18.3 mg, 58.5 μmol, 1.0 equiv) in anhydrous CH₂Cl₂ (1.7 mL) was added pTsOH·H₂O (3.1 mg, 16.4 μmol, 0.28 equiv) and a solution of ((1S,4R,5R)-4-hydroxy-6,6-dimethylbicyclo[3.1.1]hept-2-en-2-yl)methyl pivalate (16.3 mg, 64.4 mol, 1.1 equiv) in anhydrous CH₂Cl₂ (0.5 mL). The reaction was stirred at ambient temperature for 30 min. The reaction was quenched by addition of sat. aq. NaHCO₃ (2 mL). The phases were separated, and the aqueous layer was extracted with CH₂Cl₂ (3×15 mL) and EtOAc (3×15 mL). The combined organic extracts were dried over MgSO₄ and concentrated in vacuo. Purification by flash column chromatography (SiO₂, 5-10% EtOAc in hexanes) afforded the product (24.3 mg, 76%) as a light-yellow foam.

¹H NMR (400 MHz, CDCl₃) δ 7.25-7.14 (m, 3H), 7.10-6.98 (m, 2H), 6.36 (s, 2H), 6.04 (dt, J=3.3, 1.6 Hz, 1H), 5.68 (bs, 2H), 4.65 (dt, J=13.3, 1.7 Hz, 1H), 4.52 (dt, J=13.5, 2.0 Hz, 1H), 4.08-3.97 (m, 1H), 2.71 (dd, J=12.1, 2.9 Hz, 1H), 2.44-2.35 (m, 1H), 2.34-2.28 (m, 2H), 1.69-1.56 (m, 1H), 1.52 (d, J=9.7 Hz, 1H), 1.44-1.33 (m, 1H), 1.36 (s, 3H), 1.24 (s, 9H), 1.19 (s, 3H), 1.16-1.00 (m, 4H), 1.03 (s, 3H), 0.99 (s, 3H), 0.96-0.82 (m, 2H), 0.74 (t, J=6.5 Hz, 3H). ¹³C NMR (101 MHz, CDCl₃) δ 178.7, 154.9, 150.3, 149.6, 142.0, 130.2, 127.5, 126.1, 120.5, 111.8, 107.3, 66.6, 57.0, 47.5, 44.3, 41.2, 41.1, 39.1, 37.9, 31.9, 29.4, 29.4, 28.2, 28.0, 27.4, 26.0, 22.9, 22.5, 20.9, 14.2. IR (neat, vₘₐₓ/cm⁻¹): 3459, 2957,2932,2871, 1729,1707,1625,1575,1480,1465,1452,1429,1367,1326,1283,1230, 1163, 1029, 759, 704. HRMS (ESI): m/z=569.3592 [M+Na]⁺ (calc. for C₃₆H₅₀NaO₄ m/z=569.3601). [α]²⁵_{D}=+82.985±0.109 (c=1.0, CHCl₃).

### Step p) ((1S,4S,5S)-4-(2,6-dimethoxy-4-((S)-2-methyl-3-phenyloctan-2-yl)phenyl)-6,6-dimethylbicyclo[3.1.1]hept-2-en-2-yl)methyl pivalate

To a solution of ((1S,4S,5S)-4-(2,6-dihydroxy-4-((S)-2-methyl-3-phenyloctan-2-yl)phenyl)-6,6-dimethylbicyclo[3.1.1]hept-2-en-2-yl)methyl pivalate (24.3 mg, 44.4 mol, 1.0 equiv) in acetone (0.5 mL) was added (MeO)₂SO₂ (12.6 μL. 0.133 mmol, 3.0 equiv) and K₂CO₃(21.5 mg. 0.155 mmol, 3.5 equiv) and the light red suspension was stirred overnight at rt. Subsequently, (MeO)₂SO₂ (12.6 μL. 0.133 mmol, 3.0 equiv) was added and the solution stirred for 2 h at rt. The reaction mixture was diluted with Et₂O (5 mL) and water (5 mL). The layers were separated and the aqueous layer extracted with Et₂O (2×5 mL). Combined organic fractions were dried over MgSO₄, filtered and concentrated in vacuo. Purification by flash column chromatography (SiO₂; using 0-5% Et₂O in hexanes) afforded the product as a light red wax (23.4 g, 92%).

¹H NMR (400 MHz, CDCl₃) δ 7.25-7.15 (m, 3H), 7.06-6.99 (m, 2H), 6.42 (s, 2H), 5.80 (dt, J=2.8, 1.4 Hz, 1H), 4.61-4.56 (m, 1H), 4.54-4.49 (m, 1H), 4.03-3.98 (m, 1H), 3.69 (s, 6H), 2.72 (dd, J=12.0, 2.9 Hz, 1H), 2.23-2.13 (m, 2H), 2.06 (ddt, J=5.4, 3.6, 1.8 Hz, 1H), 1.75-1.54 (m, 2H), 1.51-1.39 (m, 1H), 1.31 (s, 3H), 1.27 (s, 3H), 1.22 (s, 9H), 1.15 (s, 3H), 1.12-1.03 (m, 4H), 0.99 (s, 3H), 0.96-0.83 (m, 2H), 0.75 (t, J=6.9 Hz, 3H). ¹³C NMR (101 MHz, CDCl₃) δ 178.6, 158.2, 148.7, 142.0, 137.3, 130.2, 127.4, 126.6, 126.1, 118.0, 103.9, 67.7, 57.3, 55.9, 47.6, 44.0, 41.8, 41.1, 39.1, 37.7, 31.9, 29.4, 28.9, 28.1, 27.7, 27.5, 26.5, 24.1, 22.6, 21.2, 14.2. IR (neat, vₘₐₓ/cm⁻¹): 2931, 2869, 1727, 1604, 1573, 1452, 1411, 1151, 1122. HRMS (ESI): m/z=597.3911 [M+Na]⁺ (calc. for C₃₈H₅₄NaO₄ m/z=597.3914). [α]²⁵_{D}=+86.208±0.243 (c=1.0, CHCl₃).

### Step q) ((1S,4S,5S)-4-(2,6-dimethoxy-4-((S)-2-methyl-3-phenyloctan-2-yl)phenyl)-6,6-dimethylbicyclo[3.1.1]hept-2-en-2-yl)methanol

Solution of ((1S,4S,5S)-4-(2,6-dimethoxy-4-((S)-2-methyl-3-phenyloctan-2-yl)phenyl)-6,6-dimethylbicyclo[3.1.1]hept-2-en-2-yl)methyl pivalate (23.4 mg, 40.7 mol, 1.0 equiv) in CH₂Cl₂ (0.4 mL) was cooled to 0° C. and DIBAL (1.0 M in hexanes, 86 μL. 85.4 μmol, 2.1 equiv) was added dropwise. The mixture was stirred at 0° C. for 15 min. and subsequently quenched with sat. aq. Rochelle's salt (4 mL) and diluted with Et₂O (5 mL). The mixture was vigorously stirred at rt. until the phases cleanly separated. The phases were separated and the aqueous phase extracted with Et₂O (2×5 mL). Combined organic fractions were dried over MgSO₄, filtered and concentrated in vacuo. Purification by flash column chromatography (SiO₂; using 10-15% EtOAc in hexanes) afforded the product as a white foam (17.3 mg, 87%).

¹H NMR (400 MHz, CDCl₃) δ 7.26-7.13 (m, 3H), 7.06-6.99 (m, 2H), 6.43 (s, 2H), 5.79-5.68 (m, 1H), 4.08 (s, 2H), 4.03-4.00 (m, 1H), 3.70 (s, 6H), 2.73 (dd, J=12.1, 2.9 Hz, 1H), 2.28-2.18 (m, 2H), 2.10-2.05 (m, 1H), 1.76-1.67 (m, 1H), 1.67-1.54 (m, 1H), 1.50-1.39 (m, 1H), 1.32 (s, 3H), 1.27 (s, 3H), 1.15 (s, 3H), 1.13-1.03 (m, 4H), 0.98 (s, 3H), 0.96-0.84 (m, 2H), 0.75 (t, J=6.7 Hz, 3H). ¹³C NMR (101 MHz, CDCl₃) δ 158.3, 148.7, 142.0, 142.0, 130.2, 127.4, 126.1, 123.9, 118.0, 104.0, 66.8, 57.3, 56.0, 47.6, 44.0, 41.8, 41.0, 37.6, 31.9, 29.4, 28.9, 28.1, 28.0, 26.4, 24.1, 22.6, 21.2, 14.2. IR (neat, vₘax/cm ¹): 3369, 2931, 2867, 1604, 1573, 1410, 1122. HRMS (ESI): m/z=513.3335 [M+Na]⁺ (calc. for C₃₃H₄₆NaO₃ m/z=513.3339). [α]²⁵_{D}=+123.317±0.334 (c=1.0, CHCl₃).

### Example 9

((1S,4S,5S)-4-(4-((R)-3-(4-fluorophenyl)-2-methyloctan-2-yl)-2,6-dimethoxyphenyl)-6,6-dimethylbicyclo[3.1.1]hept-2-en-2-yl)methanol

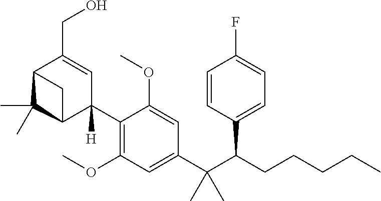 [SMILES: CCCCC[C@H](c1ccc(F)cc1)C(C)(C)c1cc(OC)c([C@H]2C=C(CO)[C@H]3C[C@@H]2C3(C)C)c(OC)c1]

### Step b) 2-(3,5-dimethoxyphenyl)-3-(4-fluorophenyl)-2-methyloctan-3-ol

### Preparation of 4-fluorophenyllithium

To a solution of 4-fluoroiodobenzene (853 μL, 7.40 mmol, 2.0 equiv) in dry Et₂O (8.0 mL) was added at −78° C. n-BuLi (1.6 M in hexane, 3.47 mL, 5.55 mmol, 1.5 equiv) dropwise and the solution was stirred at −78° C. for 30 min.

To a solution of LaCl₃ 2LiCl (0.6 M in THF, 6.17 mL, 3.70 mmol, 1.0 equiv) was added 2-(3,5-dimethoxyphenyl)-2-methyloctan-3-one (1.03 g, 3.70 mmol, 1.0 equiv) in dry THF (3.0 mL) and the resulting mixture was stirred at rt. for 1 h. The reaction mixture was cooled to 0° C. and the previously prepared solution of 4-fluorophenyllithium was added dropwise. The mixture was stirred for 30 min. at 0° C. and then was quenched with aq. sat. NH₄Cl solution (15 mL) and H₂O (15 mL). The aqueous phase was diluted with Et₂O (15 mL), the phases were separated and the aqueous phase extracted with Et₂O (3×15 mL). Combined organic extracts were dried with MgSO₄, filtered and concentrated in vacuo. The crude material was purified by flash column chromatography (SiO₂; 0-15% EtOAc in hexanes) to afford the product as a colourless oil (1.36 g, 98%).

¹H NMR (400 MHz, CDCl₃) δ 7.19 (bs, 2H), 6.94 (t, J=8.8 Hz, 2H), 6.44 (d, J=2.0 Hz, 2H), 6.36 (t, J=2.2 Hz, 1H), 3.75 (s, 6H), 2.21-2.09 (m, 1H), 1.79 (s, 1H), 1.58 (ddd, J=15.1, 11.7, 3.5 Hz, 1H), 1.35 (s, 3H), 1.24 (s, 3H), 1.21-1.04 (m, 4H), 0.92-0.74 (m, 2H), 0.79 (t, J=6.9 Hz, 3H). ¹³C NMR (101 MHz, CDCl₃) δ 161.64 (d, J=244.7 Hz), 160.1, 148.2, 138.14 (d, J=3.1 Hz), 129.54 (d, J=7.8 Hz), 113.69 (d, J=20.9 Hz), 107.5, 97.9, 80.3, 55.3, 46.7, 35.8, 32.5, 24.9, 24.8, 23.8, 22.7, 14.1. ¹⁹F NMR (377 MHz, CDCl₃) δ-117.8. IR (neat, vₘₐₓ/cm⁻¹): 3559, 2955, 2872, 1595, 1508, 1457, 1421, 1205, 1156, 1054.

HRMS (ESI): m/z=397.2156 [M+Na]⁺ (calc. for C₂₃H₃₁FNaO₃ m/z=397.2149).

### Step g) (E)-1-(3-(4-fluorophenyl)-2-methyloct-3-en-2-yl)-3,5-dimethoxybenzene

To a solution of 2-(3,5-dimethoxyphenyl)-3-(4-fluorophenyl)-2-methyloctan-3-ol (1.32 g, 3.53 mmol, 1.0 equiv) in anhydrous THF (22 mL) at −78° C. was added KHMDS (0.5 M in toluene, 17.6 mL, 8.81 mmol, 2.5 equiv) and CS₂ (3.20 mL, 52.9 mmol, 15 equiv). The yellow solution was stirred at −78° C. for 10 min, the cooling bath was removed and the mixture stirred for another 30 min. Mel (3.51 mL, 56.4 mmol, 16 equiv) was added and the mixture was first stirred at ambient temperature for 1 h, and then at 40° C. overnight. The mixture was diluted with Et₂O (25 mL) and aq. sat. NaHCO₃ (25 mL) was added. The layers were separated and the aqueous phase was extracted with Et₂O (2×25 mL). Combined organic extracts were washed with brine, dried over MgSO₄ and concentrated in vacuo. Purification by flash column chromatography (SiO₂; 0-1.5% Et₂O in hexanes) afforded the product as an amber orange liquid (1.25 g, 99%).

¹H NMR (400 MHz, CDCl₃) δ 6.87-6.77 (m, 2H), 6.63-6.57 (m, 2H), 6.49 (d, J=2.3 Hz, 2H), 6.32 (t, J=2.3 Hz, 1H), 5.72 (t, J=7.3 Hz, 1H), 3.77 (s, 6H), 1.79-1.68 (m, 2H), 1.36 (s, 6H), 1.31-1.19 (m, 4H), 0.80 (t, J=7.1 Hz, 3H). ¹³C NMR (101 MHz, CDCl₃) δ 161.5 (d, J=244.1 Hz), 160.5, 150.8, 147.5, 136.2 (d, J=3.4 Hz), 131.2 (d, J=7.6 Hz), 126.9, 114.3 (d, J=20.9 Hz), 105.7, 97.4, 55.4, 44.2, 32.2, 29.0, 28.9, 22.4, 14.1. ¹⁹F NMR (377 MHz, CDCl₃) δ-117.2. IR (neat, vₘₐₓ/cm⁻¹): 2958, 2933, 2867, 1600, 1508, 1458, 1422, 1205, 1155, 1059. HRMS (ESI): m/z=379.2043 [M+Na]⁺ (calc. for C₂₃H₂₉FNaO₂ m/z=379.2044).

### Step h) (E)-5-(3-(4-fluorophenyl)-2-methyloct-3-en-2-yl)benzene-1,3-diol

To a solution of (E)-1-(3-(4-fluorophenyl)-2-methyloct-3-en-2-yl)-3,5-dimethoxybenzene (1.25 g, 3.51 mmol, 1.0 equiv) in anhydrous CH₂Cl₂ (30 mL) at 0° C. was added BBr₃ (1.0 M in CH₂Cl₂, 10.5 mL, 10.5 mmol, 3.0 equiv) dropwise and the green solution was stirred at 0° C. for 5.5 h. The mixture was diluted with CH₂Cl₂ (25 mL) and carefully quenched with aq. sat. NaHCO₃ (25 mL). The layers were separated and the aqueous phase was extracted with CH₂Cl₂ (2×30 mL). Combined organic extracts were dried over MgSO₄ and concentrated in vacuo. Purification by flash column chromatography (SiO₂; using 0-50% EtOAc in hexanes) afforded the product as a golden oil (1.14 g, 99%).

¹H NMR (400 MHz, CDCl₃) δ 6.86-6.78 (m, 2H), 6.64-6.57 (m, 2H), 6.38 (d, J=2.2 Hz, 2H), 6.20 (t, J=2.2 Hz, 1H), 5.68 (t, J=7.2 Hz, 1H), 5.19 (bs, 2H), 1.75-1.65 (m, 2H), 1.32 (s, 6H), 1.29-1.17 (m, 4H), 0.81 (t, J=7.2 Hz, 3H). ¹³C NMR (101 MHz, CDCl₃) δ 161.5 (d, J=244.2 Hz), 156.3, 151.8, 147.2, 136.1 (d, J=3.5 Hz), 131.2 (d, J=7.6 Hz), 127.0, 114.3 (d, J=20.8 Hz), 107.1, 100.5, 43.9, 32.2, 29.0, 28.8, 22.5, 14.1. ¹⁹F NMR (377 MHz, CDCl₃) δ-117.0. IR (neat, vₘₐₓ/cm⁻¹): 3311, 2961, 2929, 2872, 1599, 1507, 1466, 1324, 1220, 1153, 993, 835. HRMS (ESI): m/z=351.1729 [M+Na]⁺ (calc. for C₂₁H₂₅FNaO₂ m/z=351.1731).

### Step i) 5-(3-(4-fluorophenyl)-2-methyloctan-2-yl)benzene-1,3-diol

To a solution of (E)-5-(3-(4-fluorophenyl)-2-methyloct-3-en-2-yl)benzene-1,3-diol (800 mg, 2.44 mmol, 1.0 equiv) in EtOAc (20 mL) was added Pd (10 wt % on C, 2.59 g, 2.44 mmol, 1.0 equiv) and the mixture was stirred for 48 h at rt in an autoclave under 35 bar H₂ pressure.

The suspension was filtered over a pad of Celite and concentrated in vacuo. Purification by flash column chromatography (SiO₂; using 20-30% EtOAc in hexanes) afforded the product as a golden foam (550 mg, 68% yield). The racemate was resolved by chiral SFC Method 3. to afford (R)-5-(3-(4-fluorophenyl)-2-methyloctan-2-yl)benzene-1,3-diol A (170 mg, 21%, % ee>99%) and (S)-5-(3-(4-fluorophenyl)-2-methyloctan-2-yl)benzene-1,3-diol B (160 mg, 20%, % ee>99%).

¹H NMR (400 MHz, CDCl₃) δ 7.02-6.95 (m, 2H), 6.95-6.88 (m, 2H), 6.36 (d, J=2.2 Hz, 2H), 6.21 (t, J=2.2 Hz, 1H), 5.03 (bs, 2H), 2.71 (dd, J=12.1, 3.0 Hz, 1H), 1.62-1.50 (m, 1H), 1.47-1.37 (m, 1H), 1.20 (s, 3H), 1.15-1.01 (m, 4H), 1.07 (s, 3H), 0.96-0.83 (m, 2H), 0.75 (t, J=6.7 Hz, 3H). ¹³C NMR (101 MHz, CDCl₃) δ 161.5 (d, J=243.7 Hz), 156.2, 153.0, 137.4 (d, J=3.2 Hz), 131.3 (d, J=7.6 Hz), 114.3 (d, J=20.8 Hz), 106.8, 100.3, 56.4, 41.6, 31.8, 29.5, 28.6, 28.0, 23.8, 22.6, 14.1. ¹⁹F NMR (377 MHz, CDCl₃) δ-117.4. IR (neat, vₘₐₓ/cm⁻¹): 3345, 2961, 2932, 2863, 1600, 1509, 1225, 1157, 993, 842.

HRMS (ESI): 353.1889 [M+Na]⁺ (calc. for C₂₁H₂₇FNaO₂ m/z=353.1887). (R)-5-(3-(4-fluorophenyl)-2-methyloctan-2-yl)benzene-1,3-diol A [α]²⁵_{D}=−40.068±0.134 (c=1.0, CHCl₃). (S)-5-(3-(4-fluorophenyl)-2-methyloctan-2-yl)benzene-1,3-diol B [α]²⁵_{D}=+40.982±0.175 (c=1.0, CHCl₃).

### Step o) ((1S,4S,5S)-4-(4-((R)-3-(4-fluorophenyl)-2-methyloctan-2-yl)-2,6-dihydroxyphenyl)-6,6-dimethylbicyclo[3.1.1]hept-2-en-2-yl)methyl pivalate

To a solution of (R)-5-(3-(4-fluorophenyl)-2-methyloctan-2-yl)benzene-1,3-diol A (24.0 mg, 72.6 mol, 1.0 equiv) and ((1S,4R,5R)-4-hydroxy-6,6-dimethylbicyclo[3.1.1]hept-2-en-2-yl)methyl pivalate (20.2 mg, 79.8 μmol, 1.1 equiv) in CH₂Cl₂ (3.0 mL) was added pTsOH·H₂O (3.8 mg. 20.3 μmol, 0.28 equiv) and the solution was stirred for 30 min. The reaction was stopped by addition of sat. aq. NaHCO₃ (3 mL) and diluted with Et₂O (5 mL). The layers were separated and the aqueous phase was extracted with Et₂O (3×5 mL). Combined organic extracts were dried over MgSO₄, filtered and concentrated in vacuoo. Purification by flash column chromatography (SiO₂; using 0-10% EtOAc in hexanes) afforded the product as a white foam (32.3 mg, 79%).

¹H NMR (400 MHz, CDCl₃) δ 7.04-6.94 (m, 2H), 6.94-6.87 (m, 2H), 6.31 (s, 2H), 6.03 (dt, J=3.1, 1.6 Hz, 1H), 5.69 (bs, 2H), 4.65 (dt, J=13.4, 1.5 Hz, 1H), 4.52 (ddd, J=13.5, 2.4, 1.6 Hz, 1H), 4.05-4.00 (m, 1H), 2.69 (dd, J=12.0, 3.0 Hz, 1H), 2.39 (dt, J=9.6, 5.6 Hz, 1H), 2.35-2.24 (m, 2H), 1.59-1.48 (m, 1H), 1.50 (d, J=9.7 Hz, 1H), 1.47-1.38 (m, 1H), 1.35 (s, 3H), 1.24 (s, 9H), 1.17 (s, 3H), 1.15-1.05 (m, 4H), 1.03 (s, 3H), 0.99 (s, 3H), 0.94-0.83 (m, 2H), 0.75 (t, J=6.7 Hz, 3H). ¹³C NMR (101 MHz, CDCl₃) δ 178.7, 161.5 (d, J=243.6 Hz), 154.9, 149.8, 149.6, 137.6 (d, J=3.2 Hz), 131.3 (d, J=7.5 Hz), 120.4, 114.2 (d, J=20.8 Hz), 111.9, 107.3, 66.6, 56.3, 47.5, 44.3, 41.2, 41.1, 39.1, 37.9, 31.8, 29.5, 28.9, 28.2, 28.0, 27.4, 26.0, 23.4, 22.6, 20.9, 14.1. ¹⁹F NMR (377 MHz, CDCl₃) δ-117.6. IR (neat, vₘₐₓ/cm⁻¹): 3453, 2956, 2928, 2871, 2857, 1729, 1707, 1625, 1575, 1509, 1225, 1160. HRMS (ESI): m/z=587.3509 [M+Na]⁺ (calc. for C₃₆H₄₉FNaO₄ m/z=587.3507). [α]²⁵_{D}=+28.542±0.383 (c=1.0, CHCl₃).

### Step p) ((1S,4S,5S)-4-(4-((R)-3-(4-fluorophenyl)-2-methyloctan-2-yl)-2,6-dimethoxyphenyl)-6,6-dimethylbicyclo[3.1.1]hept-2-en-2-yl)methyl pivalate

To a suspension of diol ((1S,4S,5S)-4-(4-((R)-3-(4-fluorophenyl)-2-methyloctan-2-yl)-2,6-dihydroxyphenyl)-6,6-dimethylbicyclo[3.1.1]hept-2-en-2-yl)methyl pivalate (32.3 mg, 57.1 μmol, 1.0 equiv) and K₂CO₃ (47.4 mg. 343 μmol, 6.0 equiv) in acetone (1.0 mL) was added (MeO)₂SO₂ (27.1 μL. 286 μmol, 5.0 equiv) and the solution was stirred at rt overnight. The reaction mixture was diluted with Et₂O (5 mL), filtered and concentrated in vacuo. Purification by flash column chromatography (SiO₂; 0-5% EtOAc in hexanes) afforded the product as a white foam (28.9 mg, 85%).

¹H NMR (400 MHz, CDCl₃) δ 7.00-6.80 (m, 4H), 6.39 (s, 2H), 5.78 (dt, J=2.8, 1.4 Hz, 1H), 4.62-4.55 (m, 1H), 4.54-4.48 (m, 1H), 4.01 (s, 1H), 3.69 (s, 6H), 2.71 (dd, J=11.6, 3.4 Hz, 1H), 2.18 (dd, J=7.2, 5.6 Hz, 2H), 2.05 (td, J=6.0, 2.9 Hz, 1H), 1.72 (d, J=7.4 Hz, 1H), 1.63-1.45 (m, 2H), 1.31 (s, 3H), 1.26 (s, 3H), 1.22 (s, 9H), 1.15 (s, 3H), 1.14-1.02 (m, 4H), 0.98 (s, 3H), 0.94-0.81 (m, 2H), 0.76 (t, J=6.8 Hz, 3H). ¹³C NMR (101 MHz, CDCl₃) δ 178.6, 161.5 (d, J=243.7 Hz), 158.3, 148.3, 137.6 (d, J=3.3 Hz), 137.4, 131.3 (d, J=7.5 Hz), 126.4, 118.1, 114.2 (d, J=20.8 Hz), 103.9, 67.6, 56.6, 55.9, 47.6, 44.0, 41.8, 41.1, 39.0, 37.7, 31.8, 29.5, 28.4, 28.0, 27.7, 27.5, 26.5, 24.4, 22.6, 21.2, 14.1.

¹⁹F NMR (376 MHz, CDCl₃) δ-117.6. IR (neat, vₘₐₓ/cm⁻¹): 2955, 2930, 2869, 1726, 1658, 1604, 1572, 1508, 1479, 1462, 1410, 1381, 1365, 1339, 1280, 1239, 1224, 1152, 11122, 1032, 1015, 957, 938, 907, 841, 770, 742, 702, 670, 546. HRMS (ESI): m/z=615.3808 [M+Na]⁺ (calc. for C₃₈H₅₃FNaO₄ m/z=615.3820). [α]²⁵_{D}=+50.994±0.086 (c=1.0, CDCl₃).

### Step q) ((1S,4S,5S)-4-(4-((R)-3-(4-fluorophenyl)-2-methyloctan-2-yl)-2,6-dimethoxyphenyl)-6,6-dimethylbicyclo[3.1.1]hept-2-en-2-yl)methanol

To a solution of ((1S,4S,5S)-4-(4-((R)-3-(4-fluorophenyl)-2-methyloctan-2-yl)-2,6-dimethoxyphenyl)-6,6-dimethylbicyclo[3.1.1]hept-2-en-2-yl)methyl pivalate (28.9 mg, 48.7 μmol, 1.0 equiv) in CH₂Cl₂ (0.55 mL) was added DIBAL (102 μL, 102 μmol, 2.1 equiv) at 0° C. After stirring the reaction for 15 minutes at 0° C., sat. aq. NH₄Cl (3 mL) was added, the layers were separated and the aqueous layer was extracted with CH₂Cl₂ (3×5 mL). The combined organic extracts were dried over MgSO₄ and concentrated in vacuo. Purification by flash column chromatography (SiO₂; 5-20% EtOAc in hexanes) afforded the product as a colourless oil (21.3 mg, 86%).

¹H NMR (400 MHz, CDCl₃) δ 7.02-6.84 (m, 4H), 6.39 (s, 2H), 5.71 (dt, J=3.0, 1.4 Hz, 1H), 4.08 (s, 2H), 4.01 (t, J=2.3 Hz, 1H), 3.69 (s, 6H), 2.71 (dd, J=11.5, 3.5 Hz, 1H), 2.27-2.17 (m, 2H), 2.10-2.03 (m, 1H), 1.72 (d, J=6.9 Hz, 1H), 1.62-1.45 (m, 2H), 1.32 (s, 3H), 1.26 (s, 3H), 1.15 (s, 3H), 1.13-1.01 (m, 4H), 0.98 (s, 3H), 0.94-0.82 (m, 2H), 0.76 (t, J=6.6 Hz, 3H). ¹³C NMR (101 MHz, CDCl₃) δ 161.5 (d, J=243.7 Hz), 158.3, 148.3, 142.1, 137.6 (d, J=3.3 Hz), 131.3 (d, J=7.5 Hz), 123.8, 118.2, 114.2 (d, J=20.8 Hz), 104.0, 66.8, 56.6, 56.0, 47.6, 44.0, 41.8, 41.0, 37.6, 31.8, 29.5, 28.4, 28.0, 28.0, 26.4, 24.4, 22.6, 21.2, 14.1. ¹⁹F NMR (376 MHz, CDCl₃) δ-117.6. IR (neat, vₘₐₓ/cm⁻¹): 3378, 2928, 2863, 1655, 1604, 1572, 1508, 1461, 1410, 1380, 1364, 1340, 1301, 1238, 1224, 1184, 1160, 1120, 1049, 1014, 986, 908, 840, 743, 701, 669, 563, 547, 525. HRMS (ESI): m/z=531.3238 [M+Na]⁺ (calc. for C₃₃H₄₅FNaO₃ m/z=531.3245). [α]²⁵_{D}=+43.736±0.139 (c=1.0, CDCl₃).

### Example 10

### ((1S,4S,5S)-4-(4-((S)-3-(4-fluorophenyl)-2-methyloctan-2-yl)-2,6-dimethoxyphenyl)-6,6-dimethylbicyclo[3.1.1]hept-2-en-2-yl)methanol

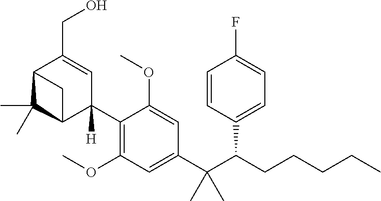 [SMILES: CCCCC[C@@H](c1ccc(F)cc1)C(C)(C)c1cc(OC)c([C@H]2C=C(CO)[C@H]3C[C@@H]2C3(C)C)c(OC)c1]

### Step o) ((1S,4S,5S)-4-(4-((S)-3-(4-fluorophenyl)-2-methyloctan-2-yl)-2,6-dihydroxyphenyl)-6,6-dimethylbicyclo[3.1.1]hept-2-en-2-yl)methyl pivalate

To a solution of (S)-5-(3-(4-fluorophenyl)-2-methyloctan-2-yl)benzene-1,3-diol B (26.6 mg, 80.5 mol, 1.0 equiv) and ((1S,4R,5R)-4-hydroxy-6,6-dimethylbicyclo[3.1.1]hept-2-en-2-yl)methyl pivalate (22.4 mg, 88.5 μmol, 1.1 equiv) in CH₂Cl₂ (3.3 mL) was added pTsOH·H₂O (4.3 mg. 22.5 μmol, 0.28 equiv) and the solution was stirred for 30 min. The reaction was stopped by addition of sat. aq. NaHCO₃ (3 mL) and diluted with Et₂O (5 mL). The layers were separated and the aqueous phase was extracted with Et₂O (3×5 mL). Combined organic extracts were dried over MgSO₄, filtered and concentrated in vacuoo. Purification by flash column chromatography (SiO₂; using 0-10% EtOAc in hexanes) afforded the product as a white foam (38.5 mg, 85%).

¹H NMR (500 MHz, CDCl₃) δ 7.03-6.96 (m, 2H), 6.96-6.87 (m, 2H), 6.32 (s, 2H), 6.03 (dt, J=3.1, 1.5 Hz, 1H), 5.70 (bs, 2H), 4.65 (ddd, J=13.5, 2.0, 1.4 Hz, 1H), 4.52 (ddd, J=13.5, 2.4, 1.6 Hz, 1H), 4.09-3.96 (m, 1H), 2.69 (dd, J=12.1, 3.0 Hz, 1H), 2.39 (dt, J=9.7, 5.6 Hz, 1H), 2.35-2.25 (m, 2H), 1.58-1.48 (m, 1H), 1.50 (d, J=9.8 Hz, 1H), 1.45-1.38 (m, 1H), 1.35 (s, 3H), 1.24 (s, 9H), 1.17 (s, 3H), 1.14-1.05 (m, 4H), 1.02 (s, 3H), 0.99 (s, 3H), 0.95-0.81 (m, 2H), 0.74 (t, J=6.6 Hz, 3H). ¹³C NMR (126 MHz, CDCl₃) δ 178.7, 161.5 (d, J=243.6 Hz), 154.9, 149.9, 149.6, 137.6 (d, J=3.2 Hz), 131.3 (d, J=7.5 Hz), 120.4, 114.3 (d, J=20.8 Hz), 111.9, 107.3, 66.6, 56.2, 47.5, 44.3, 41.2, 41.1, 39.1, 37.9, 31.8, 29.4, 28.8, 28.2, 28.0, 27.4, 26.0, 23.2, 22.6, 20.9, 14.1. ¹⁹F NMR (471 MHz, CDCl₃) δ-117.6. IR (neat, vₘₐₓ/cm⁻¹): 3453, 2956, 2929, 2871, 1728, 1707, 1625, 1575, 1509, 1225, 1160. HRMS (ESI): m/z=587.3495 [M+Na]⁺ (calc. for C₃₆H₄₉FNaO₄ m/z=587.3507). [α]²⁵_{D}=+77.406±0.191 (c=1.0, CHCl₃).

### Step p) ((1S,4S,5S)-4-(4-((S)-3-(4-fluorophenyl)-2-methyloctan-2-yl)-2,6-dimethoxyphenyl)-6,6-dimethylbicyclo[3.1.1]hept-2-en-2-yl)methyl pivalate

To a suspension of diol ((1S,4S,5S)-4-(4-((S)-3-(4-fluorophenyl)-2-methyloctan-2-yl)-2,6-dihydroxyphenyl)-6,6-dimethylbicyclo[3.1.1]hept-2-en-2-yl)methyl pivalate (38.5 mg, 68.1 μmol, 1.0 equiv) and K₂CO₃ (56.5 mg. 409 μmol, 6.0 equiv) in acetone (1.0 mL) was added (MeO)₂SO₂ (32.3 μL. 341 μmol, 5.0 equiv) and the solution was stirred at rt overnight. The reaction mixture was diluted with Et₂O (5 mL), filtered and concentrated in vacuo. Purification by flash column chromatography (SiO₂; 0-5% EtOAc in hexanes) afforded the product as a white foam (35.5 mg, 88%).

¹H NMR (400 MHz, CDCl₃) δ 6.99-6.85 (m, 4H), 6.39 (s, 2H), 5.79 (dt, J=2.9, 1.4 Hz, 1H), 4.62-4.56 (m, 1H), 4.55-4.48 (m, 1H), 4.01 (s, 1H), 3.69 (s, 6H), 2.71 (dd, J=11.7, 3.4 Hz, 1H), 2.22-2.14 (m, 2H), 2.10-2.02 (m, 1H), 1.70 (d, J=7.8 Hz, 1H), 1.63-1.45 (m, 2H), 1.31 (s, 3H), 1.26 (s, 3H), 1.22 (s, 9H), 1.15 (s, 3H), 1.14-1.02 (m, 4H), 0.99 (s, 3H), 0.94-0.81 (m, 2H), 0.76 (t, J=6.8 Hz, 3H). ¹³C NMR (101 MHz, CDCl₃) δ 178.6, 161.5 (d, J=243.7 Hz), 158.3, 148.3, 137.6 (d, J=3.2 Hz), 137.4, 131.3 (d, J=7.3 Hz), 126.5, 118.1, 114.2 (d, J=20.8 Hz), 103.9, 67.6, 56.6, 55.9, 47.6, 44.0, 41.8, 41.1, 39.0, 37.7, 31.8, 29.5, 28.4, 28.0, 27.7, 27.5, 26.5, 24.4, 22.6, 21.2, 14.1. ¹⁹F NMR (376 MHz, CDCl₃) δ-117.6. IR (neat, vₘₐₓ/cm⁻¹): 2931, 2869, 1726, 1658, 1604, 1572, 1508, 1479, 14623, 1410, 1397, 1381, 1364, 1339, 1280, 1239, 1224, 1151, 1121, 1042, 1031, 1015, 957, 938, 907, 841, 769, 757, 702, 562, 547, 525. HRMS (ESI): m/z=615. 3818 [M+Na]⁺ (calc. for C₃₈H₅₃FNaO₄ m/z=615.3820). [α]²⁵_{D}=+96.771±0.079 (c=1.0, CDCl₃).

### Step q) ((1S,4S,5S)-4-(4-((S)-3-(4-fluorophenyl)-2-methyloctan-2-yl)-2,6-dimethoxyphenyl)-6,6-dimethylbicyclo[3.1.1]hept-2-en-2-yl)methanol

To a solution of ((1S,4S,5S)-4-(4-((S)-3-(4-fluorophenyl)-2-methyloctan-2-yl)-2,6-dimethoxyphenyl)-6,6-dimethylbicyclo[3.1.1]hept-2-en-2-yl)methyl pivalate (35.5 mg, 59.8 μmol, 1.0 equiv) in CH₂Cl₂ (0.55 mL) was added DIBAL (126 μL, 126 μmol, 2.1 equiv) at 0° C. After stirring the reaction for 15 minutes at 0° C., sat. aq. NH₄Cl (3 mL) was added, the layers were separated and the aqueous layer was extracted with CH₂Cl₂ (3×5 mL). The combined organic extracts were dried over MgSO₄ and concentrated in vacuo. Purification by flash column chromatography (SiO₂; 5-20% EtOAc in hexanes) afforded the product as a colourless oil (29.1 mg, 96%).

¹H NMR (400 MHz, CDCl₃) δ 6.98-6.83 (m, 4H), 6.39 (s, 2H), 5.72 (dt, J=2.9, 1.4 Hz, 1H), 4.08 (s, 2H), 4.01 (s, 1H), 3.70 (s, 6H), 2.71 (dd, J=11.7, 3.3 Hz, 1H), 2.26-2.18 (m, 2H), 2.11-2.05 (m, 1H), 1.70 (d, J=7.6 Hz, 1H), 1.63-1.45 (m, 2H), 1.32 (s, 3H), 1.26 (s, 3H), 1.15 (s, 3H), 1.14-1.02 (m, 4H), 0.98 (s, 3H), 0.94-0.80 (m, 2H), 0.76 (t, J=6.7 Hz, 3H). ¹³C NMR (101 MHz, CDCl₃) δ 161.5 (d, J=243.7 Hz), 158.3, 148.3, 142.1, 137.6 (d, J=3.2 Hz), 131.3 (d, J=7.5 Hz), 123.7, 118.2, 114.2 (d, J=20.8 Hz), 103.9, 66.8, 56.6, 56.0, 47.6, 44.0, 41.8, 41.0, 37.6, 31.8, 29.5, 28.4, 28.0, 28.0, 26.4, 24.4, 22.6, 21.2, 14.1. ¹⁹F NMR (376 MHz, CDCl₃) δ-117.6. IR (neat, vₘₐₓ/cm⁻¹): 3379, 2929, 2864, 1655, 1604, 1572, 1508, 1462, 1410, 1381, 1364, 1340, 1301, 1238, 1224, 1184, 1160,1120, 1049, 1014, 986, 908, 841, 742, 701, 670, 563, 525. HRMS (ESI): m/z=531.3248 [M+Na]⁺ (calc. for C₃₃H₄₅FNaO₃ m/z=531.3245). [α]²⁵_{D}=+109.233±0.100(c=1.0, CDCl₃).

### Example 11

### ((1S,4S,5S)-4-(4-((R)-3-(3-fluorophenyl)-2-methyloctan-2-yl)-2,6-dimethoxyphenyl)-6,6-dimethylbicyclo[3.1.1]hept-2-en-2-yl)methanol

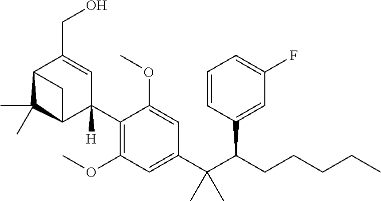 [SMILES: CCCCC[C@H](c1cccc(F)c1)C(C)(C)c1cc(OC)c([C@H]2C=C(CO)[C@H]3C[C@@H]2C3(C)C)c(OC)c1]

### Step b) 2-(3,5-dimethoxyphenyl)-3-(3-fluorophenyl)-2-methyloctan-3-ol Preparation of 3-fluorophenyllithium

To a solution of 3-fluoroiodobenzene (861 μL, 7.33 mmol, 2.0 equiv) in dry Et₂O (8.0 mL) was added at −78° C. n-BuLi (1.6 M in hexane, 3.43 mL, 5.50 mmol, 1.5 equiv) dropwise and the solution was stirred at −78° C. for 30 min.

To a solution of LaCl₃·2LiCl (0.6 M in THF, 6.11 mL, 3.66 mmol, 1.0 equiv) was added 2-(3,5-dimethoxyphenyl)-2-methyloctan-3-one (1.02 g, 3.66 mmol, 1.0 equiv) in dry THF (3.0 mL) and the resulting mixture was stirred at rt for 1 h. The reaction mixture was cooled to 0° C. and the previously prepared solution of 3-fluorophenyllithium was added dropwise. The mixture was stirred for 30 min. at 0° C. and then quenched with aq. sat. NH₄Cl solution (15 mL) and H₂O (15 mL). The mixture was diluted with Et₂O (15 mL), the phases were separated and the aqueous phase was extracted with Et₂O (3×15 mL). Combined organic extracts were dried with MgSO₄, filtered and concentrated in vacuo. The crude material was purified by flash column chromatography (SiO₂; 0-15% EtOAc in hexanes) to afford the product as a colourless oil (1.26 g, 99%).

¹H NMR (400 MHz, CDCl₃) δ 7.29-7.20 (m, 1H), 7.11-6.99 (m, 2H), 6.98-6.91 (m, 1H), 6.51 (d, J=2.3 Hz, 2H), 6.41 (t, J=2.2 Hz, 1H), 3.78 (s, 6H), 2.27-2.14 (m, 1H), 1.88 (s, 1H), 1.70-1.56 (m, 1H), 1.41 (s, 3H), 1.29 (s, 3H), 1.20 (d, J=4.2 Hz, 4H), 0.94-0.77 (m, 2H), 0.82 (t, J=6.8 Hz, 3H). ¹³C NMR (101 MHz, CDCl₃) δ 162.33 (d, J=243.6 Hz), 160.1, 148.0, 145.58 (d, J=6.6 Hz), 128.15 (d, J=8.0 Hz), 123.54 (d, J=2.7 Hz), 115.22 (d, J=22.4 Hz), 113.11 (d, J=21.1 Hz), 107.5, 98.0, 80.3, 55.3, 46.6, 35.8, 32.5, 25.0, 24.7, 23.7, 22.7, 14.1. ¹⁹F NMR (377 MHz, CDCl₃) δ-114.5. IR (neat, vₘₐₓ/cm⁻¹): 3554, 2955, 2871, 1594, 1457, 1422, 1205, 1156, 1059. HRMS (ESI): m/z=397.2144 [M+Na]⁺ (calc. for C₂₃H₃₁FNaO₃ m/z=397.2149).

### Step g) (E)-1-(3-(3-fluorophenyl)-2-methyloct-3-en-2-yl)-3,5-dimethoxybenzene

To a solution of 2-(3,5-dimethoxyphenyl)-3-(3-fluorophenyl)-2-methyloctan-3-ol (1.40 g, 3.74 mmol, 1.0 equiv) in anhydrous THF (24 mL) at −78° C. was added KHMDS (0.5 M in toluene, 18.7 mL, 9.35 mmol, 2.5 equiv) and CS₂ (3.39 mL, 56.1 mmol, 15 equiv). The yellow solution was stirred at −78° C. for 10 min, the cooling bath was removed and the mixture stirred for another 30 min. Mel (3.72 mL, 59.8 mmol, 16 equiv) was added and the mixture was first stirred at ambient temperature for 1 h, and then at 40° C. overnight. The mixture was diluted with Et₂O (25 mL) and aq. sat. NaHCO₃ (25 mL) was added. The layers were separated and the aqueous phase was extracted with Et₂O (2×25 mL). Combined organic extracts were washed with brine, dried over MgSO₄ and concentrated in vacuo. Purification by flash column chromatography (SiO₂; 0-1.2% Et₂O in hexanes) afforded the product as an amber orange liquid (1.09 g, 82%).

¹H NMR (400 MHz, CDCl₃) δ 7.10 (ddd, J=8.4, 7.6, 6.1 Hz, 1H), 6.89-6.82 (m, 1H), 6.52 (d, J=2.3 Hz, 2H), 6.50-6.46 (m, 1H), 6.41 (ddd, J=10.2, 2.7, 1.5 Hz, 1H), 6.35 (t, J=2.2 Hz, 1H), 5.73 (t, J=7.3 Hz, 1H), 3.79 (s, 6H), 1.82-1.73 (m, 2H), 1.39 (s, 6H), 1.37-1.20 (m, 4H), 0.83 (t, J=7.2 Hz, 3H). ¹³C NMR (101 MHz, CDCl₃) δ 162.2 (d, J=244.9 Hz), 160.5, 150.7, 147.4 (d, J=1.6 Hz), 142.8 (d, J=7.5 Hz), 128.7 (d, J=8.4 Hz), 127.0, 125.57 (d, J=2.8 Hz), 116.6 (d, J=20.8 Hz), 113.0 (d, J=20.9 Hz), 105.6, 97.5, 55.3, 44.1, 32.2, 29.0, 29.0, 22.4, 14.0. ¹⁹F NMR (377 MHz, CDCl₃) δ-114.6. IR (neat, vₘₐₓ/cm¹): 2957, 2933, 2873, 1598, 1458, 1423, 1205, 1155, 1058. HRMS (ESI): m/z=379.2041 [M+Na]⁺ (calc. for C₂₃H₂₉FNaO₂ m/z=379.2044).

### Step h) (E)-5-(3-(3-fluorophenyl)-2-methyloct-3-en-2-yl)benzene-1,3-diol

To a solution of (E)-1-(3-(3-fluorophenyl)-2-methyloct-3-en-2-yl)-3,5-dimethoxybenzene (1.01 g, 2.83 mmol, 1.0 equiv) in anhydrous CH₂Cl₂ (25 mL) at 0° C. was added BBr₃ (1.0 M in CH₂Cl₂, 8.50 mL, 8.50 mmol, 3.0 equiv) dropwise and the green solution was stirred at 0° C. for 5.5 h. The mixture was diluted with CH₂Cl₂ (25 mL) and carefully quenched with aq. sat. NaHCO₃ (25 mL). The layers were separated and the aqueous phase was extracted with CH₂Cl₂ (2×30 mL). Combined organic extracts were dried over MgSO₄ and concentrated in vacuo. Purification by flash column chromatography (SiO₂; using 0-50% EtOAc in hexanes) afforded the product as a golden oil (920 mg, 99%).

¹H NMR (400 MHz, CDCl₃) δ 7.08 (dd, J=8.0, 6.1 Hz, 1H), 6.83 (ddd, J=8.5, 2.6, 1.0 Hz, 1H), 6.46 (dd, J=7.6, 1.2 Hz, 1H), 6.40 (d, J=2.1 Hz, 2H), 6.39 (dd, J=2.7, 1.7 Hz, 1H), 6.20 (t, J=2.2 Hz, 1H), 5.67 (t, J=7.3 Hz, 1H), 5.27 (bs, 2H), 1.75-1.68 (m, 2H), 1.31 (s, 6H), 1.29-1.17 (m, 4H), 0.81 (t, J=7.1 Hz, 3H). ¹³C NMR (101 MHz, CDCl₃) δ 162.2 (d, J=244.7 Hz), 156.2, 151.8, 147.1 (d, J=1.7 Hz), 142.7 (d, J=7.5 Hz), 128.8 (d, J=8.4 Hz), 127.1, 125.6 (d, J=2.7 Hz), 116.6 (d, J=20.8 Hz), 113.1 (d, J=20.9 Hz), 107.1, 100.6, 43.8, 32.1, 29.0, 28.8, 22.4, 14.1. ¹⁹F NMR (377 MHz, CDCl₃) δ-114.3. IR (neat, vₘₐₓ/cm⁻¹): 3341, 2961, 2930, 2872, 1599, 1580, 1466, 1434, 1423, 1324, 1151, 993. HRMS (ESI): m/z=351.1732 [M+Na]⁺ (calc. for C₂₁H₂₅FNaO₂ m/z=351.1731).

### Step i) 5-(3-(3-fluorophenyl)-2-methyloctan-2-yl)benzene-1,3-diol

To a solution of (E)-5-(3-(3-fluorophenyl)-2-methyloct-3-en-2-yl)benzene-1,3-diol (850 mg, 2.59 mmol, 1.0 equiv) in EtOAc (25 mL) was added Pd (10 wt % on C, 2.75 g, 2.59 mmol, 1.0 equiv) and the mixture was stirred for 48 h at rt in an autoclave under 35 bar H₂ pressure. The suspension was filtered over a pad of Celite and concentrated in vacuo. Purification by flash column chromatography (SiO₂; using 20-30% EtOAc in hexanes) afforded the product as a golden foam (560 mg, 65% yield). The racemate was resolved by chiral SFC Method 4. to afford (R)-5-(3-(3-fluorophenyl)-2-methyloctan-2-yl)benzene-1,3-diol A (200 mg, 23%, % ee>99%) and (S)-5-(3-(3-fluorophenyl)-2-methyloctan-2-yl)benzene-1,3-diol B (190 mg, 22%, % ee>99%).

¹H NMR (400 MHz, CDCl₃) δ 7.21-7.11 (m, 1H), 6.92-6.74 (m, 3H), 6.43 (d, J=2.2 Hz, 2H), 6.26 (t, J=2.1 Hz, 1H), 6.08 (bs, 2H), 2.73 (dd, J=12.1, 2.8 Hz, 1H), 1.61-1.49 (m, 1H), 1.48-1.34 (m, 1H), 1.21 (s, 3H), 1.15-1.00 (m, 4H), 1.05 (s, 3H), 0.95-0.84 (m, 2H), 0.74 (t, J=6.9 Hz, 3H). ¹³C NMR (101 MHz, CDCl₃) δ 162.5 (d, J=243.9 Hz), 156.1, 153.1, 144.7 (d, J=6.7 Hz), 128.8 (d, J=8.3 Hz), 126.0 (d, J=1.2 Hz), 116.6 (d, J 10=20.8 Hz), 113.0 (d, J=21.0 Hz), 106.9, 100.5, 56.9, 41.5, 31.8, 29.4, 28.7, 28.0, 23.6, 22.5, 14.1. ¹⁹F NMR (377 MHz, CDCl₃) δ-114.4. IR (neat, vₘₐₓ/cm⁻¹): 3342, 2960, 2933, 2862, 1595, 1486, 1466, 1445, 1326, 1150, 992, 704. HRMS (ESI): 353.1882 [M+Na]⁺ (calc. for C₂₁H₂₇FNaO₂ m/z=353.1887). (R)-5-(3-(3-fluorophenyl)-2-methyloctan-2-yl)benzene-1,3-diol A [α]²⁵_{D}=−40.385±0.408 (c=1.0, CHCl₃). (S)-5-(3-(3-fluorophenyl)-2-methyloctan-2-yl)benzene-1,3-diol B [α]²⁵_{D}=+42.357±0.102 (c=1.0, CHCl₃).

### Step o) ((1S,4S,5S)-4-(4-((R)-3-(3-fluorophenyl)-2-methyloctan-2-yl)-2,6-dihydroxyphenyl)-6,6-dimethylbicyclo[3.1.1]hept-2-en-2-yl)methyl pivalate

To a solution of resorcinol (R)-5-(3-(3-fluorophenyl)-2-methyloctan-2-yl)benzene-1,3-diol A (25.5 mg, 77.1 μmol, 1.0 equiv) and pTsOH·H₂O (4.1 mg, 21.6 μmol, 0.28 equiv) in CH₂Cl₂ (3.0 mL) was added ((1S,4R,5R)-4-hydroxy-6,6-dimethylbicyclo[3.1.1]hept-2-en-2-yl)methyl pivalate (21.4 mg, 84.8 μmol, 1.1 equiv) and the solution was stirred at rt for 30 minutes. The reaction was stopped by addition of sat. aq. NaHCO₃ (3 mL), the layers were separated and the aqueous layer was extracted with Et₂O (3×5 mL). The combined organic extracts were dried over MgSO₄ and concentrated in vacuo. Purification by flash column chromatography (SiO₂; 5-15% EtOAc in hexanes) afforded the product as a colourless foam (33.6 mg, 77%).

¹H NMR (400 MHz, CDCl₃) δ 7.17 (td, J=8.0, 6.2 Hz, 1H), 6.91-6.82 (m, 2H), 6.75 (dt, J=10.4, 2.1 Hz, 1H), 6.33 (s, 2H), 6.03 (dt, J=3.2, 1.6 Hz, 1H), 5.72 (bs, 2H), 4.65 (dt,J=13.5, 1.7 Hz, 1H), 4.52 (dt, J=13.5, 2.0 Hz, 1H), 4.06-4.01 (m, 1H), 2.71 (dd, J=12.0, 2.9 Hz, 1H), 2.39 (dt, J=9.7, 5.6 Hz, 1H), 2.35-2.28 (m, 2H), 1.62-1.53 (m, 1H), 1.51 (d, J=9.7 Hz, 1H), 1.45-1.38 (m, 1H), 1.35 (s, 3H), 1.24 (s, 9H), 1.20 (s, 3H), 1.16-1.05 (m, 4H), 1.04 (s, 3H), 0.99 (s, 3H), 0.96-0.82 (m, 2H), 0.75 (t, J=6.7 Hz, 3H). ¹³C NMR (101 MHz, CDCl₃) δ 178.7, 162.5 (d, J=244.0 Hz), 155.0, 149.8, 149.6, 145.0 (d, J=6.7 Hz), 128.7 (d, J=8.3 Hz), 126.0, 120.4, 116.6 (d, J=20.8 Hz), 113.0 (d, J=21.0 Hz), 112.0, 107.3, 66.6, 56.9, 47.5, 44.3, 41.2, 41.1, 39.1, 37.9, 31.8, 29.4, 28.9, 28.1, 28.0, 27.4, 26.0, 23.3, 22.5, 20.9, 14.1. ¹⁹F NMR (376 MHz, CDCl₃) δ-114.7. IR (neat, vₘₐₓ/cm¹): 3451, 2956, 2931, 2870, 1728, 1707, 1624, 1613, 1586, 1577, 1510, 1480, 1464, 1445, 1429, 1398, 1384, 1366, 1328, 1283, 1230, 1159, 1111, 1073, 1040, 1027, 963, 935, 916, 876, 783., 758, 724, 700, 672, 587, 569, 545, 521. HRMS (ESI): m/z=587.3499 [M+Na]⁺ (calc. for C₃₆H₄₉FNaO₄ m/z=587.3507). [α]²⁵_{D}=+14.014±0.249 (c=1.0, CHCl₃).

### Step p) ((1S,4S,5S)-4-(4-((R)-3-(3-fluorophenyl)-2-methyloctan-2-yl)-2,6-dimethoxyphenyl)-6,6-dimethylbicyclo[3.1.1]hept-2-en-2-yl)methyl pivalate

To a suspension of diol ((1S,4S,5S)-4-(4-((R)-3-(3-fluorophenyl)-2-methyloctan-2-yl)-2,6-dihydroxyphenyl)-6,6-dimethylbicyclo[3.1.1]hept-2-en-2-yl)methyl pivalate (33.6 mg, 59.4 μmol, 1.0 equiv) and K₂CO₃ (49.3 mg. 357 μmol, 6.0 equiv) in acetone (0.7 mL) was added (MeO)₂SO₂ (28.2 μL, 298 μmol, 5.0 equiv) and the solution was stirred at rt overnight. The reaction mixture was diluted with Et₂O (5 mL), filtered and concentrated in vacuo. Purification by flash column chromatography (SiO₂; 0-5% EtOAc in hexanes) afforded the product as a white foam (28.3 mg, 80%).

¹H NMR (400 MHz, CDCl₃) δ 7.21-7.12 (m, 1H), 6.91-6.84 (m, 1H), 6.79 (d, J=7.7 Hz, 1H), 6.75-6.66 (m, 1H), 6.40 (s, 2H), 5.78 (dt, J=2.8, 1.4 Hz, 1H), 4.61-4.55 (m, 1H), 4.55-4.48 (m, 1H), 4.01 (s, 1H), 3.69 (s, 6H), 2.72 (dd, J=11.8, 3.2 Hz, 1H), 2.23-2.14 (m, 2H), 2.08-2.02 (m, 1H), 1.72 (d, J=7.6 Hz, 1H), 1.62-1.44 (m, 2H), 1.30 (s, 3H), 1.28 (s, 3H), 1.22 (s, 9H), 1.16 (s, 3H), 1.15-1.03 (m, 4H), 0.98 (s, 3H), 0.94-0.84 (m, 2H), 0.76 (t, J=6.8 Hz, 3H). ¹³C NMR (101 MHz, CDCl₃) δ 178.6, 162.5 (d, J=244.1 Hz), 158.3, 148.3, 145.0 (d, J=6.7 Hz), 137.4, 128.6 (d, J=8.3 Hz), 126.4, 126.0, 118.2, 116.6 (d, J=21.2 Hz), 112.9 (d, J=21.0 Hz), 103.9, 67.6, 57.2, 56.0, 47.6, 44.0, 41.8, 41.1, 39.0, 37.7, 31.8, 29.4, 28.5, 28.0, 27.7, 27.5, 26.5, 24.3, 22.6, 21.2, 14.1. ¹⁹F NMR (376 MHz, CDCl₃) δ-114.8. IR (neat, vₘₐₓ/cm⁻¹): 2955, 2930, 2869, 1726, 1656, 1604, 1586, 1573, 1480, 1455, 1410, 1397, 1382, 1365, 1338, 1280, 1239, 1149, 1120, 1032, 957, 928, 904, 871, 852, 832, 782, 724, 699, 585, 520. HRMS (ESI): m/z=615.3806 [M+Na]⁺ (calc. for C₃₈H₅₃FNaO₄ m/z=615.3820). [α]²⁵_{D}=+25.224±0.173 (c=1.0, CHCl₃).

### Step o) ((1S,4S,5S)-4-(4-((R)-3-(3-fluorophenyl)-2-methyloctan-2-yl)-2,6-dimethoxyphenyl)-6,6-dimethylbicyclo[3.1.1]hept-2-en-2-yl)methanol

To a solution of ((1S,4S,5S)-4-(4-((R)-3-(3-fluorophenyl)-2-methyloctan-2-yl)-2,6-dimethoxyphenyl)-6,6-dimethylbicyclo[3.1.1]hept-2-en-2-yl)methyl pivalate (30.2 mg, 50.1 μmol, 1.0 equiv) in CH₂Cl₂ (0.55 mL) was added DIBAL (107 μL, 107 μmol, 2.1 equiv) at 0° C. After stirring the reaction for 15 minutes at 0° C., sat. aq. NH₄Cl (3 mL) was added, the layers were separated and the aqueous layer was extracted with CH₂Cl₂ (3×5 mL). The combined organic extracts were dried over MgSO₄ and concentrated in vacuo. Purification by flash column chromatography (SiO₂; 5-20% EtOAc in hexanes) afforded the product as a colourless oil (21.8 mg, 84%).

¹H NMR (400 MHz, CDCl₃) δ 7.22-7.10 (m, 1H), 6.93-6.83 (m, 1H), 6.79 (d, J=7.7 Hz, 1H), 6.71 (d, J=10.5 Hz, 1H), 6.41 (s, 2H), 5.71 (dt, J=2.7, 1.4 Hz, 1H), 4.08 (s, 2H), 4.03-3.99 (m, 1H), 3.70 (s, 6H), 2.72 (dd, J=11.7, 3.2 Hz, 1H), 2.26-2.19 (m, 2H), 2.09-2.05 (m, 1H), 1.72 (d, J=7.9 Hz, 1H), 1.65-1.41 (m, 2H), 1.32 (s, 3H), 1.28 (s, 3H), 1.16 (s, 3H), 1.14-1.02 (m, 4H), 0.98 (s, 3H), 0.96-0.82 (m, 2H), 0.76 (t, J=6.9 Hz, 3H). ¹³C NMR (101 MHz, CDCl₃) δ 162.4 (d, J=244.1 Hz), 158.2, 148.1, 144.8 (d, J=6.7 Hz), 142.0, 128.5 (d, J=8.2 Hz), 125.9, 123.6, 118.2, 116.5 (d, J=21.1 Hz), 112.8 (d, J=21.0 Hz), 103.8, 66.7, 57.1, 55.9, 47.5, 43.8, 41.6, 40.9, 37.5, 31.7, 29.3, 28.4, 27.8, 27.8, 26.3, 24.2, 22.4, 21.1, 14.0. ¹⁹F NMR (376 MHz, CDCl₃) δ-114.8. IR (neat, vₘₐₓ/cm¹): 3381, 2928,2862, 1656, 1605, 1586, 1572, 1487, 1463, 1449, 1410, 1380, 1364, 1340, 1302, 1238, 1184, 1160, 1119, 1049, 986, 927, 871, 832, 782, 724, 699, 671, 654, 578. HRMS (ESI): m/z=531.3245 [M+Na]⁺ (calc. for C₃₃H₄₅FNaO₃ m/z=531.3245). [α]²⁵_{D}=+55.004±0.309 (c=1.0, CHCl₃).

### Example 12

### ((1S,4S,5S)-4-(4-((S)-3-(3-fluorophenyl)-2-methyloctan-2-yl)-2,6-dimethoxyphenyl)-6,6-dimethylbicyclo[3.1.1]hept-2-en-2-yl)methanol

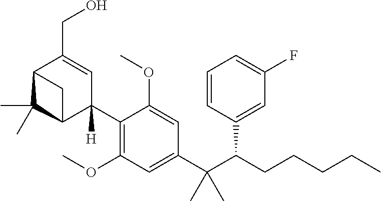 [SMILES: CCCCC[C@@H](c1cccc(F)c1)C(C)(C)c1cc(OC)c([C@H]2C=C(CO)[C@H]3C[C@@H]2C3(C)C)c(OC)c1]

### Step o) ((1S,4S,5S)-4-(4-((S)-3-(3-fluorophenyl)-2-methyloctan-2-yl)-2,6-dihydroxyphenyl)-6,6-dimethylbicyclo[3.1.1]hept-2-en-2-yl)methyl pivalate

To a solution of resorcinol (S)-5-(3-(4-fluorophenyl)-2-methyloctan-2-yl)benzene-1,3-diol B (25.5 mg, 77.1 μmol, 1.0 equiv) and pTsOH·H₂O (4.1 mg, 21.6 μmol, 0.28 equiv) in CH₂Cl₂ (3.0 mL) was added ((1S,4R,5R)-4-hydroxy-6,6-dimethylbicyclo[3.1.1]hept-2-en-2-yl)methyl pivalate (21.4 mg, 84.8 μmol, 1.1 equiv) and the solution was stirred at rt for 30 minutes. The reaction was stopped by addition of sat. aq. NaHCO₃ (3 mL), the layers were separated and the aqueous layer was extracted with Et₂O (3×5 mL). The combined organic extracts were dried over MgSO₄ and concentrated in vacuo. Purification by flash column chromatography (SiO₂; 5-15% EtOAc in hexanes) afforded the product as a colourless foam (31.6 mg, 73%).

¹H NMR (400 MHz, CDCl₃) δ 7.23-7.14 (m, 1H), 6.94-6.82 (m, 2H), 6.76 (ddd, J=10.7, 2.6, 1.6 Hz, 1H), 6.33 (s, 2H), 6.03 (dt, J=3.2, 1.6 Hz, 1H), 5.73 (bs, 2H), 4.68-4.61(m, 1H), 4.52 (ddd, J=13.5, 2.3, 1.6 Hz, 1H), 4.03 (s, 1H), 2.71 (dd, J=12.0, 2.9 Hz, 1H), 2.43-2.35 (m, 1H), 2.33-2.29 (m, 2H), 1.61-1.52 (m, 1H), 1.50 (d, J=9.7 Hz, 1H), 1.45-1.37 (m, 1H), 1.35 (s, 3H), 1.24 (s, 9H), 1.20 (s, 3H), 1.19-1.05 (m, 4H), 1.04 (s, 3H), 0.99 (s, 3H), 0.96-0.80 (m, 2H), 0.75 (t, J=6.6 Hz, 3H). ¹³C NMR (101 MHz, CDCl₃) δ 178.7, 162.5 (d, J=244.1 Hz), 155.0, 149.9, 149.6, 145.0 (d, J=6.7 Hz), 128.7 (d, J=8.2 Hz), 126.0, 120.4, 116.6 (d, J=20.7 Hz), 113.0 (d, J=21.0 Hz), 112.0, 107.2, 66.6, 56.9, 47.5, 44.3, 41.2, 41.1, 39.1, 37.9, 31.8, 29.4, 28.9, 28.2, 28.0, 27.4, 26.0, 23.2, 22.5, 20.9, 14.1. ¹⁹F NMR (376 MHz, CDCl₃) δ-114.7. IR (neat, vₘₐₓ/cm⁻¹): 3449, 2956, 2930, 2870, 1726, 1707, 1625, 1613, 1586, 1577, 1480, 1464, 1445, 1428, 1398, 1384, 1366, 1337, 1326, 1283, 1229, 1157, 1111, 1073, 1040, 1028, 962, 935, 916, 876, 783, 756, 724, 700, 668, 587, 569, 545, 521. HRMS (ESI): m/z=587.3498 [M+Na]⁺ (calc. for C₃₆H₄₉FNaO₄ m/z=587.3507). [α]²⁵_{D}=+69.607±0.141 (c=1.0, CHCl₃).

### Step p) ((1S,4S,5S)-4-(4-((S)-3-(3-fluorophenyl)-2-methyloctan-2-yl)-2,6-dimethoxyphenyl)-6,6-dimethylbicyclo[3.1.1]hept-2-en-2-yl)methyl pivalate

To a suspension of diol ((1S,4S,5S)-4-(4-((S)-3-(3-fluorophenyl)-2-methyloctan-2-yl)-2,6-dihydroxyphenyl)-6,6-dimethylbicyclo[3.1.1]hept-2-en-2-yl)methyl pivalate (31.6 mg, 55.9 μmol, 1.0 equiv) and K₂CO₃ (46.4 mg. 336 μmol, 6.0 equiv) in acetone (0.7 mL) was added (MeO)₂SO₂ (26.5 μL, 280 μmol, 5.0 equiv) and the solution was stirred at rt overnight. The reaction mixture was diluted with Et₂O (5 mL), filtered and concentrated in vacuo. Purification by flash column chromatography (SiO₂; 0-5% EtOAc in hexanes) afforded the product as a white foam (30.2 mg, 91%).

¹H NMR (400 MHz, CDCl₃) δ 7.20-7.12 (m, 1H), 6.91-6.83 (m, 1H), 6.80 (d, J=7.9 Hz, 1H), 6.71 (d, J=10.5 Hz, 1H), 6.41 (s, 2H), 5.79 (s, 1H), 4.61-4.55 (m, 1H), 4.55-4.48 (m, 1H), 4.01 (s, 1H), 3.70 (s, 6H), 2.72 (dd, J=11.7, 3.2 Hz, 1H), 2.23-2.13 (m, 2H), 2.09-2.01 (m, 1H), 1.70 (d, J=7.5 Hz, 1H), 1.63-1.43 (m, 2H), 1.31 (s, 3H), 1.28 (s, 3H), 1.22 (s, 9H), 1.17 (s, 3H), 1.15-1.04 (m, 4H), 0.98 (s, 3H), 0.94-0.85 (m, 2H), 0.76 (t, J=6.4 Hz, 3H). ¹³C NMR (101 MHz, CDCl₃) δ 178.6, 162.5 (d, J=244.1 Hz), 158.3, 148.2, 145.0 (d, J=6.8 Hz), 137.4, 128.7 (d, J=8.2 Hz), 126.4, 126.0, 118.2, 116.6 (d, J=20.0 Hz), 112.9 (d, J=21.0 Hz), 103.9, 67.6, 57.2, 55.9, 47.6, 44.0, 41.8, 41.1, 39.0, 37.7, 31.8, 29.4, 28.6, 28.0, 27.7, 27.5, 26.5, 24.3, 22.6, 21.2, 14.1. ¹⁹F NMR (376 MHz, CDCl₃) δ-114.8. IR (neat, vₘₐₓ/cm⁻¹): 2955, 2930, 2869, 1725, 1657, 1635, 1604, 1586, 1573, 1508, 1480, 1463, 1450, 1410, 1382, 1365, 1337, 1281, 1260, 1239, 1150, 1120, 1031, 957, 928, 904, 871, 832, 782, 756, 669, 654, 584, 521. HRMS (ESI): m/z=615.3804 [M+Na]⁺ (calc. for C₃₈H₅₃FNaO₄ m/z=615.3820). [α]²⁵_{D}=+73.779±0.081 (c=1.0, CHCl₃).

### Step o) ((1S,4S,5S)-4-(4-((S)-3-(3-fluorophenyl)-2-methyloctan-2-yl)-2,6-dimethoxyphenyl)-6,6-dimethylbicyclo[3.1.1]hept-2-en-2-yl)methanol

To a solution of ((1S,4S,5S)-4-(4-((S)-3-(3-fluorophenyl)-2-methyloctan-2-yl)-2,6-dimethoxyphenyl)-6,6-dimethylbicyclo[3.1.1]hept-2-en-2-yl)methyl pivalate (27.3 mg, 46.0 μmol, 1.0 equiv) in CH₂Cl₂ (0.55 mL) was added DIBAL (97.0 μL, 97.0 μmol, 2.1 equiv) at 0° C. After stirring the reaction for 15 minutes at 0° C., sat. aq. NH₄Cl (3 mL) was added, the layers were separated and the aqueous layer was extracted with CH₂Cl₂ (3×5 mL). The combined organic extracts were dried over MgSO₄ and concentrated in vacuo. Purification by flash column chromatography (SiO₂; 5-20% EtOAc in hexanes) afforded the product as a colourless oil (21.5 mg, 92%).

¹H NMR (400 MHz, CDCl₃) δ 7.19-7.13 (m, 1H), 6.91-6.83 (m, 1H), 6.79 (d, J=7.7 Hz, 1H), 6.71 (d, J=10.2 Hz, 1H), 6.41 (s, 2H), 5.72 (dt, J=2.8, 1.4 Hz, 1H), 4.08 (d, J=4.4 Hz, 2H), 4.03-3.96 (m, 1H), 3.70 (s, 6H), 2.72 (dd, J=11.8, 3.2 Hz, 1H), 2.27-2.18 (m, 2H), 2.11-2.05 (m, 1H), 1.70 (d, J=7.7 Hz, 1H), 1.61-1.40 (m, 2H), 1.32 (s, 3H), 1.27 (s, 3H), 1.16 (s, 3H), 1.15-1.02 (m, 4H), 0.98 (s, 3H), 0.96-0.81 (m, 2H), 0.76 (t, J=6.5 Hz, 3H). ¹³C NMR (101 MHz, CDCl₃) δ 162.5 (d, J=244.1 Hz), 158.3, 148.2, 145.0 (d, J=6.7 Hz), 142.1, 128.7 (d, J=8.3 Hz), 126.0, 123.7, 118.3, 116.6 (d, J=21.2 Hz), 112.9 (d, J=21.0 Hz), 103.9, 66.8, 57.2, 56.0, 47.6, 44.0, 41.8, 41.0, 37.6, 31.8, 29.4, 28.6, 28.0, 28.0, 26.4, 24.3, 22.6, 21.2, 14.1. ¹⁹F NMR (376 MHz, CDCl₃) δ-114.8. IR (neat, vₘₐₓ/cm⁻¹): 3373, 2927, 2862, 1661, 1604, 1586, 1572, 1508, 1487, 1463, 1450, 1410, 1380, 1364, 1341, 1302, 1239, 1183, 1160, 1119, 1072, 1049, 987, 927, 871, 832, 782, 724, 699, 671, 578, 522. HRMS (ESI): m/z=531.3245 [M+Na]⁺ (calc. for C₃₃H₄₅FNaO₃ m/z=531.3245). [α]²⁵_{D}=+107.650±0.171 (c=1.0, CHCl₃).

### Example 13

### ((1S,4S,5S)-4-(2,6-dimethoxy-4-(2-methyl-3-(1-methyl-1H-pyrazol-3-yl)octan-2-yl)phenyl)-6,6-dimethylbicyclo[3.1.1]hept-2-en-2-yl)methanol

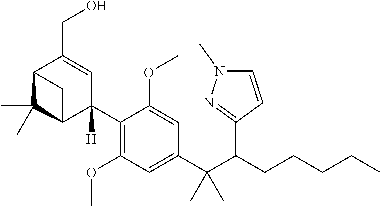 [SMILES: CCCCCC(c1ccn(C)n1)C(C)(C)c1cc(OC)c([C@H]2C=C(CO)[C@H]3C[C@@H]2C3(C)C)c(OC)c1]

Example 13 can be accessed in fashion analogous to that described for Examples 1-12.

### Example 14

### ((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methanamine

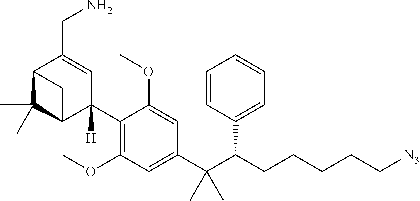 [SMILES: COc1cc(C(C)(C)[C@@H](CCCCCN=[N+]=[N-])c2ccccc2)cc(OC)c1[C@H]1C=C(CN)[C@H]2C[C@@H]1C2(C)C]

### Step c) 8-chloro-2-(3,5-dimethoxyphenyl)-2-methyl-3-phenyloctan-3-ol

Solution of 1-chloro-5-iodopentane (1.42 mL, 10.1 mmol, 1.2 equiv, CAS RN: 60274-60-4) in 3:2 pentane-Et₂O (100 mL) was cooled to −78° C. under argon atmosphere and t-BuLi (1.7 M in pentane, 11.9 mL, 20.3 mmol, 2.4 equiv) was added dropwise. The solution was stirred at −78° C. for 20 min. To this solution, 2-(3,5-dimethoxyphenyl)-2-methyl-1-phenylpropan-1-one (2.40 g, 8.44 mmol, 1.0 equiv, CAS RN: 208349-21-7) in Et₂O (2.0 mL) was added in one portion. The mixture was stirred for 5 min. at −78° C. then the cooling bath was removed and the mixture was stirred for additional 30 min. The reaction was quenched by addition of sat. aq. NH₄Cl (50 mL). The layers were separated and the aqueous phase was extracted with Et₂O (3×100 mL). Combined organic extracts were washed with brine, dried over MgSO₄ and concentrated in vacuo. Purification by flash column chromatography (SiO₂; using 5-35% Et₂O in hexanes) afforded the product as a colorless viscous liquid (2.75 g, 83%).

¹H NMR (400 MHz, CDCl₃) δ 7.31-7.18 (m, 5H), 6.43 (d, J=2.3 Hz, 2H), 6.36 (t, J=2.2 Hz, 1H), 3.74 (s, 6H), 3.42 (t, J=6.7 Hz, 2H), 2.21 (ddd, J=13.9, 12.0, 4.3 Hz, 1H), 1.81 (s, 1H), 1.70-1.53 (m, 3H), 1.37 (s, 3H), 1.36-1.28 (m, 2H), 1.24 (s, 3H), 1.22-1.09 (m, 1H), 0.95-0.78 (m, 1H). ¹³C NMR (101 MHz, CDCl₃) δ 160.1, 148.2, 142.2, 127.9, 127.1, 126.5, 107.5, 98.0, 80.4, 55.4, 46.7, 45.2, 35.7, 32.7, 27.6, 25.1, 24.8, 23.5. IR (neat, vₘₐₓ/cm¹): 3558, 2937, 2868, 2836, 1594, 1456, 1421, 1386, 1204, 1154, 707. HRMS (ESI): m/z=413.1850 [M+Na]⁺ (calc. for C₂₃H₃₁ClNaO₃ m/z=413.1854).

### Step g) (E)-1-(8-chloro-2-methyl-3-phenyloct-3-en-2-yl)-3,5-dimethoxybenzene

To a solution of 8-chloro-2-(3,5-dimethoxyphenyl)-2-methyl-3-phenyloctan-3-ol (2.70 g, 6.91 mmol, 1.0 equiv) in anhydrous THF (40 mL) at −78° C. was added KHMDS (1.0 M in THF, 17.3 mL, 17.3 mmol, 2.5 equiv) and CS₂(6.26 mL, 104 mmol, 15.0 equiv). The yellow solution was stirred at −78° C. for 10 min, the cooling bath was removed and the mixture stirred for another 30 min. Mel (6.88 mL, 111 mmol, 16.0 equiv) was added and the mixture was stirred at ambient temperature for 1 h and then at 40° C. overnight. The mixture was diluted with Et₂O (40 mL) and aq. sat. NaHCO₃ (40 mL) was added. The layers were separated and the aqueous phase was extracted with Et₂O (2×40 mL). Combined organic extracts were washed with brine, dried over MgSO₄ and concentrated in vacuo. Purification by flash column chromatography (SiO₂; using 0-2% Et₂O in hexanes) afforded the product as an amber orange liquid (2.45 g, 95%).

¹H NMR (400 MHz, CDCl₃) δ 7.17-7.11 (m, 3H), 6.70-6.63 (m, 2H), 6.50 (d, J=2.3 Hz, 2H), 6.33 (t, J=2.3 Hz, 1H), 5.65 (t, J=7.2 Hz, 1H), 3.78 (s, 6H), 3.42 (t, J=6.8 Hz, 2H), 1.82-1.74 (m, 2H), 1.72-1.64 (m, 2H), 1.50-1.41 (m, 2H), 1.37 (s, 6H). ¹³C NMR (101 MHz, CDCl₃) δ 160.4, 150.9, 149.5, 140.2, 129.7, 127.5, 126.3, 125.6, 105.8, 97.4, 55.4, 45.0, 44.3, 32.2, 29.0, 28.5, 27.2. IR (neat, vₘₐₓ/cm⁻¹): 2947, 2933, 2835, 1598, 1457, 1422, 1204, 1155, 1059, 706. HRMS (ESI): m/z=395.1745 [M+Na]⁺ (calc. for C₂₃H₂₉C₁NaO₂ m/z=395.1748).

### Step h) (E)-5-(8-chloro-2-methyl-3-phenyloct-3-en-2-yl)benzene-1,3-diol

To a solution of (E)-1-(8-chloro-2-methyl-3-phenyloct-3-en-2-yl)-3,5-dimethoxybenzene (2.50 g, 6.70 mmol, 1.0 equiv) in anhydrous CH₂Cl₂ (50 mL) at 0° C. was added BBr₃ (1.0 M in CH₂Cl₂, 20.1 mL, 20.1 mmol, 3.0 equiv) dropwise and the green solution was stirred at 0° C. for 5.5 h. The mixture was diluted with CH₂Cl₂ (40 mL) and carefully quenched with aq. sat. NaHCO₃ (40 mL). The layers were separated and the aqueous phase was extracted with CH₂Cl₂ (2×40 mL). Combined organic extracts were dried over MgSO₄ and concentrated in vacuo. Purification by flash column chromatography (SiO₂; using 0-50% EtOAc in hexanes) afforded the product as a golden oil (2.25 g, 97%).

¹H NMR (400 MHz, CDCl₃) δ 7.18-7.10 (m, 3H), 6.76-6.66 (m, 2H), 6.46 (d, J=2.2 Hz, 2H), 6.25 (t, J=2.1 Hz, 1H), 5.90 (bs, 2H), 5.64 (t, J=7.2 Hz, 1H), 3.42 (t, J=6.7 Hz, 2H), 1.82-1.71 (m, 2H), 1.71-1.61 (m, 2H), 1.51-1.38 (m, 2H), 1.33 (s, 6H). ¹³C NMR (101 MHz, CDCl₃) δ 156.0, 151.8, 149.0, 140.1, 129.6, 127.4, 126.2, 125.7, 107.1, 100.5, 45.2, 43.8, 32.0, 28.8, 28.3, 26.9. IR (neat, vₘₐₓ/cm⁻¹): 3356, 2966, 2944, 2867, 1599, 1440, 1323, 1151, 993, 705. HRMS (ESI): m/z=367.1432 [M+Na]⁺ (calc. for C₂₁H₂₅ClNaO₂ m/z=367.1435).

### Step i) 5-(8-chloro-2-methyl-3-phenyloctan-2-yl)benzene-1,3-diol

To a solution of (E)-5-(8-chloro-2-methyl-3-phenyloct-3-en-2-yl)benzene-1,3-diol (1.80 g, 5.22 mmol) in dry EtOAc (30 mL) was added Pd (10 wt % on C, 5.55 g, 5.22 mmol, 1.0 equiv) and the resulting mixture was hydrogenated at 60 bar at rt for 48 h. Then the mixture was filtered over a pad of Celite and the crude material was purified by flash column chromatography (SiO₂, 15-30% EtOAc in hexanes) to afford the product as a dark red oil (1.75 g, 97%). The racemate was resolved by chiral SFC Method 5. to afford (S)-5-(8-chloro-2-methyl-3-phenyloctan-2-yl)benzene-1,3-diol A (450 mg, 25%, % ee>99%) and (R)-5-(8-chloro-2-methyl-3-phenyloctan-2-yl)benzene-1,3-diol B (360 mg, 20%, % ee=96%).

¹H NMR (400 MHz, CDCl₃) δ 7.30-7.17 (m, 3H), 7.11-7.03 (m, 2H), 6.42 (d, J=2.2 Hz, 2H), 6.22 (t, J=2.2 Hz, 1H), 5.18 (bs, 2H), 3.38 (td, J=6.7, 2.0 Hz, 2H), 2.74 (dd, J=12.2, 2.9 Hz, 1H), 1.70-1.59 (m, 1H), 1.58-1.49 (m, 2H), 1.45-1.34 (m, 1H), 1.32-1.18 (m, 2H), 1.22 (s, 3H), 1.06 (s, 3H), 1.02-0.83 (m, 2H). ¹³C NMR (101 MHz, CDCl₃) δ 156.2, 153.2, 141.5, 130.1, 127.6, 126.3, 106.8, 100.3, 56.8, 45.4, 41.6, 32.4, 29.2, 29.1, 27.5, 26.7, 23.2. IR (neat, vₘₐₓ/cm⁻¹): 3345, 2936, 2867, 1599, 1453, 1320, 1151, 992, 705.

HRMS (ESI): m/z=369.1590 [M+Na]⁺ (calc. for C₂₁H₂₇ClNaO₂ m/z=369.1592). (S)-5-(8-chloro-2-methyl-3-phenyloctan-2-yl)benzene-1,3-diol A [α]²⁵_{D}=−34.683±0.163 (c=1.0, CHCl₃). (R)-5-(8-chloro-2-methyl-3-phenyloctan-2-yl)benzene-1,3-diol B [α]²⁵_{D}=+33.913±0.053 (c=1.0, CHCl₃).

### Step o) 2-(((1S,4S,5S)-4-(4-((S)-8-chloro-2-methyl-3-phenyloctan-2-yl)-2,6-dihydroxyphenyl)-6,6-dimethylbicyclo[3.1.1]hept-2-en-2-yl)methyl)isoindoline-1,3-dione

To a solution of (S)-5-(8-chloro-2-methyl-3-phenyloctan-2-yl)benzene-1,3-diol A (51.0 mg, 147 μmol, 1.0 equiv) and pTsOH·H₂O (8.0 mg. 42.0 μmol, 0.28 equiv) in CH₂Cl₂(6.0 mL) was added 2-(((1S,4R,5R)-4-hydroxy-6,6-dimethylbicyclo[3.1.1]hept-2-en-2-yl)methyl)isoindoline-1,3-dione (48.0 mg, 162 mol, 1.1 equiv, Chem. Eur. J., 2020, 26, 1380-1387) and the light-yellow solution was stirred at rt for 1.5 h. More 2-(((1S,4R,5R)-4-hydroxy-6,6-dimethylbicyclo[3.1.1]hept-2-en-2-yl)methyl)isoindoline-1,3-dione (10.9 mg, 36.7 μmol, 0.25 equiv) was added and the reaction was stirred for 1 h. The reaction was stopped by addition of sat. aq. NaHCO₃ (3 mL), the layers were separated and the aqueous layer was extracted with Et₂O (3×5 mL). Combined organic extracts were dried over MgSO₄ and concentrated in vacuo. Purification by flash column chromatography (SiO₂; 5-30% EtOAc in hexanes) afforded the product as a colourless foam (62.0 mg, 67%).

¹H NMR (400 MHz, CDCl₃) δ 7.93-7.86 (m, 2H), 7.76-7.70 (m, 2H), 7.28-7.14 (m, 3H), 7.11-7.03 (m, 2H), 6.49-6.31 (s, 2H), 6.05-5.98 (m, 1H), 5.90 (bs, 2H), 4.47 (ddd, J=15.5, 3.0, 1.9 Hz, 1H), 4.27-4.19 (m, 1H), 4.05-3.98 (m, 1H), 3.35 (t, J=6.8 Hz, 2H), 2.71 (dd, J=12.1, 2.9 Hz, 1H), 2.37-2.21 (m, 3H), 1.66-1.15 (m, 7H), 1.32 (s, 3H), 1.17 (s, 3H), 1.01 (s, 3H), 1.00 (s, 3H), 0.98-0.82 (m, 2H). ¹³C NMR (101 MHz, CDCl₃) δ 168.5, 154.9, 149.9, 148.1, 141.8, 134.3, 132.0, 130.1, 127.6, 126.2, 123.7, 120.7, 111.9, 107.4, 56.8, 47.3, 45.3, 44.3, 43.3, 41.3, 41.2, 37.8, 32.4, 29.6, 29.2, 28.2, 27.5, 26.7, 26.0, 22.6, 20.7. IR (neat, vₘₐₓ/cm⁻¹): 3454, 2933, 1770, 1711, 1623, 1574, 1426, 1393, 754, 729, 705. HRMS (ESI): m/z=648.2844 [M+Na]⁺ (calc. for C₃₉H₄₄ClNNaO₄ m/z=648.2851). [α]²⁵_{D}=+72.790±0.118 (c=1.0, CHCl₃).

### Step p) 2-(((1S,4S,5S)-4-(4-((S)-8-chloro-2-methyl-3-phenyloctan-2-yl)-2,6-dimethoxyphenyl)-6,6-dimethylbicyclo[3.1.1]hept-2-en-2-yl)methyl)isoindoline-1,3-dione

To a suspension of 2-(((1S,4S,5S)-4-(4-((S)-8-chloro-2-methyl-3-phenyloctan-2-yl)-2,6-dihydroxyphenyl)-6,6-dimethylbicyclo[3.1.1]hept-2-en-2-yl)methyl)isoindoline-1,3-dione (57.0 mg, 91.0 μmol, 1.0 equiv) and K₂CO₃ (75.5 mg. 546 μmol, 6.0 equiv) in acetone (0.9 mL) was added (MeO)₂SO₂ (43 μL. 455 μmol, 5.0 equiv) and the solution was stirred at rt overnight. The reaction mixture was diluted with Et₂O (5 mL), filtered and concentrated in vacuo. Purification by flash column chromatography (SiO₂; 0-15% acetone in hexanes) afforded the product as a white foam (49 mg, 82%).

¹H NMR (400 MHz, CDCl₃) δ 7.89-7.82 (m, 2H), 7.75-7.67 (m, 2H), 7.25-7.12 (m, 3H), 7.05-6.98 (m, 2H), 6.39 (s, 2H), 5.62-5.56 (m, 1H), 4.35-4.28 (m, 1H), 4.24 (ddd, J=15.4, 2.3, 1.6 Hz, 1H), 3.98-3.94 (m, 1H), 3.68 (s, 6H), 3.36 (t, J=6.7 Hz, 2H), 2.71 (dd, J=12.0, 2.9 Hz, 1H), 2.23-2.12 (m, 2H), 2.05-1.97 (m, 1H), 1.74 (d, J=8.0 Hz, 1H), 1.68-1.38 (m, 4H), 1.34-1.14 (m, 2H), 1.27 (s, 3H), 1.25 (s, 3H), 1.12 (s, 3H), 1.02-0.83 (m, 2H), 0.94 (s, 3H). ¹³C NMR (101 MHz, CDCl₃) δ 168.3, 158.3, 148.4, 141.7, 136.2, 133.9, 132.4, 130.1, 127.5, 126.2, 123.9, 123.3, 117.9, 103.7, 57.2, 55.9, 47.6, 45.2, 44.4, 42.6, 41.8, 41.2, 37.5, 32.5, 29.2, 29.0, 27.6, 27.6, 26.8, 26.4, 23.9, 20.9. IR (neat, vₘₐₓ/cm¹): 2930, 2863, 1772, 1715, 1603, 1572, 1390, 1239, 1117, 729, 704, 671. HRMS (ESI): m/z=676.3152 [M+Na]⁺ (calc. for C₄₁H₄₈ClNNaO₄ m/z=676.3164). [α]²⁵_{D}=+33.551±0.235 (c=1.0, CHCl₃).

### Step r) 2-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)isoindoline-1,3-dione

To solution of 2-(((1S,4S,5S)-4-(4-((S)-8-chloro-2-methyl-3-phenyloctan-2-yl)-2,6-dimethoxyphenyl)-6,6-dimethylbicyclo[3.1.1]hept-2-en-2-yl)methyl)isoindoline-1,3-dione (42.0 mg, 64.1 μmol, 1.0 equiv) in DMF (0.35 mL) was added NaN₃ (41.7 mg, 642 μmol, equiv) and the reaction mixture was stirred at 50° C. and for 24 h. The mixture was allowed to cool down to rt and diluted with 5% aq. LiCl (3 mL) and Et₂O (3 mL). The layers were separated and the aqueous phase was extracted with Et₂O (3×3 mL). Combined organic extracts were dried over MgSO₄, filtered and concentrated in vacuo. Purification by flash column chromatography (SiO₂, 0-10% EtOAc in hexanes) afforded the product as a white foam (38.0 mg, 90%).

¹H NMR (400 MHz, CDCl₃) δ 7.88-7.82 (m, 2H), 7.74-7.66 (m, 2H), 7.25-7.13 (m, 3H), 7.05-6.98 (m, 2H), 6.39 (s, 2H), 5.63-5.56 (m, 1H), 4.36-4.28 (m, 1H), 4.24 (ddd, J=15.4, 2.3, 1.6 Hz, 1H), 3.99-3.94 (m, 1H), 3.68 (s, 6H), 3.09 (t, J=6.9 Hz, 2H), 2.70 (dd, J=12.0, 2.9 Hz, 1H), 2.24-2.11 (m, 2H), 2.04-1.98 (m, 1H), 1.74 (d, J=8.0 Hz, 1H), 1.68-1.08 (m, 6H), 1.27 (s, 3H), 1.25 (s, 3H), 1.12 (s, 3H), 1.02-0.83 (m, 2H), 0.94 (s, 3H). ¹³C NMR (101 MHz, CDCl₃) δ 168.3, 158.3, 148.4, 141.7, 136.2, 133.9, 132.4, 130.1, 127.5, 126.2, 123.9, 123.3, 117.9, 103.6, 57.2, 55.8, 51.5, 47.6, 44.3, 42.6, 41.8, 41.2, 37.5, 29.3, 29.0, 28.7, 27.9, 27.6, 26.6, 26.4, 23.9, 20.9. IR (neat, vₘax/cm⁻¹): 2933, 2865, 2094, 1772, 1716, 1603, 1572, 1390, 1240, 1119. (ESI): m/z=661.3746 [M+H]⁺ (calc. for C₄₁H₄₉N₄O₄m/z=661.3748). [α]²⁵_{D}=+32.189±0.150 (c=1.0, CHCl₃).

### Step s) ((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methanamine

To a solution of 2-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)isoindoline-1,3-dione (36.0 mg, 54.4 μmol, 1.0 equiv) in EtOH (1.1 mL) was added N₂H₄H₂O (27 μL, 544 μmol, equiv) and (E)/(Z)-crotyl alcohol (70 μL, 817 μmol, 15 equiv) and the solution was heated to 75° C. and stirred for 2 h. Precipitation of a white solid occurred, and the mixture was filtered over cotton, which was rinsed with EtOH. The filtrate was concentrated in vacuo. Purification by flash column chromatography (SiO₂; 1% 7.0 M NH₃ in MeOH, 1-4% MeOH in CH₂Cl₂) afforded the product as a white foam (25.5 mg, 88%).

¹H NMR (400 MHz, CDCl₃) δ 7.25-7.15 (m, 3H), 7.06-7.00 (m, 2H), 6.43 (s, 2H), 5.58-5.54 (m, 1H), 4.03-3.97 (m, 1H), 3.70 (s, 6H), 3.24 (t, J=1.8 Hz, 2H), 3.11 (t, J=6.9 Hz, 2H), 2.72 (dd, J=12.1, 2.9 Hz, 1H), 2.23-2.17 (m, 1H), 2.14 (td, J=5.6, 1.4 Hz, 1H), 2.06 (ddt, J=5.8, 3.9, 1.9 Hz, 1H), 1.72 (d, J=8.4 Hz, 1H), 1.69-1.32 (m, 4H), 1.31 (s, 3H), 1.27 (s, 3H), 1.24-1.16 (m, 2H), 1.14 (s, 3H), 1.02-0.88 (m, 2H), 0.97 (s, 3H). ¹³C NMR (101 MHz, CDCl₃) δ 158.4, 148.4, 143.6, 141.7, 130.1, 127.5, 126.3, 121.0, 118.5, 104.0, 57.3, 56.0, 51.5, 47.7, 47.6, 44.7, 41.8, 41.0, 37.6, 29.3, 28.9, 28.7, 28.1, 27.9, 26.7, 26.5, 24.0, 21.2. IR (neat, vₘₐₓ/cm⁻¹): 2932, 2864, 2094, 1604, 1572, 1452, 1410, 1240, 1120. HRMS (ESI): m/z=531.3690 [M+H]⁺ (calc. for C₃₃H₄₇N₄O₂m/z=531.3694). [α]²⁵_{D}=+44.526±0.423 (c=1.0, CHCl₃).

### Example 15

### ((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine

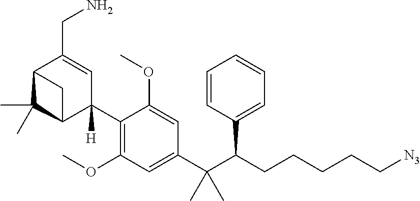 [SMILES: COc1cc(C(C)(C)[C@H](CCCCCN=[N+]=[N-])c2ccccc2)cc(OC)c1[C@H]1C=C(CN)[C@H]2C[C@@H]1C2(C)C]

### Step o) 2-(((1S,4S,5S)-4-(4-((R)-8-chloro-2-methyl-3-phenyloctan-2-yl)-2,6-dihydroxyphenyl)-6,6-dimethylbicyclo[3.1.1]hept-2-en-2-yl)methyl)isoindoline-1,3-dione

To a solution of (R)-5-(8-chloro-2-methyl-3-phenyloctan-2-yl)benzene-1,3-diol B (56.2 mg, 162 μmol, 1.0 equiv) and pTsOH·H₂O (8.6 mg. 45.3 μmol, 0.28 equiv) in CH₂Cl₂ (7.0 mL) was added 2-(((1S,4R,5R)-4-hydroxy-6,6-dimethylbicyclo[3.1.1]hept-2-en-2-yl)methyl)isoindoline-1,3-dione (53.0 mg, 178 μmol, 1.1 equiv) and the light-yellow solution was stirred at rt. for 1.5 h. More 2-(((1S,4R,5R)-4-hydroxy-6,6-dimethylbicyclo[3.1.1]hept-2-en-2-yl)methyl)isoindoline-1,3-dione (12.0 mg, 40.5 mol, 0.25 equiv) was added and the reaction was stirred for 1 h. The reaction was stopped by addition of sat. aq. NaHCO₃ (3 mL), the layers were separated and the aqueous layer was extracted with Et₂O (3×5 mL). Combined organic extracts were dried over MgSO₄ and concentrated in vacuo. Purification by flash column chromatography (SiO₂; using 5-30% EtOAc in hexanes) afforded the product as a colourless foam (72.0 mg, 71%).

¹H NMR (400 MHz, CDCl₃) δ 7.92-7.86 (m, 2H), 7.76-7.71 (m, 2H), 7.27-7.16 (m, 3H), 7.12-7.06 (m, 2H), 6.41 (s, 2H), 6.05-5.99 (m, 1H), 5.88 (bs, 2H), 4.47 (ddd, J=15.5, 3.0, 1.9 Hz, 1H), 4.27-4.18 (m, 1H), 4.05-3.99 (m, 1H), 3.36 (t, J=6.7 Hz, 2H), 2.72 (dd, J=12.1, 2.9 Hz, 1H), 2.36-2.22 (m, 3H), 1.70-1.44 (m, 4H), 1.40-1.13 (m, 3H), 1.32 (s, 3H), 1.17 (s, 3H), 1.00 (s, 3H), 0.99 (s, 3H), 0.97-0.81 (m, 2H). ¹³C NMR (101 MHz, CDCl₃) δ 168.5, 155.0, 150.1, 148.1, 141.7, 134.3, 132.1, 130.1, 127.6, 126.2, 123.7, 120.8, 111.9, 107.3, 56.7, 47.4, 45.3, 44.3, 43.3, 41.3, 41.2, 37.8, 32.4, 29.6, 29.1, 28.2, 27.5, 26.7, 26.0, 22.5, 20.7. IR (neat, vₘₐₓ/cm¹): 3454, 2931, 2867, 1770, 1710, 1623, 1574, 1426, 1392, 1340, 1112, 1024, 946, 764, 728, 705. HRMS (ESI): m/z=626.3030 [M+H]⁺ (calc. for C₃₉H₄₅C₁NO₄ m/z=626.3032). [α]²⁵_{D}=+114.140±0.504 (c=1.0, CHCl₃).

### Step p) 2-(((1S,4S,5S)-4-(4-((R)-8-chloro-2-methyl-3-phenyloctan-2-yl)-2,6-dimethoxyphenyl)-6,6-dimethylbicyclo[3.1.1]hept-2-en-2-yl)methyl)isoindoline-1,3-dione

To a suspension of 2-(((1S,4S,5S)-4-(4-((R)-8-chloro-2-methyl-3-phenyloctan-2-yl)-2,6-dihydroxyphenyl)-6,6-dimethylbicyclo[3.1.1]hept-2-en-2-yl)methyl)isoindoline-1,3-dione (70.0 mg, 118 μmol, 1.0 equiv) and K₂CO₃ (92.7 mg. 671 μmol, 6.0 equiv) in acetone (1.0 mL) was added (MeO)₂SO₂ (53 μL. 559 μmol, 5.0 equiv) and the solution was stirred at rt. overnight. The reaction mixture was diluted with Et₂O (5 mL), filtered and concentrated in vacuo. Purification by flash column chromatography (SiO₂; 0-15% acetone in hexanes) afforded the product as a white foam (59 mg, 81%).

¹H NMR (400 MHz, CDCl₃) δ 7.88-7.83 (m, 2H), 7.74-7.68 (m, 2H), 7.25-7.15 (m, 3H), 7.06-7.00 (m, 2H), 6.39 (s, 2H), 5.62-5.58 (m, 1H), 4.32 (ddd, J=15.4, 2.2, 1.6 Hz, 1H), 4.24 (ddd, J=15.4, 2.3, 1.6 Hz, 1H), 3.99-3.94 (m, 1H), 3.68 (s, 6H), 3.37 (t, J=6.8, 2H), 2.71 (dd, J=12.1, 2.9 Hz, 1H), 2.22-2.11 (m, 2H), 2.05-1.99 (m, 1H), 1.72 (d, J=8.1 Hz, 1H), 1.66-1.38 (m, 4H), 1.34-1.15 (m, 2H), 1.27 (s, 3H), 1.25 (s, 3H), 1.11 (s, 3H), 1.02-0.84 (m, 2H), 0.94 (s, 3H). ¹³C NMR (101 MHz, CDCl₃) δ 168.3, 158.3, 148.5, 141.7, 136.1, 133.9, 132.4, 130.1, 127.5, 126.2, 123.9, 123.3, 117.9, 103.6, 57.1, 55.8, 47.6, 45.2, 44.3, 42.6, 41.8, 41.2, 37.5, 32.4, 29.2, 29.0, 27.6, 27.6, 26.8, 26.4, 23.8, 20.9. IR (neat, vₘₐₓ/cm⁻¹): 2933, 2865, 1772, 1715, 1603, 1572, 1390, 1239, 1117, 730, 704. HRMS (ESI): m/z=676.3163 [M+Na]⁺ (calc. for C₄₁H₄₈ClNNaO₄ m/z=676.3164). [α]²⁵_{D}=+71.187±0.169 (c=1.0, CHCl₃).

### Step r) 2-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)isoindoline-1,3-dione

To solution of 2-(((1S,4S,5S)-4-(4-((R)-8-chloro-2-methyl-3-phenyloctan-2-yl)-2,6-dimethoxyphenyl)-6,6-dimethylbicyclo[3.1.1]hept-2-en-2-yl)methyl)isoindoline-1,3-dione (57.0 mg, 87.1 μmol, 1.0 equiv) in DMF (0.40 mL) was added NaN₃ (56.6 mg, 871 μmol, equiv) and the reaction mixture was stirred at 50° C. and for 24 h. The mixture was allowed to cool down to rt. and diluted with 5% aq. LiCl (3 mL) and Et₂O (3 mL). The layers were separated and the aqueous phase was extracted with Et₂O (3×3 mL). Combined organic extracts were dried over MgSO₄, filtered and concentrated in vacuo. Purification by flash column chromatography (SiO₂, 0-10% EtOAc in hexanes) afforded the product as a white foam (50.5 mg, 88%).

¹H NMR (400 MHz, CDCl₃) δ 7.89-7.81 (m, 2H), 7.75-7.67 (m, 2H), 7.25-7.15 (m, 3H), 7.06-6.99 (m, 2H), 6.39 (s, 2H), 5.63-5.5 (m, 1H), 4.35-4.29 (m, 1H), 4.28-4.20 (m, 1H), 4.00-3.93 (m, 1H), 3.68 (s, 6H), 3.09 (t, J=6.9 Hz, 2H), 2.71 (dd, J=12.1, 2.9 Hz, 1H), 2.23-2.10 (m, 2H), 2.06-1.98 (m, 1H), 1.72 (d, J=8.1 Hz, 1H), 1.68-1.53 (m, 1H), 1.47-1.09 (m, 5H), 1.27 (s, 3H), 1.25 (s, 3H), 1.11 (s, 3H), 1.03-0.85 (m, 2H), 0.94 (s, 3H). ¹³C NMR (101 MHz, CDCl₃) δ 168.3, 158.3, 148.5, 141.7, 136.1, 133.9, 132.4, 130.1, 127.5, 126.2, 124.0, 123.3, 117.9, 103.6, 57.2, 55.8, 51.5, 47.6, 44.3, 42.6, 41.8, 41.2, 37.5, 29.2, 29.0, 28.7, 27.9, 27.6, 26.6, 26.4, 23.8, 20.9. IR (neat, vₘₐₓ/cm⁻¹): 2933, 2864, 2094, 1772, 1715, 1603, 1572, 1424, 1410, 1390, 1239, 1118, 730, 711. HRMS (ESI): m/z=683.3565 [M+Na]⁺ (calc. for C₄₁H₄₈N₄NaO₄ m/z=683.3568). [α]²⁵_{D}=+73.648±0.128 (c=1.0, CHCl₃).

### Step s) ((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine

To a solution of 2-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)isoindoline-1,3-dione (37.0 mg, 55.9 μmol, 1.0 equiv) in EtOH (1.1 mL) was added N₂H₄H₂O (28 μL, 559 μmol, equiv) and (E)/(Z)-crotyl alcohol (72 μL, 840 μmol, 15 equiv) and the solution was heated to 75° C. and stirred for 2 h. Precipitation of a white solid occurred, and the mixture was filtered over cotton, which was rinsed with EtOH. The filtrate was concentrated in vacuo. Purification by flash column chromatography (SiO₂; 1% 7.0 M NH₃ in MeOH, 1-4% MeOH in CH₂Cl₂) afforded the product as a white foam (28.0 mg, 94%).

¹H NMR (400 MHz, CDCl₃) δ 7.26-7.15 (m, 3H), 7.03 (dt, J=6.0, 1.6 Hz, 2H), 6.43 (s, 2H), 5.58-5.55 (m, 1H), 4.01-3.98 (m, 1H), 3.71 (s, 6H), 3.23 (t, J=1.8 Hz, 2H), 3.11 (t, J=6.9 Hz, 2H), 2.72 (dd, J=12.1, 2.9 Hz, 1H), 2.20 (dt, J=8.3, 5.5 Hz, 1H), 2.13 (td, J=5.6, 1.4 Hz, 1H), 2.10-2.05 (m, 1H), 1.70 (d, J=8.3 Hz, 1H), 1.68-1.57 (m, 1H), 1.53-1.33 (m, 3H), 1.31 (s, 3H), 1.27 (s, 3H), 1.25-1.17 (m, 2H), 1.14 (s, 3H), 1.01-0.86 (m, 2H). 0.97 (s, 3H). ¹³C NMR (101 MHz, CDCl₃) δ 158.4, 148.4, 143.7, 141.7, 130.1, 127.5, 126.2, 120.9, 118.5, 104.0, 57.2, 56.0, 51.5, 47.7, 47.7, 44.7, 41.8, 41.0, 37.5, 29.3, 28.9, 28.7, 28.1, 27.9, 26.7, 26.5, 23.9, 21.2. IR (neat, vₘₐₓ/cm⁻¹): 2933, 2864, 2094, 1604, 1572, 1452, 1410, 1240, 1120. HRMS (ESI): m/z=531.3690 [M+H]⁺ (calc. for C₃₃H₄₇N₄O₂m/z=531.3694). [α]²⁵_{D}=+101.994±0.218 (c=1.0, CHCl₃).

### Example 16

### 6-((E)-2-(7-(diethylamino)-2-oxo-2H-chromen-3-yl)vinyl)-1-(6-((((1S,4S,5S)-4-(2,6-dimethoxy-4-(2-methyl-3-phenyloctan-2-yl)phenyl)-6,6-dimethylbicyclo[3.1.1]hept-2-en-2-yl)methyl)amino)-6-oxohexyl)pyridin-1-ium-3-sulfonate

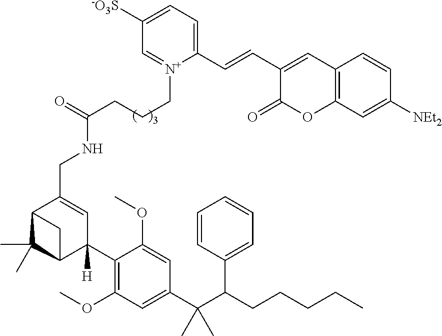

### 2-(3,5-bis(methoxymethoxy)phenyl)-2-methyl-1-phenylpropan-1-one

To a solution 2-(3,5-dimethoxyphenyl)-2-methyl-1-phenylpropan-1-one (3.45 g, 12.1 mmol, 1.0 equiv) in CH₂Cl₂ (40 mL) at −78° C. was added BBr₃ (3.44 mL, 36.4 mmol, 3.0 equiv). The solution was allowed to reach 0° C. in an ice bath and stirring was continued at 0° C. for 3.5 h. The reaction was stopped by addition of sat. aq. NaHCO₃ and the mixture was extracted with CH₂Cl₂. The combined organic extracts were washed with brine, dried over Na₂SO₄ and concentrated under reduced pressure. The crude material was purified by flash column chromatography (SiO₂, dryloading on silica; using 20 to 40% EtOAc in hexanes). The resorcinol intermediate was dissolved in anhydrous DMF (40 mL) and the solution was cooled to 0° C. NaH (50% on mineral oil, 1.75 g, 36.4 mmol, 3.0 equiv) was added and the suspension was stirred at 0° C. for 15 min. Freshly distilled MOMCl (2.76 mL, 36.4 mmol, 3.0 equiv) was added by syringe and the mixture was allowed to warm to ambient temperature. After 2 h, TLC (20% EtOAc in hexanes) showed full conversion of resorcinol intermediate and the reaction was stopped by addition of water. The mixture was extracted with Et₂O and the combined organic extracts were washed with water and brine, dried over Na₂SO₄ and concentrated under reduced pressure. Purification by flash column chromatography (SiO₂; using 10 to 20% Et₂O in hexanes) afforded the product as yellow oil (4.20 g, quantitative yield).

¹H NMR (400 MHz, CDCl₃) δ=7.56-7.51 (m, 2H), 7.37 (ddt, J=8.6, 7.0, 1.3 Hz, 1H), 7.26-7.20 (m, 2H), 6.67 (t, J=2.2 Hz, 1H), 6.64 (d, J=2.2 Hz, 2H), 5.12 (s, 4H), 3.44 (s, 6H), 1.57 (s, 6H).¹³C NMR (101 MHz, CDCl₃) δ=203.5, 158.9, 148.0, 136.4, 131.8, 129.7, 128.1, 108.0, 102.8, 94.8, 56.3, 51.6, 27.9. 1R (neat, vₘₐₓ/cm⁻¹) 2934, 1677, 1594, 1446, 1399, 1145, 1027. ESI-HRMS calcd for C₂₀H₂₅O₅[M+H]⁺ 345.1697; found 345.1695. 2-(3,5-bis(methoxymethoxy)phenyl)-8-((tert-butyldimethylsilyl)oxy)-2-methyl-3-phenyloctan-3-ol

To a solution of tert-butyl((5-iodopentyl)oxy)dimethylsilane (1.14 g, 3.48 mmol, 1.2 equiv) in 3:2 pentane-Et₂O (30 mL) at −78° C. was added t-BuLi (1.6 M in pentane, 4.54 mL, 7.26 mmol, 2.5 equiv). The solution was stirred at −78° C. for 5 min following addition, and then the mixture was allowed to reach ambient temperature and was stirred for 1 h. The reaction mixture was cooled back to −78° C., and a solution of 2-(3,5-bis(methoxymethoxy)phenyl)-2-methyl-1-phenylpropan-1-one (1.00 g, 2.90 mmol, 1.0 equiv) was added very slowly by syringe. The cooling bath was then removed, and the mixture allowed warming to ambient temperature. The reaction was stopped by addition of sat. aq. NH₄Cl. After separation of the organic layer, the aqueous phase was extracted with Et₂O and the combined organic extracts were washed with brine, dried over Na₂SO₄ and concentrated under reduced pressure. Purification by flash column chromatography (SiO₂; using 5 to 10% Et₂O in hexanes) afforded the product as yellow oil (1.03 g, 64%).

¹H NMR (500 MHz, CDCl₃) δ=7.26-7.17 (m, 5H), 6.64 (t, J=2.1 Hz, 1H), 6.62 (d, J=2.2 Hz, 2H), 5.10 (s, 4H), 3.51-3.48 (m, 2H), 3.47 (s, 6H), 2.19 (ddd, J=13.9, 12.0, 4.2 Hz, 1H), 1.58 (ddd, J=16.3, 9.8, 4.4 Hz, 1H), 1.41-1.33 (m, 2H), 1.35 (s, 3H), 1.27-1.18 (m, 2H), 1.23 (s, 3H) 1.16-1.07 (m, 1H), 0.87-0.84 (m, 1H), 0.86 (s, 9H), −0.01 (s, 6H). ¹³C NMR (126 MHz, CDCl₃) δ=157.5, 148.5, 142.3, 127.9, 127.0, 126.4, 111.0, 102.6, 94.8, 80.5, 63.3, 56.2, 46.7, 35.8, 32.9, 26.5, 26.1, 25.0, 24.8, 23.9, 18.5, −5.1. 1R (neat, vₘₐₓ/cm⁻¹) 3569, 2953, 2931, 2857, 1595, 1472, 1145, 1084, 1031.ESI-HRMS calcd for C₃₁H₅₀NaO₆Si [M+Na]⁺569.3269; found 569.3270.

### (E)-7-(3,5-bis(methoxymethoxy)phenyl)-7-methyl-6-phenyloct-5-en-1-ol

To a solution of 2-(3,5-bis(methoxymethoxy)phenyl)-8-((tert-butyldimethylsilyl)oxy)-2-methyl-3-phenyloctan-3-ol (3.1 g, 5.6 mmol, 1.0 equiv) in THF (56 mL) at −78° C. was added KHMDS (1.0 M in THF, 0.014 L, 0.014 mol, 2.5 equiv) and CS₂ (5.1 mL, 0.084 mol, equiv). The yellow solution was stirred at −78° C. for 30 min and was then allowed to warm to ambient temperature. After stirring for another 30 min, Mel (5.6 mL, 0.090 mol, 16 equiv) was added and the mixture was first stirred at ambient temperature for 1 h, and then at 50° C. overnight. Aq. sat. NaHCO₃ was added and the mixture was extracted with Et₂O. The combined organic extracts were washed with brine, dried over Na₂SO₄ and concentrated under reduced pressure. The unpurified material was taken up in THF (21 mL) and TBAF (1.0 M in THF, 0.017 L, 0.017 mol, 3.0 equiv) was added. After stirring for 4 h, the reaction was stopped by addition of aq. sat. NaHCO₃. The mixture was extracted with EtOAc and the combined organic extracts were washed with brine, dried over Na₂SO₄ and concentrated under reduced pressure. Purification by flash column chromatography (SiO₂; using 10 to 30% EtOAc in hexanes) afforded the product as yellow oil and as single olefin isomer (1.5 g, 64% over two steps).

¹H NMR (500 MHz, CDCl₃) δ=7.15-7.12 (m, 3H), 6.69-6.64 (m, 4H), 6.60 (t, J=2.2 Hz, 1H), 5.66 (t, J=7.3 Hz, 1H), 5.13 (s, 4H), 3.53-3.50 (m, 2H), 3.47 (s, 6H), 1.76 (q, J=7.2 Hz, 2H), 1.51-1.44 (m, 2H), 1.41-1.34 (m, 8H). ¹³C NMR (126 MHz, CDCl₃) δ=157.9, 151.2, 149.0, 140.4, 129.8, 127.4, 126.2, 126.2, 109.2, 102.2, 94.7, 63.0, 56.2, 44.2, 32.4, 29.0, 28.9, 26.0. ESI-HRMS calcd for C₂₅H₃₄NaO₅ [M+Na]⁺437.2298; found 437.2296.

### step f) 5-(8-hydroxy-2-methyl-3-phenyloctan-2-yl)benzene-1,3-diol

To a solution of (E)-7-(3,5-bis(methoxymethoxy)phenyl)-7-methyl-6-phenyloct-5-en-1-ol (1.5 g, 3.6 mmol, 1.0 equiv) in EtOAc (36 mL) was added Pd (10 wt % on C, 3.9 g. 3.6 mmol, 1.0 equiv) and the mixture was stirred for 7 h in an autoclave under 5 bar hydrogen. The suspension was filtered over a pad of Celite and concentrated under reduced pressure to afford the product as faintly yellowish oil (0.60 g, 61% yield).

¹H NMR (400 MHz, CD₃0D) 6=7.26-7.21 (m, 2H), 7.20-7.10 (m, 3H), 6.35 (d, J=2.2 Hz, 2H), 6.12 (t, J=2.1 Hz, 1H), 3.39 (t, J=6.6 Hz, 2H), 2.80 (dd, J=12.1, 2.8 Hz, 1H), 1.68 (dddd, J=13.7, 12.1, 9.4, 4.7 Hz, 1H), 1.48-1.38 (m, 1H), 1.36-1.24 (m, 2H), 1.20 (s, 3H), 1.16-1.06 (m, 2H), 1.03 (s, 3H), 0.92 (m, 2H). ¹³C NMR (101 MHz, CD₃0D) 6=158.9, 153.5, 143.1, 131.2, 128.5, 127.1, 106.5, 100.8, 62.9, 58.1, 42.3, 33.4, 30.4, 30.0, 29.2, 26.6, 23.4. IR (neat, vₘₐₓ/cm⁻¹) 3307, 2935, 2488, 1705, 1598, 1436, 1328, 1153. ESI-HRMS calcd for C₂₁H₂₈NaO₃ [M+Na]⁺351.1931; found 351.1928.

### step o) 2-(((1S,4S,5S)-4-(2,6-dihydroxy-4-(8-hydroxy-2-methyl-3-phenyloctan-2-yl)phenyl)-6,6-dimethylbicyclo[3.1.1]hept-2-en-2-yl)methyl)isoindoline-1,3-dione

To a solution of 5-(8-hydroxy-2-methyl-3-phenyloctan-2-yl)benzene-1,3-diol (0.38 g, 1.2 mmol, 1.0 equiv) and 2-(((1S,4R,5R)-4-hydroxy-6,6-dimethylbicyclo[3.1.1]hept-2-en-2-yl)methyl)isoindoline-1,3-dione (0.36 g, 1.2 mmol, 1.05 equiv) in CH₂Cl₂(46 mL) was added pTsOH·H₂O (0.062 g. 0.32 mmol, 0.28 equiv) and the yellow solution was stirred for 3 h. The reaction was stopped by addition on sat. aq. NaHCO₃ and the aqueous layer was extracted with CH₂Cl₂. The combined organic extracts were washed with brine, dried over Na₂SO₄ and concentrated under reduced pressure. Purification by flash column chromatography (SiO₂; using 10 to 50% EtOAc in hexanes) afforded the product as yellowish foam as a 1:1 mixture of diastereomers (0.42 g, 59%). 1:1 mixture of diastereomers—all signals reported: ¹H NMR (500 MHz, CDCl₃) δ=7.89 (ddd, J=5.4, 3.0, 0.9 Hz, 2H), 7.73 (dd, J=5.5, 3.0 Hz, 2zH), 7.23 (ddd, J=7.6, 6.4, 3.7 Hz, 2jH), 7.20-7.16 (m, 1H), 7.13-7.08 (m, 2H), 6.42 (br, 2H), 6.03-5.99 (m, 1H), 4.46(ddd, J=15.5, 3.0, 1.9 Hz, 1H), 4.23 (dt, J=15.5, 1.3 Hz, 1H), 4.05-4.01 (m, 1H), 3.58-3.45 (m, 2H), 2.79 (ddd, J=15.1, 12.1, 2.7 Hz, 1H), 2.33-2.21 (m, 3H), 1.60 (tp, J=13.2, 6.4, 5.9 Hz, 2H), 1.50-1.45 (m, 1H), 1.36 (ddd, J=13.9, 7.5, 3.2 Hz, 2H), 1.31 (s, 3H), 1.29-1.24 (m, 1H), 1.20-1.13 (m, 2H), 1.16 (d, J=2.4 Hz, 3H), 0.99 (s, 3H), 0.97 (d, J=9.0 Hz, 3H), 0.95-0.87 (m, 2H). ¹³C NMR (126 MHz, CDCl₃) δ=168.5, 168.5, 155.1, 150.2, 150.0, 148.0, 148.0, 141.8, 141.8, 134.3, 132.1, 130.3, 127.6, 127.6, 126.2, 123.7, 120.9, 120.8, 112.0, 112.0, 107.3, 63.2, 63.2, 56.2, 55.8, 47.4, 47.4, 44.4, 43.3, 41.3, 41.3, 41.2, 37.9, 32.2, 32.1, 30.0, 29.9, 28.8, 28.6, 28.2, 28.2, 27.6, 27.4, 26.0, 26.0, 25.1, 25.0, 22.4, 22.2, 20.7. IR (neat, vₘₐₓ/cm⁻¹) 3462, 2932, 1771, 1713, 1427, 1394. ESI-HRMS calcd for C₃₉H₄₅NNaO₅ [M+Na]⁺630.3190; found 630.3196. [α]²⁵_{D}=+90.727±0.124 (c=2.0, CHCl₃).

### step p) 2-(((1S,4S,5S)-4-(4-(8-hydroxy-2-methyl-3-phenyloctan-2-yl)-2,6-dimethoxyphenyl)-6,6-dimethylbicyclo[3.1.1]hept-2-en-2-yl)methyl)isoindoline-1,3-dione

To a solution of 2-(((1S,4S,5S)-4-(2,6-dihydroxy-4-(8-hydroxy-2-methyl-3-phenyloctan-2-yl)phenyl)-6,6-dimethylbicyclo[3.1.1]hept-2-en-2-yl)methyl)isoindoline-1,3-dione (0.42 g, 0.68 mmol, 1.0 equiv) in acetone (6.8 mL) was added (MeO)₂SO₂ (0.2 mL. 2.0 mmol, 3.0 equiv) and K₂CO₃ (0.33 g. 2.4 mmol, 3.5 equiv) and the purplish solution was stirred overnight. The reaction mixture was then diluted with ether and filtered over a pad of Celite, and the filtrate was concentrated under reduced pressure. Purification by flash column chromatography (SiO₂; using 10 to 30% acetone in hexanes) afforded the product as white foam as a 1:1 mixture of diastereomers (0.34 g, 77%).

1:1 mixture of diastereomers—all signals reported: ¹H NMR (400 MHz, CDCl₃) δ=7.85 (dd, J=5.4, 3.0 Hz, 2H), 7.70 (dd, J=5.4, 3.1 Hz, 2H), 7.19 (dtt, J=9.1, 5.1, 2.2 Hz, 3H), 7.02 (ddd, J=7.8, 3.5, 1.6 Hz, 2H), 6.39 (s, 2H), 5.60 (tt, J=3.1, 1.5 Hz, 1H), 4.28 (qt, J=15.4, 1.9 Hz, 2H), 3.96 (p, J=2.2 Hz, 1H), 3.68 (d, J=0.9 Hz, 6H), 3.51-3.44 (m, 2H), 2.71 (dt, J=12.1, 2.9 Hz, 1H), 2.21-2.11 (m, 2H), 2.01 (dtt, J=5.9, 4.0, 1.7 Hz, 1H), 1.72 (t, J=7.8 Hz, 1H), 1.66-1.55 (m, 1H), 1.43 (dtt, J=9.2, 6.1, 3.1 Hz, 1H), 1.38-1.29 (m, 2H), 1.27 (s, 3H), 1.25 (s, 3H), 1.19 (tt, J=9.0, 4.6 Hz, 2H), 1.11 (d, J=2.7 Hz, 3H), 0.98-0.87 (m, 2H), 0.94 (s, 3H). ¹³C NMR (101 MHz, CDCl₃) δ=168.3, 158.3, 148.6, 148.5, 141.9, 141.8, 136.2, 136.2, 133.9, 132.4, 130.2, 127.5, 127.5, 126.2, 124.0, 123.3, 117.9, 103.7, 103.7, 63.0, 57.2, 57.2, 55.9, 47.6, 47.6, 44.4, 44.3, 42.6, 41.8, 41.2, 37.5, 37.5, 32.6, 29.3, 29.0, 28.1, 27.7, 26.4, 25.6, 23.9, 23.8, 20.9. IR (neat, vₘₐₓ/cm⁻¹) 3467, 2932, 2865, 1771, 1712, 1603, 1572, 1466, 1410, 1390, 1239, 1117. ESI-HRMS calcd for C₄₁H₄₉NNaO₅ [M+Na]⁺658.3503; found 658.3496. [α]²⁵_{D}=+55.530±0.075 (c=3.0, CHC₃).

### step r) 2-(((1S,4S,5S)-4-(2,6-dimethoxy-4-(2-methyl-3-phenyloctan-2-yl)phenyl)-6,6-dimethylbicyclo[3.1.1]hept-2-en-2-yl)methyl)isoindoline-1,3-dione

To a solution of I₂ (0.036 g, 0.14 mmol, 1.5 equiv) in CH₂Cl₂ (0.5 mL) at 0° C. was added PPh₃ (0.042 g, 0.16 mmol, 1.7 equiv) and the resulting solution was stirred at 0° C. for 20 min. Imidazole (0.019 g, 0.28 mmol, 3.0 equiv) was added and stirring at 0° C. was continued for 10 min, then 2-(((1S,4S,5S)-4-(4-(8-hydroxy-2-methyl-3-phenyloctan-2-yl)-2,6-dimethoxyphenyl)-6,6-dimethylbicyclo[3.1.1]hept-2-en-2-yl)methyl)isoindoline-1,3-dione (0.060 g, 0.09 mmol, 1.0 equiv) in CH₂Cl₂ (0.5 mL) was added. The reaction was slowly allowed to warm to ambient temperature, and after 1 h TLC analysis (20% acetone in hexanes) showed full consumption of starting material. The reaction was stopped by addition of aq. sat. Na₂S₂O₃ and the mixture was extracted with CH₂Cl₂. The combined organic layers were washed with brine, dried over Na₂SO₄ and concentrated under reduced pressure. Purification by flash column chromatography (SiO₂; using 10% acetone in hexanes) afforded alkyl iodide intermediate as faintly yellow oil (0.070 g, quantitative yield). The alkyl iodide (0.070 g, 0.09 mmol, 1.0 equiv) was dissolved in toluene (1.0 mL) and AIBN (0.012 g, 0.08 mmol, 0.8 equiv) and Bu3SnH (0.15 mL, 0.56 mmol, 6.0 equiv) were added. The reaction solution was heated to 70° C. and stirred for 1 h. After TLC (20% acetone in hexanes) indicated full consumption of alkyl iodide, the reaction mixture was cooled to ambient temperature and concentrated under reduced pressure. Purification by flash column chromatography (SiO₂; using 5 to 10% acetone in hexanes) afforded the product as colorless oil as a 1:1 mixture of diastereomers (0.040 g, 68%) and minor stannane impurities.

Alkyl iodide, 1:1 mixture of diastereomers—all signals reported: ¹H NMR (400 MHz, CDCl₃) δ=7.85 (ddd, J=5.4, 3.0, 0.6 Hz, 2H), 7.74-7.67 (m, 2H), 7.24-7.14 (m, 3H), 7.01 (ddd, J=7.7, 4.0, 1.6 Hz, 2H), 6.38 (s, 2H), 5.59 (ddt, J=4.5, 3.0, 1.5 Hz, 1H), 4.27 (qt, J=15.4, 2.0 Hz, 2H), 3.96 (s, 1H), 3.68 (d, J=0.9 Hz, 6H), 3.02 (tt, J=7.0, 1.6 Hz, 2H), 2.70 (dt, J=12.1, 2.9 Hz, 1H), 2.20-2.12 (2H, assigned by HSQC), 2.02 (d, J=6.3 Hz, 1H), 1.72 (t, J=7.7 Hz, 1H), 1.65-1.56 (m, 3H), 1.46-1.37 (m, 1H), 1.34-1.21 (m, 10H), 1.11 (s, 1.5H), 1.10 (s, 1.5H), 0.93 (s, 3H). ESI-HRMS calcd for C₄1H₄₈1NNaO₄ [M+Na]⁺768.2520; found 768.2516.

2-(((1S,4S,5S)-4-(2,6-dimethoxy-4-(2-methyl-3-phenyloctan-2-yl)phenyl)-6,6-dimethylbicyclo[3.1.1]hept-2-en-2-yl)methyl)isoindoline-1,3-dione, 1:1 mixture of diastereomers—all signals reported: ¹H NMR (400 MHz, CDCl₃) δ=7.90-7.81 (m, 2H), 7.71 (dddd, J=5.5, 4.3, 2.7, 1.2 Hz, 2H), 7.23-7.11 (m, 3H), 7.01 (ddd, J=7.7, 3.4, 1.6 Hz, 2H), 6.39 (s, 2H), 5.60 (ddt, J=5.8, 2.9, 1.5 Hz, 1H), 4.28 (qt, J=15.4, 1.9 Hz, 2H), 3.96 (q, J=2.3 Hz, 1H), 3.68 (s, 6H), f2.71 (dt, J=12.0, 3.1 Hz, 1H), 2.23-2.10 (m, 2H), 2.01 (qd, J=3.7, 1.7 Hz, 1H), 1.76-1.70 (m, 1H), 1.68-1.60 (m, 1H), 1.58 (s, 1H), 1.45-1.40 (m, 1H), 1.27 (s, 3H), 1.24 (s, 3H), 1.12 (s, 1.5H), 1.11 (s, 1.5H), 1.07 (ddd, J=11.0, 6.0, 2.5 Hz, 4H), 0.94 (s, 3H), 0.92-0.88 (m, 1H), 0.73 (dt, J=7.3, 3.7 Hz, 3H). ¹³C NMR (101 MHz, CDCl₃) δ=168.3, 158.3, 148.6, 148.6, 142.0, 142.0, 136.1, 136.1, 133.9, 132.4, 130.2, 127.4, 127.4, 126.1, 124.0, 124.0, 123.3, 117.9, 117.8, 103.8, 103.7, 57.3, 57.2, 55.9, 55.8, 47.6, 44.4, 42.7, 41.8, 41.2, 37.5, 37.5, 31.8, 29.3, 29.0, 28.0, 27.7, 26.4, 24.0, 24.0, 22.6, 22.5, 20.9, 14.1. IR (neat, vₘₐₓ/cm⁻¹) 2930, 1772, 1715, 1604, 1573, 1390, 1120. ESI-HRMS calcd for C₄1H₄₉NNaO₄ [M+Na]⁺642.3554; found 642.3551. [α]²⁴D=+46.179±0.165 (c=2.0, CHCl₃).

### step s) ((1S,4S,5S)-4-(2,6-dimethoxy-4-(2-methyl-3-phenyloctan-2-yl)phenyl)-6,6-dimethylbicyclo[3.1.1]hept-2-en-2-yl)methanamine

To a solution of 2-(((1S,4S,5S)-4-(2,6-dimethoxy-4-(2-methyl-3-phenyloctan-2-yl)phenyl)-6,6-dimethylbicyclo[3.1.1]hept-2-en-2-yl)methyl)isoindoline-1,3-dione (0.040 g, 0.065 mmol, 1.0 equiv) in EtOH (1.3 mL) was added N₂H₄H₂O (0.032 mL, 0.65 mmol, 10 equiv) and (E)/(Z)-crotyl alcohol (0.083 mL, 0.97 mmol, 15 equiv) and the solution was heated to 75° C. and stirred for 3 h. Precipitation of a white solid occurred, and the mixture was filtered over a pad of Celite, which was rinsed with EtOH. The filtrate was concentrated under reduced pressure. Purification by flash column chromatography (SiO₂; using 1 to 10% MeOH in CH₂Cl₂, 1% 7N NH₃ in MeOH) afforded the product as faintly brown oil as a 1:1 mixture of diastereomers (0.030 g, 95%).

1:1 mixture of diastereomers—all signals reported: ¹H NMR (400 MHz, CDCl₃) δ=7.24-7.15 (m, 3H), 7.02 (dt, J=7.9, 1.7 Hz, 2H), 6.42 (s, 2H), 5.69 (p, J=1.8 Hz, 1H), 4.01 (s, 1H), 3.70 (s, 6H), 3.35 (s, 2H), 2.72 (dd, J=12.0, 2.9 Hz, 1H), 2.25-2.17 (m, 2H), 2.06 (qd, J=4.6, 3.9, 2.1 Hz, 1H), 1.72 (td, J=6.9, 1.9 Hz, 1H), 1.59 (tt, J=10.2, 2.7 Hz, 1H), 1.45 (dtd, J=12.9, 6.8, 4.0 Hz, 1H), 1.32 (s, 3H), 1.27 (s, 3H), 1.14 (d, J=1.5 Hz, 3H), 1.09 (tt, J=9.5, 3.7 Hz, 4H), 0.99 (s, 3H), 0.96-0.86 (m, 2H), 0.78-0.72 (m, 3H).¹³C NMR (101 MHz, CDCl₃) δ=158.3, 148.7, 148.6, 142.0, 140.6, 130.2, 127.4, 126.1, 123.8, 118.1, 118.0, 104.0, 103.9, 57.3, 56.0, 56.0, 47.6, 46.8, 44.5, 41.8, 41.1, 37.6, 31.9, 29.4, 28.9, 28.1, 28.1, 28.0, 26.4, 24.1, 24.1, 22.6, 21.3, 14.2. IR (neat, vₘₐₓ/cm⁻¹) 2931, 1604, 1572, 1464, 1452, 1410, 1239, 1121. ESI-HRMS calcd for C₃₃H₄₇NNaO₂ [M+Na]⁺512.3499; found 512.3502. [α]²⁶D=+57.496±0.142 (c=1.0, CHCl₃).

### step t) 6-((E)-2-(7-(diethylamino)-2-oxo-2H-chromen-3-yl)vinyl)-1-(6-((((S,4S,5S)-4-(2,6-dimethoxy-4-(2-methyl-3-phenyloctan-2-yl)phenyl)-6,6-dimethylbicyclo[3.1.1]hept-2-en-2-yl)methyl)amino)-6-oxohexyl)pyridin-1-ium-3-sulfonate

To a solution of ((1S,4S, 5S)-4-(2,6-dimethoxy-4-(2-methyl-3-phenyloctan-2-yl)phenyl)-6,6-dimethylbicyclo[3.1.1]hept-2-en-2-yl)methanamine (5.0 mg, 0.010 mmol, 1.0 equiv) in CH₂Cl₂ (0.1 mL) was added Fluorescence Red Mega 480 succinimidyl ester (6.2 mg, 0.010 mmol, 1.0 equiv, CAS RN: 1034442-03-9). The deep red solution was stirred at ambient temperature and under exclusion of light overnight, after which the volatiles were removed by N₂ flow. Purification by flash column chromatography (superneutral SiO₂; using 1 to 5% MeOH in CH₂Cl₂) afforded the product as dark red wax as a 1:1 mixture of diastereomers (6.0 mg, 59%). Residual N-hydroxysuccinimide was separated by preparative reverse-phase HPLC (Reprosil Gold 120 C18 125×20 mm column, flow 26.5 mL/min) to afford analytically clean product (2.0 mg, 19%).

ESI-HRMS calcd for C₅₉H₇₅N₃NaO₈S [M+Na]⁺1008.5167; found 1008.5167. Preparative HPLC H₂O (+0.1% HCOOH): MeCN (+0.1% HCOOH)=10:90 (t=0.0 min)→10:90 (t=15.0 min), t_{R}=5.0 min.

### Example 17

### N-(((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)pent-4-ynamide

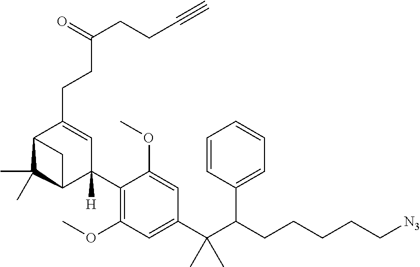 [SMILES: C#CCCC(=O)CCC1=C[C@H](c2c(OC)cc(C(C)(C)C(CCCCCN=[N+]=[N-])c3ccccc3)cc2OC)[C@@H]2C[C@H]1C2(C)C]

### Step o) 2-(((1S,4S,5S)-4-(4-(8-chloro-2-methyl-3-phenyloctan-2-yl)-2,6-dihydroxyphenyl)-6,6-dimethylbicyclo[3.1.1]hept-2-en-2-yl)methyl)isoindoline-1,3-dione

To a solution of 5-(8-chloro-2-methyl-3-phenyloctan-2-yl)benzene-1,3-diol (69.3 mg, 200 μmol, 1.0 equiv) and pTsOH·H₂O (10.6 mg. 56.0 μmol, 0.28 equiv) in CH₂Cl₂ (10 mL) was added 2-(((1S,4R,5R)-4-hydroxy-6,6-dimethylbicyclo[3.1.1]hept-2-en-2-yl)methyl)isoindoline-1,3-dione (65.3 mg, 220 μmol, 1.1 equiv) and the light-yellow solution was stirred at rt for 1.5 h. The reaction was stopped by addition of sat. aq. NaHCO₃ (5 mL), the layers were separated and the aqueous layer was extracted with Et₂O (3×10 mL). Combined organic extracts were dried over MgSO₄ and concentrated in vacuo. Purification by flash column chromatography (SiO₂; 0-20% EtOAc in hexanes) afforded the product as a colourless foam (80.0 mg, 64%).

1:1 mixture of diastereomers—all signals reported: ¹H NMR (400 MHz, CDCl₃) δ 7.95-7.85 (m, 2H), 7.80-7.67 (m, 2H), 7.29-7.14 (m, 3H), 7.12-7.03 (m, 2H), 6.55-5.14 (bs, 2H), 6.40 (bs, 2H), 6.02 (s, 1H), 4.47 (ddd, J=15.5, 3.0, 1.9 Hz, 1H), 4.29-4.18 (m, 1H), 4.01 (s, 1H), 3.42-3.31 (m, 2H), 2.71 (dd, J=12.1, 3.5 Hz, 1H), 2.37-2.20 (m, 3H), 1.74-1.34 (m, 5H), 1.32 (s, 3H), 1.30-1.20 (m, 2H), 1.17 (s, 3H), 1.01 (s, 3H), 1.00 (s, 3H), 0.96-0.80 (m, 2H). ¹³C NMR (101 MHz, CDCl₃) δ 168.5, 154.9, 150.2, 148.2, 141.8, 134.3, 132.1, 130.2, 127.6, 126.3, 123.7, 120.7, 111.9, 107.3, 56.9, 47.4, 45.3, 44.3, 43.3, 41.3, 41.2, 37.9, 32.5, 29.8, 29.2, 28.2, 27.5, 26.8, 26.0, 22.7, 20.7. IR (neat, vₘax/cm¹): 3460, 2931, 2867, 1771, 1714, 1624, 1574, 1426, 1393, 1341, 1025, 729, 705. HRMS (ESI): m/z=648.2834 [M+Na]⁺ (calc. for C₃₉H₄₄ClNNaO₄ m/z=648.2851). [α]²⁵_{D}=+80.468±0.199 (c=0.66, CHCl₃).

### Step p) 2-(((1S,4S,5S)-4-(4-(8-chloro-2-methyl-3-phenyloctan-2-yl)-2,6-dimethoxyphenyl)-6,6-dimethylbicyclo[3.1.1]hept-2-en-2-yl)methyl)isoindoline-1,3-dione

To a suspension of 2-(((1S,4S,5S)-4-(4-(8-chloro-2-methyl-3-phenyloctan-2-yl)-2,6-dihydroxyphenyl)-6,6-dimethylbicyclo[3.1.1]hept-2-en-2-yl)methyl)isoindoline-1,3-dione (30.0 mg, 47.9 μmol, 1.0 equiv) and K₂CO₃ (39.7 mg. 287 μmol, 6.0 equiv) in acetone (0.4 mL) was added (MeO)₂SO₂ (23 μL. 240 μmol, 5.0 equiv) and the solution was stirred at rt overnight. The reaction mixture was diluted with Et₂O (5 mL), filtered and concentrated in vacuo. Purification by flash column chromatography (SiO₂; 0-20% acetone in hexanes) afforded the product as a pale-yellow oil (24.5 mg, 78%).

1:1 mixture of diastereomers—all signals reported: ¹H NMR (400 MHz, CDCl₃) δ 7.89-7.81 (m, 2H), 7.75-7.67 (m, 2H), 7.24-7.13 (m, 3H), 7.05-6.99 (m, 2H), 6.39 (s, 2H), 5.62-5.57 (m, 1H), 4.34-4.29 (m, 1H), 4.27-4.20 (m, 1H), 3.98-3.94 (m, 1H), 3.68 (s, 6H), 3.39-3.32 (m, 2H), 2.70 (dd, J=12.1, 2.8 Hz, 1H), 2.21-2.12 (m, 2H), 2.05-1.98 (m, 1H), 1.72 (d, J=7.9 Hz, 1H), 1.66-1.35 (m, 4H), 1.30-1.21 (m, 2H), 1.27 (s, 3H), 1.24 (s, 3H), 1.11 (s, 3H), 0.94 (s, 3H), 0.98-0.84 (m, 2H). ¹³C NMR (101 MHz, CDCl₃) δ 168.3, 158.3, 148.4, 141.8, 136.1, 133.9, 132.4, 130.2, 127.5, 126.2, 124.0, 123.3, 118.0, 103.6, 57.2, 55.9, 47.6, 45.2, 44.4, 42.6, 41.8, 41.2, 37.5, 32.5, 29.2, 29.0, 27.7, 27.6, 26.8, 26.4, 23.9, 20.9. IR (neat, vₘₐₓ/cm⁻¹): 2932, 2864, 1771, 1714, 1603, 1572, 1465, 1451, 1389, 1239, 1116, 956, 730, 704. HRMS (ESI): m/z=676.3144 [M+Na]⁺ (calc. for C₄1H₄₈ClNNaO₄ m/z=676.3164). [α]²⁵_{D}=+57.601±0.343 (c=0.75, CHCl₃).

### Step r) 2-(((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)isoindoline-1,3-dione

To solution of 2-(((1S,4S,5S)-4-(4-(8-chloro-2-methyl-3-phenyloctan-2-yl)-2,6-dimethoxyphenyl)-6,6-dimethylbicyclo[3.1.1]hept-2-en-2-yl)methyl)isoindoline-1,3-dione (15.2 mg, 23.2 μmol, 1.0 equiv) in DMF (0.10 mL) was added NaN₃ (15.1 mg, 232 μmol, 10 equiv) and the reaction mixture was stirred at 80° C. for 24 h. The mixture was allowed to cool down to rt. and diluted with 5% aq. LiCl (5 mL) and Et₂O (5 mL). The layers were separated and the aqueous phase was extracted with Et₂O (4×5 mL). Combined organic extracts were dried over MgSO₄, filtered and concentrated in vacuo. Purification by flash column chromatography (SiO₂, 0-10% EtOAc in hexanes) afforded the product as a pale pink foam (14.7 mg, 96%).

1:1 mixture of diastereomers—all signals reported: ¹H NMR (400 MHz, CDCl₃) δ 7.92-7.82 (m, 2H), 7.76-7.68 (m, 2H), 7.24-7.14 (m, 3H), 7.06-6.98 (m, 2H), 6.38 (s, 2H), 5.66-5.55 (m, 1H), 4.35-4.28 (m, 1H), 4.27-4.20 (m, 1H), 4.00-3.93 (m, 1H), 3.68 (s, 6H), 3.09 (t, J=6.9 Hz, 2H), 2.78-2.66 (m, 1H), 2.26-2.11 (m, 2H), 2.08-1.97 (m, 1H), 1.72 (d, J=7.7 Hz, 1H), 1.68-1.54 (m, 1H), 1.50-1.29 (m, 3H), 1.27 (s, 3H), 1.26-1.23 (m, 3H), 1.23-1.13 (m, 2H), 1.12 (s, 3H), 0.94 (s, 3H), 1.02-0.82 (m, 2H). ¹³C NMR (101 MHz, CDCl₃) δ 168.34, 158.30, 148.51, 141.74, 136.16, 133.93, 132.42, 130.14, 127.53, 126.24, 123.94, 123.27, 117.94, 103.60, 57.19, 55.85, 51.49, 47.62, 44.36, 42.64, 41.78, 41.20, 37.51, 29.26, 28.98, 28.73, 27.88, 27.66, 26.65, 26.43, 23.92, 20.93. IR (neat, vₘₐₓ/cm⁻¹): 2931, 2864, 2094, 1772, 1715, 1603, 1572, 1466, 1451, 1389, 1239, 1117, 956, 729, 704. HRMS (ESI): m/z=683.3563 [M+Na]⁺ (calc. for C₄₁H₄₈N₄NaO₄m/z=683.3568). [α]²⁵_{D}=+43.225±0.114 (c=1.0, CHCl₃).

### Step s) ((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methanamine

To a solution of 2-(((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)isoindoline-1,3-dione (44.0 mg, 67.0 μmol, 1.0 equiv) in EtOH (1.3 mL) was added N₂H₄H₂O (33 μL, 670 μmol, equiv) and (E)/(Z)-crotyl alcohol (85 μL, 1.0 mmol, 15 equiv) and the solution was heated to 75° C. and stirred for 3 h. Precipitation of a white solid occurred, and the mixture was filtered over cotton, which was rinsed with EtOH. The filtrate was concentrated in vacuo. Purification by flash column chromatography (SiO₂; 1% 7.0 M NH₃ in MeOH, 1-4% MeOH in CH₂Cl₂) afforded the product as a white foam (27.0 mg, 76%).

1:1 mixture of diastereomers—all signals reported: ¹H NMR (400 MHz, CDCl₃) δ=7.28-7.14 (m, 3H), 7.03 (dt, J=7.8, 1.6 Hz, 2H), 6.43 (s, 2H), 5.57 (s, 1H), 4.02-3.97 (m, 1H), 3.70 (s, 6H), 3.24 (t, J=1.7 Hz, 2H), 3.11 (td, J=6.9, 1.1 Hz, 2H), 2.72 (dd, J=12.1, 2.9 Hz, 1H), 2.20 (dtd, J=8.3, 5.4, 1.2 Hz, 1H), 2.16-2.12 (m, 1H), 2.07 (tq, J=5.5, 1.9 Hz, 1H), 1.71 (dd, J=8.3, 5.9 Hz, 1H), 1.67-1.57 (m, 1H), 1.50-1.43 (m, 1H), 1.39 (dtd, J=8.8, 6.2, 5.7, 1.3 Hz, 2H), 1.31 (s, 3H), 1.27 (s, 3H), 1.24-1.17 (m, 2H), 1.14 (d, J=1.2 Hz, 3H), 1.00-0.88 (m, 2H), 0.97 (s, 3H). ¹³C NMR (101 MHz, CDCl₃) δ=158.4, 148.4, 148.4, 143.5, 143.5, 141.7, 130.1, 127.5, 127.5, 126.3, 121.1, 118.5, 118.5, 104.0, 104.0, 57.3, 56.0, 56.0, 51.5, 47.7, 47.6, 44.7, 41.8, 41.0, 37.6, 29.3, 29.3, 28.9, 28.7, 28.1, 27.9, 27.9, 26.7, 26.5, 24.0, 24.0,21.2. 1R (neat, vₘₐₓ/cm⁻¹) 2934, 2865, 2095, 1604, 1572, 1452, 1411, 1240, 1120. HRMS (ESI): m/z=553.3510 [M+Na]⁺ (calc. for C₃₃H₄₆N₄NaO₂ m/z=553.3513). [α]²⁵_{D}=+71.916±0.163 (c=0.7, CHCl₃).

### Step t)N-(((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)pent-4-ynamide

To a solution of 4-pentynoic acid (0.6 mg, 6.3 μmol, 1.3 equiv, CAS RN: 6089-09-4) in DMF (50 μL) was added HATU (2.8 mg, 7.2 μmol, 1.5 equiv) and i-Pr₂NEt (3.5 μL, 20 μmol, 4.0 equiv) and the reaction mixture was stirred at rt for 5 min before being added to a mixture of ((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methanamine (2.6 mg, 4.8 mol, 1.0 equiv) in DMF (50 μL). The reaction mixture was stirred at rt for 1 h and concentrated in vacuo. The crude product was purified by preparative TLC (SiO₂, 35% EtOAc in hexanes) to afford the title product as a colourless waxy solid (2.7 mg, 90%).

1:1 mixture of diastereomers—all signals reported: ¹H NMR (500 MHz, CD₂Cl₂) δ 7.26-7.16 (m, 3H), 7.11-7.05 (m, 2H), 6.46 (s, 2H), 5.63 (ddt, J=5.3, 2.9, 1.5 Hz, 1H), 5.53 (bt, J=5.4 Hz, 1H), 4.00-3.94 (m, 1H), 3.87-3.81 (m, 2H), 3.70 (s, 3H), 3.69 (s, 3H), 3.11 (t, J=6.9 Hz, 2H), 2.80-2.75 (m, 1H), 2.54-2.48 (m, 2H), 2.39 (t, J=7.1 Hz, 2H), 2.18 (dddd, J=8.4, 5.5, 4.6, 1.3 Hz, 1H), 2.10 (td, J=5.6, 1.4 Hz, 1H), 2.05-1.99 (m, 2H), 1.72-1.57 (m, 3H), 1.48-1.30 (m, 4H), 1.29 (s, 3H), 1.26 (s, 3H), 1.12 (s, 3H), 0.96 (s, 3H), 0.93 (m, 2H). ¹³C NMR (126 MHz, CD₂Cl₂) δ 170.86, 158.79, 149.39, 142.33, 138.98, 130.62, 127.94, 126.61, 124.18, 118.36, 104.28, 83.84, 69.41, 57.46, 56.32, 51.97, 48.03, 44.93, 42.23, 41.29, 38.05, 36.01, 29.74, 29.20, 29.14, 28.33, 28.09, 27.06, 26.57, 24.01, 21.27, 15.37. IR (neat, vₘₐₓ/cm⁻¹) 3310, 2927, 2857, 2094, 1650, 1604, 1572, 1452, 1411, 1365, 1302, 1261, 1239, 1184, 1120. HRMS (ESI): m/z=633.3767 [M+Na]⁺ (calc. for C₃₈H₅₀N₄NaO₃ m/z=633.3775).

### Example 18

### N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)pent-4-ynamide

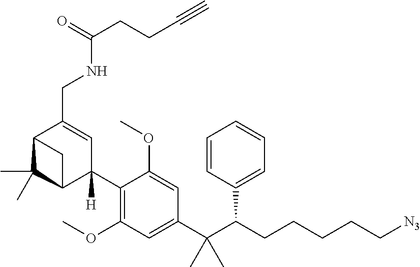 [SMILES: C#CCCC(=O)NCC1=C[C@H](c2c(OC)cc(C(C)(C)[C@@H](CCCCCN=[N+]=[N-])c3ccccc3)cc2OC)[C@@H]2C[C@H]1C2(C)C]

### Step t)N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)pent-4-ynamide

To a solution of 4-pentynoic acid (0.5 mg, 5.3 μmol, 1.3 equiv) in DMF (50 μL) was added HATU (2.4 mg, 6.2 μmol, 1.5 equiv) and i-Pr₂NEt (2.9 μL, 17 μmol, 4.0 equiv) and the reaction mixture was stirred at rt. for 5 min before being added to a mixture of ((1S, 4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methanamine (2.2 mg, 4.1 mol, 1.0 equiv) in DMF (50 μL). The reaction mixture was stirred at rt for 1 h and concentrated in vacuo. The crude product was purified by preparative TLC (SiO₂, 35% EtOAc in hexanes) to afford the title product as a colourless waxy solid (2.3 mg, 91%).

¹H NMR (400 MHz, CDCl₃) δ 7.25-7.14 (m, 3H), 7.06-6.98 (m, 2H), 6.42 (s, 2H), 5.68-5.62 (m, 1H), 5.54 (t, J=4.8 Hz, 1H), 4.02-3.95 (m, 1H), 3.89 (d, J=5.4 Hz, 2H), 3.70 (s, 6H), 3.11 (t, J=6.9 Hz, 2H), 2.72 (dd, J=12.0, 2.9 Hz, 1H), 2.55 (tdd, J=7.0, 2.7, 0.9 Hz, 2H), 2.43 (td, J=7.1, 1.0 Hz, 2H), 2.24-2.14 (m, 1H), 2.11 (td, J=5.6, 1.4 Hz, 1H), 2.10-2.02 (m, 1H), 1.99 (t, J=2.6 Hz, 1H), 1.70 (d, J=8.4 Hz, 1H), 1.69-1.11 (m, 6H), 1.29 (s, 3H), 1.27 (s, 3H), 1.14 (s, 3H), 1.01-0.79 (m, 2H), 0.97 (s, 3H). ¹³C NMR (101 MHz, CDCl₃) δ 170.8, 158.3, 148.6, 141.7, 138.3, 130.1, 127.6, 126.3, 124.2, 118.0, 103.9, 83.3, 69.5, 57.3, 56.0, 51.5, 47.5, 44.8, 44.5, 41.8, 41.0, 37.6, 35.8, 29.9, 29.3, 28.9, 28.7, 27.9, 27.9, 26.7, 26.4, 24.0, 21.2, 15.1. IR (neat, vₘₐₓ/cm⁻¹) 3308, 2925, 2853, 2094, 1652, 1603, 1571, 1452, 1410, 1261, 1120. HRMS (ESI): m/z=611.3947 [M+H]⁺ (calc. for C₃₈H₅₁N₄O₃ m/z=611.3956).

### Example 19

### N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)pent-4-ynamide

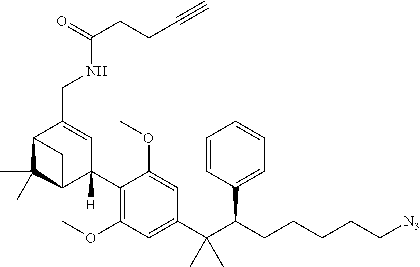 [SMILES: C#CCCC(=O)NCC1=C[C@H](c2c(OC)cc(C(C)(C)[C@H](CCCCCN=[N+]=[N-])c3ccccc3)cc2OC)[C@@H]2C[C@H]1C2(C)C]

### Step t)N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)pent-4-ynamide

To a solution of 4-pentynoic acid (0.9 mg, 9.0 μmol, 1.3 equiv) in DMF (50 μL) was added HATU (4.0 mg, 10 μmol, 1.5 equiv) and i-Pr₂NEt (4.9 μL, 28 μmol, 4.0 equiv) and the reaction mixture was stirred at rt for 5 min before being added to a mixture of ((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine (3.7 mg, 6.9 mol, 1.0 equiv) in DMF (50 μL). The reaction mixture was stirred at rt for 1 h and concentrated in vacuo. The crude product was purified by preparative TLC (SiO₂; 35% EtOAc in hexanes) to afford the title product as colourless waxy solid (4.1 mg, 96%).

¹H NMR (400 MHz, CDCl₃) δ 7.25-7.15 (m, 3H), 7.06-6.99 (m, 2H), 6.42 (s, 2H), 5.66 (dt, J=3.0, 1.5 Hz, 1H), 5.54 (s, 1H), 4.04-3.93 (m, 1H), 3.89 (d, J=5.4 Hz, 2H), 3.70 (s, 6H), 3.11 (t, J=6.9 Hz, 2H), 2.72 (dd, J=12.1, 2.9 Hz, 1H), 2.55 (tdd, J=7.0, 2.6, 1.0 Hz, 2H), 2.43 (td, J=7.1, 1.0 Hz, 2H), 2.26-2.14 (m, 1H), 2.11 (td, J=5.7, 1.4 Hz, 1H), 2.09-2.03 (m, 1H), 1.99 (t, J=2.6 Hz, 1H), 1.69 (d, J=8.4 Hz, 1H), 1.67-1.12 (m, 6H), 1.29 (s, 3H), 1.27 (s, 3H), 1.14 (s, 3H), 0.99-0.79 (m, 2H), 0.97 (s, 3H). ¹³C NMR (101 MHz, CDCl₃) δ 170.8, 158.3, 148.6, 141.7, 138.3, 130.1, 127.6, 126.3, 124.2, 118.0, 103.9, 83.3, 69.5, 57.3, 56.0, 51.5, 47.5, 44.8, 44.5, 41.8, 41.0, 37.6, 35.8, 29.9, 29.3, 28.9, 28.7, 28.0, 27.9, 26.7, 26.4, 24.0, 21.2, 15.1. IR (neat, vₘₐₓ/cm⁻¹) 3308, 2925, 2853, 2095, 1652, 1603, 1572, 1452, 1411, 1261, 1120. HRMS (ESI): m/z=611.3947 [M+H]⁺ (calc. for C₃₈H₅₁N₄O₃ m/z=611.3956).

### Example 20

### 1-(6-((((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate

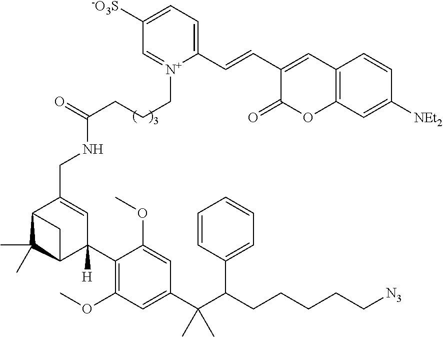

### Step t) 1-(6-((((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate

To a solution of ((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methanamine (9.0 mg, 17.0 μmol, 1.0 equiv) in DMF (50 μL) was added Fluorescence Red Mega 480 succinimidyl ester (10.0 mg, 17.0 μmol, 1.0 equiv, CAS RN: 1034442-03-9) in DMF (30 μL). The deep red solution was stirred at ambient temperature and under exclusion of light overnight, after which the mixture was concentrated in vacuo. Purification by flash column chromatography (superneutral SiO₂, 1-5% MeOH in CH₂Cl₂) afforded the product as dark red waxy solid (15.0 mg, 86%).

1:1 mixture of diastereomers—all signals reported: ¹H NMR (500 MHz, CD₂Cl₂) 6=9.02 (d, J=1.8 Hz, 1H), 8.62 (dd, J=8.6, 1.7 Hz, 1H), 8.14 (d, J=8.6 Hz, 1H), 8.10 (d, J=15.3 Hz, 1H), 7.94 (s, 1H), 7.51 (d, J=15.2 Hz, 1H), 7.44 (d, J=9.0 Hz, 1H), 7.25-7.19 (m, 2H), 7.20-7.15 (m, 1H), 7.09-7.05 (m, 2H), 6.69 (dd, J=9.0, 2.5 Hz, 1H), 6.50 (d, J=2.4 Hz, 1H), 6.44 (s, 2H), 6.12 (t, J=5.7 Hz, 1H), 5.61 (ddd, J=4.5, 3.0, 1.5 Hz, 1H), 4.64-4.56 (m, 2H), 3.94 (q, J=2.1 Hz, 1H), 3.89-3.79 (m, 2H), 3.68 (s, 6H), 3.68 (s, 4H), 3.46 (q, J=7.1 Hz, 4H), 3.09 (t, J=6.9 Hz, 2H), 2.76 (dt, J=11.8, 2.3 Hz, 1H), 2.28 (t, J=7.5 Hz, 2H), 2.18-2.13 (m, 1H), 2.12-2.04 (m, 3H), 2.00 (dtt, J=5.8, 3.9, 1.9 Hz, 1H), 1.76 (q, J=7.5 Hz, 2H), 1.68 (t, J=8.2 Hz, 2H), 1.64-1.56 (m, 2H), 1.47-1.39 (m, 1H), 1.38-1.31 (m, 2H), 1.27 (s, 3H), 1.25 (s, 3H), 1.23 (t, J=7.1 Hz, 6H), 1.20-1.12 (m, 2H), 1.09 (s, 3H), 0.97-085 (m, 2H), 0.94 (s, 3H). ¹³C NMR (126 MHz, CD₂Cl₂) 6=172.7, 172.6, 172.6, 160.6, 158.7, 157.4, 153.4, 153.1, 149.2, 147.8, 145.0, 143.0, 142.2, 141.3, 139.1, 131.2, 130.5, 127.8, 126.4, 124.8, 123.7, 118.4, 115.9, 113.8, 110.7, 109.4, 104.2, 97.1, 59.2, 57.3, 56.2, 56.2, 51.8, 48.0, 45.6, 44.8, 44.7, 44.5, 42.1, 41.1, 37.9, 36.3, 36.3, 29.8, 29.6, 29.1, 29.0, 28.2, 27.9, 26.9, 26.9, 26.5, 26.2, 25.9, 25.1, 23.9, 21.2, 12.7. IR (neat, vₘₐₓ/cm⁻¹): 2933, 2866, 2095, 1713, 1578, 1504, 1417, 1272, 1134, 1041. HRMS (ESI): m/z=1027.5356 [M+H]⁺ (calc. for C₅₉H₇₅N₆O₈S m/z=1027.5362)

### Example 21

### 1-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate

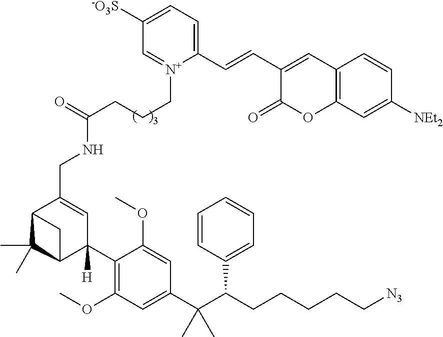

### Step t) 1-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate

To a solution of ((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methanamine (2.3 mg, 4.3 μmol, 1.0 equiv) in DMF (50 μL) was added Fluorescence Red Mega 480 succinimidyl ester (2.7 mg, 4.3 μmol, 1.0 equiv) in DMF (30 μL) and i-Pr₂NEt (0.8 μL, 4.3 μmol, 1.0 equiv). The deep red solution was stirred at ambient temperature and under exclusion of light overnight, after which the mixture was concentrated in vacuo. Purification by preparative TLC (SiO₂, 5% MeOH in CH₂Cl₂) afforded the product as dark red waxy solid (4.3 mg, 97%).

¹H NMR (500 MHz, CD₂Cl₂) δ 8.89 (s, 1H), 8.66 (d, J=8.5 Hz, 1H), 8.20-8.04 (m, 2H), 7.88 (s, 1H), 7.48-7.40 (m, 2H), 7.26-7.21 (m, 2H), 7.20-7.16 (m, 1H), 7.09-7.04 (m, 2H), 6.69 (dd, J=9.0, 2.5 Hz, 1H), 6.51 (d, J=2.4 Hz, 1H), 6.45 (s, 2H), 5.83 (t, J=4.8 Hz, 1H), 5.67-5.56 (m, 1H), 4.58-4.50 (m, 2H), 3.97-3.94 (m, 1H), 3.91-3.77 (m, 2H), 3.68 (s, 6H), 3.47 (q, J=7.1 Hz, 4H), 3.10 (t, J=6.9 Hz, 2H), 2.77 (dd, J=12.0, 2.8 Hz, 1H), 2.32-1.94 (m, 7H), 1.81-1.51 (m, 6H), 1.48-1.30 (m, 3H), 1.27 (s, 3H), 1.26 (s, 3H), 1.23 (t, J=7.1 Hz, 6H), 1.20-1.12 (m, 2H), 1.11 (d, J=3.5 Hz, 3H), 1.00-0.85 (m, 2H), 0.95 (s, 3H). ¹³C NMR (126 MHz, CD₂Cl₂) δ 172.3, 158.8, 157.6, 153.6, 149.4, 148.1, 142.6, 142.3, 139.3, 131.4, 130.6, 127.9, 126.6, 124.8, 123.8, 118.5, 115.9, 113.8, 110.9, 109.5, 104.3, 97.3, 59.4, 57.4, 56.4, 52.0, 48.1, 45.8, 44.9, 44.8, 42.2, 41.3, 38.1, 36.5, 32.5, 30.0, 29.7, 29.2, 29.1, 28.3, 28.1, 27.0, 26.6, 26.4, 25.4, 25.1, 24.0, 23.3, 21.3, 12.8. IR (neat, vₘₐₓ/cm⁻¹): 2917, 2851, 2094, 1717, 1578, 1504, 1416, 1272, 1134, 1043. HRMS (ESI): m/z=1027.5357 [M+H]⁺ (calc. for C₅₉H₇₅N₆O₈S m/z=1027.5362).

### Example 22

### 1-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate

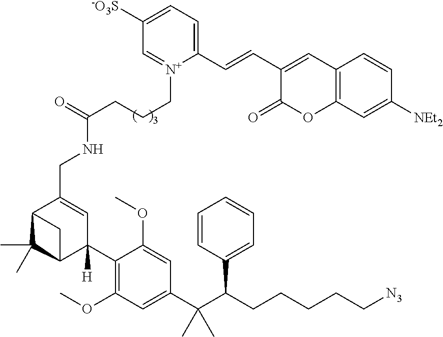

### Step t)1-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate

To a solution of ((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine (2.2 mg, 4.1 μmol, 1.0 equiv) in DMF (50 μL) was added Fluorescence Red Mega 480 succinimidyl ester (2.5 mg, 4.1 μmol, 1.0 equiv) in DMF (30 μL) and i-Pr₂NEt (0.7 μL, 4.1 μmol, 1.0 equiv). The deep red solution was stirred at ambient temperature and under exclusion of light overnight, after which the mixture was concentrated in vacuo. Purification by preparative TLC (SiO₂; 5% MeOH in CH₂Cl₂) afforded the product as dark red waxy solid (3.7 mg, 87%).

¹H NMR (500 MHz, CD₂Cl₂) δ 8.89 (s, 1H), 8.66 (d, J=8.9 Hz, 1H), 8.18-8.05 (m, 2H), 7.88 (s, 1H), 7.49-7.40 (m, 2H), 7.26-7.20 (m, 2H), 7.20-7.16 (m, 1H), 7.07 (dt, J=8.0, 1.4 Hz, 2H), 6.70 (dd, J=9.0, 2.5 Hz, 1H), 6.51 (d, J=2.5 Hz, 1H), 6.45 (s, 2H), 5.83 (t, J=5.8 Hz, 1H), 5.65-5.56 (m, 1H), 4.60-4.48 (m, 2H), 4.01-3.91 (m, 1H), 3.90-3.79 (m, 2H), 3.68 (s, 6H), 3.47 (q, J=7.1 Hz, 4H), 3.10 (td, J=7.0, 1.8 Hz, 2H), 2.76 (dd, J=12.1, 3.0 Hz, 1H), 2.25 (t, J=7.5 Hz, 2H), 2.22-1.98 (m, 5H), 1.80-1.50 (m, 6H), 1.46-1.30 (m, 3H), 1.28 (s, 3H), 1.26 (s, 3H), 1.23 (t, J=7.1 Hz, 6H), 1.19-1.12 (m, 2H), 1.10 (s, 3H), 1.00-0.84 (m, 2H), 0.95 (s, 3H). ¹³C NMR (126 MHz, CD₂Cl₂) δ 172.3, 160.7, 158.8, 157.6, 153.6, 153.1, 149.4, 148.0, 142.7, 142.3, 141.4, 139.3, 131.3, 130.6, 127.9, 126.6, 124.8, 123.8, 118.4, 115.9, 113.8, 110.9, 109.5, 104.3, 97.3, 59.4, 57.4, 56.3, 52.0, 48.1, 45.8, 44.9, 44.8, 42.2, 41.3, 38.1, 36.5, 30.0, 29.7, 29.2, 29.1, 28.3, 28.1, 27.1, 26.6, 26.4, 25.1, 24.0, 21.3, 12.8. IR (neat, vₘₐₓ/cm⁻¹): 2918, 2851, 2094, 1714, 1579, 1504, 1415, 1240, 1042. HRMS (ESI): m/z=1027.5357 [M+H]⁺ (calc. for C₅₉H₇₅N₆O₈S m/z=1027.5362).

### Example 23

### N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo [c][1,2,5]oxadiazol-4-yl)amino)heptanamide

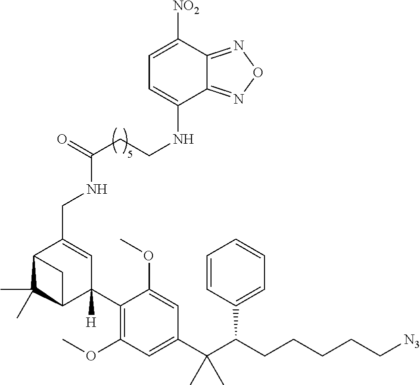

### Step t)N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)amino)heptanamide

To a solution of 7-((7-nitrobenzo[c][1,2,5]oxadiazol-4-yl)amino)heptanoic acid (2.5 mg, 8.1 μmol, 1.5 equiv) in DMF (50 μL) was added HATU (3.1 mg, 8.1 μmol, 1.5 equiv) and i-Pr₂NEt (3.8 μL, 22 μmol, 4.0 equiv) and the reaction mixture was stirred at rt. for 5 min before being added to a mixture of ((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methanamine (2.9 mg, 5.4 μmol, 1.0 equiv) in DMF (50 μL). The reaction mixture was stirred at rt. for 1 h and concentrated in vacuo. The crude product was purified by preparative TLC (SiO₂; 2% MeOH in CH₂Cl₂) to afford the title product as yellow amorphous solid (2.8 mg, 62%).

¹H NMR (500 MHz, CD₃0D) δ 8.41 (d, J=8.9 Hz, 1H), 7.25-7.19 (m, 2H), 7.19-7.14 (m, 1H), 7.12-7.03 (m, 2H), 6.50 (s, 2H), 6.25 (d, J=8.9 Hz, 1H), 5.62-5.55 (m, 1H), 4.00-3.92 (m, 1H), 3.82-3.71 (m, 2H), 3.69 (s, 6H), 3.48 (bs, 2H), 3.09 (dt, J=6.9, 1.2 Hz, 2H), 2.81 (dd, J=12.0, 2.7 Hz, 1H), 2.24 (t, J=7.2 Hz, 2H), 2.19-2.05 (m, 2H), 1.95 (td, J=5.8, 2.9 Hz, 1H), 1.82-1.61 (m, 6H), 1.55-1.38 (m, 4H), 1.36-1.12 (m, 5H), 1.27 (s, 3H), 1.25 (s, 3H), 1.10 (s, 3H), 1.03-0.86 (m, 2H), 0.95 (s, 3H). ¹³C NMR (126 MHz, CD₃0D) δ 175.8, 159.6, 150.1, 143.0, 139.9, 131.2, 128.5, 127.2, 123.9, 119.0, 104.8, 58.1, 56.3, 52.3, 45.5, 44.9, 42.8, 41.8, 38.7, 37.1, 30.8, 30.1, 29.8, 29.5, 29.3, 28.6, 28.4, 27.8, 27.3, 27.1, 26.8, 24.0, 21.4. IR (neat, vₘₐₓ/cm⁻¹) 3303, 2919, 2852, 2094, 1650, 1579, 1300, 1121. HRMS (ESI): m/z=821.4703 [M+H]⁺ (calc. for C₄₆H₆₁N₈O₆ m/z=821.4709).

### Example 24

### N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo [c][1,2,5]oxadiazol-4-yl)amino)heptanamide

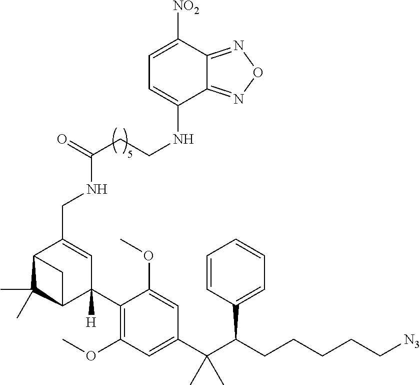

### Step t)N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)amino)heptanamide

To a solution of 7-((7-nitrobenzo[c][1,2,5]oxadiazol-4-yl)amino)heptanoic acid 2.4 mg, 7.6 μmol, 1.5 equiv) in DMF (50 μL) was added HATU (2.9 mg, 7.6 μmol, 1.5 equiv) and i-Pr₂NEt (3.5 μL, 20 μmol, 4.0 equiv) and the reaction mixture was stirred at rt. for 5 min before being added to a mixture of ((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine (2.7 mg, 5.0 μmol, 1.0 equiv) in DMF (50 μL). The reaction mixture was stirred at rt. for 1 h and concentrated in vacuo. The crude product was purified by preparative TLC (SiO₂; 2% MeOH in CH₂Cl₂) to afford the title product as yellow amorphous solid (2.5 mg, 60%).

¹H NMR (500 MHz, CD₃0D) δ 8.42 (d, J=8.8 Hz, 1H), 7.27-7.20 (m, 2H), 7.19-7.14 (m, 1H), 7.12-7.07 (m, 2H), 6.50 (s, 2H), 6.25 (d, J=8.9 Hz, 1H), 5.61-5.56 (m, 1H), 4.00-3.93 (m, 1H), 3.84-3.71 (m, 2H), 3.69 (s, 6H), 3.45 (bs, 2H), 3.08 (dt, J=6.8, 1.0 Hz, 2H), 2.81 (dd, J=12.0, 2.8 Hz, 1H), 2.24 (t, J=7.2 Hz, 2H), 2.16-2.06 (m, 2H), 2.00-1.94 (m, 1H), 1.81-1.62 (m, 6H), 1.52-1.40 (m, 4H), 1.40-1.11 (m, 5H), 1.26 (s, 3H), 1.25 (s, 3H), 1.11 (s, 3H), 1.03-0.85 (m, 2H), 0.95 (s, 3H). ¹³C NMR (126 MHz, CD₃0D) δ 175.8, 159.6, 150.1, 143.0, 139.9, 131.3, 128.5, 127.2, 123.8, 119.0, 104.7, 58.0, 56.2, 52.3, 45.5, 44.9, 42.8, 41.8, 38.7, 37.1, 30.1, 29.8, 29.5, 29.4, 28.6, 28.4, 27.8, 27.3, 27.1, 26.8, 24.0, 21.4. IR (neat, vₘₐₓ/cm⁻¹) 3319, 2924, 2853, 2095, 1579, 1300, 1121. HRMS (ESI): m/z=821.4697 [M+H]⁺ (calc. for C₄₆H₆₁N₈O₆ m/z=821.4709).

### Example 25

### N-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-bis(dimethylamino)-3-oxo-3H-spiro[isobenzofuran-1,9′-xanthene]-5-carboxamide

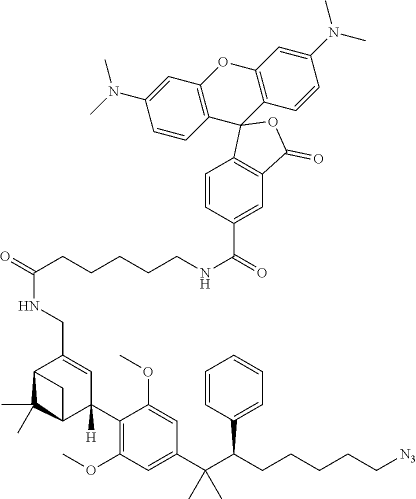 [SMILES: COc1cc(C(C)(C)[C@H](CCCCCN=[N+]=[N-])c2ccccc2)cc(OC)c1[C@H]1C=C(CNC(=O)CCCCCNC(=O)c2ccc3c(c2)C(=O)OC32c3ccc(N(C)C)cc3Oc3cc(N(C)C)ccc32)[C@H]2C[C@@H]1C2(C)C]

### Step u) tert-butyl (6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamate

To a solution of 6-(Boc-amino)hexanoic acid (9.9 mg, 42.6 μmol, 1.3 equiv) in DMF (0.1 mL) was added HATU (13.7 mg, 36.0 μmol, 1.1 equiv) and i-Pr₂NEt (20 μL, 115 μmol, 3.5 equiv) and the reaction mixture was stirred at 0° C. for 5 min before being added to a mixture of ((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine (17.4 mg, 32.7 mol, 1.0 equiv) in DMF (0.1 mL). The reaction mixture was stirred at rt for 1 h, diluted with EtOAc (15 mL) and quenched with water (10 mL). The phases were separated and the aqueous layer extracted with EtOAc (2×15 mL). The combined organic extracts were dried with MgSO₄, filtered and concentrated in vacuo. The crude material was purified by flash column chromatography (SiO₂; 0-15% (3× CV), 15%-25% (5×CV), 25% (5×CV), 25%-40% (5×CV) using (EtOAc:EtOH 3:1) in hexanes) to afford the title product as light yellow waxy solid (21.0 mg, 86%).

¹H NMR (400 MHz, CDCl₃) δ 7.25-7.14 (m, 3H), 7.08-6.99 (m, 2H), 6.42 (s, 2H), 5.63 (dt, J=2.9, 1.4 Hz, 1H), 5.34 (t, J=5.5 Hz, 1H), 4.53 (s, 1H), 4.01-3.94 (m, 1H), 3.94-3.80 (m, 2H), 3.70 (s, 6H), 3.10 (t, J=6.9 Hz, 4H), 2.72 (dd, J=12.1, 2.9 Hz, 1H), 2.24-2.14 (m, 3H), 2.13-2.01 (m, 2H), 1.74-1.57 (m, 5H), 1.55-1.11 (m, 8H), 1.44 (s, 9H), 1.29 (s, 3H), 1.27 (s, 3H), 1.14 (s, 3H), 1.02-0.87 (m, 2H), 0.96 (s, 3H). ¹³C NMR (101 MHz, CDCl₃) δ 172.7, 158.3, 156.1, 148.6, 141.7, 138.6, 130.1, 127.5, 126.3, 124.0, 118.0, 103.9, 79.2, 57.2, 56.0, 51.5, 47.5, 44.6, 44.5, 41.8, 40.9, 40.5, 37.6, 36.9, 30.0, 29.3, 28.9, 28.7, 28.6, 27.9, 27.9, 26.7, 26.6, 26.4, 25.6, 23.9, 21.2. IR (neat, vₘax/cm⁻¹) 3323, 2932, 2864, 2095, 1698, 1650, 1604, 1572, 1518, 1452, 1240, 1121. HRMS (ESI): m/z=744.5057 [M+H]⁺ (calc. for C₄₄H₆₆N₅O₅ m/z=744.5058).

### Step v) N-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-bis(dimethylamino)-3-oxo-3H-spiro[isobenzofuran-1, 9′-xanthene]-5-carboxamide

To a solution of tert-butyl (6-((((1S,4S, 5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamate (3.0 mg, 4.0 μmol, 1.0 equiv) in Et₂O (50 μL) was added HCl (2.0 M in Et₂O, 50 μL) and the solution was stirred for 1 h at rt. The mixture was concentrated in vacuo and the hydrochloride salt used immediately in the next step without further purification.

To 5-Carboxytetramethylrhodamine (2.2 mg, 5.0 μmol, 1.25 equiv, CAS RN: 150322-05-7) was added i-Pr₂NEt (3.5 μL, 20 μmol, 5.0 equiv) and anhydrous DMF (20 μL) and the mixture was stirred for 1 min. Subsequently a solution of the crude hydrochloride (2.7 mg, 4.0 μmol, 1.0 equiv) in anhydrous DMF (50 μL) and HATU (1.9 mg, 5.0 μmol, 1.25 equiv) were added and the solution was stirred for 1 h at rt. The mixture was concentrated in vacuo and purified by Preparative reverse-phase HPLC (Dr. Maisch Reprosil Gold 120 C4 150×20 mm, flow 26.5 mLmin⁻¹, gradient of 50%-90% acetonitrile (+0.1% HCOOH) in water (+0.1% HCOOH) over 30 min, column temperature 25° C.) to yield the title compound as a red lyophilized powder (1.8 mg, 43%).

HRMS (ESI): m/z=1056.5958 [M+H]⁺ (calc. for C₆₄H₇₈N₇O₇ m/z=1056.5957).

Preparative reverse-phase HPLC: (Dr. Maisch Reprosil Gold 120 C4 150×20 mm, flow 26.5 mL/min, column temperature 25° C., H₂O (+0.1% HCOOH): MeCN (+0.1% HCOOH)=50:50 (t=0.0 min)→50:50 (t=1.0 min)→10:90 (t=24.0 min)→10:90 (t=26.0 min)→50:50 (t=30.0 min), t_{R}=9.20 min. Analytical UPLC: (Acquity UPLC® BEH C18 1.7 μm 2.1×50 mm, H₂O (+0.1% HCOOH): MeCN (+0.1% HCOOH)=98:2 (t=0.0 min)→98:2 (t=0.5 min)→2:98 (t=4.0 min)→2:98 (t=5.0 min)→98:2 (t=6.5 min), tR=4.18 min.

### Example 26

### N-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-dihydroxy-3-oxo-3H-spiro[isobenzofuran-1,9′-xanthene]-6-carboxamide

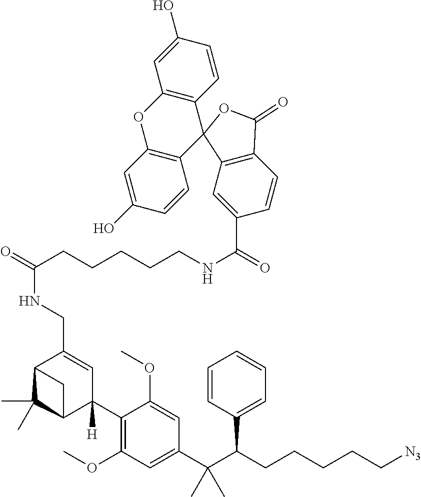 [SMILES: COc1cc(C(C)(C)[C@H](CCCCCN=[N+]=[N-])c2ccccc2)cc(OC)c1[C@H]1C=C(CNC(=O)CCCCCNC(=O)c2ccc3c(c2)C2(OC3=O)c3ccc(O)cc3Oc3cc(O)ccc32)[C@H]2C[C@@H]1C2(C)C]

### Step v) N-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-dihydroxy-3-oxo-3H-spiro[isobenzofuran-1,9′-xanthene]-6-carboxamide

To a solution of tert-butyl (6-((((1S,4S, 5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamate (3.0 mg, 4.0 μmol, 1.0 equiv) in Et₂O (50 μL) was added HCl (2.0 M in Et₂O, 50 μL) and the solution was stirred for 1 h at rt. The mixture was concentrated in vacuo and the hydrochloride salt used immediately in the next step without further purification.

To 6-Carboxyfluorescein (1.9 mg, 5.0 μmol, 1.25 equiv, CAS RN: 3301-79-9) was added i-Pr₂NEt (3.5 μL, 20 μmol, 5.0 equiv) and anhydrous DMF (20 μL) and the mixture was stirred for 5 min. Subsequently a solution of the crude hydrochloride (2.7 mg, 4.0 μmol, 1.0 equiv) in anhydrous DMF (50 μL) and HATU (1.9 mg, 5.0 μmol, 1.25 equiv) were added and the solution was stirred for 1 h at rt. The mixture was concentrated in vacuo and purified by preparative TLC (SiO₂; 1% AcOH, 7% MeOH in CH₂Cl₂) and further by preparative reverse-phase HPLC (Dr. Maisch Reprosil Gold 120 C4 150×20 mm, flow 26.5 mL/min, gradient of 60%-90% acetonitrile (+0.1% HCOOH) in water (+0.1% HCOOH) over 30 min, column temperature 25° C.) to yield the title compound as a yellow lyophilized powder (1.5 mg, 37%).

HRMS (ESI): m/z=1002.5012 [M+H]⁺ (calc. for C₆₀H₆₈N₅O₉ m/z=1002.5012).

Preparative reverse-phase HPLC: (Dr. Maisch Reprosil Gold 120 C4 150×20 mm, flow 26.5 mL/min, column temperature 25° C., H₂O (+0.1% HCOOH): MeCN (+0.10% HCOOH)=40:60 (t=0.0 min)→40:60 (t=1.0 min)→10:90 (t=24.0 min)→10:90 (t=26.0 min)
40:60 (t=30.0 min), t_{R}=10.81 min. Analytical UPLC: (Acquity UPLC® BEH C18 1.7 μm 2.1×50 mm, H₂O (+0.1% HCOOH): MeCN (+0.1% HCOOH)=98:2 (t=0.0 min)→98:2 (t=0.5 min)→2:98 (t=4.0 min)→2:98 (t=5.0 min)→98:2 (t=6.5 min), tR=4.70 min.

### Example 27

### 1-(6-((6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)amino)-6-oxohexyl)-2-((1E,3E)-5-((E)-3,3-dimethyl-5-sulfo-1-(3-sulfopropyl)indolin-2-ylidene)penta-1,3-dien-1-yl)-3-methyl-3-(4-sulfobutyl)-3H-indol-1-ium-5-sulfonate

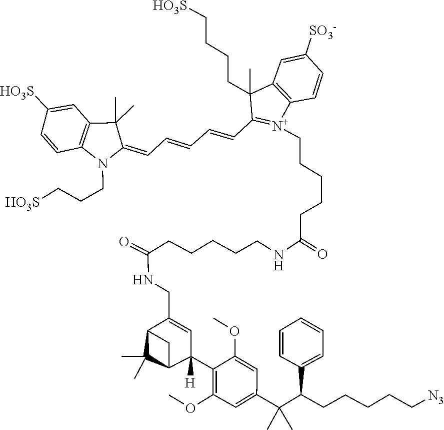

### Step v) 1-(6-((6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)amino)-6-oxohexyl)-2-((1E,3E)-5-((E)-3,3-dimethyl-5-sulfo-1-(3-sulfopropyl)indolin-2-ylidene)penta-1,3-dien-1-yl)-3-methyl-3-(4-sulfobutyl)-3H-indol-1-ium-5-sulfonate

To a solution of tert-butyl (6-((((1S,4S, 5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamate (7.0 mg, 9.4 μmol, 1.8 equiv) in Et₂O (100 μL) was added HCl (2.0 M in Et₂O, 100 μL) and the solution was stirred for 1 h at rt. The mixture was concentrated in vacuo and the hydrochloride salt used immediately in the next step without further purincation.

To AlexaFluor647-OSu (5.0 mg, 5.2 μmol, 1.0 equiv, JenaBioscience) was added a solution of the crude hydrochloride (6.5 mg, 9.4 μmol, 1.8 equiv) in anhydrous DMF (100 μL) and i-Pr₂NEt (2.7 μL, 16 μmol, 3.0 equiv) and the deep blue solution was stirred at rt for 16 h. To reach full conversion of the hydrolyzed OSu-ester, HATU (0.5 mg, 1.3 μmol, 0.25 equiv) and i-Pr₂NEt (1.0 μL, 5.7 μmol, 1.1 equiv) were added and the solution was stirred for additional 30 min upon which full conversion was confirmed. The mixture was concentrated in vacuo and directly purified by preparative TLC (SiO₂, 20% H₂O, 40% i-PrOH in EtOAc) and a second time (8-15% MeOH in CH₂Cl₂) to remove the coeluting HOAt contaminant. The product was furnished as a deep blue wax (5.2 mg, 70%).

¹H NMR (500 MHz, CD₃0D) δ 8.55 (s, 1H), 8.41-8.28 (m, 2H), 7.92-7.87 (m, 4H), 7.42 (d, J=8.3 Hz, 1H), 7.33 (d, J=8.3 Hz, 1H), 7.25-7.20 (m, 2H), 7.20-7.14 (m, 1H), 7.13-7.08 (m, 2H), 6.73 (t, J=12.4 Hz, 1H), 6.54-6.52 (m, 1H), 6.52 (s, 2H), 6.37 (d, J=13.8 Hz, 1H), 5.60-5.57 (m, 1H), 4.35 (t, J=8.1 Hz, 2H), 4.13 (t, J=7.4 Hz, 2H), 3.99-3.96 (m, 1H), 3.84-3.71 (m, 2H), 3.69 (s, 6H), 3.66-3.63 (m, 2H), 3.16-3.06 (m, 4H), 3.00 (t, J=6.6 Hz, 2H), 2.83 (dd, J=12.0, 2.5 Hz, 1H), 2.65-2.60 (m, 2H), 2.23 (t, J=7.4 Hz, 4H), 2.20-2.12 (m, 5H), 2.09 (t, J=5.6 Hz, 1H), 2.03-1.96 (m, 1H), 1.86-1.80 (m, 1H), 1.77 (s, 3H), 1.76 (s, 3H), 1.73 (s, 3H), 1.72-1.41 (m, 8H), 1.39-1.15 (m, 12H), 1.29 (s, 3H), 1.28 (s, 3H), 1.12 (s, 3H), 1.04-0.97 (m, 2H), 0.95 (s, 3H). ¹³C NMR (126 MHz, CDCl₃) δ 175.8, 170.3, 159.6, 155.2 (HSQC), 131.3, 128.5, 128.3, 127.2, 123.9, 121.4, 111.4 (HSQC), 111.1 (HSQC), 105.6 (HSQC), 104.8, 71.3, 58.0, 56.3, 52.3, 52.1 (HSQC), 48.9 (HSQC), 48.6 (HSQC), 45.5, 44.9, 44.7 (HSQC), 43.7 (HSQC), 42.8, 41.8, 40.2, 39.4, 38.7, 37.1, 36.8, 33.1, 30.8, 30.5, 30.1, 29.5, 28.6, 28.4, 28.2, 27.9, 27.6, 27.3, 26.9, 26.6, 26.0, 24.0, 23.7, 21.4. IR (neat, vₘₐₓ/cm⁻¹) 3424, 2923, 2853, 2095, 1642, 1602, 1572, 1492, 1464, 1382, 1330, 1180. HRMS (ESI): m/z=1520.6240 [M+Na]⁺ (calc. for C₇₆H₁₀₃N₇NaO₁₆S₄ m/z=1520.6236).

### Example 28

### N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-6-(3-(5,5-difluoro-7-(1H-pyrrol-2-yl)-5H-514,614-dipyrrolo[1,2-c:2′,1′-f][1,3,2]diazaborinin-3-yl)propanamido)hexanamide

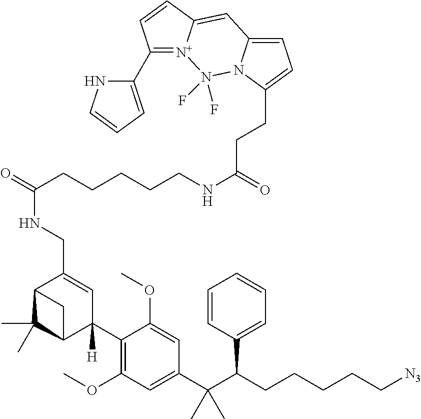

### Step v)N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-6-(3-(5,5-difluoro-7-(1H-pyrrol-2-yl)-5H-514,614-dipyrrolo[1,2-c:2′,1′-f][1,3,2]diazaborinin-3-yl)propanamido)hexanamide

To a solution of tert-butyl (6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamate (7.3 mg, 9.8 μmol, 1.2 equiv) in Et₂O (100 μL) was added HCl (2.0 M in Et₂O, 100 μL) and the solution was stirred for 1 h at rt. The mixture was concentrated in vacuo and the hydrochloride salt used immediately in the next step without further purification.

To BODIPY 576/589-COOH (2.7 mg, 8.2 μmol, 1.0 equiv, CAS RN: 150173-78-7) was added a solution of the crude hydrochloride (6.3 mg, 9.8 μmol, 1.2 equiv) in anhydrous DMF (100 μL), followed by i-Pr₂NEt (7.2 μL, 41 μmol, 5.0 equiv) and HATU (3.9 mg, 10 μmol, 1.25 equiv), and the deep purple solution was stirred at rt for 45 min. The mixture was concentrated in vacuo and directly purified by preparative TLC (SiO₂; 2% MeOH in CH₂Cl₂) and a second time (SiO₂; 7.5% EtOH, 22.5% EtOAc in hexanes) to yield the product as a dark purple solid (4.7 mg, 60%).

¹H NMR (500 MHz, CD₂Cl₂) δ 10.38 (s, 1H), 7.27-7.21 (m, 2H), 7.20-7.16 (m, 2H), 7.11-7.06 (m, 3H), 7.05 (s, 1H), 7.01 (ddd, J=3.9, 2.5, 1.4 Hz, 1H), 6.90 (d, J=4.6 Hz, 1H), 6.89 (d, J=4.0 Hz, 1H), 6.46 (s, 2H), 6.41-6.36 (m, 1H), 6.31 (d, J=4.0 Hz, 1H), 5.70 (t, J=5.5 Hz, 1H), 5.59 (dt, J=2.9, 1.5 Hz, 1H), 5.39 (t, J=5.7 Hz, 1H), 3.99-3.91 (m, 1H), 3.89-3.75 (m, 2H), 3.69 (s, 6H), 3.28 (t, J=7.7 Hz, 2H), 3.21 (td, J=7.0, 5.8 Hz, 2H), 3.10 (t, J=6.9 Hz, 2H), 2.77 (dd, J=11.9, 2.7 Hz, 1H), 2.59 (dd, J=8.2, 7.1 Hz, 2H), 2.20-2.11 (m, 3H), 2.08 (td, J=5.6, 1.4 Hz, 1H), 2.02 (tt, J=5.8, 1.9 Hz, 1H), 1.70-1.20 (m, 12H), 1.67 (d, J=8.3 Hz, 1H), 1.28 (s, 3H), 1.26 (s, 3H), 1.11 (s, 3H), 1.00-0.91 (m, 2H). 0.95 (s, 3H). ¹³C NMR (126 MHz, CD₂Cl₂) δ 172.8, 171.6, 158.8, 156.5, 150.8, 149.4, 142.3, 139.3, 137.8, 134.1, 132.2, 130.6, 127.9, 127.2, 126.6, 126.2, 124.1, 123.9, 120.8, 118.4, 118.2, 117.3, 112.0, 104.3, 57.4, 56.3, 52.0, 48.0, 44.9, 44.7, 42.2, 41.3, 39.7, 38.1, 37.1, 36.0, 29.8, 29.2, 29.1, 28.3, 28.1, 27.1, 26.9, 26.6, 25.8, 25.2, 24.0, 21.3. ¹⁹F NMR (471 MHz, CD₂Cl₂) δ-140.3 (d, J=36.3 Hz),−140.5 (d, J=36.1 Hz). ¹¹B NMR (160 MHz, CD₂Cl₂) δ 1.54 (t, J=36.3 Hz). IR (neat, vₘₐₓ/cm⁻¹) 3301, 2926, 2855, 2094, 1648, 1604, 1574, 1484, 1462, 1107. HRMS (ESI): m/z=955.5567 [M+H]⁺ (calc. for C₅₅H₇₀BF₂N₈O₄ m/z=955.5576).

### Example 29

### 3′,6′-diamino-5-((6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamoyl)-3-oxo-3H-spiro[isobenzofuran-1,9′-xanthene]-4′,5′-disulfonic acid

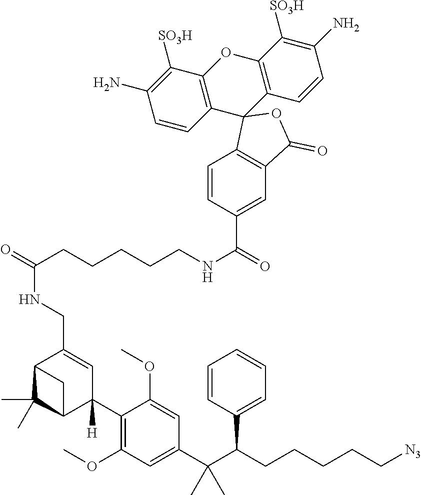 [SMILES: COc1cc(C(C)(C)[C@H](CCCCCN=[N+]=[N-])c2ccccc2)cc(OC)c1[C@H]1C=C(CNC(=O)CCCCCNC(=O)c2ccc3c(c2)C(=O)OC32c3ccc(N)c(S(=O)(=O)O)c3Oc3c2ccc(N)c3S(=O)(=O)O)[C@H]2C[C@@H]1C2(C)C]

### Step v) 3′,6′-diamino-5-((6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamoyl)-3-oxo-3H-spiro[isobenzofuran-1,9′-xanthene]-4′,5′-disulfonic acid

To a solution of tert-butyl (6-((((1S,4S, 5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamate (9.0 mg, 12.1 μmol, 1.3 equiv) in Et₂O (100 μL) was added HCl (2.0 M in Et₂O, 100 μL) and the solution was stirred for 1 h at rt. The mixture was concentrated in vacuo and the hydrochloride salt used immediately in the next step without further purification.

To AZDye488-COOH (5.0 mg, 9.3 μmol, 1.0 equiv, Fluoroprobes CAT No: 1012-5) was added a solution of the crude hydrochloride (8.3 mg, 12.1 μmol, 1.3 equiv) in anhydrous DMSO (200 μL), followed by i-Pr₂NEt (8.2 μL, 47 μmol, 5.0 equiv) and HATU (4.3 mg, 11.2 μmol, 1.2 equiv) and the deep red solution was stirred at rt for 1 h. The mixture was concentrated in vacuo and directly purified by preparative TLC (SiO₂, 40% i-PrOH, 20% H₂O in EtOAc) and a second time by Preparative reverse-phase HPLC (Dr. Maisch Reprosil Gold 120 C4 150×20 mm, flow 26.5 mLmin⁻¹, gradient of 50%-90% acetonitrile (+0.1% HCOOH) in water (+0.1% HCOOH) over 30 min, column temperature 25° C.) to yield the title compound as a red lyophilized powder (2.7 mg, 25%).

¹H NMR (600 MHz, (CD₃)₂SO) δ 8.79 (s, 1H), 8.44 (bs, 1H), 8.24 (d, J=7.9 Hz, 1H), 7.80 (t, J=6.1 Hz, 1H), 7.34 (bs, 1H), 7.27-7.22 (m, 2H), 7.21-7.13 (m, 3H), 6.81 (bs, 2H), 6.54 (s, 2H), 6.42 (bs, 2H), 5.45 (dt, J=3.3, 1.6 Hz, 1H), 3.89-3.83 (m, 1H), 3.74-3.69 (m, 1H), 3.68 (s, 6H), 3.62 (dd, J=15.2, 5.8 Hz, 1H), 3.51 (t, J=1.2 Hz, 2H), 3.12 (td, J=6.8, 1.7 Hz, 2H), 2.87 (dd, J=11.9, 2.7 Hz, 1H), 2.15-2.07 (m, 3H), 2.07-2.01 (m, 1H), 1.93 (t, J=5.6 Hz, 1H), 1.63 (d, J=8.1 Hz, 1H), 1.60-1.52 (m, 4H), 1.37-1.18 (m, 6H), 1.24 (s, 3H), 1.20 (s, 3H), 1.16-1.04 (m, 2H), 1.01 (s, 3H), 0.91 (s, 3H), 0.88-0.77 (m, 2H). ¹³C NMR (151 MHz, (CD₃)₂SO) δ 174.5, 171.8, 164.5, 157.8, 148.8, 141.5, 139.3, 136.1, 129.9, 127.4, 126.0, 121.1, 117.1, 103.6, 69.8, 55.9, 55.3, 50.5, 47.0, 43.4, 42.8, 41.3, 40.4, 40.2, 36.9, 35.5, 29.1, 28.9, 28.3, 27.9, 27.2, 27.1, 26.2, 26.2, 25.6, 25.3, 22.6, 20.8. IR (neat, vₘₐₓ/cm⁻¹) 3410, 3304, 3192, 2922, 2852, 2096, 1737, 1598, 1556, 1424, 1242.

HRMS (ESI): m/z=1182.4283 [M+Na]⁺ (calc. for C₆₀H₆₉N₇NaO₁₃S₂m/z=1182.4287).

Preparative reverse-phase HPLC (Dr. Maisch Reprosil 100 C4, 5 μm, 150×20 mm, flow 26.5 mL/min, column temperature 25° C., H₂O (+0.10% HCOOH): MeCN (+0.1% HCOOH)=50:50 (t=0.0 min)→50:50 (t=1.0 min)→10:90 (t=24.0 min)→10:90 (t=26.0 min)→50:50 (t=30.0 min), t_{R}=9.60 min. Analytical HPLC (Dr. Maisch Reprosil 100 C4, 5 μm, 150×4.6 mm, flow 1.4 mL/min, column temperature 25° C., H₂O (+0.1% HCOOH): MeCN (+0.1% HCOOH)=50:50 (t=0.0 min)→50:50 (t=1.0 min)→10:90 (t=12.0 min)→10:90 (t=14.0 min)→50:50 (t=15.0 min), t_{R}=8.22 min.

### Example 30

### N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo [c][1,2,5]oxadiazol-4-yl)oxy)heptanamide

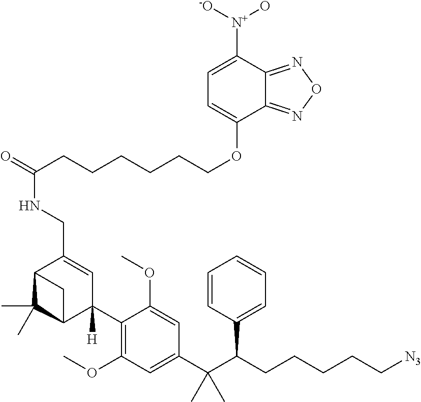

### Step t)N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)oxy)heptanamide

((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine (2.9 mg, 5.4 mol, 1.0 equiv) and i-Pr₂NEt (2.9 μL, 16.3 mol, 3.0 equiv) were stirred at rt in anhydrous CH₂Cl₂(50 L) for 15 min before a solution of the respective acid 7-((7-nitrobenzo[c][1,2,5]oxadiazol-4-yl)oxy)heptanoic acid (2.2 mg, 7.1 mol, 1.3 equiv, EP22192302.2) in anhydrous DMF (50 μL), pre-stirred at 0° C. for 15 min with HATU (3.1 mg, 8.1 mol, 1.5 equiv) was added in one portion. The combined reaction mixture was stirred at rt for 45 min. and subsequently concentrated in vacuoo. The crude product was purified by preparative TLC (SiO₂, 70% EtOAc in hexanes) to afford the product as a light yellow wax.

¹H NMR (600 MHz, C₆D₆) δ 7.67 (d, J=8.3 Hz, 1H), 7.20-7.17 (m, 2H), 7.13-7.09 (m, 1H), 7.08-7.04 (m, 2H), 6.58 (s, 2H), 5.74 (dt, J=2.9, 1.5 Hz, 1H), 5.35 (d, J=8.3 Hz, 1H), 4.73 (t, J=6.0 Hz, 1H), 4.42-4.35 (m, 1H), 3.98-3.90 (m, 2H), 3.52 (s, 6H), 3.47 (t, J=6.4 Hz, 2H), 2.83 (dd, J=12.1, 2.9 Hz, 1H), 2.51 (td, J=6.7, 2.3 Hz, 2H), 2.39 (tt, J=6.4, 2.1 Hz, 1H), 2.26 (dt, J=8.5, 5.6 Hz, 1H), 2.17 (td, J=5.7, 5.3, 1.4 Hz, 1H), 2.05 (d, J 10=8.5 Hz, 1H), 1.93-1.81 (m, 2H), 1.70-1.58 (m, 3H), 1.54-1.47 (m, 3H), 1.31 (s, 3H), 1.30 (s, 3H), 1.29-1.24 (m, 4H), 1.21 (s, 3H), 1.15 (s, 3H), 0.99-0.78 (m, 6H). ¹³C NMR (151 MHz, C6D6) δ 171.3, 159.0, 154.3, 149.0, 145.4, 144.3, 141.9, 139.6, 133.2, 130.4, 130.0, 127.9, 126.7, 123.0, 118.4, 103.9, 103.9, 70.5, 57.5, 55.6, 51.2, 48.1, 44.8, 44.4, 42.0, 41.0, 38.2, 36.3, 29.6, 29.4, 28.8, 28.7, 28.3, 28.1, 26.7, 26.5, 25.7, 25.5, 23.8, 21.4. IR (neat, vₘₐₓ/cm-1) 3310, 2932, 2861, 2094, 1642, 1547, 1452, 1319, 1103. HRMS (ESI): m/z=844.4364 [M+Na]⁺ (calc. for C46H₅₉N₇NaO₇m/z=844.4368).

### Example 31

### 1-(6-((2-(2-(4-(3-((4-((7-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate

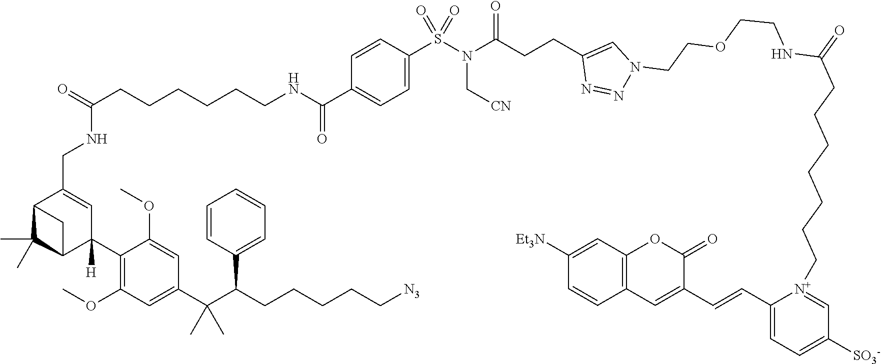

### Step t) 1-(6-((2-(2-(4-(3-((4-((7-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate

To a solution of (E)-1-(6-((2-(2-(4-(3-((4-((7-(tert-butoxy)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-(2-(7-(diethylamino)-2-oxo-2H-chromen-3-yl)vinyl)pyridin-1-ium-3-sulfonate (3.0 mg, 2.6 μmol, 1.0 equiv, EP22192302.2) in dry CH₂Cl₂ (200 μL) was added TFA (100 μL) and the resulting plum coloured reaction mixture was allowed to stir for 30 min at rt. Then, the solvents were removed in vacuoo, the residue was redissolved in CH₂Cl₂ and co-evaporated (×3). The residue was then dissolved in dry DMF (50 μL). TFA (0.5 M solution in dry DMF, 17.5 μL, 8.7 μmol, 3.3 equiv) and i-Pr₂NEt (0.5 M solution in dry DMF, 28.1 μL, 14.0 μmol, 5.3 equiv) were added and the resulting colourless solution was cooled to 0° C. Then, HATU (0.1 M solution in DMF, 29.2 μL, 2.9 μmol, 1.1 equiv) was added and the reaction was stirred at 0° C. for 15 min. Then amine ((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine (1.5 mg, 2.7 mol, 1.1 equiv) in CH₂Cl₂ (50 μL) was added. The reaction mixture was allowed to warm up to ambient temperature and stirred for further 30 min. The reaction mixture was then concentrated in vacuoo, the residue taken up in CH₂Cl₂ and directly loaded onto a silica plate. Purification by preparative TLC (SiO₂, 6% MeOH in CH₂Cl₂) afforded the product (2.5 mg, 59%) as a red solid.

HRMS (ESI): m/z=1586.7561 [M+H]⁺ (calc. for C84H₁₀₈N₁₃O₁₄S₂ m/z=1586.7575).

Analytical UPLC: (Acquity UPLC® BEH C18 1.7 μm 2.1×50 mm, H₂O (+0.1% HCOOH):MeCN (+0.1% HCOOH)=98:2 (t=0.0 min)→98:2 (t=0.5 min)→2:98 (t=4.0 min)→2:98 (t=5.0 min)→98:2 (t=6.5 min), t_{R}=4.65 min.

### Example 32

### (S)-5-(2-methyl-8-((4-nitrobenzoyl)oxy)-3-phenyloctan-2-yl)-1,3-phenylene bis(4-nitrobenzoate)

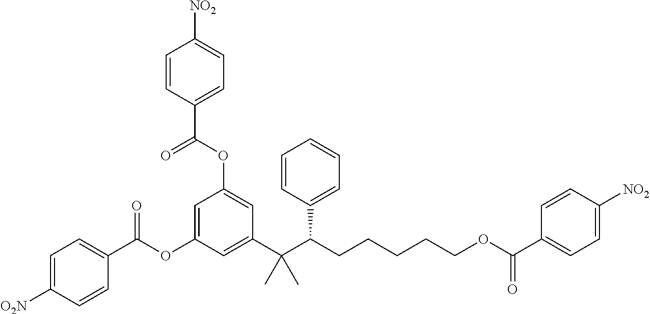 [SMILES: CC(C)(c1cc(OC(=O)c2ccc([N+](=O)[O-])cc2)cc(OC(=O)c2ccc([N+](=O)[O-])cc2)c1)[C@@H](CCCCCOC(=O)c1ccc([N+](=O)[O-])cc1)c1ccccc1]

### Step b) (S)-5-(8-hydroxy-2-methyl-3-phenyloctan-2-yl)benzene-1,3-diol

A suspension of (S)-5-(8-chloro-2-methyl-3-phenyloctan-2-yl)benzene-1,3-diol (32.0 mg, 92.2 μmol, 1.0 equiv) in H₂O (2.0 mL) was heated at 150° C. under microwave irradiation for 3 h. The vial was washed into a separatory funnel with EtOAc (10 mL) and CH₂Cl₂(10 mL). The phases were separated, and the aqueous phase was extracted with CH₂Cl₂ (2×10 mL). The combined organic layers were dried over Na₂SO₄, filtered, and concentrated in vacuo to afford (S)-5-(8-hydroxy-2-methyl-3-phenyloctan-2-yl)benzene-1,3-diol (30.1 mg, 91.6 μmol, 99%) as an off-white foam.

¹H NMR (400 MHz, CDCl₃) δ 7.25-7.16 (m, 3H), 7.15-7.08 (m, 2H), 6.48 (d, J=2.0 Hz, 2H), 6.25 (t, J=2.1 Hz, 2H), 3.52 (tdd, J=10.5, 6.6, 4.0 Hz, 2H), 2.80 (d, J=12.0 Hz, 1H), 1.69-1.52 (m, 1H), 1.40-1.09 (m, 10H), 0.99 (s, 3H), 0.96-0.81 (m, 2H). ¹³C NMR (101 MHz, CDCl₃) δ 156.5, 153.5, 141.6, 130.3, 127.7, 126.3, 106.7, 100.5, 63.3, 55.8, 41.6, 31.8, 30.1, 28.5, 27.2, 24.7, 22.2. IR (neat, vₘₐₓ/cm¹): 3307, 2935, 2488, 1705, 1598, 1436, 1328, 1153. HRMS (ESI): m/z=351.1928 [M+Na]⁺ (calc. for C21H₂₈NaO₃ m/z=351.1931). [α]²⁵_{D}=−32.414±0.253 (c=0.5, CHCl₃).

### Step c) (S)-5-(2-methyl-8-((4-nitrobenzoyl)oxy)-3-phenyloctan-2-yl)-1,3-phenylene bis(4-nitrobenzoate)

To a solution of (S)-5-(8-hydroxy-2-methyl-3-phenyloctan-2-yl)benzene-1,3-diol (17.5 mg, 53.3 μmol, 1.0 equiv), NEt₃ (37.7 mg, 52.0 μL, 373.0 μmol, 7.0 equiv) and DMAP (1.3 mg, 10.7 μmol, 0.2 equiv) in dry CH₂Cl₂ (0.8 mL) was added in one portion solid 4-nitrobenzoyl chloride (49.4 mg, 266.0 μmol, 5.0 equiv). The yellow reaction mixture was allowed to stir for 14 h at rt before being loaded directly onto silica. Purification by flash column chromatography (SiO₂; 15% EtOAc in hexanes) afforded (S)-17 (35.0 mg, 45.1 μmol, 85% yield) as a white solid. Slow evaporation from a solution of cyclohexane, EtOAc, Et₂O afforded needle crystals suitable for X-ray crystallography.

¹H NMR (500 MHz, CDCl₃) δ 8.41-8.35 (m, 8H), 8.27-8.23 (m, 2H), 8.15-8.12 (m, 2H), 7.28-7.17 (m, 3H), 7.13 (d, J=2.1 Hz, 2H), 7.10-7.06 (m, 3H), 4.24 (t, J=6.7 Hz, 2H), 2.80 (dd, J=12.1, 2.8 Hz, 1H), 1.78-1.69 (m, 1H), 1.66-1.58 (m, 2H), 1.54-1.46 (m, 1H), 1.40-1.31 (m, 4H), 1.30-1.23 (m, 1H), 1.20 (s, 3H), 1.13-0.98 (m, 2H). ¹³C NMR (126 MHz, CDCl₃) δ 164.8, 163.2, 153.1, 151.2, 150.7, 140.9, 136.0, 134.8, 131.5, 130.8, 130.0, 127.9, 126.6, 124.0, 123.6, 118.1, 112.8, 66.1, 57.3, 42.1, 29.4, 28.7, 28.5, 28.0, 26.0, 23.8. IR (neat, vₘₐₓ/cm⁻¹): 3112, 2937, 2862, 1744, 1722, 1608, 1526, 1467, 1432, 1410, 1348, 1319, 1256, 1171, 1127, 1104, 1073, 1014, 959, 906, 872, 855, 839, 779, 714. HRMS (ESI): m/z=798.2270 [M+Na]⁺ (calc. for C42H₃₇N₃Na₀₁₂ m/z=798.2269).

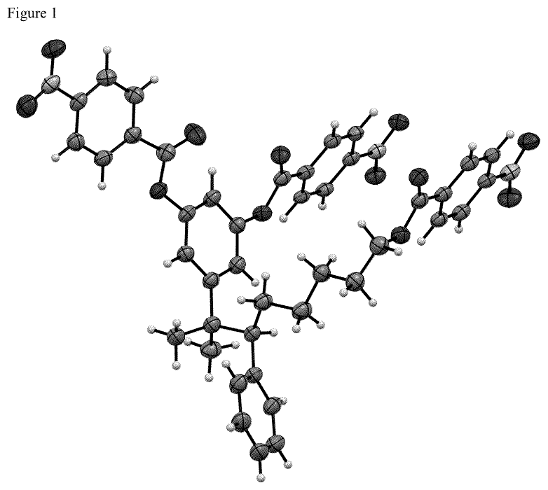

CRYSTALLOGRAPHIC DATA (See FIG. 1 for the crystal structure)

| Bond precision: | C—C = | 0.0064 A | Wavelength = 1.54184 | | | |
|---|---|---|---|---|---|---|
| Cell: | a = | 12.6822 (2) | b = | 6.9531 (1) | c = | 42.7059 (7) |
| | alpha = | 90 | beta = | 95.895 (1) | gamma = | 90 |

| Temperature: | 100 K. | |
|---|---|---|
| | Calculated | Reported |
| Volume | 3745.92 (10) | 3745.92 (10) |
| Space group | P 21 | P 1 21 1 |
| Hall group | P 2yb | P 2yb |
| Moiety formula | C42 H37 N3 O12 | C42 H37 N3 O12 |
| Sum formula | C42 H37 N3 O12 | C42 H37 N3 O12 |
| Mr | 775.75 | 775.74 |
| Dx, g cm−3 | 1.375 | 1.376 |
| Z | 4 | 4 |
| Mu (mm−1) | 0.851 | 0.851 |
| F000 | 1624.0 | 1624.0 |
| F000' | 1629.54 | |
| h, k, lmax | 15, 8, 53 | 15, 8, 53 |
| Nref | 15418 [8371] | 14274 |
| Tmin, Tmax | 0.974, 0.989 | 0.779, 1.000 |
| Tmin' | 0.787 | |

Correction method = # Reported T Limits: Tmin = 0.779 Tmax = 1.000

| AbsCorr = | GAUSSIAN | | |
|---|---|---|---|
| Data completeness = | 1.71/0.93 | Theta(max) = | 74.953 |
| R(reflections) = | 0.0543 (10459) | wR2(reflections) = | 0.1483 (14274) |
| S = | 1.028 | Npar = | 1031 |

CCDC Deposition number: 2310248

### Example 33

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropyl- | 20 | mg |
| methylcellulose | 425 | mg |

### Example 34

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula (I) 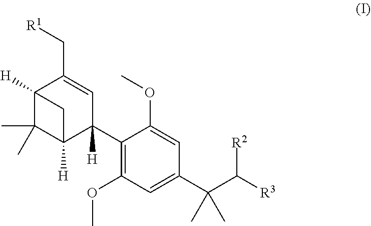
or a pharmaceutically acceptable salt thereof, wherein:
R¹ is selected from hydroxy, amino, and a group 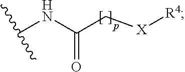
X is selected from a covalent bond, —O—, —NR^{x}—, —NR^{x}C(O)—(CH₂)_{q}—, —C(O)NR^{x}—, —C(O)—(CH₂)_{q}—, —C(O)—(CH₂)_{q}—NR^{x}C(O)—, —C(O)—CH₂—(CH₂)_{q}—C(O)O—, —C(O)—CH₂—O—, and —C(O)—CH₂—S—;
R^{x} is selected from hydrogen and methyl;
p is an integer selected from 1, 2, 3, 4, 5, and 6;
q is an integer selected from 0, 1, 2, 3, 4, and 5;
R² and R³ are each independently selected from C₁-C₆-alkyl, halo-C₁-C₆-alkyl, C₁-C₆-alkoxy, halo-C₁-C₆-alkoxy, C₆-C₁₀-aryl, and 5- to 14-membered heteroaryl; wherein said C₆-C₁₀-aryl and 5- to 14-membered heteroaryl are optionally substituted with 1-3 substituents independently selected from halogen, cyano, hydroxy, amino, C₁-C₆-alkyl, halo-C₁-C₆-alkyl, C₁-C₆-alkoxy, and halo-C₁-C₆-alkoxy;
R⁴ is selected from: 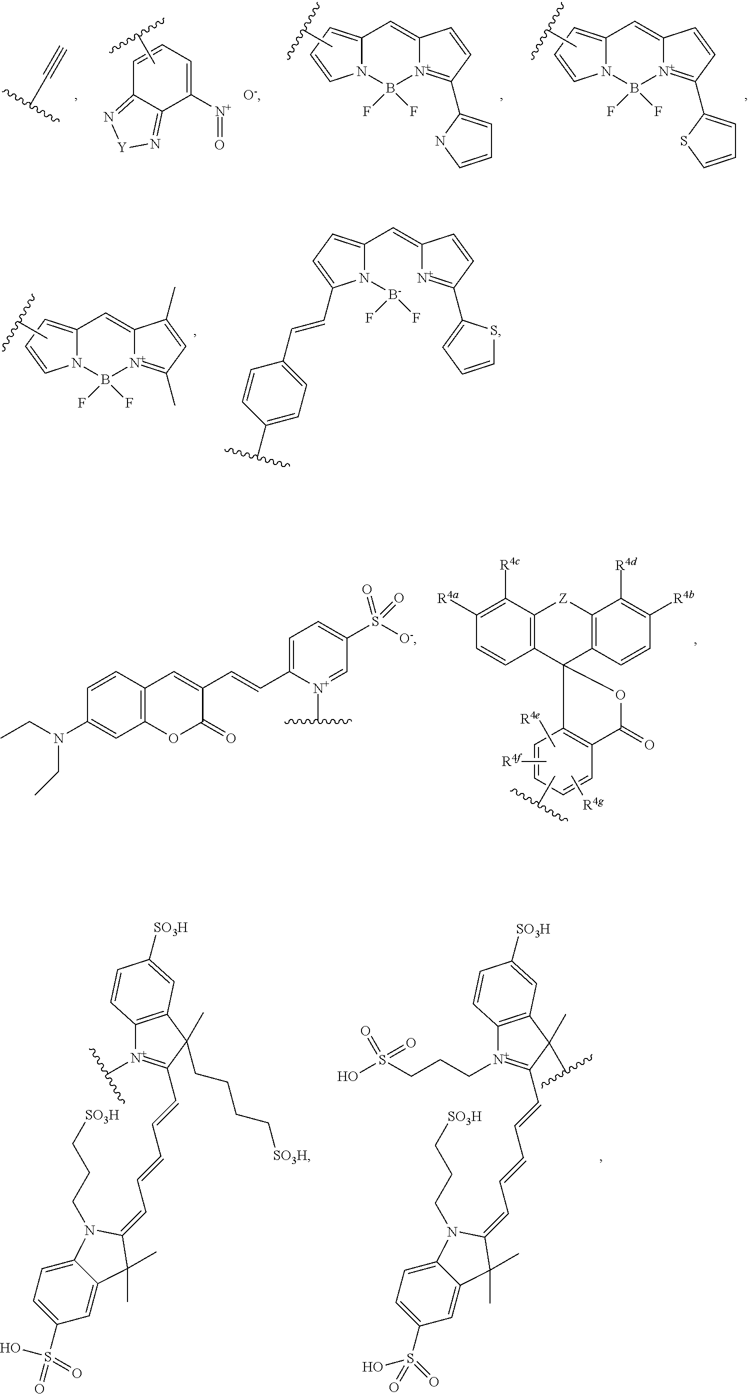 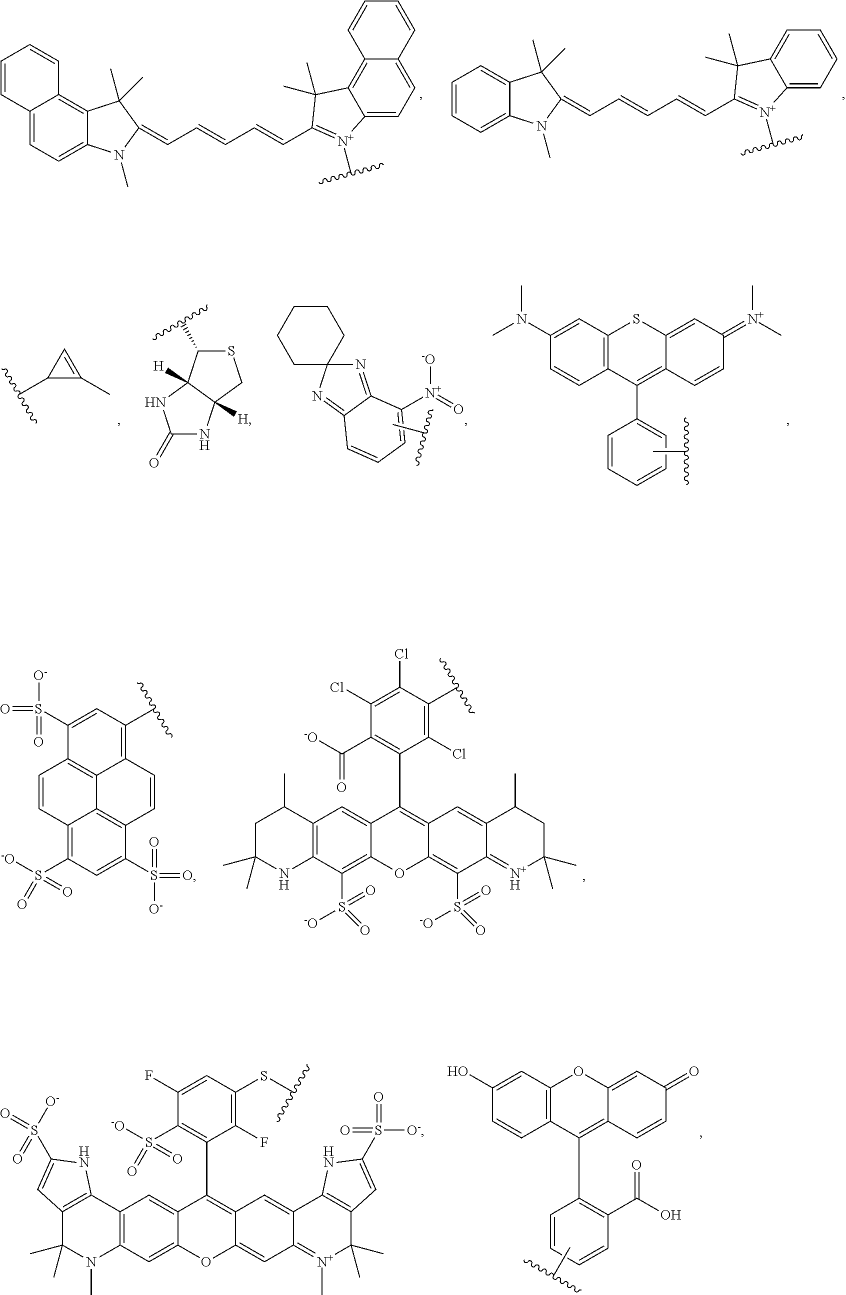 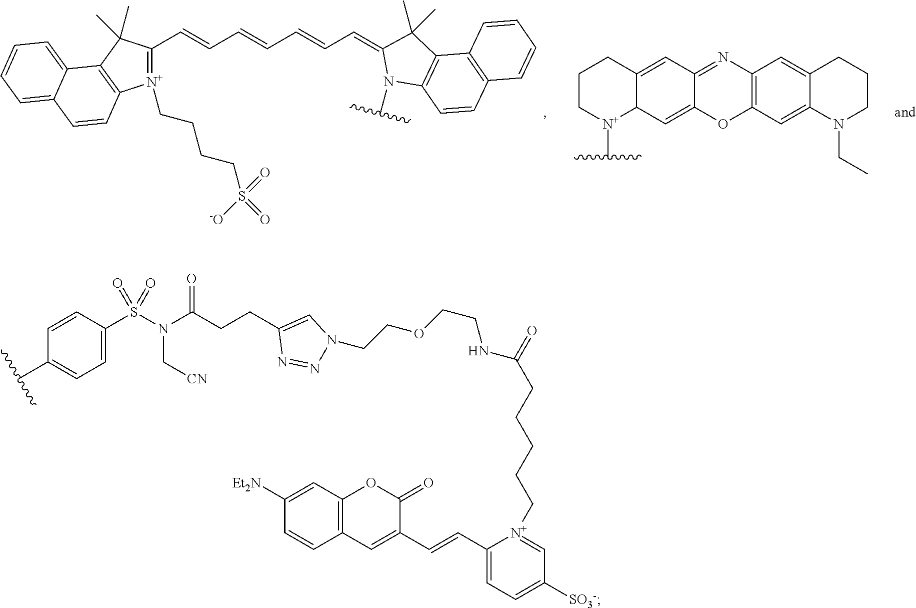
R^{4a} and R^{4b} are each independently selected from hydroxy, amino, dimethylamino, and azetidinyl;
R^{4c} and R^{4d} are each independently selected from hydrogen and —SO₃H;
R^{4e}, R^{4f}, and R^{4g} are each independently selected from hydrogen and halogen;
Y is selected from Se, S, O, C(CH₃)₂, C(CD₃)₂, NCH₂C≡CH and
Z is selected from O, S and Si(CH₃).

2. The compound of formula (I) according to claim 1, or a pharmaceutically acceptable salt thereof, wherein R¹ is selected from hydroxy and amino.

3. The compound of formula (I) according to claim 1, or a pharmaceutically acceptable salt thereof, wherein:
R¹ is a group 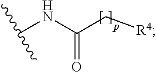
R⁴ is selected from: 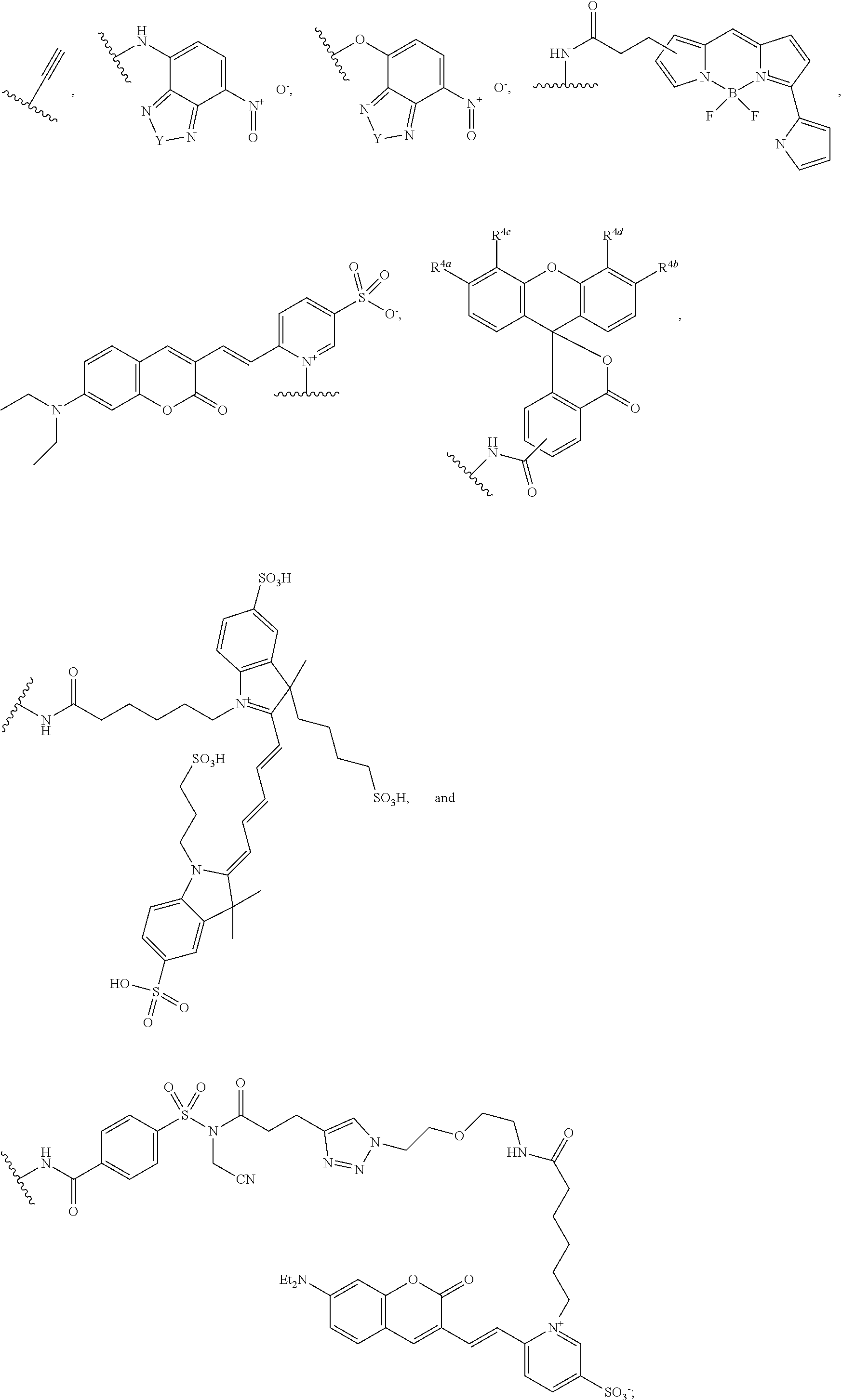
p is an integer selected from 2, 3, 4, 5, and 6;
R^{4a} and R^{4b} are each independently selected from hydroxy, amino and dimethylamino;
R^{4c} and R^{4d} are each independently selected from hydrogen and —SO₃H; and
Y is O.

4. The compound of formula (I) according to claim 3, or a pharmaceutically acceptable salt thereof, wherein:
R¹ is a group 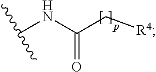
R⁴ is selected from: 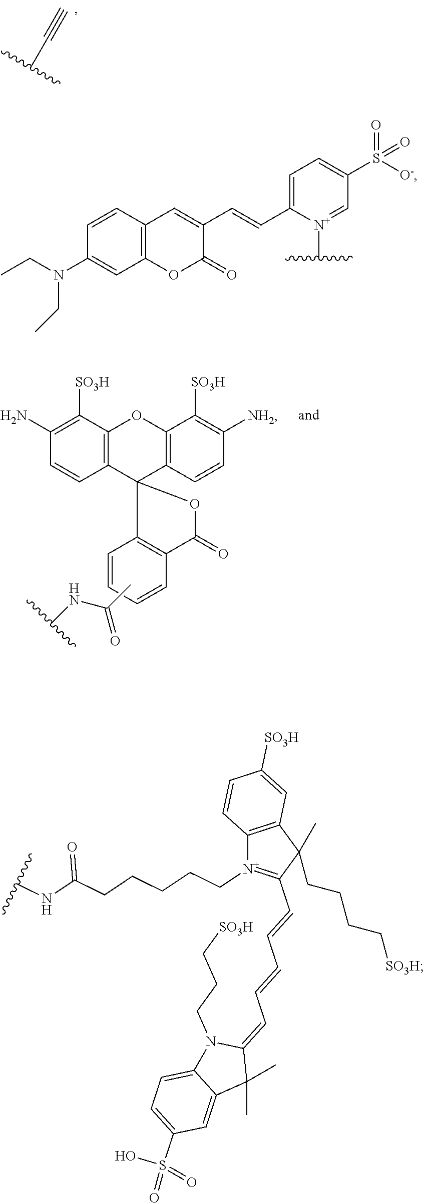
and
p is an integer selected from 2 and 5.

5. The compound of formula (I) according to claim 4, or a pharmaceutically acceptable salt thereof, wherein R¹ is selected from:

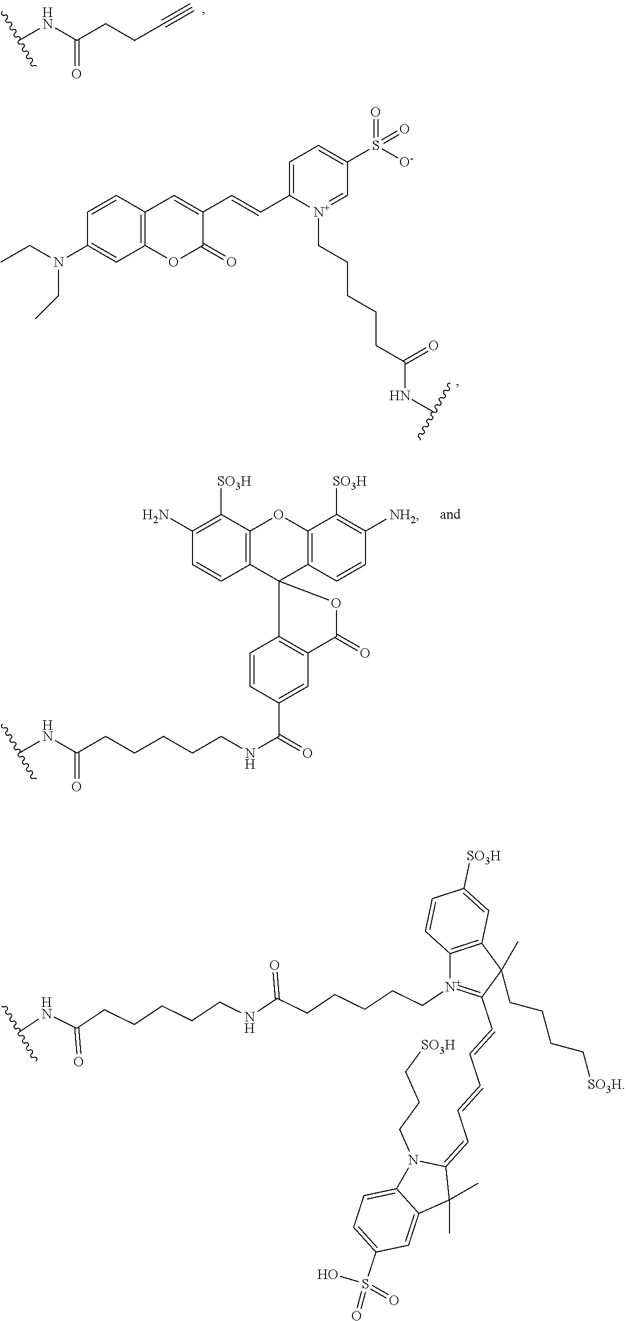

6. The compound of formula (I) according to claim 1, or a pharmaceutically acceptable salt thereof, wherein R² is selected from C₁-C₆-alkyl, halo-C₁-C₆-alkyl, C₆-C₁₀-aryl, and 5- to 14-membered heteroaryl; wherein said C₆-C₁₀-aryl and 5- to 14-membered heteroaryl are optionally substituted with 1 substituent selected from halogen and C₁-C₆-alkyl.

7. The compound of formula (I) according to claim 6, or a pharmaceutically acceptable salt thereof, wherein R² is selected from C₁-C₆-alkyl, halo-C₁-C₆-alkyl, phenyl, and 5- to 6-membered heteroaryl comprising 1-3 heteroatoms independently selected from N, O and S, the remaining atoms being carbon; wherein said phenyl and 5- to 6-membered heteroaryl are optionally substituted with 1 substituent selected from halogen and C₁-C₆-alkyl.

8. The compound of formula (I) according to claim 7, or a pharmaceutically acceptable salt thereof, wherein R² is selected from ethyl, 1-propyl, 2-propyl, CF₃, phenyl, 4-fluorophenyl, 3-fluorophenyl, and 1-methylpyrazol-3-yl.

9. The compound of formula (I) according to claim 8, or a pharmaceutically acceptable salt thereof, wherein R² is C₆-C₁₀-aryl.

10. The compound of formula (I) according to claim 9, or a pharmaceutically acceptable salt thereof, wherein R² is phenyl.

11. The compound of formula (I) according to claim 1, or a pharmaceutically acceptable salt thereof, wherein R³ is selected from C₁-C₆-alkyl and azido-C₁-C₆-alkyl.

12. The compound of formula (I) according to claim 11, or a pharmaceutically acceptable salt thereof, wherein R³ is selected from pentyl and 5-azidopentyl.

13. The compound of formula (I) according to claim 1, or a pharmaceutically acceptable salt thereof, wherein:
R¹ is selected from hydroxy, amino, and a group 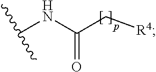
R² is selected from C₁-C₆-alkyl, halo-C₁-C₆-alkyl, phenyl, and 5- to 6-membered heteroaryl comprising 1-3 heteroatoms independently selected from N, O and S, the remaining atoms being carbon; wherein said phenyl and 5- to 6-membered heteroaryl are optionally substituted with 1 substituent selected from halogen and C₁-C₆-alkyl;
R³ is selected from C₁-C₆-alkyl and azido-C₁-C₆-alkyl;
R⁴ is selected from: 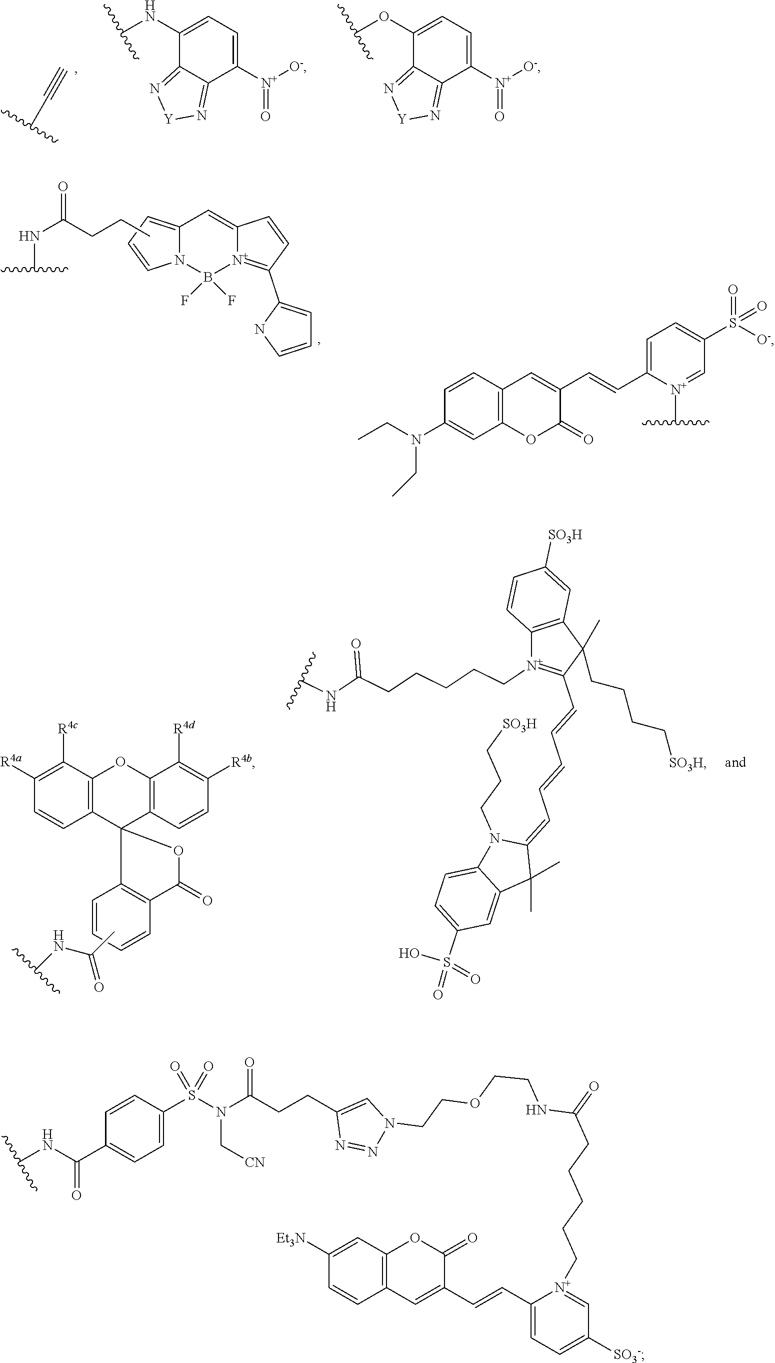
p is an integer selected from 2, 3, 4, 5, and 6;
R^{4a} and R^{4b} are each independently selected from hydroxy, amino and dimethylamino;
R^{4c} and R^{4d} are each independently selected from hydrogen and —SO₃H; and
Y is O.

14. The compound of formula (I) according to claim 13, or a pharmaceutically acceptable salt thereof, wherein:
R¹ is selected from hydroxy, amino, and a group 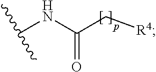
R² is selected from ethyl, 1-propyl, 2-propyl, CF₃, phenyl, 4-fluorophenyl, 3-fluorophenyl, and 1-methylpyrazol-3-yl;
R³ is selected from pentyl and 5-azidopentyl;
R⁴ is selected from: 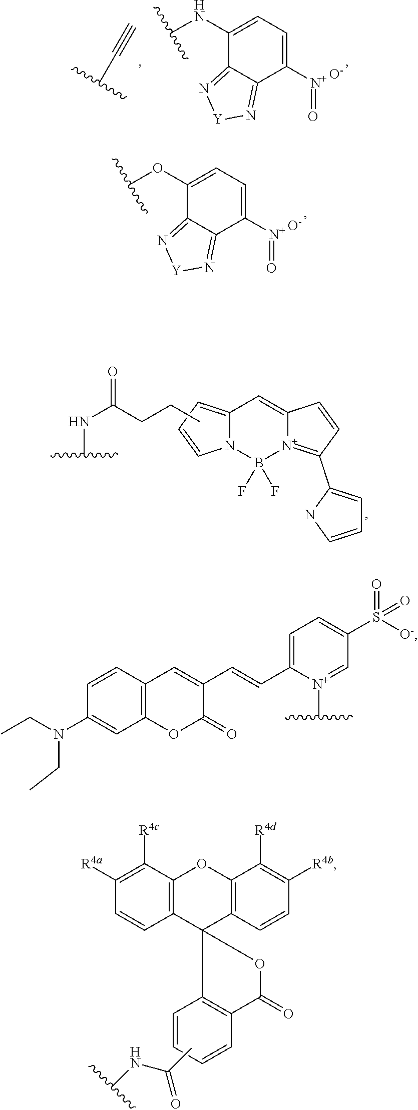 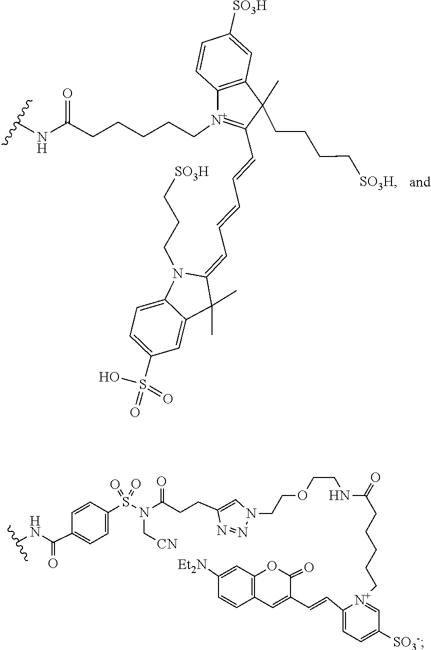
p is an integer selected from 2, 3, 4, 5, and 6;
R^{4a} and R^{4b} are each independently selected from hydroxy, amino and dimethylamino;
R^{4c} and R^{4d} are each independently selected from hydrogen and —SO₃H; and
Y is O.

15. The compound of formula (I) according to claim 13, or a pharmaceutically acceptable salt thereof, wherein:
R¹ is a group 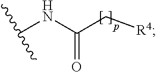
R² is C₆-C₁₀-aryl;
R³ is selected from C₁-C₆-alkyl and azido-C₁-C₆-alkyl;
R⁴ is selected from: 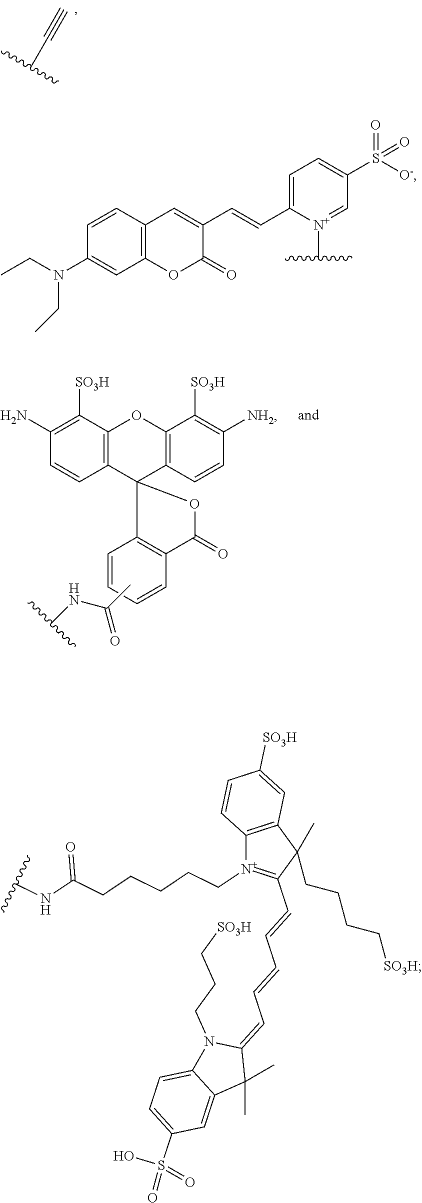
and
p is an integer selected from 2 and 5.

16. The compound of formula (I) according to claim 15, or a pharmaceutically acceptable salt thereof, wherein:
R¹ is selected from: 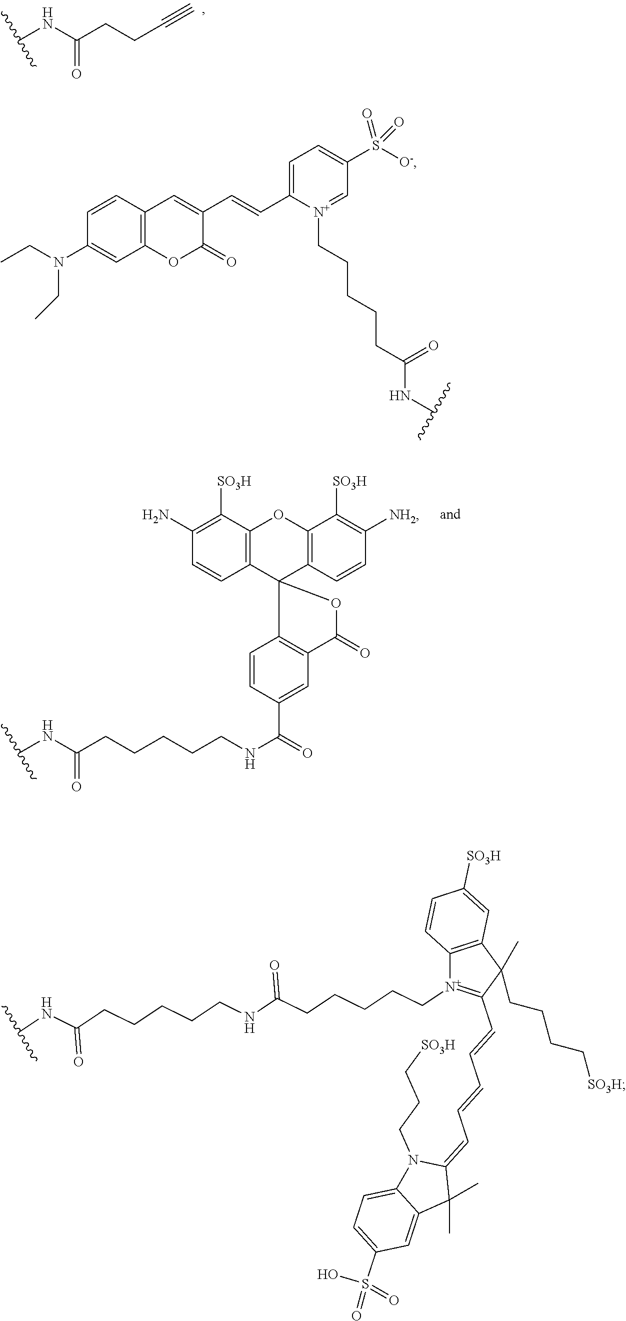
R² is phenyl; and
R³ is selected from pentyl and 5-azidopentyl.

17. The compound of formula (I) according to claim 1, or a pharmaceutically acceptable salt thereof, wherein the compound of formula (I) is selected from:
1-(6-((((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate;
6-((E)-2-(7-(diethylamino)-2-oxo-2H-chromen-3-yl)vinyl)-1-(6-((((1S,4S,5S)-4-(2,6-dimethoxy-4-(2-methyl-3-phenyloctan-2-yl)phenyl)-6,6-dimethylbicyclo[3.1.1]hept-2-en-2-yl)methyl)amino)-6-oxohexyl)pyridin-1-ium-3-sulfonate;
N-(((1S,4S,5S)-4-(4-(8-azido-2-methyl-3-phenyloctan-2-yl)-2,6-dimethoxyphenyl)-6,6-dimethylbicyclo[3.1.1]hept-2-en-2-yl)methyl)pent-4-ynamide;
((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methanamine;
((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methanamine;
N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)amino)heptanamide;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)amino)heptanamide;
N-(((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)pent-4-ynamide;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)pent-4-ynamide;
1-(6-((((1S,4S,5S)-4-(4-((R)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate;
1-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-6-((E)-2-(7-(diethylamino)-2-oxo-2H-chromen-3-yl)vinyl)pyridin-1-ium-3-sulfonate;
N-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-bis(dimethylamino)-3-oxo-3H-spiro[isobenzofuran-1,9′-xanthene]-5-carboxamide;
N-(6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)-3′,6′-dihydroxy-3-oxo-3H-spiro[isobenzofuran-1,9′-xanthene]-6-carboxamide;
1-(6-((6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)amino)-6-oxohexyl)-2-((1E,3E)-5-((E)-3,3-dimethyl-5-sulfo-1-(3-sulfopropyl)indolin-2-ylidene)penta-1,3-dien-1-yl)-3-methyl-3-(4-sulfobutyl)-3H-indol-1-ium-5-sulfonate;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-6-(3-(5,5-difluoro-7-(1H-pyrrol-2-yl)-5H-514,614-dipyrrolo[1,2-c:2′,1′-f][1,3,2]diazaborinin-3-yl)propanamido)hexanamide;
3′,6′-diamino-5-((6-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-6-oxohexyl)carbamoyl)-3-oxo-3H-spiro[isobenzofuran-1,9′-xanthene]-4′,5′-disulfonic acid;
((1S,4S,5S)-4-(4-((R)-3-(4-fluorophenyl)-2-methyloctan-2-yl)-2,6-dimethoxyphenyl)-6,6-dimethylbicyclo[3.1.1]hept-2-en-2-yl)methanol;
((1S,4S,5S)-4-(4-((S)-3-(4-fluorophenyl)-2-methyloctan-2-yl)-2,6-dimethoxyphenyl)-6,6-dimethylbicyclo[3.1.1]hept-2-en-2-yl)methanol;
((1S,4S,5S)-4-(4-((R)-3-(3-fluorophenyl)-2-methyloctan-2-yl)-2,6-dimethoxyphenyl)-6,6-dimethylbicyclo[3.1.1]hept-2-en-2-yl)methanol;
((1S,4S,5S)-4-(4-((S)-3-(3-fluorophenyl)-2-methyloctan-2-yl)-2,6-dimethoxyphenyl)-6,6-dimethylbicyclo[3.1.1]hept-2-en-2-yl)methanol;
((1S,4S,5S)-4-(4-((R)-3-ethyl-2-methyloctan-2-yl)-2,6-dimethoxyphenyl)-6,6-dimethylbicyclo[3.1.1]hept-2-en-2-yl)methanol;
((1S,4S,5S)-4-(4-(3-ethyl-2-methyloctan-2-yl)-2,6-dimethoxyphenyl)-6,6-dimethylbicyclo[3.1.1]hept-2-en-2-yl)methanol;
((1S,4S,5S)-4-(2,6-dimethoxy-4-(2-methyl-3-propyloctan-2-yl)phenyl)-6,6-dimethylbicyclo[3.1.1]hept-2-en-2-yl)methanol;
((1S,4S,5S)-4-(4-(3-isopropyl-2-methyloctan-2-yl)-2,6-dimethoxyphenyl)-6,6-dimethylbicyclo[3.1.1]hept-2-en-2-yl)methanol;
((1S,4S,5S)-4-(2,6-dimethoxy-4-((R)-2-methyl-3-(trifluoromethyl)octan-2-yl)phenyl)-6,6-dimethylbicyclo[3.1.1]hept-2-en-2-yl)methanol;
((1S,4S,5S)-4-(2,6-dimethoxy-4-((S)-2-methyl-3-(trifluoromethyl)octan-2-yl)phenyl)-6,6-dimethylbicyclo[3.1.1]hept-2-en-2-yl)methanol;
((1S,4S,5S)-4-(2,6-dimethoxy-4-((R)-2-methyl-3-phenyloctan-2-yl)phenyl)-6,6-dimethylbicyclo[3.1.1]hept-2-en-2-yl)methanol;
((1S,4S,5S)-4-(2,6-dimethoxy-4-((S)-2-methyl-3-phenyloctan-2-yl)phenyl)-6,6-dimethylbicyclo[3.1.1]hept-2-en-2-yl)methanol;
((1S,4S,5S)-4-(2,6-dimethoxy-4-(2-methyl-3-(1-methyl-1H-pyrazol-3-yl)octan-2-yl)phenyl)-6,6-dimethylbicyclo[3.1.1]hept-2-en-2-yl)methanol;
N-(((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)-7-((7-nitrobenzo[c][1,2,5]oxadiazol-4-yl)oxy)heptanamide; and
1-(6-((2-(2-(4-(3-((4-((7-((((1S,4S,5S)-4-(4-((S)-8-azido-2-methyl-3-phenyloctan-2-yl)-2,6-dimethoxyphenyl)-6,6-dimethylbicyclo[3.1.1]hept-2-en-2-yl)methyl)amino)-7-oxoheptyl)carbamoyl)-N-(cyanomethyl)phenyl)sulfonamido)-3-oxopropyl)-1H-1,2,3-triazol-1-yl)ethoxy)ethyl)amino)-6-oxohexyl)-6-((E)-2-(7-(diethylamino)-2-oxo-2H-chromen-3-yl)vinyl)pyridin-1-ium-3-sulfonate.

18. (canceled)

19. A pharmaceutical composition comprising a compound of formula (I) according to claim 2, or a pharmaceutically acceptable salt thereof, and a therapeutically inert carrier.

20. A method of treating or preventing a disease or disorder that is associated with CB₂R in a subject in need thereof, said method comprising administering a therapeutically effective amount of a compound of formula (I) according to claim 2, or a pharmaceutically acceptable salt thereof, to said subject in need.

21-23. (canceled)

24. The method according to claim 20, wherein said disease or disorder that is associated with CB₂R is selected from hypertension, inflammation, peripheral pain, neuropathic pain, gastrointenstinal disorders, autoimmune diseases, pain, atherosclerosis, age-related macular degeneration, diabetic retinopathy, glaucoma, diabetes mellitus, inflammatory bowel disease, ischemia-reperfusion injury, acute liver failure, liver fibrosis, lung fibrosis, kidney fibrosis, systemic fibrosis, acute allograft rejection, chronic allograft nephropathy, diabetic nephropathy, glomerulonephropathy, cardiomyopathy, heart failure, myocardial ischemia, myocardial infarction, systemic sclerosis, thermal injury, burning, hypertrophic scars, keloids, gingivitis pyrexia, liver cirrhosis, tumors, regulation of bone mass, neurodegeneration, stroke, transient ischemic attack, uveitis, renal fibrosis, arthritis, neuroinflammation, asthma, osteoporosis, psychiatric diseases, psychosis, cancer, encephalitis, malaria, immunological disorders, rheumatoid arthritis, and allergies.

25-26. (canceled)

27. A method of imaging cannabinoid receptor 2 (CB₂R), comprising contacting said cannabinoid receptor 2 (CB₂R) with a compound of formula (I) according to claim 1, or a pharmaceutically acceptable salt thereof.

28. (canceled)
